(19) United States
(12) Patent Application Publication
(10) Pub. No.: US 2024/0199614 A1
(43) Pub. Date: Jun. 20, 2024

(54) MACROCYCLIC EGFR INHIBITORS FOR THE TREATMENT OF CANCER

(71) Applicant: Theseus Pharmaceuticals, Inc., Cambridge, MA (US)

(72) Inventors: Wei-Sheng HUANG, Cambridge, MA (US); William C. SHAKESPEARE, Cambridge, MA (US); Charles J. EYERMANN, Cambridge, MA (US); David C. DALGARNO, Cambridge, MA (US)

(73) Assignee: Theseus Pharmaceuticals, Inc., Cambridge, MA (US)

(21) Appl. No.: 18/552,302

(22) PCT Filed: Mar. 25, 2022
(86) PCT No.: PCT/US22/21999

§ 371 Date: Sep. 25, 2023

Related U.S. Application Data
(60) Provisional application No. 63/166,464, filed on Mar. 26, 2021.

(51) Int. Cl. C07D 471/22; A61K 31/437; A61K 31/444; A61K 31/4545; A61K 31/496; A61K 31/499; A61K 31/5377; A61P 35/00; C07D 498/22

(52) U.S. Cl. CPC C07D 471/22; A61K 31/437; A61K 31/444; A61K 31/4545; A61K 31/496; A61K 31/499; A61K 31/5377; A61P 35/00; C07D 498/22

(57) ABSTRACT

Described herein are macrocyclic compounds of Formula (I), which can inhibit kinases such as EGFR, including mutant forms such as T790M EGFR mutants. Also described herein are pharmaceutical compositions comprising a compound of Formula (I), or any pharmaceutically acceptable form thereof, processes for their preparation, and use in therapy for the prevention or treatment of cancer. In particular, compounds described herein can be effective for treating EGFR-driven cancers including non-small cell lung cancer (NSCLC).

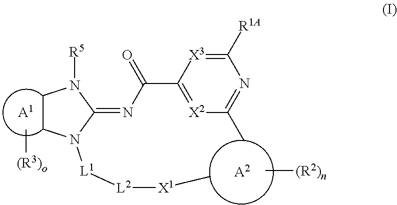

### CROSS-REFERENCE TO RELATED APPLICATIONS

The present application claims benefit of U.S. Provisional Application No. 63/166,464, filed Mar. 26, 2021, which is hereby incorporated by reference in its entirety.

### FIELD OF THE INVENTION

Described herein are macrocyclic compounds that can be used as kinase inhibitors. In particular, compounds described herein can inhibit epidermal growth factor receptor (EGFR), including mutant forms of EGFR. Compounds described herein can be effective for treating various disorders that include cancers such as EGFR-driven cancers (e.g., non-small cell lung cancer (NSCLC) characterized by mutant EGFR).

### BACKGROUND

Signal transduction refers to the transmission of stimulatory or inhibitory signals into and within a cell leading, often via a cascade of signal transmission events, to a biological response within the cell. Defects in various components of signal transduction pathways have been found to account for a large number of diseases, including numerous forms of cancer, inflammatory disorders, metabolic disorders, vascular and neuronal diseases.

Signal transduction is often mediated by certain proteins called kinases. Kinases can generally be classified into protein kinases and lipid kinases, and certain kinases exhibit dual specificities. For example, epidermal growth factor receptor (EGFR) belongs to a family of receptor tyrosine kinases (RTKs) that include EGFR/ERBB1, HER2/ERBB2/NEU, HER3/ERBB3, and HER4/ERBB4. The binding of a ligand, such as epidermal growth factor (EGF), induces a conformational change in EGFR that facilitates receptor homo- or heterodimer formation, leading to activation of EGFR tyrosine kinase activity. Activated EGFR then phosphorylates its substrates, resulting in activation of multiple downstream pathways within the cell, including the PI3K-AKT-mTOR pathway, which is involved in cell survival, and the RAS-RAF-MEK-ERK pathway, which is involved in cell proliferation. (Chong et al. Nature Med. 2013; 19(11):1389-1400).

Certain cancers are characterized by mutations of EGFR, which results in increased cell proliferation. Tyrosine kinase inhibitor (TKI) therapies that inhibit EGFR can lead to clinical responses; however, mutations in EGFR can also confer resistance to such therapies.

New therapeutic methods therefore remain necessary for treating cancers associated with defective signal transduction pathways, including EGFR-driven cancers.

### SUMMARY OF THE INVENTION

Described herein are new compounds that can be effective inhibitors of EGFR. Such compounds can be useful for treating various diseases and disorders, including EGFR-driven cancers such as non-small cell lung cancer (NSCLC) characterized by mutant EGFR.

A first aspect of the invention relates to compounds of Formula (I):

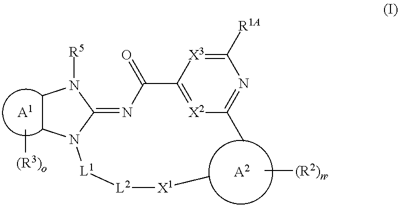

or a pharmaceutically acceptable salt thereof, wherein
A¹ is independently phenylene or 5- or 6-membered heteroarylene;
A² is independently phenyl, naphthyl, or a 5- to 13-membered heteroaryl;
X¹ is independently O or X^{1A};
X^{1A} is a covalent bond, S, NR⁴, C₁₋₆ alkylene, C₂₋₆ alkenylene, or C₂₋₆ alkynylene;
each of X² and X³ is independently N or CR^{1B};
L¹ is independently a covalent bond or C₁₋₆ alkylene;
L² is independently a covalent bond, C₂₋₆ alkenylene, C₂₋₆ alkynylene, C₃₋₆ cycloalkylene, 3- to 10-membered heterocyclylene, phenylene, or 5- or 6-membered heteroarylene;
each R^{1A} and R^{1B} is independently H, OH, CN, halogen, C₁₋₆ aliphatic, C₁₋₆ alkoxy, NR⁶R⁷, C(O)R⁸, CO₂R⁸, C(O)NR⁶R⁷, NR⁹C(O)R⁸, NR⁹CO₂R⁸, NR⁹C(O)NR⁶R⁷, or R¹⁰; each R² and R³, when present, is independently OH, CN, halogen, C₁₋₆ aliphatic, C₁₋₆ alkoxy, NR⁶R⁷, C(O)R⁸, CO₂R′, C(O)NR⁶R⁷, NR⁹C(O)R⁸, NR⁹CO₂R⁸, NR⁹C(O)NR⁶R⁷, R¹⁰, OR¹⁰, CH₂R¹⁰, CH₂CH₂R¹⁰, OCH₂R¹⁰, or OCH₂CH₂R¹⁰;
each R⁴ is independently H, a N-protecting group, or C₁₋₆ alkyl;
R⁵ is hydrogen;
each R⁶, R⁷, and R⁹ is independently H or C₁₋₆ alkyl; or R⁶ and R⁷, together with the nitrogen atom to which they are attached, form a 3- to 10-membered heterocyclyl; or R⁶ and R⁹, together with the atoms to which they are attached, form a 3- to 10-membered heterocyclyl;
R⁸ is independently C₁₋₆ aliphatic, C₃-C₁₀ cycloaliphatic, 3- to 10-membered heterocyclyl, phenyl, naphthyl, or a 5- to 12-membered heteroaryl, or R⁸ and R⁹, together with the atoms to which they are attached, form a 3- to 10-membered heterocyclyl;
R¹⁰ is independently C₃-C₁₀ cycloaliphatic, 3- to 10-membered heterocyclyl, phenyl, naphthyl, or a 5- to 12-membered heteroaryl;
each of n and o is independently 0, 1, or 2; and
wherein X¹ is O, and both of X² and X³ are not N, then A² is naphthyl or a bicyclic 8- to 12-membered heteroaryl.

In embodiments, at least one of X² and X³ is N.

In embodiments, a compound has a structure according to Formula (II),

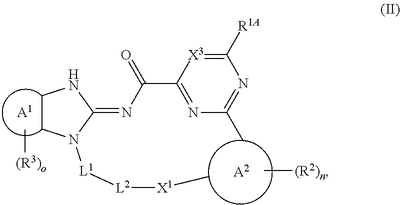

or a pharmaceutically acceptable salt thereof.

In embodiments, a compound has a structure according to Formula (III),

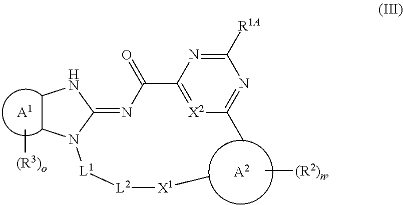

or a pharmaceutically acceptable salt thereof.

In embodiments, a compound has a structure according to Formula (IV),

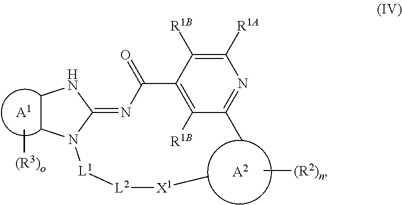

or a pharmaceutically acceptable salt thereof.

In embodiments, each R^{1B} is H.

In embodiments, X¹ is X^{1A}.

In embodiments, a compound has a structure according to Formula (V),

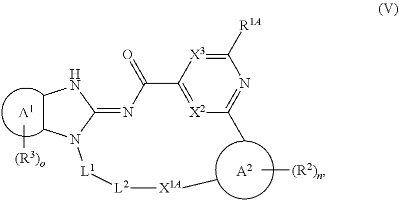

or a pharmaceutically acceptable salt thereof.

In embodiments, a compound has a structure according to Formula (VI),

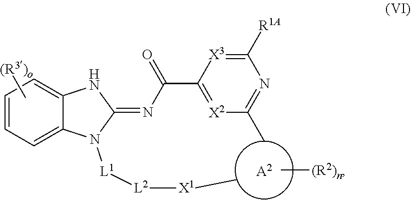

or a pharmaceutically acceptable salt thereof.

In embodiments, a compound has a structure according to Formula (VI-1),

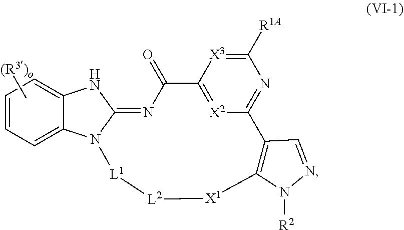

or a pharmaceutically acceptable salt thereof.

In embodiments, a compound has a structure according to Formula (VI-2),

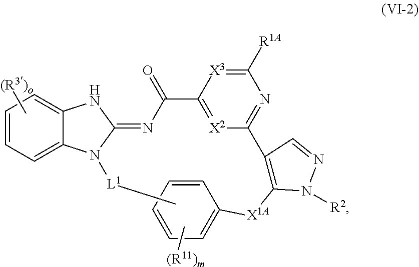

or a pharmaceutically acceptable salt thereof, wherein
each R¹¹ is independently OH, CN, halogen, C₁₋₆ alkyl, or C₁₋₆ alkoxy; and
m is 0, 1, or 2.

In embodiments, a compound has a structure according to Formula (VI-3),

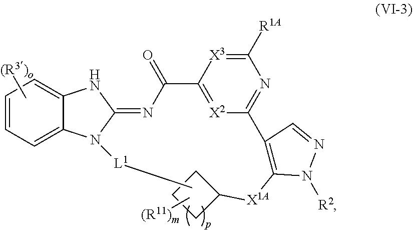

or a pharmaceutically acceptable salt thereof, wherein
each R¹¹ is independently OH, oxo, CN, halogen, C₁₋₆ alkyl, or C₁₋₆ alkoxy;
m is 0, 1, or 2; and
p is 0, 1, 2, or 3.

In embodiments, X^{1A} is a covalent bond or C₁₋₆ alkylene.

In embodiments, a compound has a structure according to Formula (VI-4),

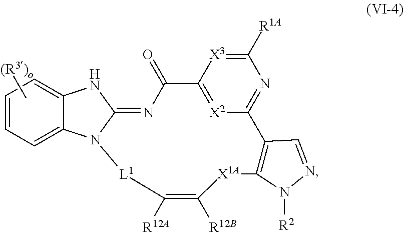

or a pharmaceutically acceptable salt thereof, wherein
X^{1A} is independently a covalent bond or C₁₋₆ alkylene; and
each R^{12A} and R^{12B} is independently H or C₁₋₆ alkyl, or R^{12A} and R^{12B} combine to form a cyclopentene or cyclohexene.

In embodiments, a compound has a structure according to Formula (VI-5),

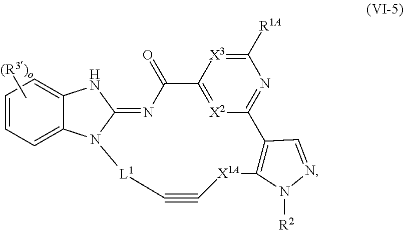

or a pharmaceutically acceptable salt thereof, wherein
X^{1A} is a covalent bond or C₁₋₆ alkylene; and
L¹ is C₁₋₆ alkylene.

In embodiments, R² is CH₃.

In embodiments, L is linear or branched C₁₋₆ alkylene, and wherein said alkylene is unsubstituted or comprises a —OH group.

In embodiments, a compound has a structure according to Formula (VII),

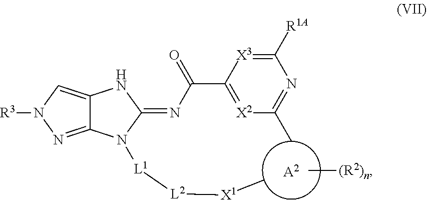

or a pharmaceutically acceptable salt thereof.

In embodiments, a compound has a structure according to Formula (VIII),

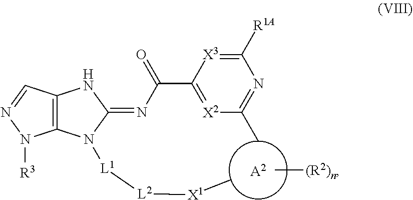

or a pharmaceutically acceptable salt thereof.

In embodiments, A² is a monocyclic 5-to-6-membered heteroaryl.

In embodiments, A² is pyridyl, pyrimidyl, pyrazolyl, thiazolyl, oxazolyl, or imidazolyl, and wherein A² is optionally substituted by a methyl, halogen, or CN.

In embodiments, A² is phenyl, naphthyl, or a bicyclic 8- to 12-membered heteroaryl.

In embodiments, A² is a nitrogen-containing, bicyclic 8- to 12-membered heteroaryl that is indolyl, benzimidazolyl, indazolyl, isoindolyl, pyrrolopyrimidyl, pyrrolopyridinyl, pyrazolopyrimidyl, pyrazolopyridinyl, benzotriazolyl, quinolyl, or isoquinolyl.

In embodiments, X¹ is X^{1A}.

In embodiments, X^{1A} is a covalent bond.

In embodiments, X^{1A} is S or NR⁴.

In embodiments, X^{1A} is C₁₋₆ alkylene, C₂₋₆ alkenylene, or C₂₋₆ alkynylene.

In embodiments, X¹ is O.

In embodiments, X² and/or X³ is N.

In embodiments, each of X² and X³ is CR^{1B}.

In embodiments, each of X² and X³ is CH.

In embodiments, L¹ is a covalent bond, unsubstituted branched C₁₋₆ alkylene, or linear C₁₋₆ alkylene optionally comprising a —OH substituent.

In embodiments, L¹ is a covalent bond.

In embodiments, L¹ is unsubstituted branched C₁₋₆ alkylene, or linear C₁₋₆ alkylene optionally comprising a —OH substituent.

In embodiments, L² is C₂₋₆ alkenylene or C₂₋₆ alkynylene.

In embodiments, L² is C₃₋₆ cycloalkylene or 3- to 10-membered heterocyclylene.

In embodiments, L² is phenylene, or 5- or 6-membered heteroarylene.

In embodiments, L² is a covalent bond.

In embodiments,
X¹ is X^{1A}, wherein X^{1A} is a covalent bond, C₁₋₆ alkylene, C₂₋₆ alkenylene, or C₂₋₆ alkynylene;
L¹ is independently a covalent bond or C₁₋₆ alkylene;
L² is independently a covalent bond, C₂₋₆ alkenylene, C₂₋₆ alkynylene; C₃₋₆ cycloalkylene, 3- to 10-membered heterocyclylene, phenylene, or 5- or 6-membered heteroarylene; and
where at least one of X^{A}, L¹, and L² is a covalent bond.

In embodiments, one of X^{1A} and L¹ is a covalent bond and the other is C₁₋₆ alkylene; and L² is a covalent bond.

In embodiments, each of L¹ and L² is a covalent bond.

In embodiments, each of X^{1A} and L² is a covalent bond.

In embodiments, each of X^{1A} and L¹ is a covalent bond.

In embodiments, a compound has a structure according to Formula (IX),

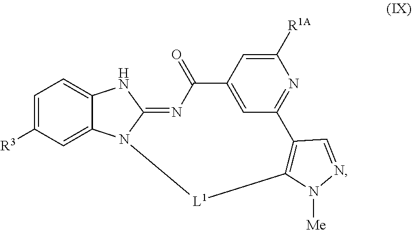

or a pharmaceutically acceptable salt thereof, wherein
L¹ is C₁-C₆ alkylene optionally substituted by 1, 2, or 3 R¹³;
each R¹³ is independently unsubstituted C₁-C₃ alkyl; and
R^{1A} is independently unsubstituted C₁-C₆ alkyl or C₁-C₆ haloalkyl.

In embodiments, a compound has a structure according to Formula (X),

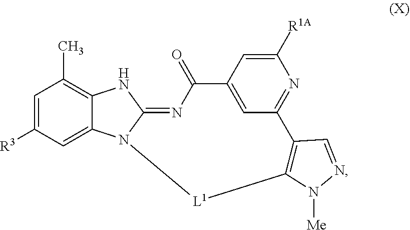

or a pharmaceutically acceptable salt thereof, wherein
L¹ is C₁-C₆ alkylene optionally substituted by 1, 2, or 3 R¹³;
each R¹³ is independently unsubstituted C₁-C₃ alkyl; and
R^{1A} is independently unsubstituted C₁-C₆ alkyl or C₁-C₆ haloalkyl.

In embodiments, a compound has a structure according to Formula (XI),

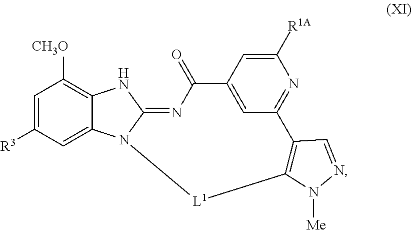

or a pharmaceutically acceptable salt thereof, wherein
L¹ is C₁-C₆ alkylene optionally substituted by 1, 2, or 3 R¹³;
each R¹³ is independently unsubstituted C₁-C₃ alkyl; and
R^{1A} is independently unsubstituted C₁-C₆ alkyl or C₁-C₆ haloalkyl.

In embodiments, L¹ is selected from the following group of substructures:

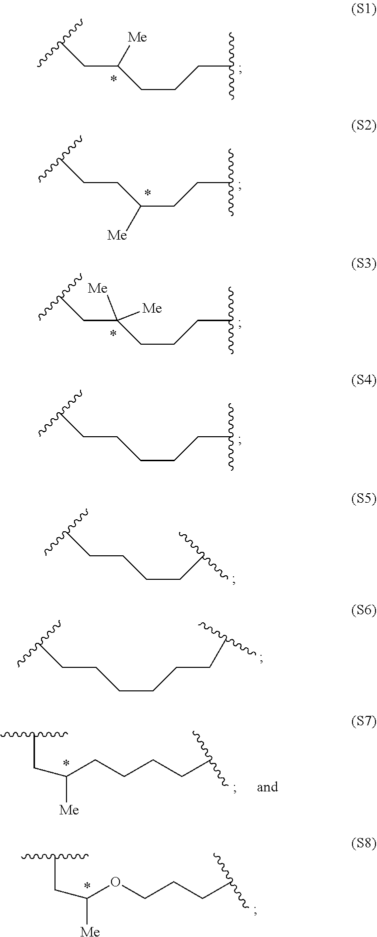

wherein a carbon marked by an asterisk (*) is racemic or has the (R)- or (S)-stereochemistry.

In embodiments, a compound has a structure according to:

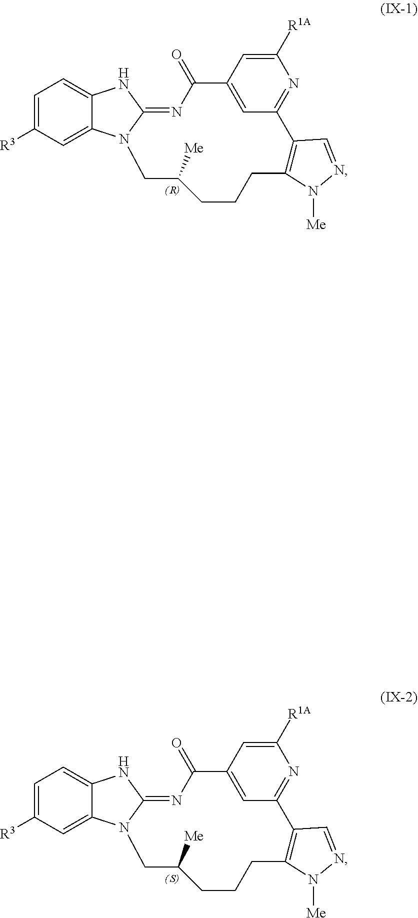

or a pharmaceutically acceptable salt thereof.

In embodiments, a compound has a structure according to:

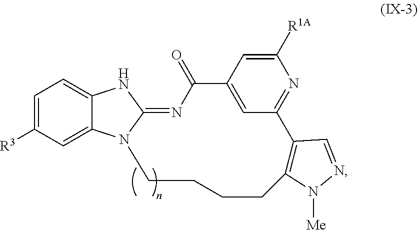

or a pharmaceutically acceptable salt thereof, wherein n is 1, 2, or 3.

In embodiments, a compound has a structure according to:

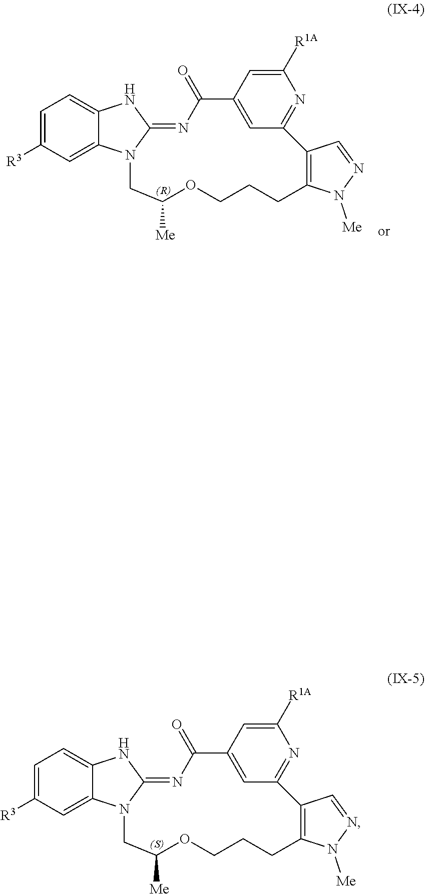

or a pharmaceutically acceptable salt thereof.

In embodiments, a compound has a structure according to Formula (XII),

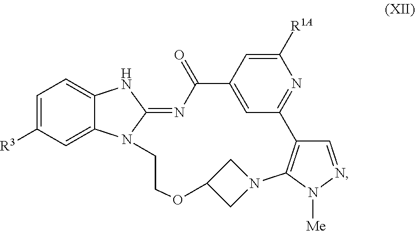

or a pharmaceutically acceptable salt thereof, wherein
R^{1A} is independently unsubstituted C₁-C₆ alkyl or C₁-C₆ haloalkyl.

In embodiments, a compound has a structure according to Formula (XIII),

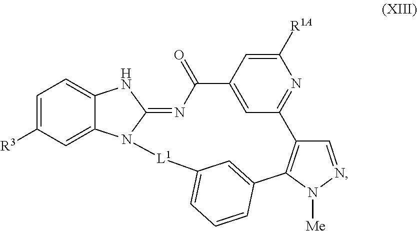

or a pharmaceutically acceptable salt thereof, wherein
L¹ is C₁-C₃ alkylene optionally substituted by 1 or 2 R¹³;
each R¹³ is independently unsubstituted C₁-C₃ alkyl; and
R^{1A} is independently unsubstituted C₁-C₆ alkyl or C₁-C₆ haloalkyl.

In embodiments, L¹ is —CH₂— or —CH₂CHCH₃—.

In embodiments, a compound has a structure according to Formula (XIV),

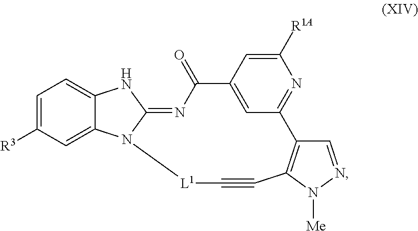

or a pharmaceutically acceptable salt thereof, wherein
L¹ is C₂-C₄ alkylene optionally substituted by 1 or 2 R¹³;
each R¹³ is independently unsubstituted C₁-C₃ alkyl; and
R^{1A} is independently unsubstituted C₁-C₆ alkyl or C₁-C₆ haloalkyl.

In embodiments, L¹ is —(CH₂)₃— or —(CH₂)₄—.

In embodiments, R^{1A} is CH₃, CH₂F, CHF₂, or CF₃.

In embodiments, R^{1A} is CH₃.

In embodiments, R³ is
halogen;
NR⁶R⁷, wherein R⁶ and R⁷, together with the nitrogen atom to which they are attached, form a 5- to 7-membered heterocyclyl;
NR⁶R⁷, wherein each R⁶ and R⁷ is independently C₁-C₆ alkyl;
phenyl;
pyridyl;
C(O)R⁸, wherein R⁸ is a 5- to 6-membered nitrogen-containing heterocyclyl;
R¹⁰, wherein R¹⁰ is a 5- to 6-membered nitrogen-containing heterocyclyl;
OR¹⁰, wherein R¹⁰ is a 5- to 6-membered nitrogen-containing heterocyclyl;
CH₂R¹⁰, wherein R¹⁰ is a 5- to 6-membered nitrogen-containing heterocyclyl;
CH₂CH₂R¹⁰, wherein R¹⁰ is a 5- to 6-membered nitrogen-containing heterocyclyl; or
OCH₂CH₂R¹⁰, wherein R¹⁰ is a 5- to 6-membered nitrogen-containing heterocyclyl.

In embodiments, R³ is halogen.

In embodiments, R³ is NR⁶R⁷, where R⁶ and R⁷, together with the nitrogen atom to which they are attached, form a 5- to 6-membered heterocyclyl.

In embodiments, R³ is unsubstituted or substituted pyrrolidine, morpholine, piperidine, or piperazine.

In embodiments, R³ is C(O)R⁸, wherein R⁸ is unsubstituted or substituted pyrrolidine, morpholine, piperidine, or piperazine.

In embodiments, R³ is unsubstituted or substituted phenyl or pyridyl.

In embodiments, R³ is R¹⁰, OR¹⁰, CH₂R¹⁰, CH₂CH₂R¹⁰, or OCH₂CH₂R¹⁰, wherein R¹⁰ is unsubstituted or substituted pyrrolidine, morpholine, piperidine, or piperazine.

In embodiments, R³ is selected from the group consisting of

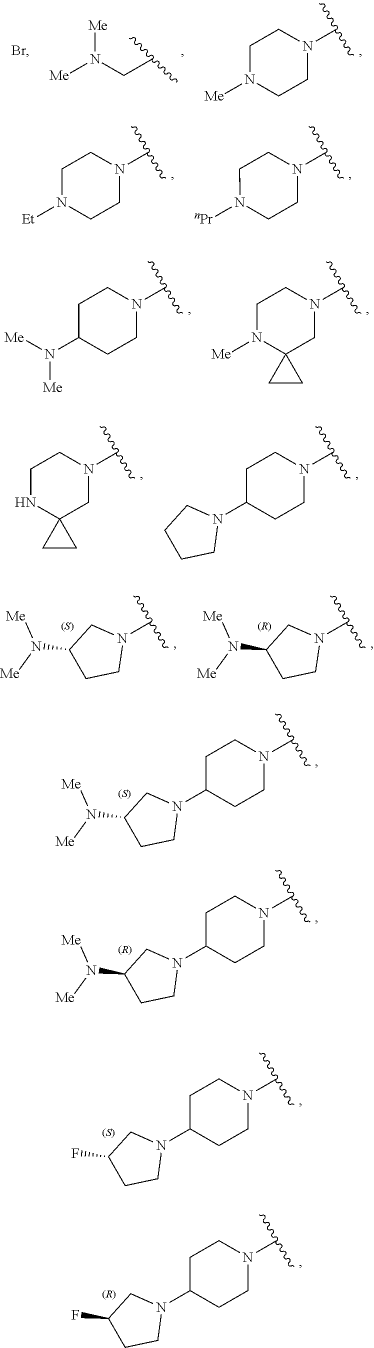

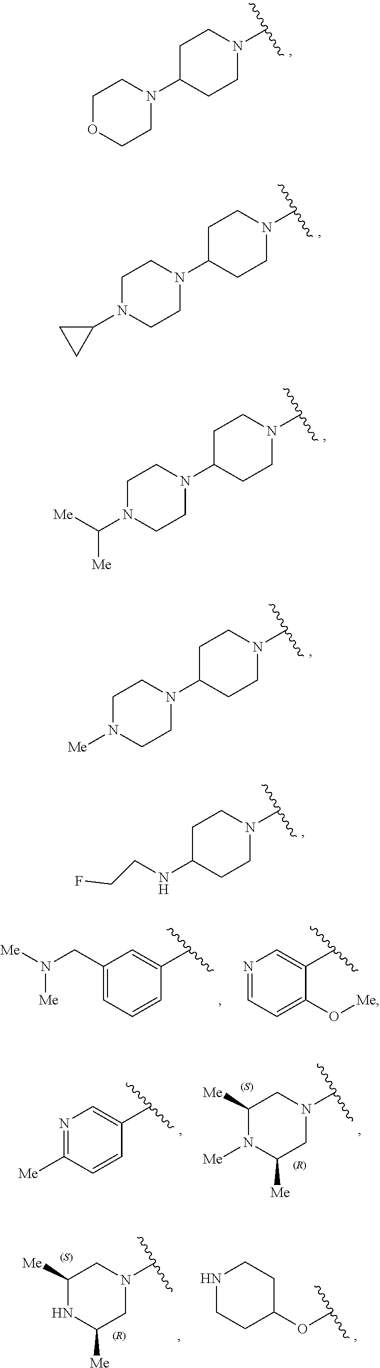

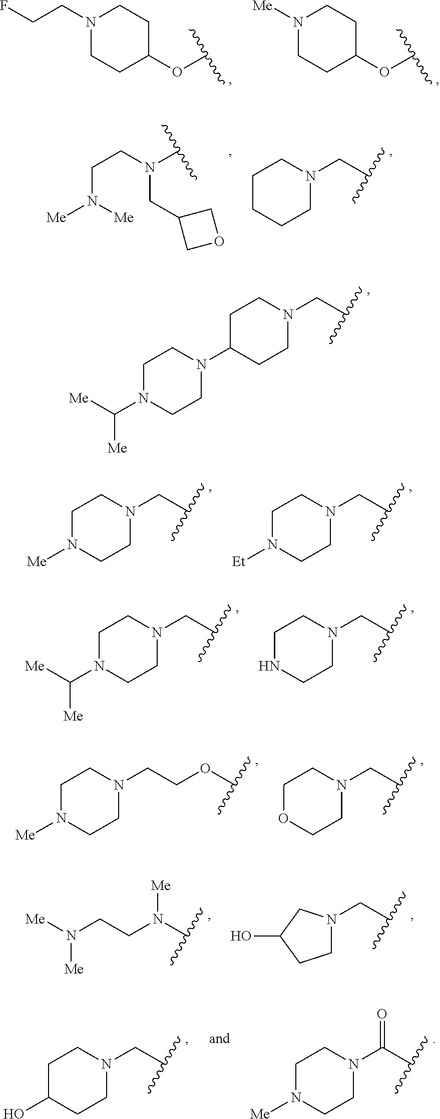

In embodiments, a compound is selected from the group consisting of Compounds (1)-(71), or a pharmaceutically acceptable salt thereof.

In another aspect, the invention features a pharmaceutical composition comprising any compound described herein, or a pharmaceutically acceptable salt thereof.

In another aspect, the invention features a method of treating cancer comprising administering to a human in need thereof an effective amount of any compound described herein, or a pharmaceutically acceptable salt thereof, in a pharmaceutical composition.

In embodiments, a cancer is a lung cancer.

In embodiments, a cancer is non-small cell lung cancer.

In embodiments, a cancer (e.g., a lung cancer such as non-small cell lung cancer) is an EGFR-driven cancer.

In embodiments, a cancer (e.g., a lung cancer such as non-small cell lung cancer) is characterized by an EGFR mutation.

### BRIEF DESCRIPTION OF THE DRAWINGS

FIG. 1 shows the exemplary synthetic schemes for preparing a compound of Formula (I) (X¹═O and X²═N).

FIG. 2 shows the exemplary synthetic schemes for preparing a compound of Formula (I) (X¹=covalent bond).

FIG. 3 shows the exemplary synthetic schemes for preparing a compound of Formula (I) (X¹═CH₂, alkenylene, or alkynylene).

FIG. 4 shows the exemplary synthetic schemes for preparing a compound of Formula (I) (X¹═NH, or N-alkyl).

FIG. 5 shows the general synthetic schemes for preparing Compound (32).

FIG. 6 shows the general synthetic schemes for preparing Compound (10).

FIG. 7 shows the general synthetic schemes for preparing Compound (16).

FIG. 8 shows the general synthetic schemes for preparing Compound (41).

FIG. 9 shows the general synthetic schemes for preparing Compound (33).

FIG. 10 shows the general synthetic schemes for preparing Compound (55).

FIG. 11 shows the general synthetic schemes for preparing Compound (63).

### DETAILED DESCRIPTION OF THE INVENTION

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, a bovine, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used in the description and the appended claims, the singular forms “a,” “an,” and “the” include plural referents unless the context clearly dictates otherwise. Thus, for example, reference to “a composition” includes mixtures of two or more such compositions.

Throughout the description and claims of this specification the word “comprise” and other forms of the word, such as “comprising” and “comprises,” means including but not limited to, and is not intended to exclude, for example, other additives, components, integers, or steps.

“Optional” or “optionally” means that the subsequently described event or circumstance may or may not occur, and that the description includes instances where the event or circumstance occurs and instances where it does not.

Improve, increase, or reduce: As used herein, the terms “improve,” “increase,” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio. Accordingly, pharmaceutically acceptable relates to substances that are not biologically or otherwise undesirable, i.e., the material can be administered to an individual along with the relevant active compound without causing clinically unacceptable biological effects or interacting in a deleterious manner with any of the other components of the pharmaceutical composition in which it is contained.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄-alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quarternization of an amine using an appropriate electrophile, e.g., an alkyl halide, to form a quarternized alkylated amino salt.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Whenever a term (e.g., alkyl or aryl) or either of their prefix roots (e.g., alk- or ar-) appear in a name of a substituent the name is to be interpreted as including those limitations provided herein. For example, affixing the suffix “-ene” to a group indicates the group is a divalent moiety, e.g., arylene is the divalent moiety of aryl, heteroarylene is the divalent moiety of heteroaryl, and heterocycloalkylene is the divalent moiety of heterocycloalkyl. Similarly, affixing the suffix “-oxy” to a group indicates the group is attached to the parent molecular structure through an oxygen atom (—O—).

Aliphatic: As used herein, the term aliphatic refers to hydrocarbons and includes both saturated and unsaturated hydrocarbons. An aliphatic may be linear, branched, or cyclic. For example, C₁-C₂₀ aliphatics can include C₁-C₂₀ alkyls (e.g., linear or branched C₁-C₂₀ saturated alkyls), C₂-C₂₀ alkenyls (e.g., linear or branched C₄-C₂₀ dienyls, linear, or branched C₆-C₂₀ trienyls, and the like), and C₂-C₂₀ alkynyls (e.g., linear or branched C₂-C₂₀ alkynyls). C₁-C₂ aliphatics can include C₃-C₂₀ cyclic aliphatics (e.g., C₃-C₂₀ cycloalkyls, C₄-C₂₀ cycloalkenyls, or C₅-C₂₀ cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the aliphatic is unsubstituted. In some embodiments, the aliphatic does not include any heteroatoms.

Alkyl: As used herein, the term “alkyl” means acyclic linear and branched hydrocarbon groups, e.g. “C₁-C₂₀ alkyl” refers to alkyl groups having 1-20 carbons and “C₁-C₄ alkyl” refers to alkyl groups having 1-4 carbons. Alkyl groups include C₁-C₂₀ alkyl, C₁-C₁₅ alkyl, C₁-C₁₀ alkyl, C₁-C₄ alkyl, and C₁-C₃ alkyl). In embodiments, an alkyl group is C₁-C₄ alkyl. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or—SO₂R′, wherein each instance of R′ independently is C₁-C₂₀ aliphatic (e.g., C₁-C₂ alkyl, C₁-C₁₅ alkyl, C₁-C₁₀ alkyl, C₁-C₄ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkyl is substituted (e.g., with 1, 2, 3, 4, 5, or 6 substituent groups as described herein). In some embodiments, an alkyl group is substituted with a —OH group and may also be referred to herein as a “hydroxyalkyl” group, where the prefix denotes the —OH group and “alkyl” is as described herein. In some embodiments, an alkyl group is substituted with a —OR′ group.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms.

Alkenyl: As used herein, “alkenyl” means any linear or branched hydrocarbon chains having one or more unsaturated carbon-carbon double bonds that may occur in any stable point along the chain, e.g. “C₂-C₂₀ alkenyl” refers to an alkenyl group having 2-20 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In some embodiments, the alkenyl comprises 1, 2, or 3 carbon-carbon double bond. In some embodiments, the alkenyl comprises a single carbon-carbon double bond. In some embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or—SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkenyl is unsubstituted. In some embodiments, the alkenyl is substituted (e.g., with 1, 2, 3, 4, 5, or 6 substituent groups as described herein). In some embodiments, an alkenyl group is substituted with a —OH group and may also be referred to herein as a “hydroxyalkenyl” group, where the prefix denotes the —OH group and “alkenyl” is as described herein.

Alkynyl: As used herein, “alkynyl” means any hydrocarbon chain of either linear or branched configuration, having one or more carbon-carbon triple bonds occurring in any stable point along the chain, e.g. “C₂-C₂₀ alkynyl” refers to an alkynyl group having 2-20 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In some embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or—SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkynyl is unsubstituted. In some embodiments, the alkynyl is substituted (e.g., with 1, 2, 3, 4, 5, or 6 substituent groups as described herein).

Alkoxy: The term “alkoxy” refers to the group —O-alkyl, including from 1 to 10 carbon atoms of a straight, branched, saturated cyclic configuration and combinations thereof, attached to the parent molecular structure through an oxygen. Examples include methoxy, ethoxy, propoxy, isopropoxy, butoxy, t-butoxy, pentoxy, cyclopropyloxy, cyclohexyloxy and the like. “Lower alkoxy” refers to alkoxy groups containing one to six carbons. In some embodiments, C₁₋₄ alkoxy is an alkoxy group which encompasses both straight and branched chain alkyls of from 1 to 4 carbon atoms. Unless stated otherwise in the specification, an alkoxy group can be optionally substituted by one or more substituents (e.g., as described herein for alkyl). The terms “alkenoxy” and “alkynoxy” mirror the above description of “alkoxy” wherein the prefix “alk” is replaced with “alken” or “alkyn” respectively, and the parent “alkenyl” or “alkynyl” terms are as described herein.

Amide: The term “amide” or “amido” refers to a chemical moiety with formula —C(O)N(R′)₂, —C(O)N(R′)—, —NR′C(O)R⁸, or —NR′C(O)—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Amino: The term “amino” or “amine” refers to a —N(R′)₂ group, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated otherwise in the specification, each of which moiety can itself be optionally substituted as described herein, or two R′ can combine with the nitrogen atom to form a 3-, 4-, 5-, 6-, or 7-membered ring. In embodiments, an amino group is —NHR′, where R′ is aryl (“arylamino”), heteroaryl (“heteroarylamino”), or alkyl (“alkylamino”).

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, wherein at least one ring in the system is aromatic, and wherein each ring in the system contains 4 to 7 ring members. In some embodiments, an aryl group has 6 ring carbon atoms (“C₆ aryl,” e.g., phenyl). In some embodiments, an aryl group has 10 ring carbon atoms (“C₁₀ aryl,” e.g., naphthyl such as 1-naphthyl and 2-naphthyl). In some embodiments, an aryl group has 14 ring carbon atoms (“C₁₄ aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

Arylalkyl: The term “arylalkyl” refers to an -(alkylene)-aryl radical where aryl and alkylene are as disclosed herein and which are optionally substituted by one or more of the exemplary substituent groups described herein. The “arylalkyl” group is bonded to the parent molecular structure through the alkylene moiety. The term “arylalkoxy” refers to an —O-[arylalkyl] radical (—O-[(alkylene)-aryl]), which is attached to the parent molecular structure through the oxygen.

Arylene: The term “arylene” as used herein refers to an aryl group that is divalent (that is, having two points of attachment to the molecule). Exemplary arylenes include phenylene (e.g., unsubstituted phenylene or substituted phenylene).

Cyclic: The term “cyclic” as used herein, refers to any covalently closed structure. Cyclic moieties include, for example, carbocycles (e.g., aryls and cycloalkyls), heterocycles (e.g., heteroaryls and heterocycloalkyls), aromatics (e.g. aryls and heteroaryls), and non-aromatics (e.g., cycloalkyls and heterocycloalkyls). In some embodiments, cyclic moieties are optionally substituted. In some embodiments, cyclic moieties form part of a ring system.

Cycloaliphatic: The term “cycloaliphatic” refers to a monocyclic or polycyclic radical that contains only carbon and hydrogen, and can be saturated or partially unsaturated. Fully saturated cycloaliphatics can be termed “cycloalkyl”. Partially unsaturated cycloalkyl groups can be termed “cycloalkenyl” if the carbocycle contains at least one double bond, or “cycloalkynyl” if the carbocycle contains at least one triple bond. Cycloaliphatic groups include groups having from 3 to 13 ring atoms (e.g., C₃₋₁₃ cycloalkyl). Whenever it appears herein, a numerical range such as “3 to 10” refers to each integer in the given range; e.g., “3 to 10 carbon atoms” means that the cycloaliphatic group (e.g., cycloalkyl) can consist of 3 carbon atoms, 4 carbon atoms, 5 carbon atoms, etc., up to and including 10 carbon atoms. The term “cycloaliphatic” also includes bridged and spiro-fused cyclic structures containing no heteroatoms. The term also includes monocyclic or fused-ring polycyclic (i.e., rings which share adjacent pairs of ring atoms) groups. Polycyclic cycloaliphatic groups include bicycles, tricycles, tetracycles, and the like. In some embodiments, “cycloalkyl” can be a C₃₋₅ cycloalkyl group. In some embodiments, “cycloalkyl” can be a C₃₋₅ cycloalkyl group. Illustrative examples of cycloaliphatic groups include, but are not limited to the following moieties: C₃₋₆ cycloaliphatic groups include, without limitation, cyclopropyl (C₃), cyclobutyl (C₄), cyclopentyl (C₅), cyclopentenyl (C₅), cyclohexyl (C), cyclohexenyl (C), cyclohexadienyl (C) and the like. Examples of C₃₋₇ cycloaliphatic groups include norbornyl (C₇). Examples of C₃₋₈ cycloaliphatic groups include the aforementioned C₃₋₇ carbocyclyl groups as well as cycloheptyl (C₇), cycloheptadienyl (C₇), cyclohept-atrienyl (C₇), cyclooctyl (C₈), bicyclo[2.2.1]heptanyl, bicyclo[2.2.2]octanyl, and the like. Examples of C₃₋₁₃ cycloaliphatic groups include the aforementioned C₃₋₈ carbocyclyl groups as well as octahydro-1H indenyl, decahydronaphthalenyl, spiro[4.5]decanyl, and the like.

Cyano: The term “cyano” refers to a —CN group.

Deuterium: The term “deuterium” is also called heavy hydrogen. Deuterium is isotope of hydrogen with a nucleus consisting of one proton and one neutron, which is double the mass of the nucleus of ordinary hydrogen (one proton). In embodiments, deuterium can also be identified as ²H.

Ester: The term “ester” refers to a group of formula —C(O)OR′ or —R′OC(O)—, where R′ is selected from alkyl, alkenyl, alkynyl, heteroalkyl (bonded through a chain carbon), cycloalkyl, aryl, arylalkyl, heteroaryl, heteroarylalkyl, or heterocycloalkyl as described herein.

Halogen or Halo: As used herein, the term “halogen” or “halo” means fluorine, chlorine, bromine, or iodine.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl. Accordingly, the term “heteroalkoxy” refers to the group —O-heteroalkyl, where the group is attached to the parent molecular structure via the oxygen.

Heteroalkylene: The term “heteroalkylene,” as used herein, represents a divalent form of a heteroalkyl group as described herein.

Heteroaryl: The term “heteroaryl,” as used herein, refers to a monocyclic, bicyclic, or tricyclic carbocyclic ring system having a total of six to fourteen ring members, wherein said ring system has a single point of attachment to the rest of the molecule, wherein at least one ring in the system is aromatic, wherein each ring in the system contains 4 to 7 ring members, and wherein at least one ring atom is a heteroatom such as, but not limited to, nitrogen and oxygen. Examples of heteroaryl groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Accordingly, the term “heteroaryloxy” refers to the group —O-heteroaryl, where the group is attached to the parent molecular structure via the oxygen.

Heteroarylene: The term “heteroalkylene,” as used herein, represents a divalent form of a heteroaryl group as described herein.

Heteroarylalkyl: The term “heteroarylalkyl” refers to an -(alkylene)-heteroaryl radical where heteroaryl and alkylene are as disclosed herein and which are optionally substituted by one or more of the exemplary substituent groups described herein. The “heteroarylalkyl” group is bonded to the parent molecular structure through the alkylene moiety. The term “heteroarylalkoxy” refers to an —O-[heteroarylalkyl] radical (—O-[(alkylene)-heteroaryl]), which is attached to the parent molecular structure through the oxygen.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. Examples of heterocycloalkyl groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. The heterocycloalkyl group can be substituted or unsubstituted.

Heterocycle: The term “heterocycle” or “heterocyclyl” refers to heteroaryl and heterocycloalkyl as used herein, refers to groups containing one to four heteroatoms each selected from O, S and N, wherein each heterocycle group has from 4 to 10 atoms in its ring system, and with the proviso that the ring of said group does not contain two adjacent O or S atoms. Herein, whenever the number of carbon atoms in a heterocycle is indicated (e.g., C₁-C₆-heterocycle), at least one other atom (the heteroatom) must be present in the ring. Designations such as “C₁-C₆-heterocycle” refer only to the number of carbon atoms in the ring and do not refer to the total number of atoms in the ring. In some embodiments, it is understood that the heterocycle ring has additional heteroatoms in the ring. Designations such as “4-6-membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In some embodiments, in heterocycles that have two or more heteroatoms, those two or more heteroatoms are the same or different from one another. In some embodiments, heterocycles are optionally substituted. In some embodiments, binding to a heterocycle is at a heteroatom or via a carbon atom. Heterocycloalkyl groups include groups having only 4 atoms in their ring system, but heteroaryl groups must have at least 5 atoms in their ring system. The heterocycle groups include benzo-fused ring systems. An example of a 4-membered heterocycle group is azetidinyl (derived from azetidine). An example of a 5-membered heterocycle group is thiazolyl. An example of a 6-membered heterocycle group is pyridyl, and an example of a 10-membered heterocycle group is quinolinyl. In some embodiments, the foregoing groups, as derived from the groups listed above, are C-attached or N-attached where such is possible. For instance, in some embodiments, a group derived from pyrrole is pyrrol-1-yl (N-attached) or pyrrol-3-yl (C-attached). Further, in some embodiments, a group derived from imidazole is imidazol-1-yl or imidazol-3-yl (both N-attached) or imidazol-2-yl, imidazol-4-yl or imidazol-5-yl (all C-attached). The heterocycle groups include benzo-fused ring systems and ring systems substituted with one or two oxo (═O) moieties such as pyrrolidin-2-one. In some embodiments, depending on the structure, a heterocycle group is a monoradical or a diradical (i.e., a heterocyclene group). The heterocycles described herein are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alynyl, carboxy, cyano, formyl, haloalkoxy, haloalkyl, halogen, hydroxyl, hydroxyalkylene, mercapto, nitro, amino, and amido moities.

Isotope: The term “isotope” refers to a variant of a particular chemical element which differs in neutron number, and consequently in nucleon number. All isotopes of a given element have the same number of protons but different numbers of neutrons in each atom.

Nitro: The term “nitro” refers to a —NO₂ group.

Sulfonamide: The term “sulfonamide” or sulfonamido” refers to the following groups: —S(═O)₂—(R′)₂, —N(R′)—S(═O)₂—R⁸, —S(═O)₂—N(R′)—, or —N(R′)—S(═O)₂—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Nitrogen protecting group: In certain embodiments, the substituent present on a nitrogen atom is a nitrogen protecting group (also referred to as an amino protecting group). Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamanty1)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pme), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

Moiety: The term “moiety” refers to a specific segment or functional group of a molecule. Chemical moieties are often recognized chemical entities embedded in or appended to a molecule.

Molecular groups herein may be substituted or unsubstituted (e.g., as described herein). The term “substituted” means that the specified group or moiety bears one or more substituents: at least one hydrogen present on a group atom (e.g., a carbon or nitrogen atom) is replaced with a permissible substituent, e.g., a substituent which upon substitution for the hydrogen results in a stable compound, e.g., a compound which does not spontaneously undergo transformation such as by rearrangement, cyclization, elimination, or other reaction. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In embodiments, a group described herein is substituted. In embodiments, a group described herein is unsubstituted. In cases where a specified moiety or group is not expressly noted as being optionally substituted or substituted with any specified substituent, it is understood that such a moiety or group is intended to be unsubstituted.

A wide variety of substituents are well known, and methods for their formation and introduction into a variety of parent groups are also well known. Representative substituents include but are not limited to alkyl, cycloalkyl, alkenyl, cycloalkenyl, alkynyl, arylalkyl, alkylaryl, aryl, heteroaryl, heterocycloalkyl, hydroxyalkyl, arylalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, alkoxy, hydroxy, halo (e.g., —Cl and —Br), nitro, oximino, —COOR⁵⁰, —COR⁵⁰, —SO₀₋₂R⁵⁰, —SO₂NR⁵⁰R⁵¹, —NR⁵²SO₂R⁵⁰, ═C(R⁵⁰R⁵¹), ═N—OR⁵⁰, ═N—CN, ═C(halo)₂, ═S, ═O, —CON(R⁵⁰R⁵¹), —OCOR⁵⁰, —OCON(R⁵⁰R⁵¹), —N(R⁵²)CO(R⁵⁰), —N(R⁵²)COOR⁵⁰, —N(R⁵²)CON(R⁵⁰(R⁵¹), —P(OR⁵⁰)₂, —P(O)R⁵⁰R⁵¹, and —P(O)OR⁵⁰OR⁵¹, wherein R⁵⁰, R⁵¹ and R⁵² may be independently selected from the following: a hydrogen atom and a branched or straight-chain, C₁₋₆-alkyl, C₃₋₆-cycloalkyl, C₄₋₆-heterocycloalkyl, heteroaryl and aryl group, with or without substituents. When permissible, R⁵⁰ and R⁵¹ can be joined together to form a carbocyclic or heterocyclic ring system.

In preferred embodiments, the substituent is selected from halogen, —COR′, —CO₂H, —CO₂R′, —CN, —OH, —OR′, —OCOR′, —OCO₂R′, —NH₂, —NHR′, —N(R′)₂, —SR′, and —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In certain embodiments thereof, R′ independently is an unsubstituted alkyl (e.g., unsubstituted C₁-C₂₀ alkyl, C₁-C₁₅ alkyl, C₁-C₁₀ alkyl, or C₁-C₃ alkyl). Preferably, R′ independently is unsubstituted C₁-C₃ alkyl.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

### Compounds of the Invention

Described herein are new compounds that can be effective inhibitors of EGFR. Such compounds can be useful for treating various diseases and disorders, including EGFR-driven cancers such as non-small cell lung cancer (NSCLC) characterized by mutant EGFR. Exemplary compounds are described herein.

Compounds of Formulas (I)-(XIV)

In one aspect, provided herein are compounds having a structure according to Formula (I):

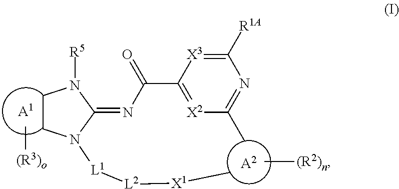

or a pharmaceutically acceptable salt thereof, wherein
A¹ is independently phenylene or 5- or 6-membered heteroarylene;
A² is independently phenyl, naphthyl, or a 5- to 13-membered heteroaryl;
X¹ is independently O or X^{1A};
X^{1A} is a covalent bond, S, NR⁴, C₁₋₆ alkylene, C₂₋₆ alkenylene, or C₂₋₆ alkynylene;
each of X² and X³ is independently N or CR^{1B};
L¹ is independently a covalent bond or C₁₋₆ alkylene;
L² is independently a covalent bond, C₂₋₆ alkenylene, C₂₋₆ alkynylene, C₃₋₆ cycloalkylene, 3- to 10-membered heterocyclylene, phenylene, or 5- or 6-membered heteroarylene;
each R^{1A} and R^{1B} is independently H, OH, CN, halogen, C₁₋₆ aliphatic, C₁₋₆ alkoxy, NR⁶R⁷, C(O)R⁸, CO₂R′, C(O)NR⁶R⁷, NR⁹C(O)R⁸, NR⁹CO₂R⁸, NR⁹C(O)NR⁶R⁷, or R¹⁰;
each R² and R³, when present, is independently OH, CN, halogen, C₁₋₆ aliphatic, C₁₋₆ alkoxy, NR⁶R⁷, C(O)R⁸, CO₂R¹, C(O)NR⁶R⁷, NR⁹C(O)R⁸, NR⁹CO₂R⁸, NR⁹C(O)NR⁶R⁷, R¹⁰, OR¹⁰, CH₂R¹⁰, CH₂CH₂R¹⁰, OCH₂R¹⁰, or OCH₂CH₂R¹⁰;
each R⁴ is independently H, a N-protecting group, or C₁₋₆ alkyl;
R⁵ is hydrogen;
each R⁶, R⁷, and R⁹ is independently H or C₁₋₆ alkyl; or R⁶ and R⁷, together with the nitrogen atom to which they are attached, form a 3- to 10-membered heterocyclyl, or R⁶ and R⁹, together with the atoms to which they are attached, form a 3- to 10-membered heterocyclyl;
R⁸ is independently C₁₋₆ aliphatic, C₃-C₁₀ cycloaliphatic, 3- to 10-membered heterocyclyl, phenyl, naphthyl, or a 5- to 12-membered heteroaryl, or R⁸ and R⁹, together with the atoms to which they are attached, form a 3- to 10-membered heterocyclyl;
R¹⁰ is independently C₃-C₁₀ cycloaliphatic, 3- to 10-membered heterocyclyl, phenyl, naphthyl, or a 5- to 12-membered heteroaryl; and
each of n and o is independently 0, 1, or 2.

In embodiments, when X¹ is O, and both of X² and X³ are not N, then A² is naphthyl or a bicyclic 8- to 12-membered heteroaryl.

In embodiments, X² is N. In embodiments, X² is CR^{1B} (e.g., CH).

In embodiments, X³ is N. In embodiments, X³ is CR^{1B} (e.g., CH).

In embodiments, each of X² and X³ is N. In embodiments, each of X² and X³ is CR^{1B}. In embodiments, each of X² and X³ is CH. In embodiments, one of X² and X³ is N, and the other is CR^{1B} (e.g., CH).

In embodiments, at least one of X² and X³ is N.

In embodiments, X¹ is X^{1A}.

In embodiments, X^{1A} is a covalent bond.

In embodiments, X^{1A} is S or NR⁴.

In embodiments, X^{1A} is C₁₋₆ alkylene, C₂₋₆ alkenylene, or C₂₋₆ alkynylene.

In embodiments, L¹ is a covalent bond.

In embodiments, L¹ is unsubstituted branched C₁₋₆ alkylene, or linear C₁₋₆ alkylene optionally comprising a —OH substituent.

In embodiments,
X¹ is X^{1A}, wherein X^{1A} is a covalent bond, C₁₋₆ alkylene, C₂₋₆ alkenylene, or C₂₋₆ alkynylene;
L¹ is independently a covalent bond or C₁₋₆ alkylene;
L² is independently a covalent bond, C₂₋₆ alkenylene, C₂₋₆ alkynylene, C₃₋₆ cycloalkylene, 3- to 10-membered heterocyclylene, phenylene, or 5- or 6-membered heteroarylene; and
where at least one of X^{A}, L¹, and L² is a covalent bond.

In embodiments, one X^{1A} and L¹ is a covalent bond and the other is C₁₋₆ alkylene; and L² is a covalent bond.

In embodiments, each of L¹ and L² is a covalent bond.

In embodiments, each of X^{1A} and L² is a covalent bond.

In embodiments, each of X^{1A} and L¹ is a covalent bond.

In embodiments, a compound of Formula (I) has a structure according to Formula (II),

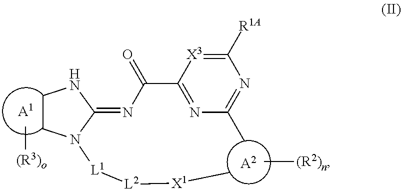

or a pharmaceutically acceptable salt thereof.

In embodiments, A¹, A², R^{A}, R², R³, L¹, L², X¹, X³, n and o are according to any embodiment described herein.

In embodiments, a compound of Formula (I) has a structure according to Formula (III).

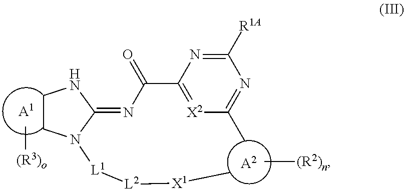

or a pharmaceutically acceptable salt thereof.

In embodiments, A¹, A², R^{1A}, R², R³, L¹, L², X¹, X², n and o are according to any embodiment described herein.

In embodiments, a compound of Formula (I) has a structure according to Formula (IV).

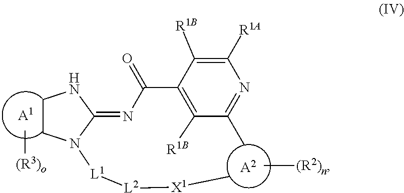

or a pharmaceutically acceptable salt thereof.

In embodiments, A¹, A², R^{1A}, R^{1B}, R², R³, L¹, L², X¹, n and o are according to any embodiment described herein.

In embodiments, X¹ is O.

In embodiments, X¹ is XIA.

In embodiments, X¹ is not O.

In embodiments, X^{1A} is a covalent bond.

In embodiments, X^{1A} is S or NR⁴.

In embodiments, X^{1A} is C₁₋₆ alkylene, C₂₋₆ alkenylene, or C₂₋₆ alkynylene.

In embodiments, a compound of Formula (I) has a structure according to Formula (V).

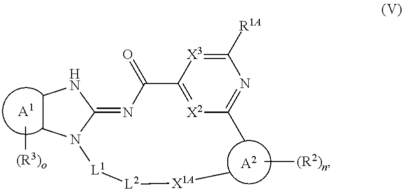

or a pharmaceutically acceptable salt thereof.

In embodiments, A¹, A², R^{1A}, R², R³, L¹, L², X^{1A}, X², X³, n and o are according to any embodiment described herein.

In embodiments, a compound of Formula (I) has a structure according to Formula (VI).

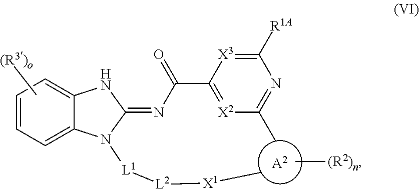

or a pharmaceutically acceptable salt thereof.

In embodiments, A², R^{1A}, R², R³, L¹, L², X¹, X², X³, n and o are according to any embodiment described herein.

In embodiments, R² is CH₃.

In embodiments, L¹ is a C₁₋₆ alkylene (e.g., CH₂, (CH₂)₂, (CH₂)₃, (CH₂)₄, (CH₂)₅, or (CH₂)₆). In embodiments, L¹ is a branched C₁₋₆ alkylene. In embodiments, L¹ is a linear C₁₋₆ alkylene. In embodiments, L¹ is unsubstituted C₁₋₆ alkylene. In embodiments, L¹ is unsubstituted branched C₁₋₆ alkylene. In embodiments, L¹ is unsubstituted linear C₁₋₆ alkylene. In embodiments, L¹ is substituted C₁₋₆ alkylene (e.g., comprising an OH group). In embodiments, L¹ is substituted branched C₁₋₆ alkylene (e.g., comprising an OH group). In embodiments, L¹ is substituted linear C₁₋₆ alkylene (e.g., comprising an OH group).

In embodiments, a compound of has a structure according to Formula (VI-1).

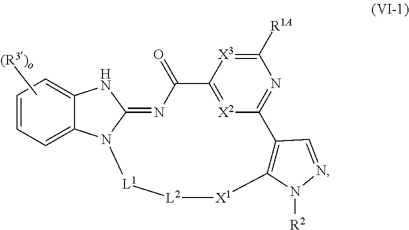

or a pharmaceutically acceptable salt thereof.

In embodiments, R^{1A}, R², R³, L¹, L², X¹, X², X³, and o are according to any embodiment described herein.

In embodiments, R² is CH₃.

In embodiments, L¹ is a C₁₋₆ alkylene (e.g., CH₂, (CH₂)₂, (CH₂)₃, (CH₂)₄, (CH₂)₅, or (CH₂)₆). In embodiments, L¹ is a branched C₁₋₆ alkylene. In embodiments, L¹ is a linear C₁₋₆ alkylene. In embodiments, L¹ is unsubstituted C₁₋₆ alkylene. In embodiments, L¹ is unsubstituted branched C₁₋₆ alkylene. In embodiments, L¹ is unsubstituted linear C₁₋₆ alkylene. In embodiments, L¹ is substituted C₁₋₆ alkylene (e.g., comprising an OH group). In embodiments, L¹ is substituted branched C₁₋₆ alkylene (e.g., comprising an OH group). In embodiments, L¹ is substituted linear C₁₋₆ alkylene (e.g., comprising an OH group).

In embodiments, a compound of has a structure according to Formula (VI-2).

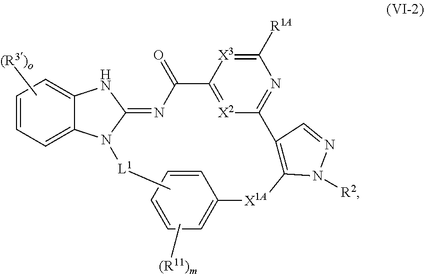

or a pharmaceutically acceptable salt thereof, wherein
each R¹¹ is independently OH, CN, halogen, C₁₋₆ alkyl, or C₁₋₆ alkoxy; and
m is 0, 1, or 2.

In embodiments, R^{1A}, R², R³, L¹, X^{1A}, X², X³, and o are according to any embodiment described herein.

In embodiments, R¹¹ is OH. In embodiments, R¹¹ is CN. In embodiments, R¹¹ is halogen (e.g., F, Cl, Br, or I). In embodiments, R¹¹ is C₁₋₆ alkyl. In embodiments, R¹¹ is C₁₋₆ alkoxy. In embodiments, R¹¹ is unsubstituted C₁₋₆ alkyl. In embodiments, R¹¹ is unsubstituted C₁₋₆ alkoxy. In embodiments, R¹¹ is substituted C₁₋₆ alkyl (e.g., comprising 1, 2, or 3 substituent groups). In embodiments, R¹¹ is substituted C₁₋₆ alkoxy (e.g., comprising 1, 2, or 3 substituent groups).

In embodiments, m is 0. In embodiments, m is 1. In embodiments, m is 2. In embodiments, m is 1 or 2.

In embodiments, R² is CH₃.

In embodiments, X^{1A} is a covalent bond.

In embodiments, X^{1A} is C₁₋₆ alkylene (e.g., CH₂, (CH₂)₂, (CH₂)₃, (CH₂)₄, (CH₂)₅, or (CH₂)₆). In embodiments, a C₁₋₆ alkylene is unsubstituted. In embodiments, a C₁₋₆ alkylene is substituted (e.g., comprising 1, 2, or 3 substituent groups such as OH, oxo (═O), or unsubstituted C₁₋₃ alkyl). In embodiments, X^{1A} is a branched C₁₋₆ alkylene. In embodiments, X^{1A} is a linear C₁₋₆ alkylene. In embodiments, X^{1A} is unsubstituted branched C₁₋₆ alkylene. In embodiments, X^{1A} is unsubstituted linear C₁₋₆ alkylene. In embodiments, X^{1A} is substituted branched C₁₋₆ alkylene (e.g., comprising 1, 2, or 3 substituent groups such as OH, oxo (═O), or unsubstituted C₁₋₃ alkyl)). In embodiments, X^{1A} is substituted linear C₁₋₆ alkylene (e.g., comprising 1, 2, or 3 substituent groups such as OH, oxo (═O), or unsubstituted C₁₋₃ alkyl)).

In embodiments, L¹ is a C₁₋₆ alkylene (e.g., CH₂, (CH₂)₂, (CH₂)₃, (CH₂)₄, (CH₂)₅, or (CH₂)₆). In embodiments, L¹ is a branched C₁₋₆ alkylene. In embodiments, L¹ is a linear C₁₋₆ alkylene. In embodiments, L¹ is unsubstituted C₁₋₆ alkylene. In embodiments, L¹ is unsubstituted branched C₁₋₆ alkylene. In embodiments, L¹ is unsubstituted linear C₁₋₆ alkylene. In embodiments, L¹ is substituted C₁₋₆ alkylene (e.g., comprising an OH group). In embodiments, L¹ is substituted branched C₁₋₆ alkylene (e.g., comprising an OH group). In embodiments, L¹ is substituted linear C₁₋₆ alkylene (e.g., comprising an OH group).

In embodiments, a compound of has a structure according to Formula (VI-3).

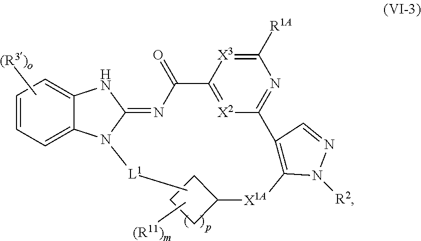

or a pharmaceutically acceptable salt thereof, wherein
each R¹¹ is independently OH, oxo, CN, halogen, C₁₋₆ alkyl, or C₁₋₆ alkoxy;
m is 0, 1, or 2; and
p is 0, 1, 2, or 3.

In embodiments, R^{1A}, R², R³, L¹, X^{1A}, X², X³, and o are according to any embodiment described herein.

In embodiments, R¹¹ is OH. In embodiments, R¹¹ is oxo (═O). In embodiments, R¹¹ is CN. In embodiments, R¹¹ is halogen (e.g., F, Cl, Br, or I). In embodiments, R¹¹ is C₁₋₆ alkyl. In embodiments, R¹¹ is C₁₋₆ alkoxy. In embodiments, R¹¹ is unsubstituted C₁₋₆ alkyl. In embodiments, R¹¹ is unsubstituted C₁₋₆ alkoxy. In embodiments, R¹¹ is substituted C₁₋₆ alkyl (e.g., comprising 1, 2, or 3 substituent groups). In embodiments, R¹¹ is substituted C₁₋₆ alkoxy (e.g., comprising 1, 2, or 3 substituent groups).

In embodiments, m is 0. In embodiments, m is 1. In embodiments, m is 2. In embodiments, m is 1 or 2.

In embodiments, p is 0. In embodiments, p is 1. In embodiments, p is 2. In embodiments, p is 3.

In embodiments, R² is CH₃.

In embodiments, X^{1A} is a covalent bond.

In embodiments, X^{1A} is C₁₋₆ alkylene (e.g., CH₂, (CH₂)₂, (CH₂)₃, (CH₂)₄, (CH₂)₅, or (CH₂)₆). In embodiments, a C₁₋₆ alkylene is unsubstituted. In embodiments, a C₁₋₆ alkylene is substituted (e.g., comprising 1, 2, or 3 substituent groups such as OH, oxo (═O), or unsubstituted C₁₋₃ alkyl). In embodiments, X^{1A} is a branched C₁₋₆ alkylene. In embodiments, X^{1A} is a linear C₁₋₆ alkylene. In embodiments, X^{1A} is unsubstituted branched C₁₋₆ alkylene. In embodiments, X^{1A} is unsubstituted linear C₁₋₆ alkylene. In embodiments, X^{1A} is substituted branched C₁₋₆ alkylene (e.g., comprising 1, 2, or 3 substituent groups such as OH, oxo (═O), or unsubstituted C₁₋₃ alkyl)). In embodiments, X^{1A} is substituted linear C₁₋₆ alkylene (e.g., comprising 1, 2, or 3 substituent groups such as OH, oxo (═O), or unsubstituted C₁₋₃ alkyl)).

In embodiments, L¹ is a C₁₋₆ alkylene (e.g., CH₂, (CH₂)₂, (CH₂)₃, (CH₂)₄, (CH₂)₅, or (CH₂)₆). In embodiments, L¹ is a branched C₁₋₆ alkylene. In embodiments, L¹ is a linear C₁₋₆ alkylene. In embodiments, L¹ is unsubstituted C₁₋₆ alkylene. In embodiments, L¹ is unsubstituted branched C₁₋₆ alkylene. In embodiments, L¹ is unsubstituted linear C₁₋₆ alkylene. In embodiments, L¹ is substituted C₁₋₆ alkylene (e.g., comprising an OH group). In embodiments, L¹ is substituted branched C₁₋₆ alkylene (e.g., comprising an OH group). In embodiments, L¹ is substituted linear C₁₋₆ alkylene (e.g., comprising an OH group).

In embodiments, a compound of has a structure according to Formula (VI-4).

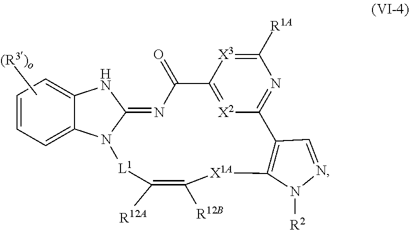

or a pharmaceutically acceptable salt thereof, wherein
X^{1A} is independently a covalent bond or C₁₋₆ alkylene; and
each R^{12A} and R^{12B} is independently H or C₁₋₆ alkyl, or R^{12A} and R^{12B} combine to form a cyclopentene or cyclohexene.

In embodiments, R^{1A}, R², R³, L¹, X^{1A}, X², X³, and o are according to any embodiment described herein.

In embodiments, X^{1A} is a covalent bond.

In embodiments, X^{1A} is C₁₋₆ alkylene (e.g., CH₂, (CH₂)₂, (CH₂)₃, (CH₂)₄, (CH₂)₅, or (CH₂)₆). In embodiments, a C₁₋₆ alkylene is unsubstituted. In embodiments, a C₁₋₆ alkylene is substituted (e.g., comprising 1, 2, or 3 substituent groups such as OH, oxo (═O), or unsubstituted C₁₋₃ alkyl). In embodiments, X^{1A} is a branched C₁₋₆ alkylene. In embodiments, X^{1A} is a linear C₁₋₆ alkylene. In embodiments, X^{1A} is unsubstituted branched C₁₋₆ alkylene. In embodiments, X^{1A} is unsubstituted linear C₁₋₆ alkylene. In embodiments, X^{1A} is substituted branched C₁₋₆ alkylene (e.g., comprising 1, 2, or 3 substituent groups such as OH, oxo (═O), or unsubstituted C₁₋₃ alkyl)). In embodiments, X^{1A} is substituted linear C₁₋₆ alkylene (e.g., comprising 1, 2, or 3 substituent groups such as OH, oxo (═O), or unsubstituted C₁₋₃ alkyl)).

In embodiments, R^{12A} is H. In embodiments, R^{12A} is C₁₋₆ alkyl. In embodiments, R^{12A} is unsubstituted C₁₋₆ alkyl. In embodiments, R^{12A} is substituted C₁₋₆ alkyl (e.g., comprising 1, 2, or 3 substituent groups).

In embodiments, R^{12B} is H. In embodiments, R^{12B} is C₁₋₆ alkyl. In embodiments, R^{12B} is unsubstituted C₁₋₆ alkyl. In embodiments, R^{12B} is substituted C₁₋₆ alkyl (e.g., comprising 1, 2, or 3 substituent groups).

In embodiments, R^{12A} and R^{12B} combine to form a cyclopentene or cyclohexene. In embodiments, a cyclopentene or cyclohexene is unsubstituted. In embodiments, a cyclopentene or cyclohexene is substituted (e.g., comprising 1, 2, or 3 substituent groups).

In embodiments, R² is CH₃.

In embodiments, L¹ is a C₁₋₆ alkylene (e.g., CH₂, (CH₂)₂, (CH₂)₃, (CH₂)₄, (CH₂)₅, or (CH₂)₆). In embodiments, L¹ is a branched C₁₋₆ alkylene. In embodiments, L¹ is a linear C₁₋₆ alkylene. In embodiments, L¹ is unsubstituted C₁₋₆ alkylene. In embodiments, L¹ is unsubstituted branched C₁₋₆ alkylene. In embodiments, L¹ is unsubstituted linear C₁₋₆ alkylene. In embodiments, L¹ is substituted C₁₋₆ alkylene (e.g., comprising an OH group). In embodiments, L¹ is substituted branched C₁₋₆ alkylene (e.g., comprising an OH group). In embodiments, L¹ is substituted linear C₁₋₆ alkylene (e.g., comprising an OH group).

In embodiments, a compound of has a structure according to Formula (VI-5).

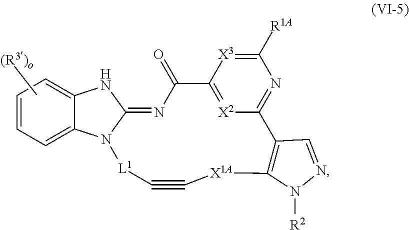

or a pharmaceutically acceptable salt thereof, wherein
X^{1A} is a covalent bond or C₁₋₆ alkylene; and
L¹ is C₁₋₆ alkylene.

In embodiments, R^{1A}, R², R³, L¹, X^{1A}, X², X³, and o are according to any embodiment described herein.

In embodiments, X^{1A} is a covalent bond.

In embodiments, X^{1A} is C₁₋₆ alkylene (e.g., CH₂, (CH₂)₂, (CH₂)₃, (CH₂)₄, (CH₂)₅, or (CH₂)₆). In embodiments, a C₁₋₆ alkylene is unsubstituted. In embodiments, a C₁₋₆ alkylene is substituted (e.g., comprising 1, 2, or 3 substituent groups such as OH, oxo (═O), or unsubstituted C₁₋₃ alkyl). In embodiments, X^{1A} is a branched C₁₋₆ alkylene. In embodiments, X^{1A} is a linear C₁₋₆ alkylene. In embodiments, X^{1A} is unsubstituted branched C₁₋₆ alkylene. In embodiments, X^{1A} is unsubstituted linear C₁₋₆ alkylene. In embodiments, X^{1A} is substituted branched C₁₋₆ alkylene (e.g., comprising 1, 2, or 3 substituent groups such as OH, oxo (═O), or unsubstituted C₁₋₃ alkyl)). In embodiments, X^{1A} is substituted linear C₁₋₆ alkylene (e.g., comprising 1, 2, or 3 substituent groups such as OH, oxo (═O), or unsubstituted C₁₋₃ alkyl)).

In embodiments, L¹ is a C₁₋₆ alkylene (e.g., CH₂, (CH₂)₂, (CH₂)₃, (CH₂)₄, (CH₂)₅, or (CH₂)₆). In embodiments, L¹ is a branched C₁₋₆ alkylene. In embodiments, L¹ is a linear C₁₋₆ alkylene. In embodiments, L¹ is unsubstituted C₁₋₆ alkylene. In embodiments, L¹ is unsubstituted branched C₁₋₆ alkylene. In embodiments, L¹ is unsubstituted linear C₁₋₆ alkylene. In embodiments, L¹ is substituted C₁₋₆ alkylene (e.g., comprising an OH group). In embodiments, L¹ is substituted branched C₁₋₆ alkylene (e.g., comprising an OH group). In embodiments, L¹ is substituted linear C₁₋₆ alkylene (e.g., comprising an OH group).

In embodiments, R² is CH₃.

In embodiments, a compound of Formula (I) has a structure according to Formula (VII).

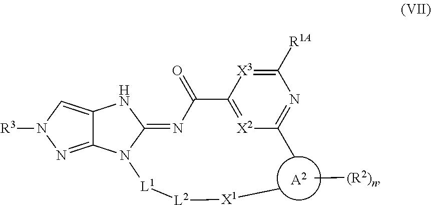

or a pharmaceutically acceptable salt thereof.

In embodiments, A², R^{1A}, R², R³, L¹, L², X¹, X², X³, and n are according to any embodiment described herein.

In embodiments, a compound of Formula (I) has a structure according to Formula (VIII).

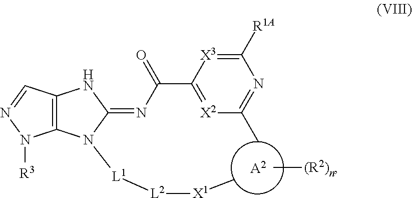

or a pharmaceutically acceptable salt thereof.

In embodiments, A², R^{1A}, R², R³, L¹, L², X¹, X², X³, and n are according to any embodiment described herein.

In embodiments, a compound has a structure according to Formula (IX),

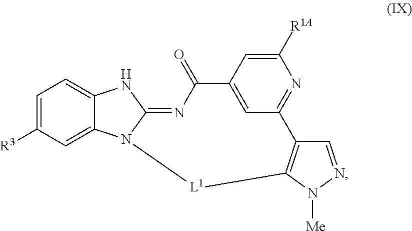

or a pharmaceutically acceptable salt thereof.

In embodiments, R³, L¹, and R^{1A} are according to any embodiment described herein.

In embodiments,
L¹ is C₁-C₆ alkylene optionally substituted by 1, 2, or 3 R¹³;
each R¹³ is independently unsubstituted C₁-C₃ alkyl; and
R^{1A} is independently unsubstituted C₁-C₆ alkyl or C₁-C₆ haloalkyl.

In embodiments, a compound has a structure according to Formula (X),

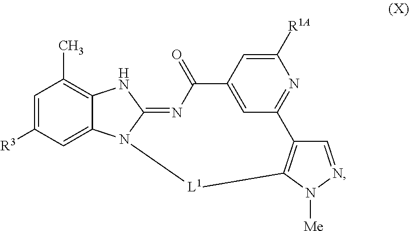

or a pharmaceutically acceptable salt thereof.

In embodiments, R³, L¹, and R^{1A} are according to any embodiment described herein.

In embodiments,
L¹ is C₁-C₆ alkylene optionally substituted by 1, 2, or 3 R¹³;
each R¹³ is independently unsubstituted C₁-C₃ alkyl; and
R^{1A} is independently unsubstituted C₁-C₆ alkyl or C₁-C₆ haloalkyl.

In embodiments, a compound has a structure according to Formula (XI),

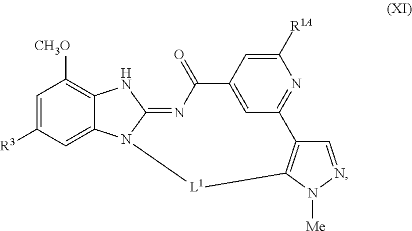

or a pharmaceutically acceptable salt thereof.

In embodiments, R³, L¹, and R^{1A} are according to any embodiment described herein.

In embodiments,
L¹ is C₁-C₆ alkylene optionally substituted by 1, 2, or 3 R¹³;
each R¹³ is independently unsubstituted C₁-C₃ alkyl; and
R^{1A} is independently unsubstituted C₁-C₆ alkyl or C₁-C₆ haloalkyl.

In embodiments, a compound has a structure according to:

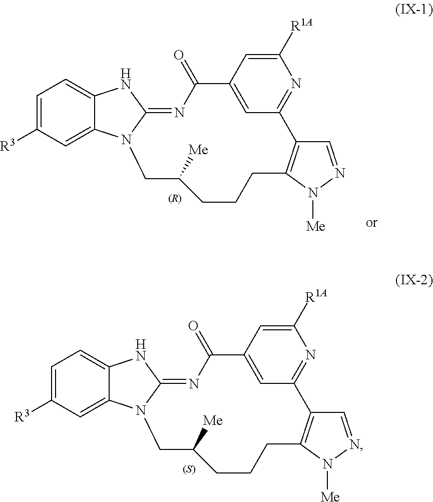

or a pharmaceutically acceptable salt thereof.

In embodiments, R³ and R^{1A} are according to any embodiment described herein.

In embodiments, a compound has a structure according to:

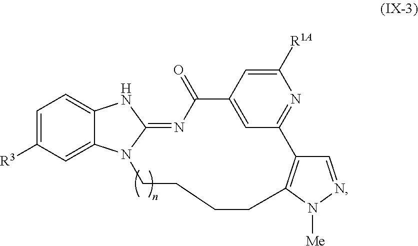

or a pharmaceutically acceptable salt thereof.

In embodiments, R³, R^{1A}, and n are according to any embodiment described herein.

In embodiments, n is 1, 2, or 3.

In embodiments, a compound has a structure according to:

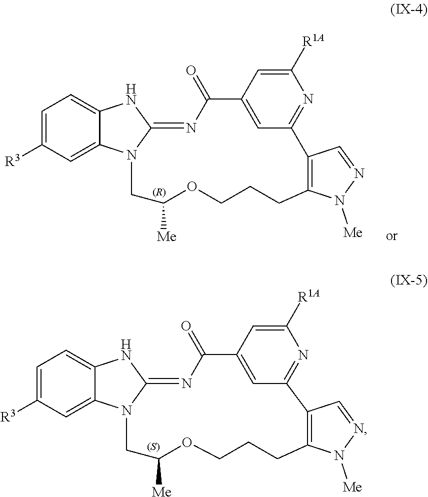

or a pharmaceutically acceptable salt thereof.

In embodiments, R³ and R^{1A} are according to any embodiment described herein.

In embodiments, a compound has a structure according to Formula (XII),

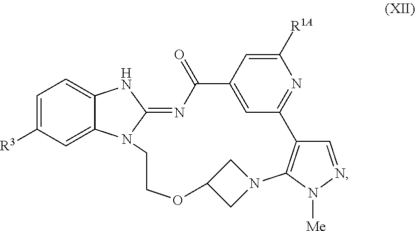

or a pharmaceutically acceptable salt thereof.

In embodiments, R³ and R^{1A} are according to any embodiment described herein.

In embodiments, R^{1A} is independently unsubstituted C₁-C₆ alkyl or C₁-C₆ haloalkyl.

In embodiments, a compound has a structure according to Formula (XIII),

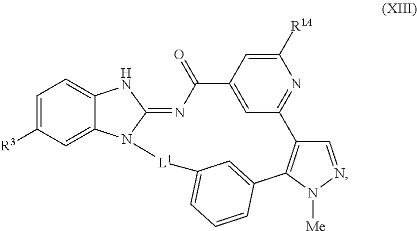

or a pharmaceutically acceptable salt thereof.

In embodiments, R³, L¹, and R^{1A} are according to any embodiment described herein.

In embodiments,
L¹ is C₁-C₃ alkylene optionally substituted by 1 or 2 R¹³;
each R¹³ is independently unsubstituted C₁-C₃ alkyl; and
R^{1A} is independently unsubstituted C₁-C₆ alkyl or C₁-C₆ haloalkyl.

In embodiments, L¹ is —CH₂— or —CH₂CHCH₃—.

In embodiments, a compound has a structure according to Formula (XIV),

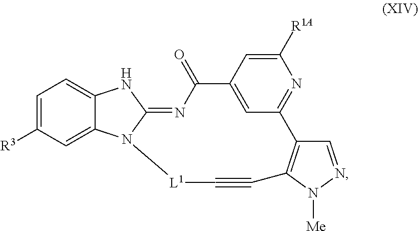

or a pharmaceutically acceptable salt thereof.

In embodiments, R³, L¹, and R^{1A} are according to any embodiment described herein.

In embodiments,
L¹ is C₂-C₄ alkylene optionally substituted by 1 or 2 R¹³;
each R¹³ is independently unsubstituted C₁-C₃ alkyl; and
R^{1A} is independently unsubstituted C₁-C₆ alkyl or C₁-C₆ haloalkyl.

### Exemplary Embodiments of Structural Features

Provided herein are exemplary embodiments of structural features which may be present in any formula described herein (e.g., any of one Formulas (I)-(XIV)). An exemplary embodiment of a structural feature may occur in combination with any other exemplary structural feature described herein.

In embodiments, X² is N. In embodiments, X² is CR^{1B} (e.g., CH).

In embodiments, X³ is N. In embodiments, X³ is CR^{1B} (e.g., CH).

In embodiments, each of X² and X³ is N.

In embodiments, each of X² and X³ is CR^{1B}. In embodiments, each of X² and X³ is CH.

In embodiments, one of X² and X³ is N, and the other is CR^{1B} (e.g., CH).

In embodiments, at least one of X² and X³ is N.

In embodiments, A¹ is phenylene. In embodiments, A¹ is unsubstituted phenylene. In embodiments, A¹ is substituted phenylene (e.g., comprising 1 or 2 substituents as described herein).

In embodiments, A¹ is 5- or 6-membered heteroarylene. Exemplary 5- to 6-membered heteroarylene includes but is not limited to pyridylene, pyrimidylene, pyrazolylene, thiazolylene, oxazolylene, and imidazolylene. In embodiments, A¹ is unsubstituted 5- or 6-membered heteroarylene. In embodiments, A¹ is substituted 5- or 6-membered heteroarylene (e.g., comprising 1 or 2 substituents as described herein).

In embodiments, A¹ is pyrazolylene. In embodiments, A¹ is unsubstituted pyrazolylene. In embodiments, A¹ is substituted pyrazolylene (e.g., comprising 1 or 2 substituents as described herein). In embodiments, A¹ is N-substituted pyrazolylene (e.g., N-methyl pyrazolylene).

In embodiments,

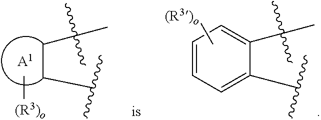

In embodiments,

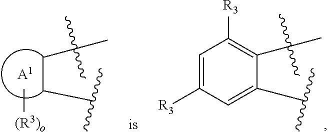

where each R³ is independently any embodiment described herein.

In embodiments,

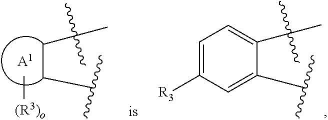

where R³ is independently any embodiment described herein.

In embodiments,

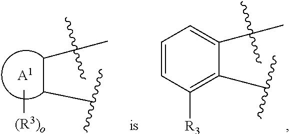

where R³ is independently any embodiment described herein.

In embodiments,

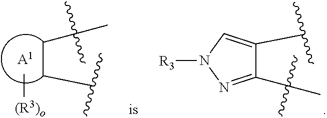

In embodiments,

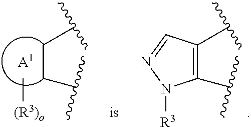

In embodiments, A² is phenyl. In embodiments, A² is unsubstituted phenyl. In embodiments, A² is substituted phenyl (e.g., comprising 1 or 2 substituents as described herein).

In embodiments, A² is naphthyl. In embodiments, A² is unsubstituted naphthyl. In embodiments, A² is substituted naphthyl (e.g., comprising 1 or 2 substituents as described herein).

In embodiments, A² is 5- to 13-membered heteroaryl (e.g., monocyclic or bicyclic heteroaryl). In embodiments, A² is a monocyclic 5- to 6-membered heteroaryl. Exemplary monocyclic 5- to 6-membered heteroaryls include but are not limited to pyridyl, pyrimidyl, pyrazolyl, thiazolyl, oxazolyl, and imidazolyl. In embodiments, A² is a bicyclic 8- to 12-membered heteroaryl (e.g., nitrogen-containing, bicyclic 8- to 12-membered heteroaryl). Exemplary bicyclic 8- to 12-membered heteroaryls include but are not limited to indolyl, benzimidazolyl, indazolyl, isoindolyl, pyrrolopyrimidyl, pyrrolopyridinyl, pyrazolopyrimidyl, pyrazolopyridinyl, benzotriazolyl, quinolyl, and isoquinolyl. In embodiments, A² is pyrazolyl.

In embodiments, A² is unsubstituted 5- to 13-membered heteroaryl (e.g., unsubstituted monocyclic or bicyclic heteroaryl). In embodiments, A² is unsubstituted monocyclic 5- to 6-membered heteroaryls. In embodiments, A² is unsubstituted pyridyl, unsubstituted pyrimidyl, unsubstituted pyrazolyl, unsubstituted thiazolyl, unsubstituted oxazolyl, or unsubstituted imidazolyl. In embodiments, A² is unsubstituted bicyclic 8- to 12-membered heteroaryl (e.g., unsubstituted nitrogen-containing, bicyclic 8- to 12-membered heteroaryl). In embodiments, A² is unsubstituted indolyl, unsubstituted benzimidazolyl, unsubstituted indazolyl, unsubstituted isoindolyl, unsubstituted pyrrolopyrimidyl, unsubstituted pyrrolopyridinyl, unsubstituted pyrazolopyrimidyl, unsubstituted pyrazolopyridinyl, unsubstituted benzotriazolyl, unsubstituted quinolyl, or unsubstituted isoquinolyl. In embodiments, A² is unsubstituted pyrazolyl.

In embodiments, A² is substituted 5- to 13-membered heteroaryl (e.g., substituted monocyclic or bicyclic heteroaryl comprising 1 or 2 substituents as described herein). In embodiments, A² is substituted monocyclic 5- to 6-membered heteroaryls. In embodiments, A² is substituted pyridyl, substituted pyrimidyl, substituted pyrazolyl, substituted thiazolyl, substituted oxazolyl, or substituted imidazolyl. In embodiments, A² is substituted bicyclic 8- to 12-membered heteroaryl (e.g., substituted nitrogen-containing, bicyclic 8- to 12-membered heteroaryl). In embodiments, A² is substituted indolyl, substituted benzimidazolyl, substituted indazolyl, substituted isoindolyl, substituted pyrrolopyrimidyl, substituted pyrrolopyridinyl, substituted pyrazolopyrimidyl, substituted pyrazolopyridinyl, substituted benzotriazolyl, substituted quinolyl, or substituted isoquinolyl. Exemplary substituent groups include but are not limited to methyl, halogen (e.g. F, Cl, Br, or I), and CN. In embodiments, A² is substituted pyrazolyl (e.g, N-substituted pyrazolyl such as N-methyl pyrazolyl).

In embodiments,

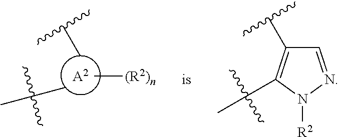

In embodiments,

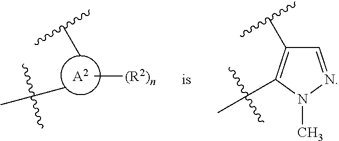

In embodiments, when X¹ is O, and both of X² and X³ are not N, then A² is naphthyl or a bicyclic 8- to 12-membered heteroaryl. In embodiments, X¹ is not O.

In embodiments, R^{1A} is H. In embodiments, R^{1A} is OH. In embodiments, R^{1A} is CN. In embodiments, R^{1A} is halogen (e.g., F, Cl, Br, or I). In embodiments, R^{1A} is C₁₋₆ aliphatic. In embodiments, R^{1A} is unsubstituted C₁₋₆ aliphatic. In embodiments, R^{1A} is substituted C₁₋₆ aliphatic (e.g., comprising 1, 2, or 3 substituent groups). In embodiments, R^{1A} is C₁₋₆ alkoxy. In embodiments, R^{1A} is unsubstituted C₁₋₆ alkoxy. In embodiments, R^{1A} is substituted C₁₋₆ alkoxy (e.g., comprising 1, 2, or 3 substituent groups). In embodiments, R^{1A} is NR⁶R⁷. In embodiments, R^{1A} is C(O)R⁸. In embodiments, R^{1A} is CO₂R⁸. In embodiments, R^{1A} is C(O)NR⁶R⁷. In embodiments, R^{1A} is NR⁹C(O)R⁸. In embodiments, R^{1A} is NR⁹CO₂R⁸. In embodiments, R^{1A} is NR⁹C(O)NR⁶R⁷. In embodiments, R^{1A} is R¹⁰.

In embodiments, R^{1A} is CH₃, CH₂F, CHF₂, or CF₃.

In embodiments, R^{1A} is CH₃.

In embodiments, R^{1A} is CH₂F, CHF₂, or CF₃.

In embodiments, R^{1B} is H.

In embodiments, R^{1B} is OH. In embodiments, R^{1B} is CN. In embodiments, R^{1B} is halogen (e.g., F, Cl, Br, or I). In embodiments, R^{1B} is C₁₋₆ aliphatic. In embodiments, R^{1B} is unsubstituted C₁₋₆ aliphatic. In embodiments, R^{1B} is substituted C₁₋₆ aliphatic (e.g., comprising 1, 2, or 3 substituent groups). In embodiments, R^{1B} is C₁₋₆ alkoxy. In embodiments, R^{1B} is unsubstituted C₁₋₆ alkoxy. In embodiments, R^{1B} is substituted C₁₋₆ alkoxy (e.g., comprising 1, 2, or 3 substituent groups). In embodiments, R^{1B} is NR⁶R⁷. In embodiments, R^{1B} is C(O)R⁸. In embodiments, R^{1B} is CO₂R′. In embodiments, R^{1B} is C(O)NR⁶R⁷. In embodiments, R^{1B} is NR⁹C(O)R⁸. In embodiments, R^{1B} is NR⁹CO₂R⁸. In embodiments, R^{1B} is NR⁹C(O)NR⁶R⁷. In embodiments, R^{1B} is R¹⁰.

In embodiments, R² is OH. In embodiments, R² is CN. In embodiments, R² is halogen (e.g., F, Cl, Br, or I). In embodiments, R² is C₁₋₆ aliphatic (e.g., methyl). In embodiments, R² is unsubstituted C₁₋₆ aliphatic (e.g., methyl). In embodiments, R² is substituted C₁₋₆ aliphatic (e.g., comprising 1, 2, or 3 substituent groups). In embodiments, R² is C₁₋₆ alkoxy. In embodiments, R² is unsubstituted C₁₋₆ alkoxy. In embodiments, R² is substituted C₁₋₆ alkoxy (e.g., comprising 1, 2, or 3 substituent groups). In embodiments, R² is NR⁶R⁷. In embodiments, R² is C(O)R⁸. In embodiments, R² is CO₂R⁸. In embodiments, R² is C(O)NR⁶R⁷. In embodiments, R² is NR⁹C(O)R⁸. In embodiments, R² is NR⁹CO₂R⁸. In embodiments, R² is NR⁹C(O)NR⁶R⁷. In embodiments, R² is R¹⁰. In embodiments, R² is OR¹⁰. In embodiments, R² is CH₂R¹⁰. In embodiments, R² is CH₂CH₂R¹⁰. In embodiments, R² is OCH₂R¹⁰. In embodiments, R² is OCH₂CH₂R¹⁰.

In embodiments, R² is methyl.

In embodiments, R³ is OH. In embodiments, R³ is CN. In embodiments, R³ is halogen (e.g., F, Cl, Br, or I). In embodiments, R³ is C₁₋₆ aliphatic. In embodiments, R³ is unsubstituted C₁₋₆ aliphatic. In embodiments, R³ is substituted C₁₋₆ aliphatic (e.g., comprising 1, 2, or 3 substituent groups). In embodiments, R³ is C₁₋₆ alkoxy. In embodiments, R³ is unsubstituted C₁₋₆ alkoxy. In embodiments, R³ is substituted C₁₋₆ alkoxy (e.g., comprising 1, 2, or 3 substituent groups). In embodiments, R³ is NR⁶R⁷. In embodiments, R³ is C(O)R⁸. In embodiments, R³ is CO₂R⁸. In embodiments, R³ is C(O)NR⁶R⁷. In embodiments, R³ is NR⁹C(O)R⁸. In embodiments, R³ is NR⁹CO₂R⁸. In embodiments, R³ is NR⁹C(O)NR⁶R⁷. In embodiments, R³ is R¹⁰. In embodiments, R³ is OR¹⁰. In embodiments, R³ is CH₂R¹⁰. In embodiments, R³ is CH₂CH₂R¹⁰. In embodiments, R³ is OCH₂R¹⁰. In embodiments, R³ is OCH₂CH₂R¹⁰.

In embodiments, R³ is
halogen;
NR⁶R⁷, wherein R⁶ and R⁷, together with the nitrogen atom to which they are attached, form a 5- to 7-membered heterocyclyl;
NR⁶R⁷, wherein each R⁶ and R⁷ is independently C₁-C₆ alkyl;
phenyl;
pyridyl;
C(O)R⁸, wherein R¹ is a 5- to 6-membered nitrogen-containing heterocyclyl;
R¹⁰, wherein R¹⁰ is a 5- to 6-membered nitrogen-containing heterocyclyl;
OR¹⁰, wherein R¹⁰ is a 5- to 6-membered nitrogen-containing heterocyclyl;
CH₂R¹⁰, wherein R¹⁰ is a 5- to 6-membered nitrogen-containing heterocyclyl;
CH₂CH₂R¹⁰, wherein R¹⁰ is a 5- to 6-membered nitrogen-containing heterocyclyl; or
OCH₂CH₂R¹⁰, wherein R¹⁰ is a 5- to 6-membered nitrogen-containing heterocyclyl.

In embodiments, R³ is halogen.

In embodiments, R³ is NR⁶R⁷, where R⁶ and R⁷, together with the nitrogen atom to which they are attached, form a 5- to 6-membered heterocyclyl.

In embodiments, R³ is NR⁶R⁷, wherein each R⁶ and R⁷ is independently C₁-C₆ alkyl (e.g., one R⁶ and R⁷ is unsubstituted C₁-C₆ alkyl, and the other is C₁-C₆ alkyl comprising an amino group, a monoalkylamino group, or a dialkylamino group).

In embodiments, R³ is unsubstituted or substituted phenyl or pyridyl. In embodiments, R³ is unsubstituted or substituted phenyl. In embodiments, R³ is unsubstituted or substituted pyridyl.

In embodiments, R³ is unsubstituted or substituted pyrrolidine, morpholine, piperidine, or piperazine.

In embodiments, R³ is C(O)R⁸, wherein R⁸ is unsubstituted or substituted pyrrolidine, morpholine, piperidine, or piperazine.

In embodiments, R³ is R¹⁰, OR¹⁰, CH₂R¹⁰, CH₂CH₂R¹⁰, or OCH₂CH₂R¹⁰, wherein R¹⁰ is unsubstituted or substituted pyrrolidine, morpholine, piperidine, or piperazine.

In embodiments, R³ is selected from the group consisting of

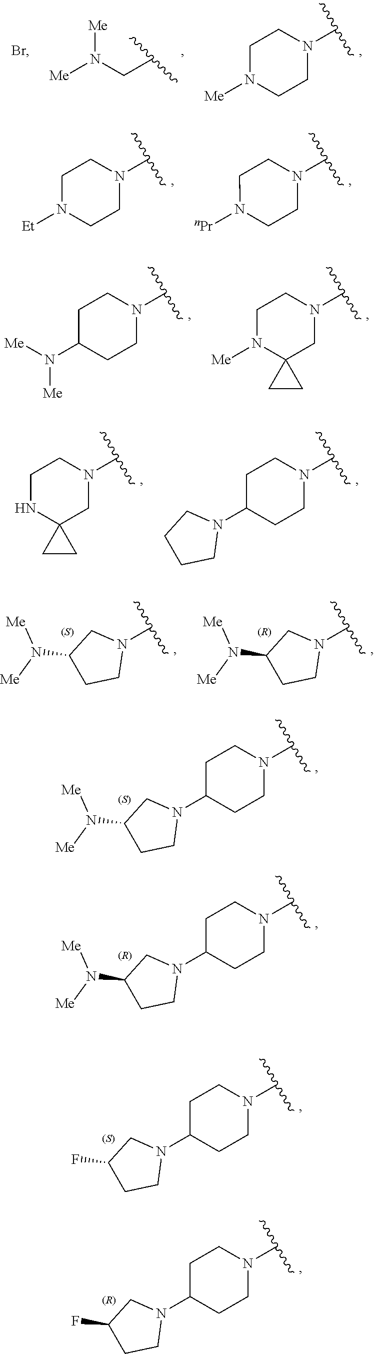

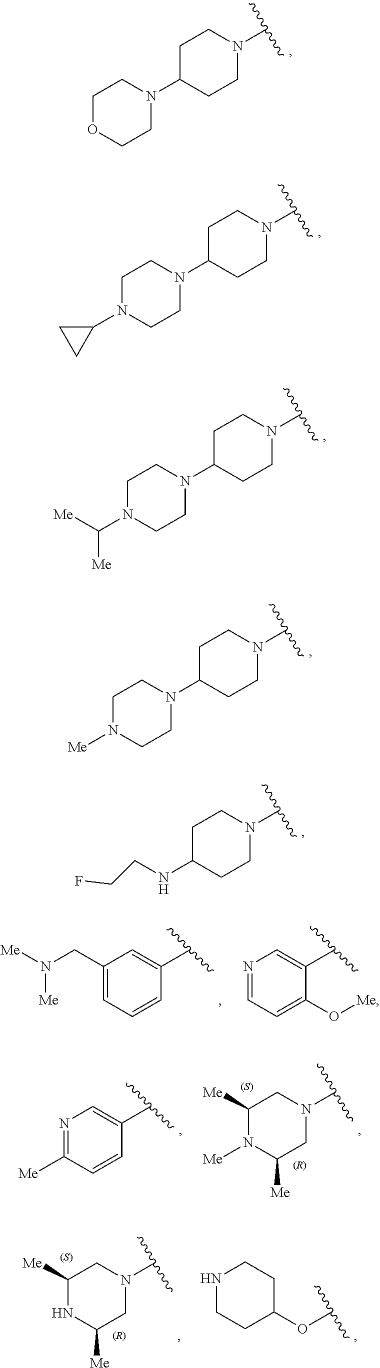

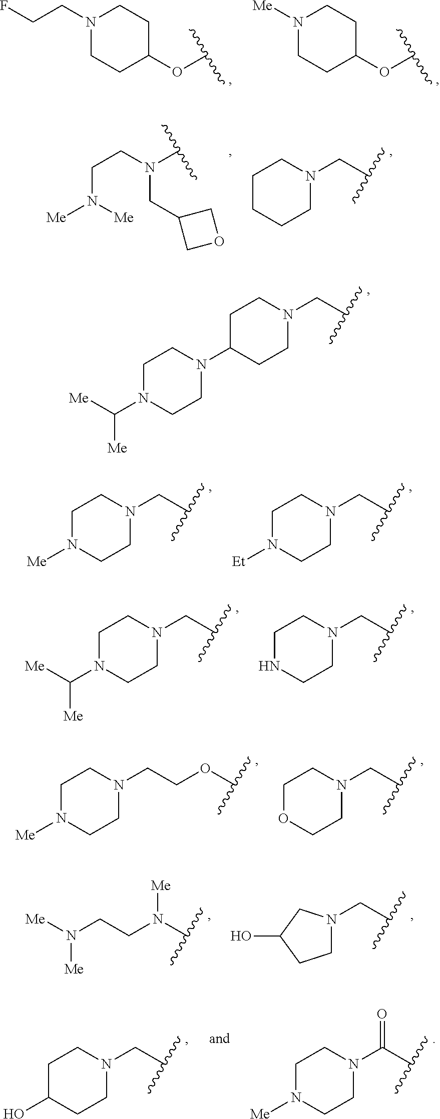

In embodiments, R³ is an unsubstituted group.

In embodiments, R³ is a group comprising 1, 2, 3, or 4 substituent groups. In embodiments, a substituent group is selected from halogen (e.g., F, Cl, Br, or I), C₁₋₆ aliphatic (e.g., methyl, monofluoromethyl, difluoromethyl, trifluoromethyl, ethyl, monofluoroethyl, propyl (e.g., n-propyl or isopropyl), butyl (e.g., n-butyl, sec-butyl, isobutyl, or tert-butyl)), amino (—NH₂), monoalkylamino (e.g., —NHCH₃), dialkylamino (e.g., —N(CH₃)₂), oxo (═O), C₃₋₆ cycloaliphatic (e.g., cyclopropyl, cyclobutyl, cyclopentyl, or cyclohexyl), or 3- to 6-membered heterocyclyl (e.g., oxetanyl, pyrrolidinyl, piperidinyl, piperazinyl, or morpholino), pyridyl, or phenyl. In embodiments, a cyclic group (e.g., a C₃₋₆ cycloaliphatic, a 3- to 6-membered heterocyclyl, a pyridyl, or a phenyl) comprises 1, 2, or 3 substituent groups (e.g., 1, 2, or 3 substituent groups selected from halogen (e.g., F, Cl, Br, or I), C₁₋₆ aliphatic (e.g., methyl, monofluoromethyl, difluoromethyl, trifluoromethyl, ethyl, monofluoroethyl, propyl (e.g., n-propyl or isopropyl), butyl (e.g., n-butyl, sec-butyl, isobutyl, or tert-butyl)), amino (—NH₂), monoalkylamino (e.g., —NHCH₃), dialkylamino (e.g., —N(CH₃)₂), and oxo (═O).

In embodiments, R⁴ is H. In embodiments, R⁴ is an N-protecting group (e.g., an amide group, a carbamate group, or a sulfonamide group). In embodiments, R⁴ is C₁₋₆ alkyl. In embodiments, a C₁₋₆ alkyl is unsubstituted. In embodiments, a C₁₋₆ alkyl is substituted (e.g., comprising 1, 2, or 3 substituent groups).

In embodiments, R⁵ is H.

In embodiments, R⁶ is H. In embodiments, R⁶ is C₁₋₆ alkyl. In embodiments, a C₁₋₆ alkyl is unsubstituted. In embodiments, a C₁₋₆ alkyl is substituted (e.g., comprising 1, 2, or 3 substituent groups).

In embodiments, R⁷ is H. In embodiments, R⁷ is C₁₋₆ alkyl. In embodiments, a C₁₋₆ alkyl is unsubstituted. In embodiments, a C₁₋₆ alkyl is substituted (e.g., comprising 1, 2, or 3 substituent groups).

In embodiments, R⁹ is H. In embodiments, R⁹ is C₁₋₆ alkyl. In embodiments, a C₁₋₆ alkyl is unsubstituted. In embodiments, a C₁₋₆ alkyl is substituted (e.g., comprising 1, 2, or 3 substituent groups).

In embodiments, R⁶ and R⁷, together with the nitrogen atom to which they are attached, form a 3- to 10-membered heterocyclyl (e.g., monocyclic or bicyclic heterocyclyl). In embodiments, a 3- to 10-membered heterocyclyl is unsubstituted. In embodiments, a 3- to 10-membered heterocyclyl is substituted (e.g., comprising 1, 2, or 3 substituent groups).

In embodiments, R⁶ and R⁹, together with the atoms to which they are attached, form a 3- to 10-membered heterocyclyl (e.g., monocyclic or bicyclic heterocyclyl). In embodiments, a 3- to 10-membered heterocyclyl is unsubstituted. In embodiments, a 3- to 10-membered heterocyclyl is substituted (e.g., comprising 1, 2, or 3 substituent groups).

In embodiments, R⁸ is C₁₋₆ aliphatic. In embodiments, R⁸ is C₃-C₁₀ cycloaliphatic (e.g., monocyclic or bicyclic cycloaliphatic). In embodiments, R⁸ is 3- to 10-membered heterocyclyl (e.g., monocyclic or bicyclic heterocyclyl). In embodiments, R⁸ is phenyl. In embodiments, R⁸ is naphthyl. In embodiments, R⁸ is 5- to 12-membered heteroaryl (e.g., monocyclic or bicyclic heteroaryl).

In embodiments, R⁸ is unsubstituted C₁₋₆ aliphatic. In embodiments, R⁸ is unsubstituted C₃-C₁₀ cycloaliphatic. In embodiments, R⁸ is unsubstituted 3- to 10-membered heterocyclyl. In embodiments, R⁸ is unsubstituted phenyl. In embodiments, R⁸ is unsubstituted naphthyl. In embodiments, R⁸ is unsubstituted 5- to 12-membered heteroaryl.

In embodiments, R⁸ is substituted C₁₋₆ aliphatic. In embodiments, R⁸ is substituted C₃-C₁₀ cycloaliphatic. In embodiments, R⁸ is substituted 3- to 10-membered heterocyclyl. In embodiments, R⁸ is substituted phenyl. In embodiments, R⁸ is substituted naphthyl. In embodiments, R⁸ is substituted 5- to 12-membered heteroaryl. In embodiments, a substituted group comprises 1, 2, or 3 substituent groups as described herein.

In embodiments, R⁸ and R⁹, together with the atoms to which they are attached, form a 3- to 10-membered heterocyclyl (e.g., monocyclic or bicyclic heterocyclyl). In embodiments, a 3- to 10-membered heterocyclyl is unsubstituted. In embodiments, a 3- to 10-membered heterocyclyl is substituted (e.g., comprising 1, 2, or 3 substituent groups).

In embodiments, R¹⁰ is C₃-C₁₀ cycloaliphatic (e.g., monocyclic or bicyclic cycloaliphatic). In embodiments, R¹⁰ is 3- to 10-membered heterocyclyl (e.g., monocyclic or bicyclic heterocyclyl). In embodiments, R¹⁰ is phenyl. In embodiments, R¹⁰ is naphthyl. In embodiments, R¹⁰ is 5- to 12-membered heteroaryl (e.g., monocyclic or bicyclic heteroaryl).

In embodiments, R¹⁰ is unsubstituted C₃-C₁₀ cycloaliphatic. In embodiments, R¹⁰ is unsubstituted 3- to 10-membered heterocyclyl. In embodiments, R¹⁰ is unsubstituted phenyl. In embodiments, R¹⁰ is unsubstituted naphthyl. In embodiments, R¹⁰ is unsubstituted 5- to 12-membered heteroaryl.

In embodiments, R¹⁰ is substituted C₃-C₁₀ cycloaliphatic. In embodiments, R¹⁰ is substituted 3- to 10-membered heterocyclyl. In embodiments, R¹⁰ is substituted phenyl. In embodiments, R¹⁰ is substituted naphthyl. In embodiments, R¹⁰ is substituted 5- to 12-membered heteroaryl. In embodiments, a substituted group comprises 1, 2, or 3 substituent groups as described herein.

In embodiments, n is 0. In embodiments, n is 1. In embodiments, n is 2. In embodiments, n is 1 or 2.

In embodiments, o is 0. In embodiments, o is 1. In embodiments, o is 2. In embodiments, o is 1 or 2.

In embodiments, X¹ is O. In embodiments, X¹ is X^{1A}.

In embodiments, X¹ is not O.

In embodiments, X^{1A} is a covalent bond.

In embodiments, X^{1A} is S.

In embodiments, X^{1A} is NR⁴ (e.g., NH, NCH₃, or nitrogen with a protecting group as described herein).

In embodiments, X^{1A} is C₁₋₆ alkylene (e.g., CH₂, (CH₂)₂, (CH₂)₃, (CH₂)₄, (CH₂)₅, or (CH₂)₆). In embodiments, a C₁₋₆ alkylene is unsubstituted. In embodiments, a C₁₋₆ alkylene is substituted (e.g., comprising 1, 2, or 3 substituent groups such as OH, oxo (═O), or unsubstituted C₁₋₃ alkyl). In embodiments, X^{1A} is a branched C₁₋₆ alkylene. In embodiments, X^{1A} is a linear C₁₋₆ alkylene. In embodiments, X^{1A} is unsubstituted branched C₁₋₆ alkylene. In embodiments, X^{1A} is unsubstituted linear C₁₋₆ alkylene. In embodiments, X^{1A} is substituted branched C₁₋₆ alkylene (e.g., comprising 1, 2, or 3 substituent groups such as OH, oxo (═O), or unsubstituted C₁₋₃ alkyl). In embodiments, X^{1A} is substituted linear C₁₋₆ alkylene (e.g., comprising 1, 2, or 3 substituent groups such as OH, oxo (═O), or unsubstituted C₁₋₃ alkyl).

In embodiments, X^{1A} is C₂₋₆ alkenylene (e.g., C₂H₄, C₃H₆, C₄HS, C₅H₁₀, or C₆H₁₂). In embodiments, a C₂₋₆ alkenylene is unsubstituted. In embodiments, a C₂₋₆ alkenylene is substituted (e.g., comprising 1, 2, or 3 substituent groups such as OH, oxo (═O), or unsubstituted C₁₋₃ alkyl).

In embodiments, X^{1A} is C₂₋₆ alkynylene (e.g., C₂H₂, C₃H₄, C₄H₆, C₅H₈, or C₆H₁₀). In embodiments, a C₂₋₆ alkynylene is unsubstituted. In embodiments, a C₂₋₆ alkynylene is substituted (e.g., comprising 1, 2, or 3 substituent groups such as OH, oxo (═O), or unsubstituted C₁₋₃ alkyl).

In embodiments, L¹ is a covalent bond.

In embodiments, L¹ is a C₁₋₆ alkylene (e.g., CH₂, (CH₂)₂, (CH₂)₃, (CH₂)₄, (CH₂)₅, or (CH₂)₆). In embodiments, L¹ is a branched C₁₋₆ alkylene. In embodiments, L¹ is a linear C₁₋₆ alkylene. In embodiments, L¹ is unsubstituted C₁₋₆ alkylene. In embodiments, L¹ is unsubstituted branched C₁₋₆ alkylene. In embodiments, L¹ is unsubstituted linear C₁₋₆ alkylene. In embodiments, L¹ is substituted C₁₋₆ alkylene (e.g., comprising 1, 2, or 3 substituent groups such as OH, oxo (═O), or unsubstituted C₁₋₃ alkyl). In embodiments, L¹ is substituted branched C₁₋₆ alkylene (e.g., comprising 1, 2, or 3 substituent groups such as OH, oxo (═O), or unsubstituted C₁₋₃ alkyl). In embodiments, L¹ is substituted linear C₁₋₆ alkylene (e.g., comprising 1, 2, or 3 substituent groups such as OH, oxo (═O), or unsubstituted C₁₋₃ alkyl).

In embodiments, L¹ is —(CH₂)₃— or —(CH₂)₄—.

In embodiments, L¹ is selected from the following group of substructures:

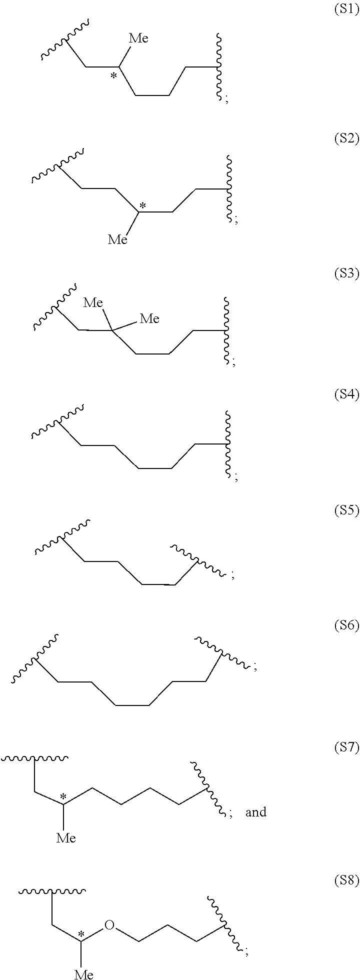

wherein a carbon marked by an asterisk (*) is racemic or has the (R)- or (S)-stereochemistry. In embodiments, a carbon marked by an asterisk (*) is racemic. In embodiments, a carbon marked by an asterisk (*) has the (R)-stereochemistry. In embodiments, a carbon marked by an asterisk (*) has the (S)-stereochemistry.

In embodiments, L¹ is substructure (S1). In embodiments, L¹ is substructure (S2). In embodiments, L¹ is substructure (S3). In embodiments, L¹ is substructure (S4). In embodiments, L¹ is substructure (S5). In embodiments, L¹ is substructure (S6). In embodiments, L¹ is substructure (S7). In embodiments, L¹ is substructure (S8).

In embodiments, L² is a covalent bond.

In embodiments, L² is a C₂₋₆ alkenylene (e.g., C₂H₄, C₃H₆, C₄HS, C₅H₁₀, or C₆H₁₂). In embodiments, L² is unsubstituted C₂₋₆ alkenylene. In embodiments, L² is substituted C₂₋₆ alkenylene (e.g., comprising 1, 2, or 3 substituent groups). In embodiments, L² is C₂₋₆ alkenylene substituted with C₁₋₆ alkyl. In embodiments, the substituent groups on L², together with the atoms to which they are attached, form a cycloalkenylene (e.g., cyclopentylene or cyclohexylene).

In embodiments, L² is a C₂₋₆ alkynylene (e.g., C₂H₂, C₃H₄, C₄H₆, C₅H₈, or C₆H₁₀). In embodiments, L² is unsubstituted C₂₋₆ alkynylene. In embodiments, L² is substituted C₂₋₆ alkynylene (e.g., comprising 1, 2, or 3 substituent groups).

In embodiments, L² is a C₃₋₆ cycloalkylene (e.g., cyclopropylene, cyclobutylene, cyclopentylene, or cyclohexylene). In embodiments, L² is unsubstituted C₃₋₆ cycloalkylene. In embodiments, L² is substituted C₃₋₆ cycloalkylene (e.g., comprising 1, 2, or 3 substituent groups). Exemplary substituent groups include but are not limited to OH, oxo (═O), CN, halogen, C₁₋₆ alkyl, and C₁₋₆ alkoxy.

In embodiments, L² is a 3- to 10-membered heterocyclylene (e.g., monocyclic or bicyclic heterocyclylene). In embodiments, L² is unsubstituted 3- to 10-membered heterocyclylene. In embodiments, L² is substituted 3- to 10-membered heterocyclylene (e.g., comprising 1, 2, or 3 substituent groups). Exemplary substituent groups include but are not limited to OH, CN, halogen (e.g., F, Cl, Br, or I), C₁₋₆ alkyl, and C₁₋₆ alkoxy.

In embodiments, L² is a phenylene. In embodiments, L² is unsubstituted phenylene. In embodiments, L² is substituted phenylene (e.g., comprising 1, 2, or 3 substituent groups). Exemplary substituent groups include but are not limited to OH, CN, halogen (e.g., F, Cl, Br, or I), C₁₋₆ alkyl, and C₁₋₆ alkoxy.

In embodiments, L² is a 5- or 6-membered heteroarylene. In embodiments, L² is unsubstituted 5- or 6-membered heteroarylene. In embodiments, L² is substituted 5- or 6-membered heteroarylene (e.g., comprising 1, 2, or 3 substituent groups). Exemplary substituent groups include but are not limited to OH, CN, halogen (e.g., F, Cl, Br, or I), C₁₋₆ alkyl, and C₁₋₆ alkoxy.

In embodiments, -L¹-L²-X^{1A}— is

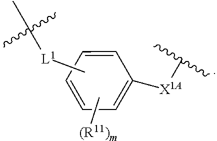

In embodiments, -L¹-L²-X^{1A}— is

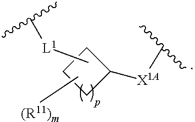

In embodiments, -L¹-L²-X^{1A}— is

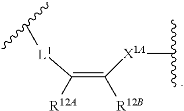

In embodiments, -L¹-L²-X^{1A}— is

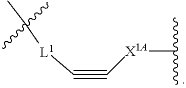

In embodiments, -L¹-L²-X^{1A}— is substructure (S1). In embodiments, -L¹-L²-X^{1A}— is substructure (S2). In embodiments, -L¹-L²-X^{1A}— is substructure (S3). In embodiments, -L¹-L²-X^{1A}— is substructure (S4). In embodiments, -L¹-L²-X^{1A}— is substructure (S5). In embodiments, -L¹-L²-X^{1A}— is substructure (S6). In embodiments, -L¹-L²-X^{1A}— is substructure (S7). In embodiments, -L¹-L²-X^{1A}— is substructure (S8).

Deuterated Compounds

Compounds described herein can comprise atoms that exhibit their natural isotopic abundances, or one or more of the atoms may be artificially enriched in a particular isotope having the same atomic number, but an atomic mass or mass number different from the atomic mass or mass number predominately found in nature. The term “isotopologue” refers to a species that has the same chemical structure and formula as a specific compound provided herein, with the exception of the positions of isotopic substitution and/or level of isotopic enrichment at one or more positions, e.g., hydrogen vs. deuterium. The present invention is meant to include all suitable isotopic variations of the compounds of the compounds described herein. For example, different isotopic forms of hydrogen (H) include protium (¹H), deuterium (²H), and tritium (³H), as well as compositions enriched in isotopologues of any compound described herein.

In embodiments, one or more of the hydrogens of the compounds described herein is replaced by a deuterium. When a position is designated as “H” or “hydrogen”, the position is understood to have hydrogen at its natural abundance isotopic composition. When a position is designated as “²H” or “deuterium”, the position is understood to have deuterium at an abundance that is at least 3340 times greater than the natural abundance of deuterium, which is 0.015% (i.e., the term “²H” or “deuterium” indicates at least 50.1% incorporation of deuterium). Accordingly, the invention also features compositions enriched in deuterated compounds.

In embodiments, compositions of any compound provided herein may have an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.500 deuterium incorporation).

### Exemplary Compounds

Exemplary compounds include those of Table 1.

TABLE 1
Exemplary Compounds

| | | | example # |
|---|---|---|---|
| | | ESI-MS | followed for |
| | | m/z | compound |
| No. | Structure | [M + H]⁺ | synthesis |
| (1) | 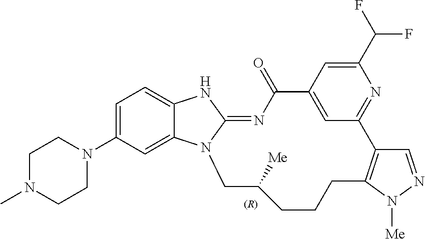 | — | — |
| (2) | 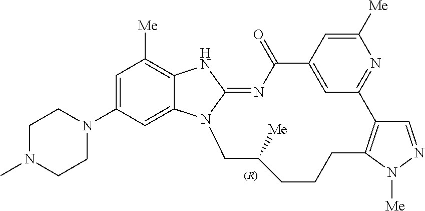 | 527.2 | 1 |
| (3) | 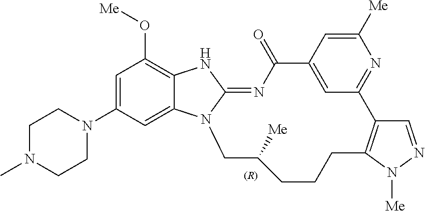 | 543.2 | 1 |
| (4) | 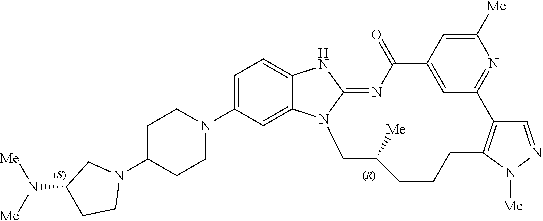 | 610.4 | 1 |
| (5) | 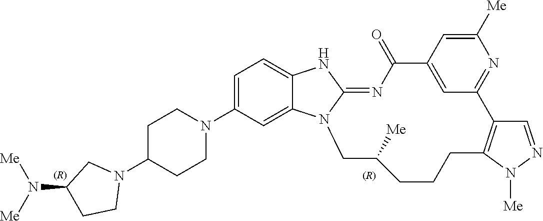 | 610.3 | 1 |
| (6) | 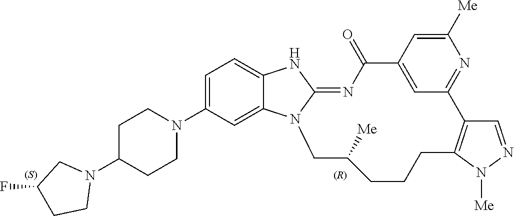 | 585.3 | 1 |
| (7) | 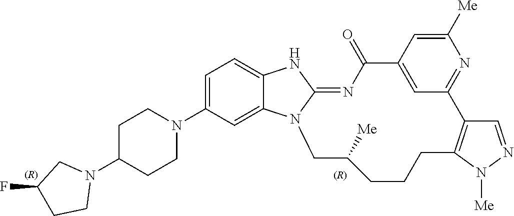 | 505.3 | 1 |
| (8) | 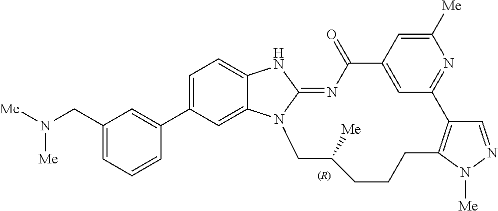 | 548.3 | 2 |
| (9) | 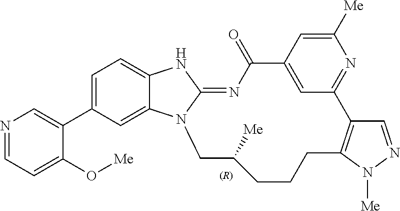 | 522.2 | 2 |
| (10) | 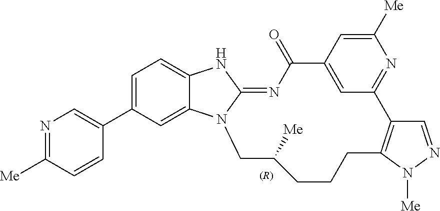 | 506.2 | 2 |
| (11) | 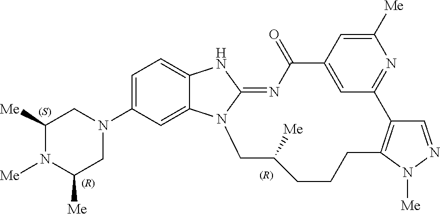 | 541.3 | 1 |
| (12) | 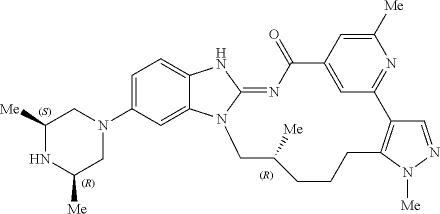 | 527.2 | 1 |
| (13) | 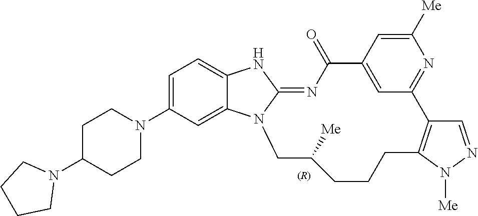 | 567.3 | 1 |
| (14) | 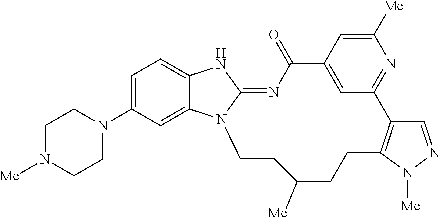 [SMILES: Cc1cc2cc(n1)-c1cnn(C)c1CCC(C)CCN1/C(=N/C2=O)Nc2ccc(N3CCN(C)CC3)cc21] | 513.3 | 1 |
| (15) | 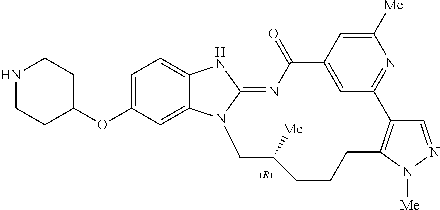 | 514.2 | 3 |
| (16) | 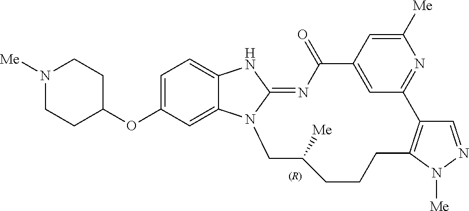 | 528.3 | 3 |
| (17) | 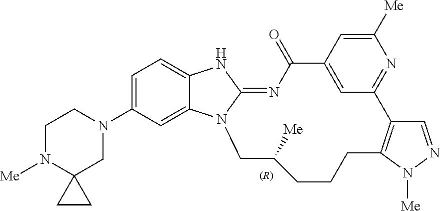 | 539.3 | 1 |
| (18) | 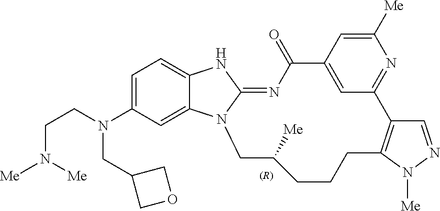 | 571.3 | 1 |
| (19) | 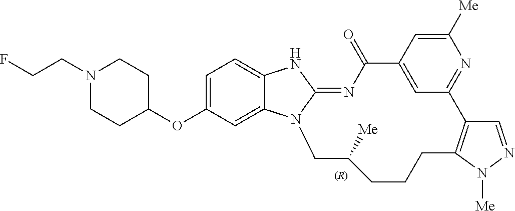 | 560.3 | 3 |
| (20) | 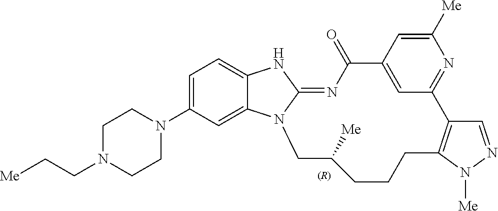 | 541.3 | 1 |
| (21) | 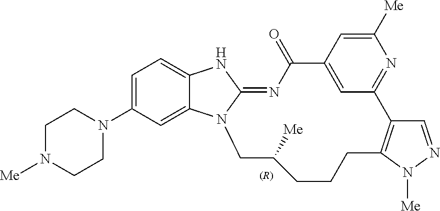 | 513.3 | 1 |
| (22) | 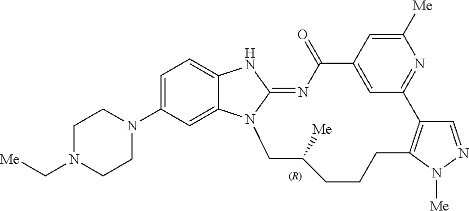 | 527.3 | 1 |
| (23) | 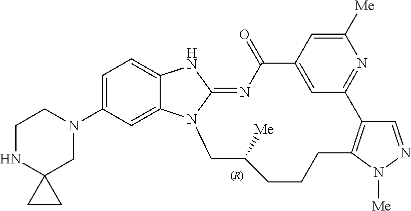 | 525.3 | 1 |
| (24) | 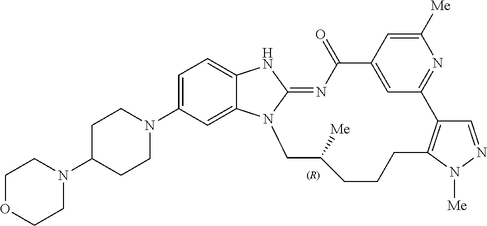 | 583.3 | 1 |
| (25) | 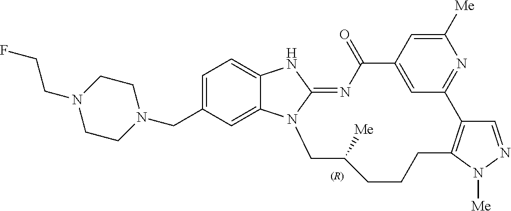 | 559.3 | 4 |
| (26) | 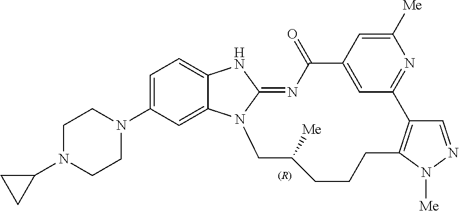 | 539.3 | 1 |
| (27) | 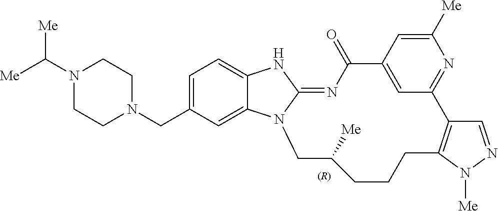 | 555.4 | 4 |
| (28) | 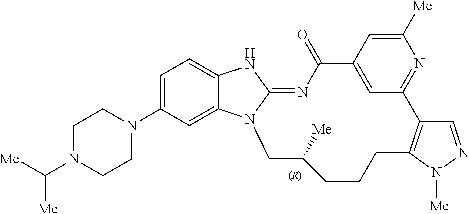 | 541.3 | 1 |
| (29) | 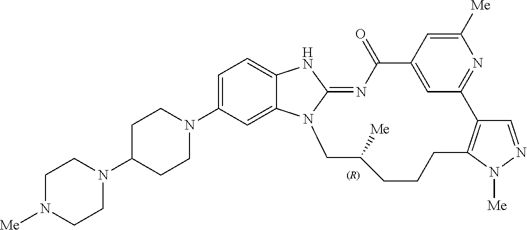 | 596.4 | 1 |
| (30) | 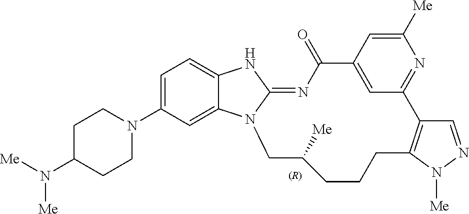 | 541.2 | 1 |
| (31) | 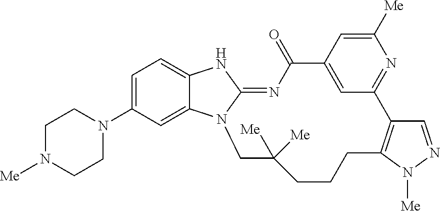 [SMILES: Cc1cc2cc(n1)-c1cnn(C)c1CCCC(C)(C)CN1/C(=N/C2=O)Nc2ccc(N3CCN(C)CC3)cc21] | 527.3 | 1 |
| (32) | 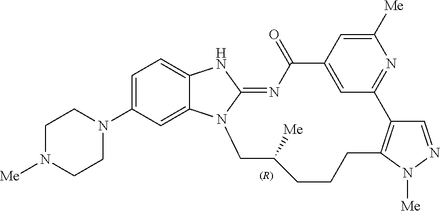 | 513.3 | 1 |
| (33) | 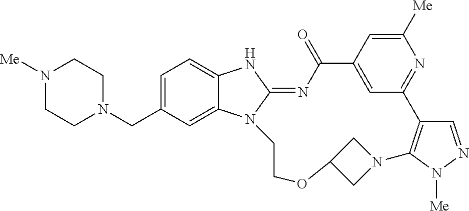 [SMILES: Cc1cc2cc(n1)-c1cnn(C)c1N1CC(C1)OCCN1/C(=N/C2=O)Nc2ccc(CN3CCN(C)CC3)cc21] | 542.2 | 5 |
| (34) | 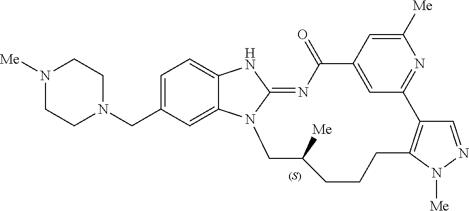 | 527.3 | 4 |
| (35) | 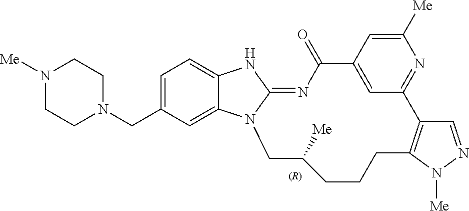 | 527.1 | 4 |
| (36) | 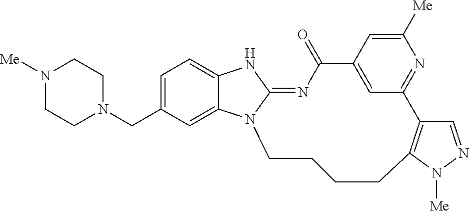 [SMILES: Cc1cc2cc(n1)-c1cnn(C)c1CCCCN1/C(=N/C2=O)Nc2ccc(CN3CCN(C)CC3)cc21] | 499.2 | 4 |
| (37) | 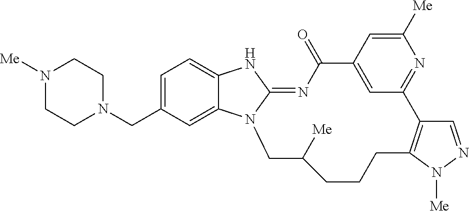 [SMILES: Cc1cc2cc(n1)-c1cnn(C)c1CCCC(C)CN1/C(=N/C2=O)Nc2ccc(CN3CCN(C)CC3)cc21] | 527.1 | 4 |
| (38) | 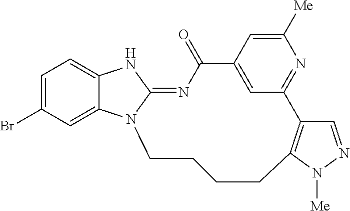 [SMILES: Cc1cc2cc(n1)-c1cnn(C)c1CCCCN1/C(=N/C2=O)Nc2ccc(Br)cc21] | — | — |
| (39) | 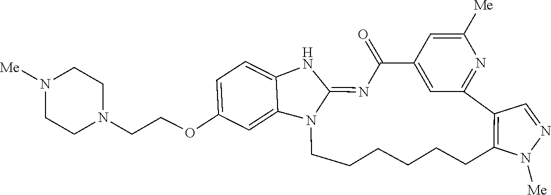 [SMILES: Cc1cc2cc(n1)-c1cnn(C)c1CCCCCCN1/C(=N/C2=O)Nc2ccc(OCCN3CCN(C)CC3)cc21] | 557.1 | 3 |
| (40) | 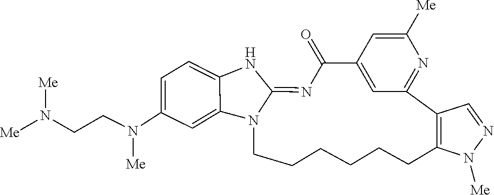 [SMILES: Cc1cc2cc(n1)-c1cnn(C)c1CCCCCCN1/C(=N/C2=O)Nc2ccc(N(C)CCN(C)C)cc21] | 515.2 | 1 |
| (41) | 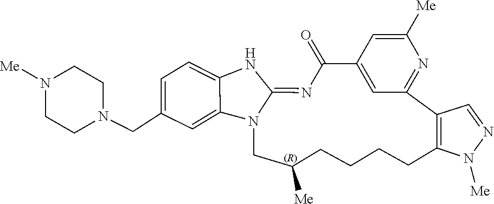 | 541.2 | 4 |
| (42) | 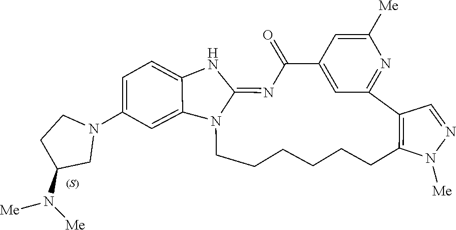 | 527.2 | 1 |
| (43) | 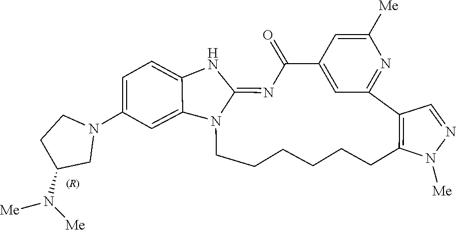 | 527.2 | 1 |
| (44) | 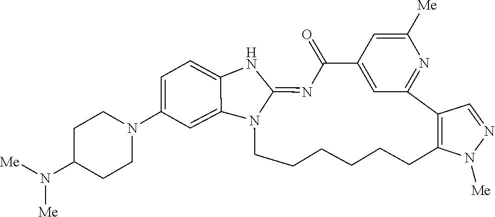 [SMILES: Cc1cc2cc(n1)-c1cnn(C)c1CCCCCCN1/C(=N/C2=O)Nc2ccc(N3CCC(N(C)C)CC3)cc21] | 541.2 | 1 |
| (45) | 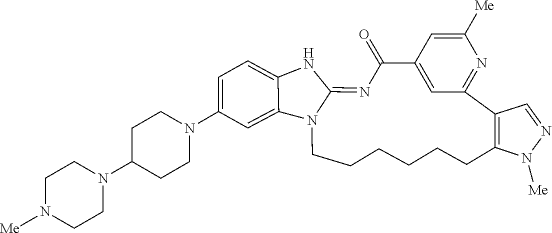 [SMILES: Cc1cc2cc(n1)-c1cnn(C)c1CCCCCCN1/C(=N/C2=O)Nc2ccc(N3CCC(N4CCN(C)CC4)CC3)cc21] | 596.3 | 1 |
| (46) | 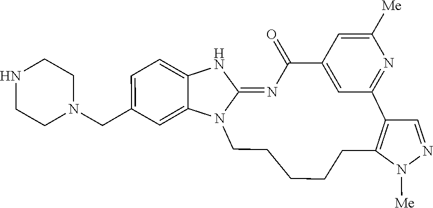 [SMILES: Cc1cc2cc(n1)-c1cnn(C)c1CCCCCN1/C(=N/C2=O)Nc2ccc(CN3CCNCC3)cc21] | 499.2 | 4 |
| (47) | 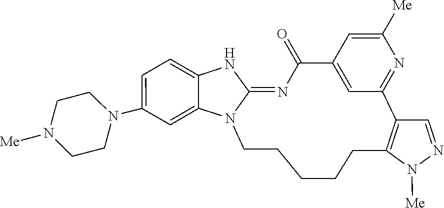 [SMILES: Cc1cc2cc(n1)-c1cnn(C)c1CCCCCN1/C(=N/C2=O)Nc2ccc(N3CCN(C)CC3)cc21] | 499.2 | 1 |
| (48) | 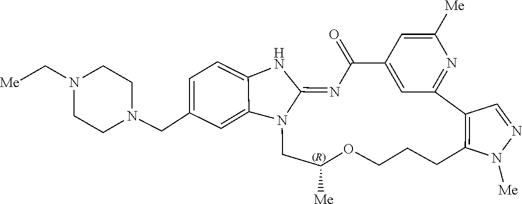 | 557.3 | 6 |
| (49) | 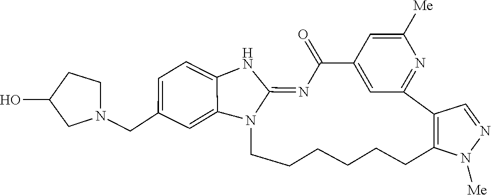 [SMILES: Cc1cc2cc(n1)-c1cnn(C)c1CCCCCCN1/C(=N/C2=O)Nc2ccc(CN3CCC(O)C3)cc21] | 514.2 | 4 |
| (50) | 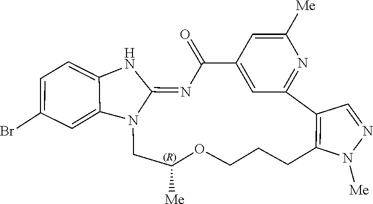 | — | — |
| (51) | 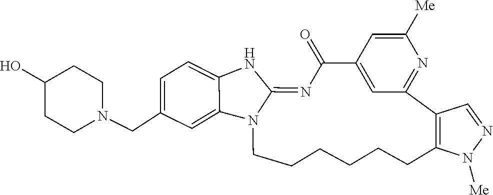 [SMILES: Cc1cc2cc(n1)-c1cnn(C)c1CCCCCCN1/C(=N/C2=O)Nc2ccc(CN3CCC(O)CC3)cc21] | 528.2 | 4 |
| (52) | 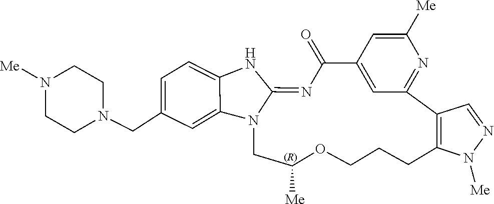 | 543.2 | 6 |
| (53) | 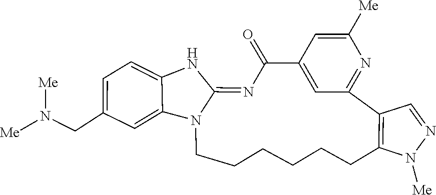 [SMILES: Cc1cc2cc(n1)-c1cnn(C)c1CCCCCCN1/C(=N/C2=O)Nc2ccc(CN(C)C)cc21] | 472.3 | 4 |
| (54) | 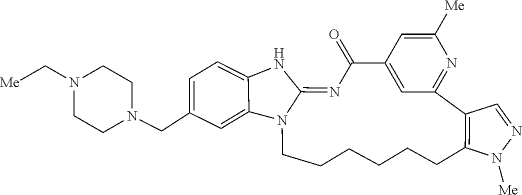 [SMILES: CCN1CCN(Cc2ccc3c(c2)N2CCCCCCc4c(cnn4C)-c4cc(cc(C)n4)C(=O)/N=C/2N3)CC1] | 541.2 | 4 |
| (55) | 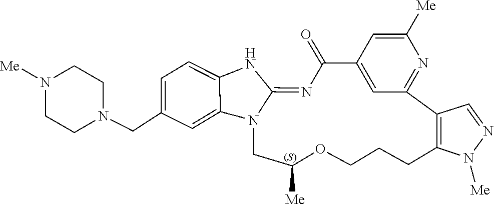 | 543.2 | 6 |
| (56) | 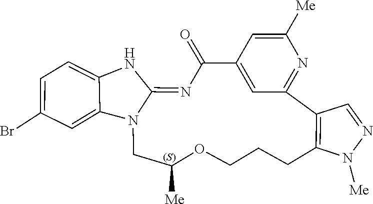 | — | — |
| (57) | 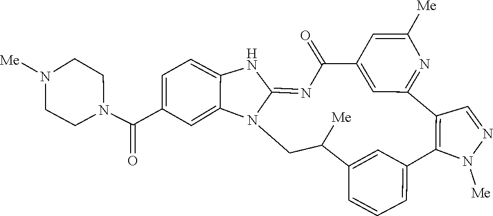 [SMILES: Cc1cc2cc(n1)-c1cnn(C)c1-c1cccc(c1)C(C)CN1/C(=N/C2=O)Nc2ccc(C(=O)N3CCN(C)CC3)cc21] | 575.1 | 7 |
| (58) | 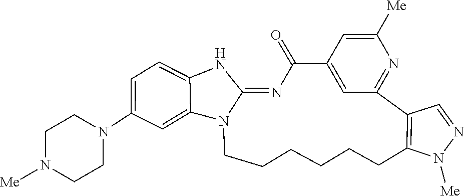 [SMILES: Cc1cc2cc(n1)-c1cnn(C)c1CCCCCCN1/C(=N/C2=O)Nc2ccc(N3CCN(C)CC3)cc21] | 513.2 | 1 |
| (59) | 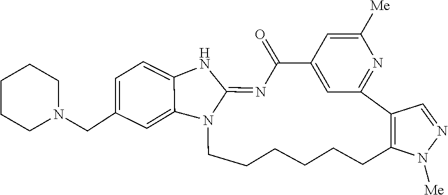 [SMILES: Cc1cc2cc(n1)-c1cnn(C)c1CCCCCCN1/C(=N/C2=O)Nc2ccc(CN3CCCCC3)cc21] | 512.2 | 4 |
| (60) | 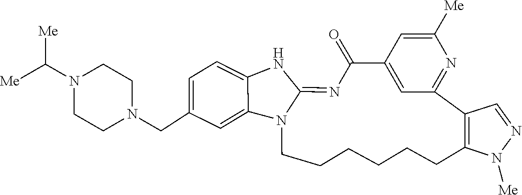 [SMILES: Cc1cc2cc(n1)-c1cnn(C)c1CCCCCCN1/C(=N/C2=O)Nc2ccc(CN3CCN(C(C)C)CC3)cc21] | 555.5 | 4 |
| (61) | 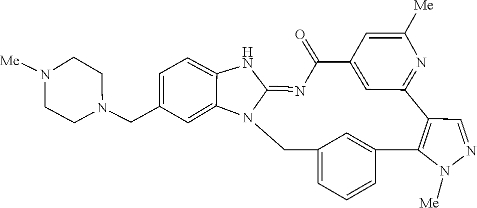 [SMILES: Cc1cc2cc(n1)-c1cnn(C)c1-c1cccc(c1)CN1/C(=N/C2=O)Nc2ccc(CN3CCN(C)CC3)cc21] | 533.1 | 7 |
| (62) | 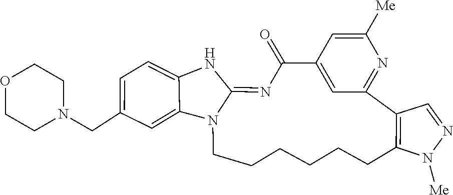 [SMILES: Cc1cc2cc(n1)-c1cnn(C)c1CCCCCCN1/C(=N/C2=O)Nc2ccc(CN3CCOCC3)cc21] | 514.1 | 4 |
| (63) | 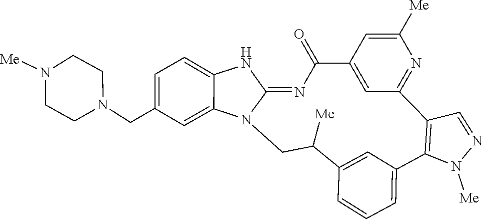 [SMILES: Cc1cc2cc(n1)-c1cnn(C)c1-c1cccc(c1)C(C)CN1/C(=N/C2=O)Nc2ccc(CN3CCN(C)CC3)cc21] | 561.1 | 7 |
| (64) | 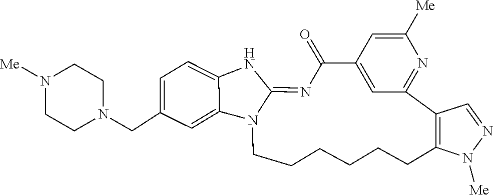 [SMILES: Cc1cc2cc(n1)-c1cnn(C)c1CCCCCCN1/C(=N/C2=O)Nc2ccc(CN3CCN(C)CC3)cc21] | 527.4 | 4 |
| (65) | 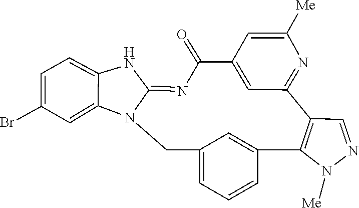 [SMILES: Cc1cc2cc(n1)-c1cnn(C)c1-c1cccc(c1)CN1/C(=N/C2=O)Nc2ccc(Br)cc21] | — | — |
| (66) | 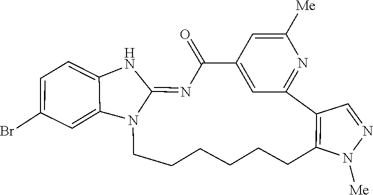 [SMILES: Cc1cc2cc(n1)-c1cnn(C)c1CCCCCCN1/C(=N/C2=O)Nc2ccc(Br)cc21] | — | — |
| (67) | 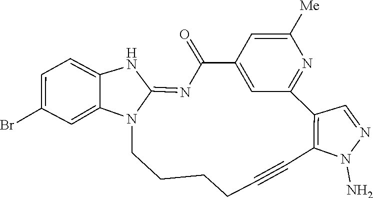 [SMILES: Cc1cc2cc(n1)-c1cnn(N)c1C#CCCCCN1/C(=N/C2=O)Nc2ccc(Br)cc21] | — | — |
| (68) | 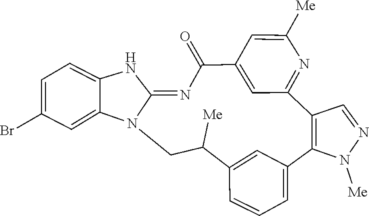 [SMILES: Cc1cc2cc(n1)-c1cnn(C)c1-c1cccc(c1)C(C)CN1/C(=N/C2=O)Nc2ccc(Br)cc21] | — | — |
| (69) | 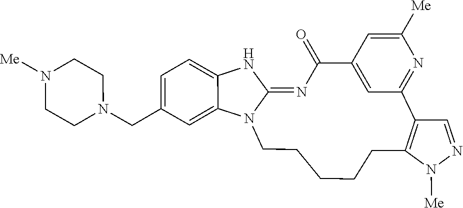 [SMILES: Cc1cc2cc(n1)-c1cnn(C)c1CCCCCN1/C(=N/C2=O)Nc2ccc(CN3CCN(C)CC3)cc21] | 513.2 | 4 |
| (70) | 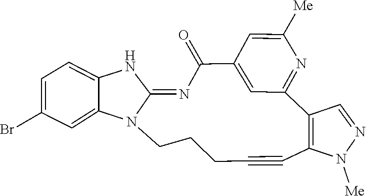 [SMILES: Cc1cc2cc(n1)-c1cnn(C)c1C#CCCCN1/C(=N/C2=O)Nc2ccc(Br)cc21] | — | — |
| (71) | 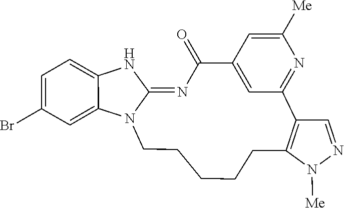 [SMILES: Cc1cc2cc(n1)-c1cnn(C)c1CCCCCN1/C(=N/C2=O)Nc2ccc(Br)cc21] | — | — |

In embodiments, compounds described herein can be potent, reversible inhibitors of kinases such as EGFR. Accordingly, in embodiments, compounds described herein (e.g., any compound of Formulas (I)-(XIV), including as exemplified by any of Compounds (1)-(71)) do not comprise functional groups selected from acrylamides, vinyl sulfonates, quinones, alkynyl amides, propargylic acid derivatives, u-halo ketones, thiocyanates, nitriles, epoxides, sulfonyl fluorides, and cyclic 1,3-diketones as permitted groups for any variable in that structure.

Synthetic Methods

Compounds described herein can be prepared according to methods known in the art. For example, the exemplary synthetic methods described in the instant Examples can be used to prepare still other compounds of the invention.

Accordingly, disclosed compounds can generally be synthesized by an appropriate combination of generally well known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Aldrich Chemical Company (Milwaukee, Wis.) or can be readily prepared by those skilled in the art using commonly employed synthetic methodology.

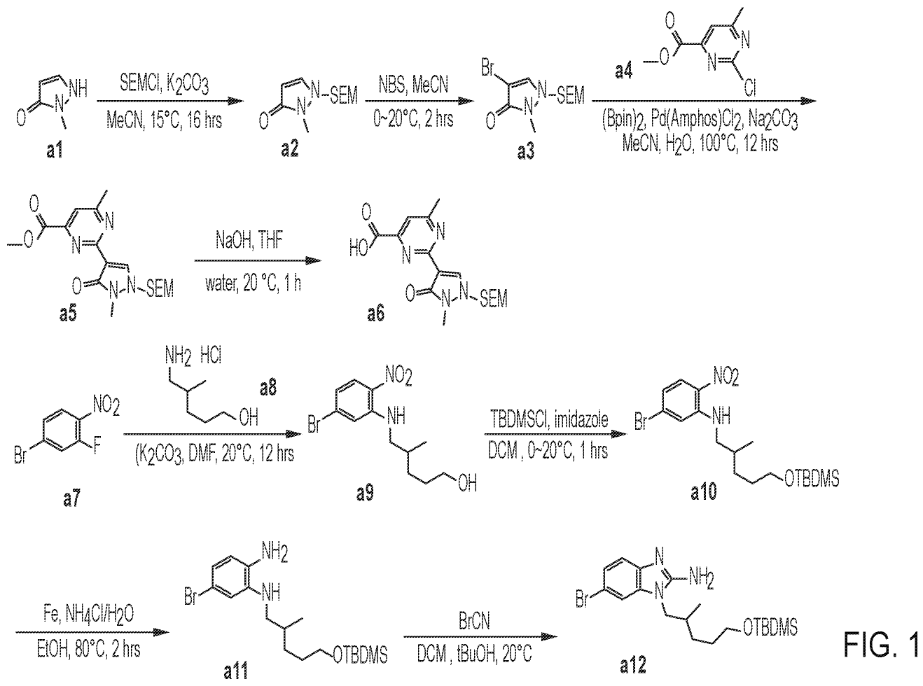

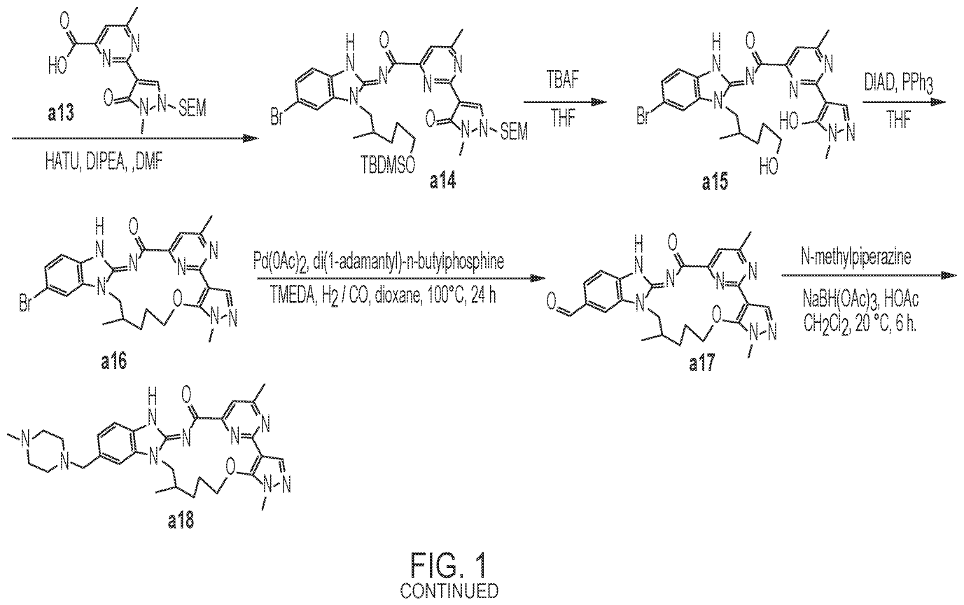

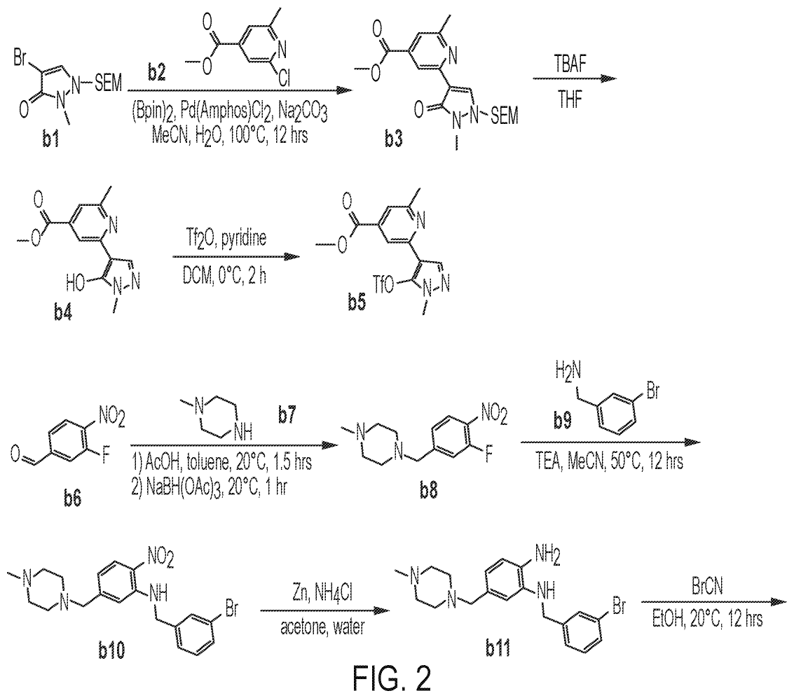

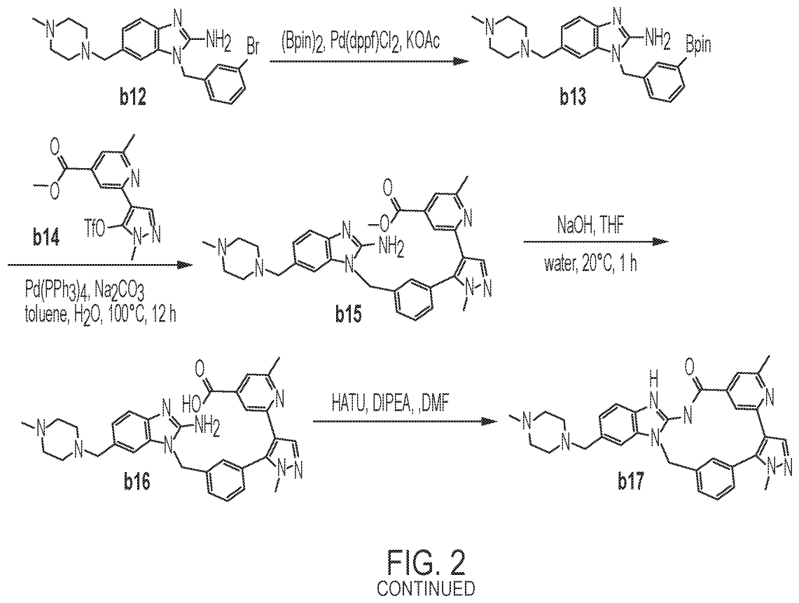

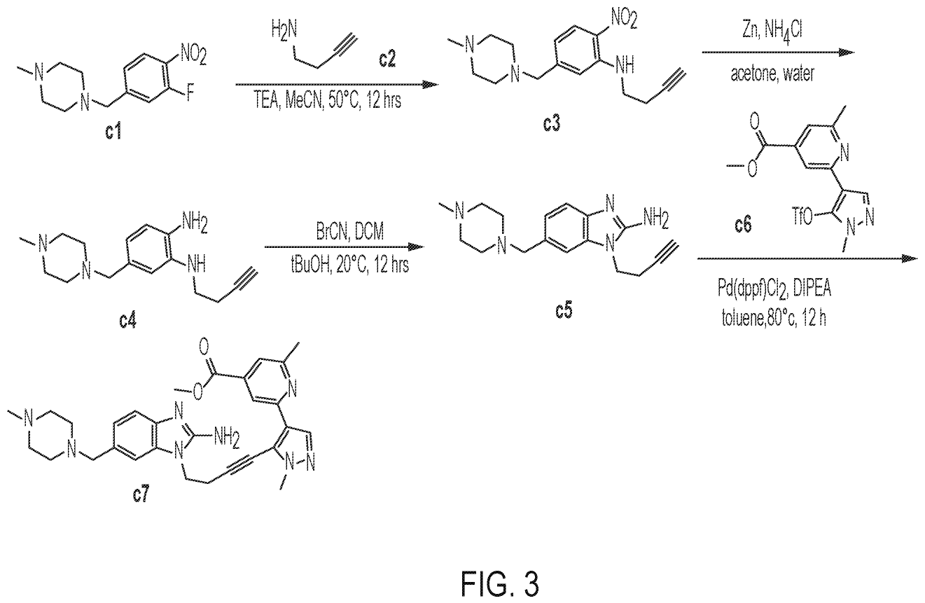

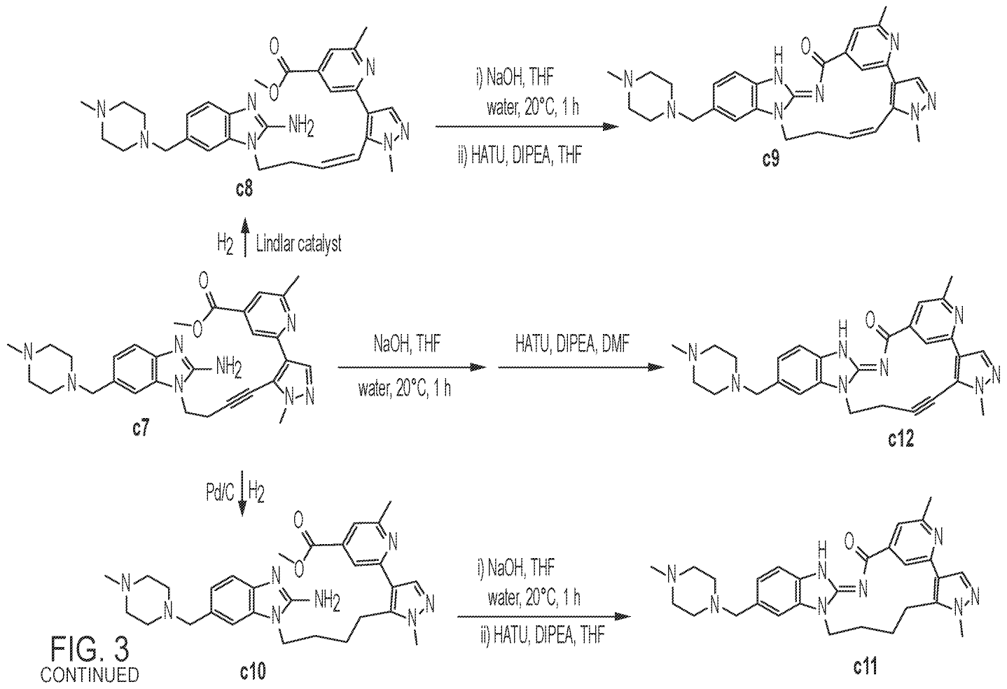

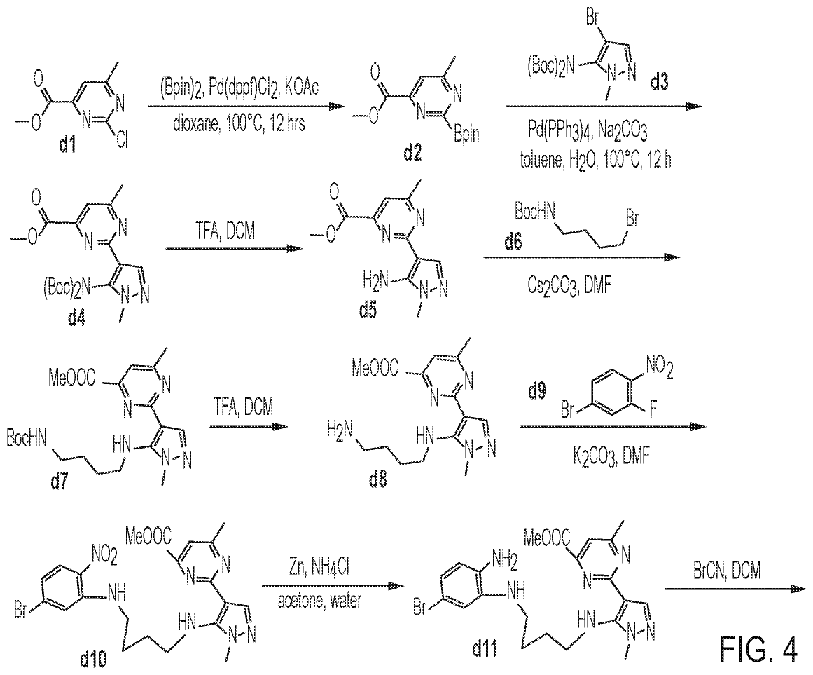

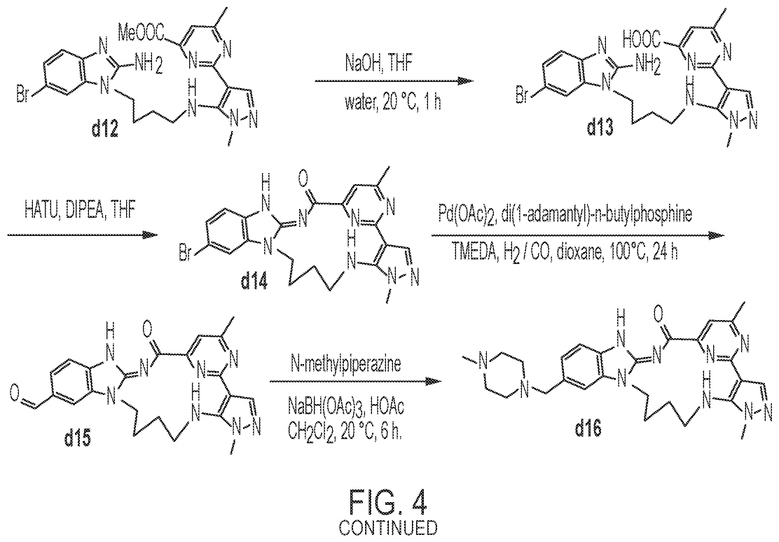

Exemplary synthetic schemes for preparing certain compounds according to the invention are provided in FIGS. 1-4.

### Pharmaceutical Compositions

In another exemplary aspect, the invention features pharmaceutical compositions comprising any compound herein, or a pharmaceutically acceptable form thereof. In embodiments, a pharmaceutical composition comprises a therapeutically effective amount of any compound described herein, or any pharmaceutically acceptable form thereof.

In embodiments, a pharmaceutically acceptable form of a compound includes any pharmaceutically acceptable salts, hydrates, solvates, isomers, prodrugs, and isotopically labeled derivatives thereof.

In embodiments, a pharmaceutical composition comprises any compound described herein, or a pharmaceutically acceptable salt thereof.

In embodiments, a pharmaceutical composition comprises a pharmaceutically acceptable excipient.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

Accordingly, in some embodiments, provided herein are pharmaceutical compositions comprising one or more compounds as disclosed herein, or a pharmaceutically acceptable form thereof (e.g., pharmaceutically acceptable salts, hydrates, solvates, isomers, prodrugs, and isotopically labeled derivatives), and one or more pharmaceutically acceptable excipients, carriers, including inert solid diluents and fillers, diluents, including sterile aqueous solution and various organic solvents, permeation enhancers, solubilizers and adjuvants. In some embodiments, a pharmaceutical composition described herein includes a second active agent such as an additional therapeutic agent, (e.g., a chemotherapeutic).

Accordingly, the present teachings also provide pharmaceutical compositions that include at least one compound described herein, or any pharmaceutically acceptable salt thereof, and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, PA (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the composition and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known 5-hydroxytryptamine receptor 7 activity modulators. Pharmaceutical compositions in the form of oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations described herein can utilize standard delay or time-release formulations to alter the absorption of the compound(s). An oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

In embodiments, a pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Kits

In some embodiments, provided herein are kits. The kits can include a compound or pharmaceutically acceptable form thereof, or pharmaceutical composition as described herein, in suitable packaging, and written material that can include instructions for use, discussion of clinical studies, listing of side effects, and the like. Kits are well suited for the delivery of solid oral dosage forms such as tablets or capsules. Such kits can also include information, such as scientific literature references, package insert materials, clinical trial results, and/or summaries of these and the like, which indicate or establish the activities and/or advantages of the pharmaceutical composition, and/or which describe dosing, administration, side effects, drug interactions, or other information useful to the health care provider. Such information can be based on the results of various studies, for example, studies using experimental animals involving in vivo models and studies based on human clinical trials.

### Therapeutic Methods

Compounds of the present teachings can be useful for the treatment or inhibition of a pathological condition or disorder in a mammal, for example, a human subject. The present teachings accordingly provide methods of treating or inhibiting a pathological condition or disorder by providing to a mammal a compound of the present teachings (including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Accordingly, compounds described herein can be particularly useful in treating diseases or disorders associated with defects in various components of signal transduction pathways and which are responsive to modulation (e.g., inhibition) of protein kinases. In embodiments, a compound described herein modulates (e.g., inhibitors) a protein kinase that is abl, Akt, bcr-abl, Blk, Brk, c-kit, c-met, c-src, CDKT, CDK2, CDK3, CDK4, CDK5, CDK6, CDK7, CDK8, CDK9, CDK10, cRafl, CSK, EGFR, ErbB2, ErbB3, ErbB4, Erk, Pak, fes, FGFR1, FGFR2, FGFR3, FGFR4, FGFR5, Fgr, fit-1, Fps, Frk, Fyn, Hck, IGF-1R, INS-R, Jak, KDR, Lck, Lyn, MEK, p38, PDGFR, PIK, PKC, PYK2, ros, tie, tie2, TRK or Zap70. In embodiments, a compound described herein modulates (e.g., inhibits) a wild-type form of a kinase (e.g., EGFR). In embodiments, a compound described herein modulates (e.g., inhibits) a mutant form of a kinase (e.g., EGFR).

In embodiments, a compound described herein, or any pharmaceutically acceptable form thereof such as a pharmaceutically acceptable salt thereof, modulates (e.g., inhibits) a kinase that is a tyrosine kinase (e.g., KIT, erb2, PDGFR, EGFR, VEGFR, src, or abl).

In embodiments, a compound described herein, or any pharmaceutically acceptable form thereof such as a pharmaceutically acceptable salt thereof, modulates (e.g., inhibits) a kinase that is a serine/threonine kinase (e.g., mTorC1, mTorC2, ATM, ATR, DNA-PK, or Akt).

In embodiments, a compound described herein, or any pharmaceutically acceptable form thereof such as a pharmaceutically acceptable salt thereof, can be used to treat or prevent a disease or disorder that is responsive to modulation (e.g., inhibition) of a protein kinase (e.g., abl, Akt, bcr-abl, Blk, Brk, c-kit, c-met, c-src, CDK1, CDK2, CDK3, CDK4, CDK5, CDK6, CDK7, CDK8, CDK9, CDK10, cRafl, CSK, EGFR, ErbB2, ErbB3, ErbB4, Erk, Pak, fes, FGFR1, FGFR2, FGFR3, FGFR4, FGFR5, Fgr, fit-1, Fps, Frk, Fyn, Hck, IGF-1R, INS-R, Jak, KDR, Lck, Lyn, MEK, p38, PDGFR, PIK, PKC, PYK2, ros, tie, tie2, TRK or Zap70).

In embodiments, a compound described herein, or any pharmaceutically acceptable form thereof such as a pharmaceutically acceptable salt thereof, can be used to treat or prevent a disease or disorder that is responsive to modulation (e.g., inhibition) of a tyrosine kinase (e.g., KIT, erb2, PDGFR, EGFR, VEGFR, src, or abl).

In embodiments, a compound described herein, or any pharmaceutically acceptable form thereof such as a pharmaceutically acceptable salt thereof, can be used to treat or prevent a disease or disorder that is responsive to modulation (e.g., inhibition) of a serine/threonine kinase (e.g., mTorC1, mTorC2, ATM, ATR, DNA-PK, or Akt).

In embodiments, a compound described herein modulates (e.g., inhibits) a wild-type form of a kinase (e.g., EGFR). In embodiments, a compound described herein modulates (e.g., inhibits) a mutant form of a kinase (e.g., EGFR).

Selective Inhibition of Kinases

The term “selective inhibition” or “selectively inhibit” as applied to a biologically active agent refers to the agent's ability to selectively reduce the target signaling activity as compared to off-target signaling activity, via direct or interact interaction with the target.

In some embodiments, a compound described herein, or any pharmaceutically acceptable salt thereof, selectively inhibits a kinase or kinase form over other kinases or other kinase forms. In embodiments, a compound selectively inhibits a mutant kinase form over the wild-type of the same kinase.

In embodiments, a compound described herein, or any pharmaceutically acceptable salt thereof, selectively inhibits a kinase (e.g., EGFR) over other kinases.

In embodiments, a compound described herein, or any pharmaceutically acceptable salt thereof, selectively inhibits a kinase form (e.g., mutant EGFR) over other kinase forms (e.g., wild-type EGFR).

By way of non-limiting example, the ratio of selectivity can be greater than a factor of about 10, greater than a factor of about 20, greater than a factor of about 30, greater than a factor of about 40, greater than a factor of about 50, greater than a factor of about 60, greater than a factor of about 70, greater than a factor of about 80, greater than a factor of about 100, greater than a factor of about 120, or greater than a factor of about 150, where selectivity can be measured by in vitro assays known in the art. Non-limiting examples of assays to measure selectivity include enzymatic assays, cellular proliferation assays, and EGFR phosphorylation assays. In one embodiment, selectivity can be determined by cellular proliferation assays. In another embodiment, selectivity can be determined by EGFR phosphorylation assays. In some embodiments, the mutant EGFR inhibitory activity of a compound as disclosed herein can be less than about 1000 nM, less than about 100 nM, less than about 50 nM, less than about 30 nM, or less than about 10 nM.

In embodiments, the IC₅₀ of a kinase inhibitor compound can be less than about 100 nM, less than about 50 nM, less than about 10 nM, less than about 1 nM, less than about 0.5 nM, or less than about 1 pM.

Determination of IC₅₀ values can be performed according to methods known in the art.

In embodiments, a compound described herein, or any pharmaceutically acceptable form thereof such as a pharmaceutically acceptable salt thereof, can be used to treat or prevent a disease or disorder that is cancer, an inflammatory disorder, a metabolic disorder, vascular disease, or neuronal disease.

Compounds described herein, or any pharmaceutically acceptable form thereof, or any pharmaceutical composition thereof, can be useful for treating diseases and disorders associated with abnormal cell proliferation.

In embodiments, a compound described herein, or a pharmaceutically acceptable form thereof (e.g., a pharmaceutically acceptable salt thereof), or a pharmaceutical composition thereof, can be used to treat cancer.

Cancer

The compositions and methods provided herein can potentially be useful for the treatment of cancer including tumors such as astrocytic, breast, cervical, colorectal, endometrial, esophageal, gastric, head and neck, hepatocellular, laryngeal, lung, oral, ovarian, prostate and thyroid carcinomas and sarcomas.

In embodiments, a cancer is a cardiac cancer such as sarcoma (angiosarcoma, fibrosarcoma, rhabdomyosarcoma, liposarcoma), myxoma, rhabdomyoma, fibroma, lipoma or teratoma.

In embodiments, a cancer is a lung cancer such as bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, or mesothelioma.

In embodiments, a cancer is a gastrointestinal cancer such as: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma).

In embodiments, a cancer is a cancer of the genitourinary tract such as: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma).

In embodiments a cancer is a liver cancer such as hepatoma (hepatocellular carcinoma), cholangiocarcinoma, hepatoblastoma, angiosarcoma, hepatocellular adenoma, hemangioma.

In embodiments, a cancer is a bone cancer such as: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors.

In embodiments a cancer is a cancer of the central nervous system (CNS) such as: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma).

In embodiments, a cancer is a gynecological cancer such as: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma).

In embodiments, a cancer is a hematological cancer such as: blood (myeloid leukemia (acute and chronic), acute lymphoblastic leukemia, chronic lymphocytic leukemia, myeloproliferative diseases, multiple myeloma, myelodysplasia syndrome), Hodgkin's disease, non-Hodgkin's lymphoma (malignant lymphoma).

In embodiments, a cancer is a skin cancer such as: malignant melanoma, basal cell carcinoma, squamous cell carcinoma, Kaposi's sarcoma, moles dysplastic nevi, lipoma, angioma, dermatofibroma, keloids, psoriasis.

In embodiments, a cancer is a cancer of the adrenal glands such as neuroblastoma. Thus, the term “cancerous cell” as provided herein, includes a cell afflicted by any one of or related to the above identified conditions.

In embodiments, a cancer is an EGFR-driven cancer (e.g., as described herein). In embodiments, an EGFR-driven cancer is non-small cell lung cancer (NSCLC), squamous cell carcinoma, adenocarcinoma, adenocarcinoma, bronchioloalveolar carcinoma (BAC), BAC with focal invasion, adenocarcinoma with BAC features, and large cell carcinoma; neural tumors, such as glioblastomas; pancreatic cancer; head and neck cancers (e.g., squamous cell carcinoma); breast cancer; colorectal cancer; epithelial cancer, including squamous cell carcinoma; ovarian cancer; prostate cancer; or adenocarcinomas.

In embodiments, a cancer is an EGFR mutant cancer (e.g., as described herein). In embodiments, an EGFR mutant cancer is non-small cell lung cancer (NSCLC), squamous cell carcinoma, adenocarcinoma, adenocarcinoma, bronchioloalveolar carcinoma (BAC), BAC with focal invasion, adenocarcinoma with BAC features, and large cell carcinoma; neural tumors, such as glioblastomas; pancreatic cancer; head and neck cancers (e.g., squamous cell carcinoma); breast cancer; colorectal cancer; epithelial cancer, including squamous cell carcinoma; ovarian cancer; prostate cancer; or adenocarcinomas.

In one embodiment, the compositions and methods provided herein are useful for the treatment of lung cancer and pancreatic cancer, most specifically, non-small cell lung cancer (NSCLC).

In embodiments, a cancer is refractory to TKI therapies (e.g., erlotinib, gefitinib, dacomitinib, afatinib, osimertinib).

Lung Cancer

In embodiments, a cancer is a lung cancer.

Lung cancer is the most common cause of cancer mortality globally and the second most common cancer in both men and women. About 14% of all new cancers are lung cancers. In the United States (US), there are projected to be 222,500 new cases of lung cancer (116,990 in men and 105,510 in women) and 155,870 deaths from lung cancer (84,590 in men and 71,280 in women) in 2017.

The two major forms of lung cancer are non-small cell lung cancer (NSCLC) and small cell lung cancer. NSCLC is a heterogeneous disease that consists of adenocarcinoma, large-cell carcinoma, and squamous cell carcinoma (sqNSCLC), and comprises approximately 80% to 85% of all lung cancers. Squamous cell carcinoma of the lung accounts for 20% to 30% of NSCLC. Despite advances in early detection and standard treatment, NSCLC is often diagnosed at an advanced stage, has poor prognosis, and is the leading cause of cancer deaths worldwide.

Platinum-based doublet therapy, maintenance chemotherapy, and anti-angiogenic agents in combination with chemotherapy have contributed to improved patient outcomes in advanced NSCLC.

In embodiments, an advanced lung cancer is stage III cancer or stage IV cancer. In embodiments, an advanced lung cancer is stage III cancer. In embodiments, an advanced lung cancer is stage IV cancer. In embodiments, an advanced lung cancer is locally advanced. In embodiments, an advanced lung cancer is metastatic.

In embodiments, a lung cancer is small cell lung cancer (SCLC).

In embodiments, a lung cancer is non-small cell lung cancer (NSCLC) such as adenocarcinoma, large-cell carcinoma, or squamous cell carcinoma (sqNSCLC). In embodiments, a NSCLC is lung adenocarcinoma. In embodiments, a NSCLC is large cell carcinoma of the lung. In embodiments, a NSCLC is squamous cell carcinoma of the lung (sqNSCLC).

In embodiments, a lung cancer (e.g., NSCLC) is an EGFR-mutant lung cancer (e.g., EGFR-mutant NSCLC). In embodiments, a cancer is NSCLC (e.g., advanced NSCLC) with an identified EGFR mutation.

EGFR Driven and EGFR Mutant Cancers

The invention features compounds which can be useful for treating patients who have an EGFR-driven cancer, including cancers which are, or have become, refractory to erlotinib, gefitinib, dacomitinib, afatinib, osimertinib, or cancers which bear an EGFR mutation identified herein, by administering a compound of formula (I) to a subject.

That is, compounds described herein can be effective inhibitors of mutant forms of EGFR, such as single, double, or mutant EGFR having L858R (“L”), T790M (“T”), C797S (“C”), and/or Exon19 (Del19 or “D”) mutations, or any combination thereof. Such inhibitors can be particularly beneficial in therapy of patients who have developed mutations after receiving certain other cancer therapies. For example, a patient may present with single mutants (D, L) but after certain treatments, a patient may develop secondary and even (e.g., after osimertinib treatment) tertiary mutations. Accordingly, new inhibitors that have activity against cancers characterized by single, double, and/or triple mutant EGFR can confer great benefit to cancer patients, including those who have developed resistance to previous therapies.

EGFR-driven cancers which can be treated using the compositions and method of the invention include, for example, EGFR mutants including one or more deletions, substitutions, or additions in the amino acid or nucleotide sequences of EGFR, or fragments thereof.

An EGFR-driven cancer may result from an EGFR fusion. For example, the N-terminal of EGFR can be linked to various fusion partners such as RAD51. Cancers (e.g., lung cancers) characterized by an EGFR-fusion (e.g., an EGFR-RAD51 fusion) may be particularly suitable for therapy using any compound described herein, or any pharmaceutically acceptable form (e.g., a pharmaceutically acceptable salt) thereof.

Mutations in EGFR can occur in any part of the EGFR sequence. Generally, EGFR mutants arise from mutations in the kinase domain (i.e., exons 18-24 in the EGFR sequence) or in the extracellular domain (i.e., exons 2-16 in the EGFR sequence).

A mutation in EGFR can be an activating mutation, which lead to a ligand-independent activation of TK activity. A mutation in EGFR can also be a resistance mutation, which can confer resistance to TKI therapies such as resistance to one or more of erlotinib, gefitinib, dacomitinib, afatinib, or osimertinib.

For example, mutations typically occur in the kinase domain, including one or more of a point mutation in exon 18 (e.g., L688P, V689M, P694L/S, N700D, L703V, E709K/Q/A/G/V, I715S, L718P, G719C/A/S/R, or S720P/F), a deletion in exon 19 that may or may not include an insertion (e.g., delG719, delE746_E749, delE746_A750, delE746_A750 insRP, delE746_A750 insQP, delE746_T751, delE746 T751insA/I/V, delE746_T751insVA, delE746_S752, delE746_S752insA/V/D, delE746_P53insLS, delL747_E749, delL747_A750, delL747_A750insP, delL747_T751, delL747_T751insP/S/Q, delL747_T751insPI, delL747_5752, delL747_S752insQ, delL747_P753, delL747_P753insS/Q, delL747_L754insSR, delE749_A750, delE749_A750 insRP, delE749_T751, delT751_I759, delT751_I759insS/N, or delS752_1759), a duplication in exon 19 (e.g., K739_I44dupKIPVAI), a point mutation in exon 19 (e.g., L730F, W731Stop, P733L, G735S, V742A, E746V/K, A750P, T751I, S752Y, P753S, A754P, or D761Y), an in-frame insertion in exon 20 (e.g., D761_E762insEAFQ, A767_S768insTLA, V769_D770insY, V769_D770insCV, V769_D770insASV, D770_N771insD/G, D770_N771insNPG, D770_N771insSVQ, P772_H773insN/V, P772_H773insYNP, or V774_C775insHV), a deletion in exon 20 that may or may not include an insertion (e.g., delM766_A767, delM766_A767insAI, delA767_V769, delD770, or delP772_H773insNP), a duplication in exon 20 (e.g., S768_D770dupSVD, A767_V769dupASV, or H773dupH), a point mutation in exon 20 (e.g., D761N, A763V, V765A/M, S768I, V769L/M, S768I, P772R, N771T, H773R/Y/L, V774M, R776G/H/C, G779S/F, T783A, T784F, L792P, L798H/F, T790M, R803W, K806E, or L814P), or a point mutation in exon 21 (e.g., G810S, N826S, L833V, H835L, L838V, A839T, K846R, T847I, H850N, V851I/A, I853T, L858M/R, A859T, L861Q/R, G863D, A864T, E866K, or G873E).

In lung cancer, activation mutants are typical.

In embodiments, a mutation is a resistance mutation. In particular, drug resistance in 50% of lung cancers arises from the T790M point mutation. Other exemplary resistance mutation include point mutations such as: C797X (e.g., C797S, C797G, or C797N); G796X (e.g., G796R, G796S, or G796D); L792X (e.g. L792H, L792F, L792R, or L792Y); G724S; L718X (e.g., L718P, L718Q, or L718V); S768I; or G719A.

In glioblastoma, mutations typically, but not exclusively, occur in the extracellular domain, including EGFR variant I (EGFRvI) lacking the extracellular domain and resembling the v-erbB oncoprotein; EGFRvII lacking 83 amino acids from domain IV; and EGFRvIII lacking amino acids 30-297 from domains I and II, which is the most common amplification and is reported in 30-50% of glioblastomas and 5% of squamous cell carcinoma. Other mutations for glioblastoma include one or more of point mutations in exon 2 (e.g., D46N or G63R), exon 3 (e.g., R108K in domain I), exon 7 (e.g., T263P or A289D/T/V in domain II), exon 8 (e.g., R324L or E330K), exon 15 (e.g., P596L or G598V in domain IV), or exon 21 (L861Q in the kinase domain).

EGFR mutants also include those with a combination of two or more mutations, as described herein. Exemplary combinations include S768I and G719A; S768I and V769L; H773R and W731Stop; R776G and L858R; R776H and L861Q; T790M and L858R; T790M and delE746_A750; R803W and delE746_T751insVA; delL747_E749 and A750P; delL747_S752 and E746V; delL747_S752 and P753S; P772_H773insYNP and H773Y; P772_H773insNP and H773Y; and D770_N771insG and N771T. Other exemplary combinations include any including T790M (e.g., T790M and L858R or T790M and delE746_A750.

EGFR mutants can be either activation mutants or resistant mutants. Activation mutants include those with substitutions that increase drug sensitivity (e.g., G719C/S/A, delE746_A750, or L858R). Resistant mutants include those with substitutions that increase drug resistance (e.g., T790M or any combination including T790M).

In embodiments, an EGFR mutation is a deletion in exon19 (del19). In embodiments, an EGFR mutation is a T790M mutation. In embodiments, an EGFR mutation is a L858R mutation. In embodiments, an EGFR mutation is a C797S mutation. In embodiments, an EGFR-driven cancer (e.g., non-small cell lung cancer) is characterized by at least one of these mutations. In embodiments, an EGFR-driven cancer (e.g., non-small cell lung cancer) is characterized by at least two of these mutations. In embodiments, an EGFR-driven cancer (e.g., non-small cell lung cancer) is characterized by at least three of these mutations.

EGFR-driven cancers include those having any mutant described herein. For example, EGFRvIII is commonly found in glioblastoma and has also been reported in breast, ovarian, prostate, and lung carcinomas. Exemplary EGFR-driven cancers: glioblastoma, lung cancer (e.g., squamous cell carcinoma, non-small cell lung cancer, adenocarcinoma, bronchioloalveolar carcinoma (BAC), BAC with focal invasion, adenocarcinoma with BAC features, and large cell carcinoma), pancreatic cancer, head and neck cancers (e.g., squamous cell carcinoma), breast cancer, colorectal cancer, epithelial cancer (e.g., squamous cell carcinoma), ovarian cancer, and prostate cancer.

In particular, the invention described herein would benefit patient populations having higher risk for TKI-resistant mutations. About 8,000 to 16,000 new cases per year can be estimated based on: incidence of non-small cell lung cancer (about 160,000 new cases in the U.S.), the response to erlotinib in the general population (about 10%, resulting in a sensitive population of 16,000), the presence of activation mutations (10-20% in white and 30-40% in Asian population, resulting in a sensitive population of 16,000-32,000), acquisition of secondary resistance (most if not all patients, resulting in a sensitive population of 16,000-32,000), and percentage of patients carrying the T790M point mutations (about 50%, resulting in a sensitive population of 8,000-16,000). Patients having TKI-resistant mutations include those patients having cancers resistant to one or more of erlotinib, gefitinib, dacomitinib, afatinib, osimertinib, CL-387,785, BIBW 2992 (CAS Reg. No. 439081-18-2), CI-1033, neratinib (HKI-272), MP-412 (AV-412), PF-299804, AEE78, and XL64.

In particular, the inventions relate to treatment of EGFR-driven cancers having the T790M point mutation. Generally, irreversible inhibitors (e.g., CI-1033, neratinib (HKI-272), and PF-299804) are less potent in cell lines having the T790M mutation and do not inhibit T790M at clinically achievable concentrations. Since the ATP Km of T790M and WT are similar, concentrations that inhibit the mutant will inhibit the WT and result in gastrointestinal and cutaneous events.

An EGFR mutant also includes other amino acid and nucleotide sequences of EGFR with one or more deletions, substitutions, or additions, such as point mutations, that retain or increase tyrosine kinase or phosphorylation activity. Where the mutant is a protein or polypeptide, preferable substitutions are conservative substitutions, which are substitutions between amino acids similar in properties such as structural, electric, polar, or hydrophobic properties. For example, the substitution can be conducted between basic amino acids (e.g., Lys, Arg, and His), or between acidic amino acids (e.g., Asp and Glu), or between amino acids having non-charged polar side chains (e.g., Gly, Asn, Gln, Ser, Thr, Tyr, and Cys), or between amino acids having hydrophobic side chains (e.g., Ala, Val, Leu, Ile, Pro, Phe, and Met), or between amino acids having branched side chains (e.g., Thr, Val, Leu, and Ile), or between amino acids having aromatic side chains (e.g., Tyr, Trp, Phe, and His).

Where the mutant is a nucleic acid, the DNA encoding an EGFR mutant protein may comprise a nucleotide sequence capable of hybridizing to a complement sequence of the nucleotide sequence encoding an EGFR mutant, as defined herein, under stringent conditions. As used herein, the stringent conditions include low, medium or high stringent conditions. An example of the stringent conditions includes hybridization at approximately 42-55° C. in approximately 2-6×SSC, followed by wash at approximately 50-65° C. in approximately 0.1-1×SSC containing approximately 0.1-0.2% SDS, where 1×SSC is a solution containing 0.15 M NaCl and 0.015 M Na citrate, pH 7.0. Wash can be performed once or more. In general, stringent conditions may be set at a temperature approximately 5° C. lower than a melting temperature (Tm) of a specific nucleotide sequence at defined ionic strength and pH.

The amino acid and nucleotide sequences of EGFR and DNAs encoding them are available from known databases such as NCBI GenBank (USA), EMBL (Europe), etc. For example, GenBank accession numbers for EGFR [Homo sapiens] include MIM131550, AA128420, NM 005228, NP_005219.2, and GeneID: 1956.

EGFR-Selective Inhibition

In some embodiments, a compound described herein, or any pharmaceutically acceptable salt thereof, selectively inhibits EGFR (including any mutant EGFR described herein) over other kinases.

In some embodiments, a compound described herein, or any pharmaceutically acceptable salt thereof, selectively inhibits mutant EGFR (e.g., any mutant EGFR described herein) over wild-type EGFR. In embodiments, a compound described herein selectively inhibits EGFR characterized by a mutation that is: a deletion in exon19 (del19), a T790M mutation, a L858R mutation, and/or a C797S mutation, or any combination thereof. Such inhibitors can be effective in ameliorating diseases and disorders associated with mutant EGFR activity.

By way of non-limiting example, the ratio of selectivity can be greater than a factor of about 10, greater than a factor of about 20, greater than a factor of about 30, greater than a factor of about 40, greater than a factor of about 50, greater than a factor of about 60, greater than a factor of about 70, greater than a factor of about 80, greater than a factor of about 100, greater than a factor of about 120, or greater than a factor of about 150, where selectivity can be measured by in vitro assays known in the art. Non-limiting examples of assays to measure selectivity include enzymatic assays, cellular proliferation assays, and EGFR phosphorylation assays. In one embodiment, selectivity can be determined by cellular proliferation assays. In another embodiment, selectivity can be determined by EGFR phosphorylation assays. In some embodiments, the mutant EGFR inhibitory activity of a compound as disclosed herein can be less than about 1000 nM, less than about 100 nM, less than about 50 nM, less than about 30 nM, or less than about 10 nM.

In embodiments, the IC₅₀ of a subject compound for mutant EGFR inhibition can be less than about 100 nM, less than about 50 nM, less than about 10 nM, less than about 1 nM, less than about 0.5 nM, or less than about 1 pM.

Characterization of EGFR-Driven Cancers

The compositions and methods of the invention can be used to treat subjects having an EGFR-driven cancer (i.e., cancers characterized by EGFR mutant expression or overexpression). EGFR mutant expression or overexpression can be determined in a diagnostic or prognostic assay by evaluating levels of EGFR mutants in biological sample, or secreted by the cell (e.g., via an immunohistochemistry assay using anti-EGFR antibodies or anti-p-EGFR antibodies; FACS analysis, etc.). Alternatively, or additionally, one can measure levels of EGFR mutant-encoding nucleic acid or mRNA in the cell, e.g., via fluorescent in situ hybridization using a nucleic acid based probe corresponding to an EGFR mutant-encoding nucleic acid or the complement thereof; (FISH; see WO98/45479, published October, 1998), Southern blotting, Northern blotting, or polymerase chain reaction (PCR) techniques, such as real time quantitative PCR (RT-PCR). One can also study EGFR mutant expression by measuring shed antigen in a biological sample, such as serum, e.g., using antibody-based assays (see also, e.g., U.S. Pat. No. 4,933,294, issued Jun. 12, 1990; WO91/05264, published Apr. 18, 1991; U.S. Pat. No. 5,401,638, issued Mar. 28, 1995; and Sias et al., J. Immunol. Methods 132:73 (1990)). Aside from the above assays, various in vivo assays are available to the skilled practitioner. For example, one can expose cells within the body of the mammal to an antibody which is optionally labeled with a detectable label, e.g., a radioactive isotope, and binding of the antibody to cells in the mammal can be evaluated, e.g., by external scanning for radioactivity or by analyzing a biopsy taken from a mammal previously exposed to the antibody.

Examples of biological properties that can be measured in isolated cells include mRNA expression, protein expression, and DNA quantification. Additionally, the DNA of cells isolated by the methods of the invention can be sequenced, or certain sequence characteristics (e.g., polymorphisms and chromosomal abnormalities) can be identified using standard techniques, e.g., FISH or PCR. The chemical components of cells, and other analytes, may also be assayed after isolation. Cells may also be assayed without lysis, e.g., using extracellular or intracellular stains or by other observation, e.g., morphology or growth characteristics in various media.

While any hybridization technique can be used to detect the gene rearrangements, one preferred technique is fluorescent in situ hybridization (FISH). FISH is a cytogenetic technique which can be used to detect and localize the presence or absence of specific DNA or RNA sequences on chromosomes. FISH incorporates the use of fluorescently labeled nucleic acid probes which bind only to those parts of the chromosome with which they show a high degree of sequence similarity. Fluorescence microscopy can be used to find out where the fluorescent probe bound to the chromosome. The basic steps of FISH are outlined below. Exemplary FISH probes include Vysis EGFR SpectrumOrange/CEP SpectrumGreen Probe (Abbott, Downers Grove, IL), which hybridizes to band 7p12; and ZytoLight SPEC EGFR/CEN 7 Dual Color Probe (ZytoVision), which hybridizes to the alpha-satellite sequences of the centromere of chromosome 7.

For FISH, a probe is constructed that is long enough to hybridize specifically to its target (and not to similar sequences in the genome), but not too large to impede the hybridization process. Probes are generally labeled with fluorophores, with targets for antibodies, with biotin, or any combination thereof. This can be done in various ways, for example using random priming, nick translation, and PCR using tagged nucleotides.

Generally, a sample or aliquot of a population of cells is used for FISH analysis. For example, in one method of preparation, cells are trypsinized to disperse into single cells, cytospun onto glass slides, and then fixed with paraformaldehyde before storing in 70% ethanol. For preparation of the chromosomes for FISH, the chromosomes are firmly attached to a substrate, usually glass. After preparation, the probe is applied to the chromosome RNA and starts to hybridize. In several wash steps, all unhybridized or partially hybridized probes are washed away. If signal amplification is necessary to exceed the detection threshold of the microscope (which depends on many factors such as probe labeling efficiency, the kind of probe, and the fluorescent dye), fluorescent tagged antibodies or strepavidin are bound to the tag molecules, thus amplifying the fluorescence.

An epifluorescence microscope can be used for observation of the hybridized sequences. The white light of the source lamp is filtered so that only the relevant wavelengths for excitation of the fluorescent molecules arrive onto the sample. Emission of the fluorochromes happens, in general, at larger wavelengths, which allows one to distinguish between excitation and emission light by mean of another optical filter. With a more sophisticated filter set, it is possible to distinguish between several excitation and emission bands, and thus between several fluorochromes, which allows observation of many different probes on the same strand.

Depending on the probes used, FISH can have resolution ranging from huge chromosomes or tiny (˜100 kilobase) sequences. The probes can be quantified simply by counting dots or comparing color.

Allele-specific quantitative real time-PCR may also be used to identify a nucleic acid encoding a mutant EGFR protein (see, for e.g., Diagnostic Innovations DxS BCR-ABL T3151 Mutation Test Kit, and Singer et al., Methods in Molec. Biol. 181:145 (2001)). This technique utilizes Taq DNA polymerase, which is extremely effective at distinguishing between a match and a mismatch at the 3′-end of the primer (when the 3′-base is mismatched, no efficient amplification occurs). Using this technique, the 3′-end of the primer may be designed to specifically hybridize to a nucleic acid sequence that corresponds to a codon that encodes a mutant amino acid in an EGFR mutant, as described herein. In this way, the specific mutated sequences can be selectively amplified in a patient sample. This technique further utilizes a Scorpion probe molecule, which is a bifunctional molecule containing a PCR primer, a fluorophore, and a quencher. The fluorophore in the probe interacts with a quencher, which reduces fluorescence. During a PCR reaction, when the Scorpion probe binds to the amplicon, the fluorophore and quencher in the Scorpion probe become separated, which leads to an increase in fluorescence from the reaction tube. Any of the primers described herein may be used in allele-specific quantitative real time PCR.

A biological sample can be analyzed to detect a mutation in an EGFR gene, or expression levels of an EGFR gene, by methods that are known in the art. For example, methods such as direct nucleic acid sequencing, altered hybridization, aberrant electrophoretic gel migration, binding or cleavage mediated by mismatch binding proteins, single-strand conformational polymorphism (SSCP) analysis, or restriction fragment length polymorphism (RFLP) analysis of PCR products derived from a patient sample can be used to detect a mutation in an EGFR gene; ELISA can be used to measure levels of EGFR polypeptide; and PCR can be used to measure the level of an EGFR nucleic acid molecule.

Any of these techniques may be used to facilitate detection of a mutation in a candidate gene, and each is well known in the art; examples of particular techniques are described, without limitation, in Orita et al. (Proc. Natl. Acad. Sci. USA 86:2766 (1989)) and Sheffield et al. (Proc. Natl. Acad. Sci. USA 86:232 (1989)). Furthermore, expression of the candidate gene in a biological sample (e.g., a biopsy) may be monitored by standard Northern blot analysis or may be aided by PCR (see, e.g., Ausubel et al., Current Protocols in Molecular Biology, John Wiley & Sons, New York, NY (1995); PCR Technology: Principles and Applications for DNA Amplification, H. A. Ehrlich, Ed., Stockton Press, NY; Yap et al., Nucl. Acids. Res. 19:4294 (1991)).

One skilled in the art may identify in a nucleic acid or protein sequence a residue (e.g., amino acid or nucleotide) or codon that corresponds to a residue or codon in wild-type EGFR or EGFR mutants using a number of sequence alignment software programs (e.g., NCBI BLAST website). Such software programs may allow for gaps in the alignment of the compared sequences. Using such software, one skilled in the art may identify a nucleotide, amino acid, or amino acid that corresponding to a specific nucleotide, amino acid, or codon in wild-type EGFR or EGFR mutants.

Levels of EGFR expression (e.g., DNA, mRNA, or protein) in a biological sample can be determined by using any of a number of standard techniques that are well known in the art or described herein. Exemplary biological samples include plasma, blood, sputum, pleural effusion, bronchoalveolar lavage, or biopsy, such as a lung biopsy and lymph node biopsy. For example, EGFR expression in a biological sample (e.g., a blood or tissue sample) from a patient can be monitored by standard northern blot analysis or by quantitative PCR (see, e.g., Ausubel et al., supra; PCR Technology: Principles and Applications for DNA Amplification, H. A. Ehrlich, Ed., Stockton Press, NY; Yap et al., Nucl. Acids. Res. 19:4294 (1991)).

Combination Therapies

In some embodiments, provided herein are methods for combination therapies in which an agent known to modulate other pathways, or other components of the same pathway, or even overlapping sets of target enzymes are used in combination with a compound as provided herein, or a pharmaceutically acceptable form (e.g., pharmaceutically acceptable salts, hydrates, solvates, isomers, prodrugs, and isotopically labeled derivatives) thereof. In one aspect, such therapy includes, but is not limited to, the combination of the subject compound with chemotherapeutic agents, therapeutic antibodies, and radiation treatment, to provide a synergistic or additive therapeutic effect.

When administered as a combination, the therapeutic agents can be formulated as separate compositions that are administered at the same time or sequentially at different times, or the therapeutic agents can be given as a single composition. The phrase “combination therapy”, in referring to the use of a disclosed compound together with another pharmaceutical agent, means the coadministration of each agent in a substantially simultaneous manner as well as the administration of each agent in a sequential manner, in either case, in a regimen that will provide beneficial effects of the drug combination. Coadministration includes, inter alia, the simultaneous delivery, e.g., in a single tablet, capsule, injection or other dosage form having a fixed ratio of these active agents, as well as the simultaneous delivery in multiple, separate dosage forms for each agent respectively. Thus, the administration of disclosed compounds can be in conjunction with additional therapies known to those skilled in the art in the prevention or treatment of cancer, such as radiation therapy or cytostatic agents, cytotoxic agents, other anti-cancer agents and other drugs to ameliorate symptoms of the cancer or side effects of any of the drugs.

In some embodiments, treatment can be provided in combination with one or more other cancer therapies, include surgery, radiotherapy (e.g., gamma-radiation, neutron beam radiotherapy, electron beam radiotherapy, proton therapy, brachytherapy, and systemic radioactive isotopes, etc.), endocrine therapy, biologic response modifiers (e.g., interferons, interleukins, and tumor necrosis factor (TNF)), hyperthermia, cryotherapy, agents to attenuate any adverse effects (e.g., antiemetics), and other cancer chemotherapeutic drugs. The other agent(s) can be administered using a formulation, route of administration and dosing schedule the same or different from that used with the compounds provided herein.

In embodiments, combination therapy comprises administration of a compound described herein, or any pharmaceutically acceptable form thereof (e.g., any pharmaceutically acceptable salt thereof), or a pharmaceutical composition thereof, in combination with anti-cancer drugs (e.g., antiproliferative agents, anti-angiogenic agents and other chemotherapeutic agents).

In embodiments, combination therapy comprises administration of a compound described herein, or any pharmaceutically acceptable form thereof (e.g., any pharmaceutically acceptable salt thereof), or a pharmaceutical composition thereof, in combination with an amount of an anti-cancer agent (e.g., a chemotherapeutic agent).

### Examples

### Exemplary Synthetic Methods

Exemplary synthetic methods for preparing compounds of the invention are described herein.

### Example 1: Preparation of Compound (32)

### The synthesis of (10R,20E)-5,10,25-trimethyl-15-(4-methylpiperazin-1-yl)-4,5,12,19,21,26-hexazapentacyclo[21.3.1.02,6.012,20.013,18]heptacosa-1(27),2(6), 3,13(18),14,16,20,23,25-nonaen-22-one (Compound (32)

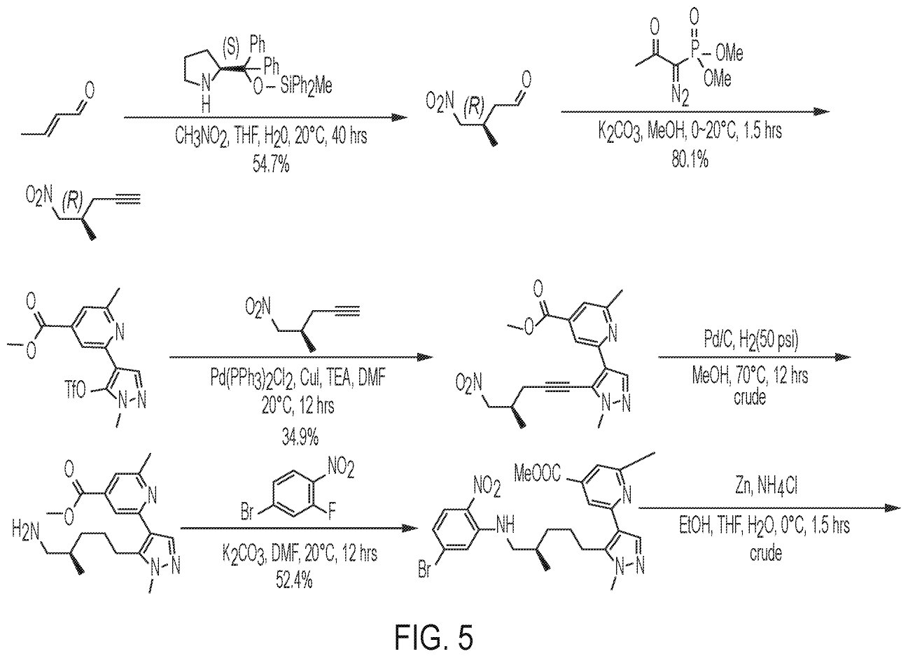

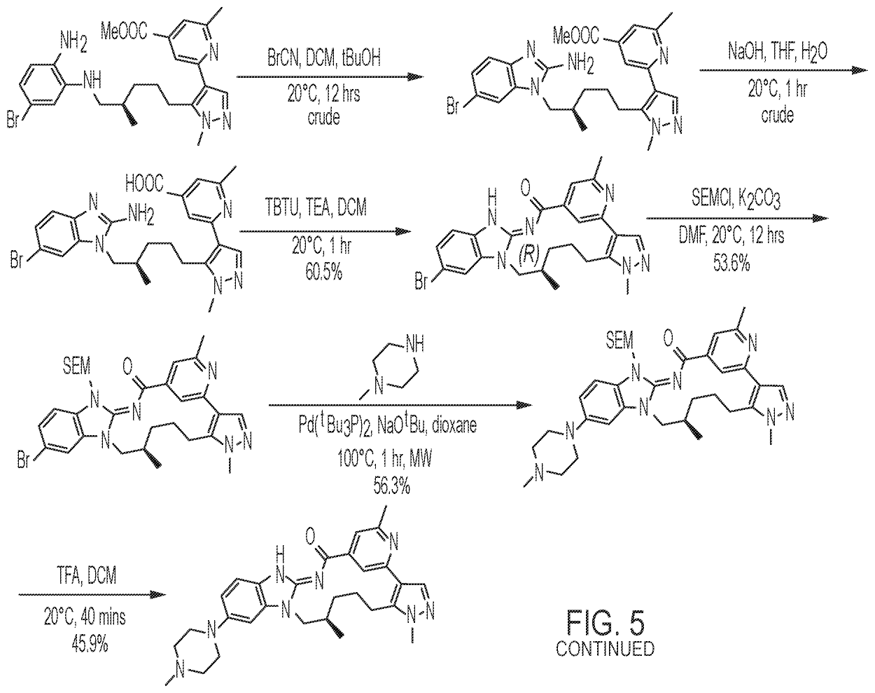

The general synthetic schemes for preparing Compound (32) according to the invention are provided in FIG. 5.

### Step 1: Synthesis of (3R)-3-methyl-4-nitro-butanal

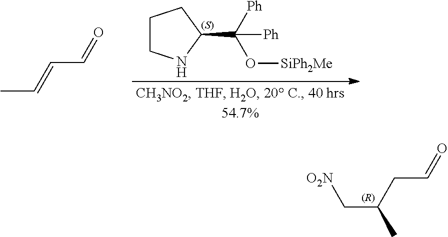

To a solution of (E)-but-2-enal (19.54 g, 0.279 mol, 1.0 eq) and [diphenyl-[(2S)-pyrrolidin-2-yl]methoxy]-methyl-diphenyl-silane (9.03 g, 20.1 mmol, 0.072 eq) in THF (630.0 mL) and H₂O (70.0 mL) was added nitromethane (51.05 g, 836 mmol, 3.0 eq). The mixture was stirred at 20° C. for 40 hours. The resulting mixture was dried over MgSO₄, filtered and concentrated under reduced pressure. The residue was purified by column chromatography (silica, Petroleum ether/Ethyl acetate=100/1 to 10/1) to afford (3R)-3-methyl-4-nitro-butanal (20 g, 54.7% yield) as yellow oil.

¹H NMR (400 MHz, CDCl₃) δ ppm 9.77 (s, 1H), 4.46-4.33 (m, 2H), 2.88 (qd, J=6.8, 13.2 Hz, 1H), 2.71-2.61 (m, 1H), 2.57-2.48 (m, 1H), 1.11 (d, J=6.8 Hz, 3H).

### Step 2: Synthesis of (4R)-4-methyl-5-nitro-pent-1-yne

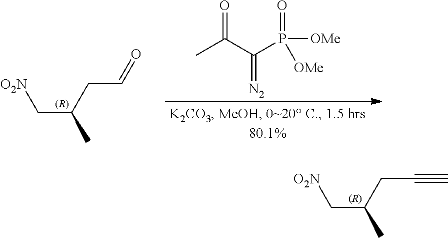

To a solution of (3R)-3-methyl-4-nitro-butanal (6.57 g, 50.1 mmol, 1.0 eq) in MeOH (250.0 mL) was added 1-diazo-1-dimethoxyphosphoryl-propan-2-one (12.51 g, 65.1 mmol, 1.3 eq) and K₂CO₃ (20.77 g, 150 mmol, 3.0 eq) at 0° C. The mixture was stirred at 20° C. for 1.5 hours under N2. The reaction mixture was quenched by addition aq. saturated NH₄Cl (30 mL) at 0° C., and extracted with MTBE (30 mL*2). The combined organic layers were washed with saturated NaHCO₃aqueous solution (20 mL*1), dried over anhydrous MgSO₄, filtered and concentrated under reduced pressure at 15° C. The residue was purified by column chromatography (silica, DCM/MTBE=1/0 to 1/1) to afford (4R)-4-methyl-5-nitro-pent-1-yne (6.3 g, 80.1% yield, 81% purity) as a yellow solid.

¹H NMR (400 MHz, CDCl₃) δ ppm 4.50 (dd, J=6.4, 12.4 Hz, 1H), 4.29 (dd, J=7.2, 12.4 Hz, 1H), 2.56 (sxtd, J=6.4, 13.2 Hz, 1H), 2.39-2.23 (m, 2H), 2.06 (t, J=2.4 Hz, 1H), 1.11 (d, J=6.8 Hz, 3H).

### Step 3: Synthesis of methyl 2-methyl-6-[I-methyl-5-[(4R)-4-methyl-5-nitro-pent-1-ynyl]pyrazol-4-yl]pyridine-4-carboxylate

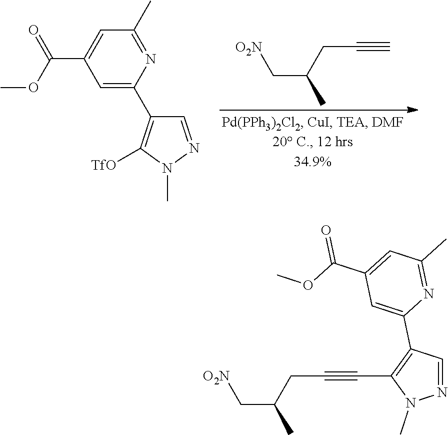

A mixture of methyl 2-methyl-6-[1-methyl-5-(trifluoromethylsulfonyloxy)pyrazol-4-yl]pyridine-4-carboxylate (14 g, 36.9 mmol, 1.0 eq), (4R)-4-methyl-5-nitro-pent-1-yne (6.3 g, 40.1 mmol, 81% purity, 1.09 eq), Pd(PPh₃)₂Cl₂ (2.59 g, 3.69 mmol, 0.1 eq), CuI (1.41 g, 7.38 mmol, 0.2 eq) and Et₃N (111 mmol, 15.4 mL, 3.0 eq) in DMF (200.0 mL) was degassed and purged with N2 for 3 times, and then the mixture was stirred at 20° C. for 12 hours under N2 atmosphere. The reaction mixture was diluted with aq. saturated NH₄Cl (100 mL) and extracted with EtOAc (300 mL*2). The combined organic layers were washed with H₂O (300 mL*2), brine (300 mL*1), dried over anhydrous Na₂SO₄, filtered and concentrated under reduced pressure. The residue was purified by flash chromatography (ISCO®; 120 g SepaFlash® Silica Flash Column, Petroleum ethergradient/EtOAc with EtOAc from 0˜40%, 80 mL/min, 254 nm) to afford methyl 2-methyl-6-[1-methyl-5-[(4R)-4-methyl-5-nitro-pent-1-ynyl]pyrazol-4-yl]pyridine-4-carboxylate (5.5 g, 34.9% yield, 83% purity) as a yellow solid.

¹H NMR (400 MHz, CDCl₃) δ ppm 8.26 (s, 1H), 8.12 (s, 1H), 7.56 (s, 1H), 4.60 (dd, J=6.4, 12.4 Hz, 1H), 4.42 (dd, J=6.4, 12.4 Hz, 1H), 3.97 (d, J=8.4 Hz, 6H), 2.76 (s, 3H), 2.66 (s, 3H), 1.26-1.23 (m, 3H).

LCMS (ESI) [M+H]⁺ m/z: calcd 357.1, found 357.0.

HPLC: 83.41%@220 nm, 92.53%@254 nm.

Chiral SFC: 92.6% ee

### Step 4: Synthesis of methyl 2-[5-[(4R)-5-amino-4-methyl-pentyl]-1-methyl-pyrazol-4-yl]-6-methyl-pyridine-4-carboxylate

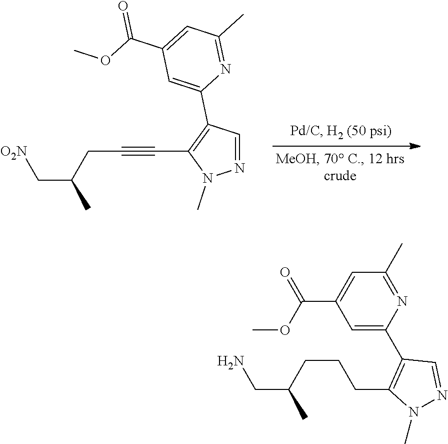 [SMILES: COC(=O)c1cc(C)nc(-c2cnn(C)c2C#CC[C@@H](C)C[N+](=O)[O-])c1 ; COC(=O)c1cc(C)nc(-c2cnn(C)c2CCC[C@@H](C)CN)c1]

To a solution of methyl 2-methyl-6-[1-methyl-5-[(4R)-4-methyl-5-nitro-pent-1-ynyl]pyrazol-4-yl]pyridine-4-carboxylate (2 g, 5.61 mmol, 1.0 eq) in MeOH (40.0 mL) was added Pd/C (2 g, 5.61 mmol, 10% purity, 1.0 eq). The suspension was degassed and purged with H₂ for 3 times. The mixture was stirred under H₂ (50 psi) at 70° C. for 12 hours. The reaction mixture was filtered and concentrated under reduced pressure to afford methyl 2-[5-[(4R)-5-amino-4-methyl-pentyl]-1-methyl-pyrazol-4-yl]-6-methyl-pyridine-4-carboxylate (1.85 g, crude) as yellow oil. ¹H NMR (400 MHz, CDCl₃) δ ppm 7.86 (s, 1H), 7.79 (s, 1H), 7.49 (s, 1H), 3.96 (s, 3H), 3.87 (s, 3H), 3.49 (s, 2H), 3.14-3.04 (m, 2H), 2.60 (s, 3H), 1.52-1.45 (m, 2H), 1.33-1.17 (m, 2H), 1.13 (d, J=6.4 Hz, 1H), 0.89 (d, J=6.8 Hz, 3H).

LCMS (ESI) [M+H]⁺ m/z: calcd 331.2, found 331.1.

### Step 5: Synthesis of methyl 2-[5-[(4R)-5-(5-bromo-2-nitro-anilino)-4-methyl-pentyl]-1-methyl-pyrazol-4-yl]-6-methyl-pyridine-4-carboxylate

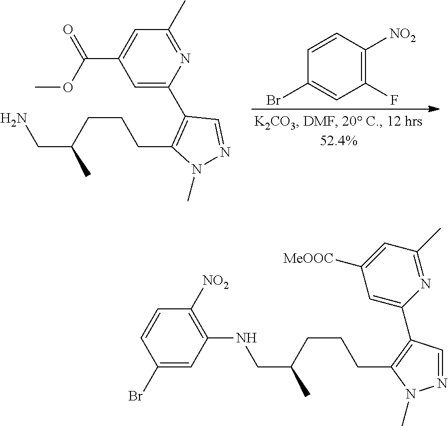 [SMILES: COC(=O)c1cc(C)nc(-c2cnn(C)c2CCC[C@@H](C)CN)c1 ; O=[N+]([O-])c1ccc(Br)cc1F ; COC(=O)c1cc(C)nc(-c2cnn(C)c2CCC[C@@H](C)CNc2cc(Br)ccc2[N+](=O)[O-])c1]

To a solution of methyl 2-[5-[(4R)-5-amino-4-methyl-pentyl]-1-methyl-pyrazol-4-yl]-6-methyl-pyridine-4-carboxylate (1.85 g, 5.60 mmol, 1.0 eq) and 4-bromo-2-fluoro-1-nitro-benzene (1.23 g, 5.60 mmol, 1.0 eq) in DMF (30.0 mL) was added K₂CO₃ (2.32 g, 16.8 mmol, 3.0 eq). The mixture was stirred at 20° C. for 12 hours. The reaction mixture was diluted with H₂O (60 mL) and extracted with EtOAc (80 mL*2). The combined organic layers were washed with H₂O (100 mL*1) and brine (100 mL*1), dried over anhydrous Na₂SO₄, filtered and concentrated under reduced pressure. The residue was purified by flash chromatography (ISCO®; 20 g SepaFlash® Silica Flash Column, Petroleum ethergradient/EtOAc with EtOAc from 0˜40%, 40 mL/min, 254 nm) to afford methyl 2-[5-[(4R)-5-(5-bromo-2-nitro-anilino)-4-methyl-pentyl]-1-methyl-pyrazol-4-yl]-6-methyl-pyridine-4-carboxylate (1.57 g, 52.4% yield, 99% purity) as a yellow solid.

¹H NMR (400 MHz, CDCl₃) δ ppm 8.16-8.08 (m, 1H), 8.03 (d, J=9.2 Hz, 1H), 7.86 (s, 1H), 7.80 (s, 1H), 7.47 (s, 1H), 6.96 (s, 1H), 6.75 (br d, J=9.2 Hz, 1H), 3.96 (s, 3H), 3.88 (s, 3H), 3.19-3.04 (m, 4H), 2.57 (s, 3H), 1.98-1.85 (m, 1H), 1.82-1.66 (m, 2H), 1.60-1.54 (m, 1H), 1.44-1.34 (m, 1H), 1.04 (d, J=6.4 Hz, 3H).

LCMS (ESI) [M+H]⁺ m/z: calcd 530.1, found 532.1.

HPLC: 99.13%@220 nm, 99.55%@254 nm.

Chiral SFC: 84.1% ee.

### Step 6: Synthesis of methyl 2-[5-[(4R)-5-(2-amino-5-bromo-anilino)-4-methyl-pentyl]-1-methyl-pyrazol-4-yl]-6-methyl-pyridine-4-carboxylate

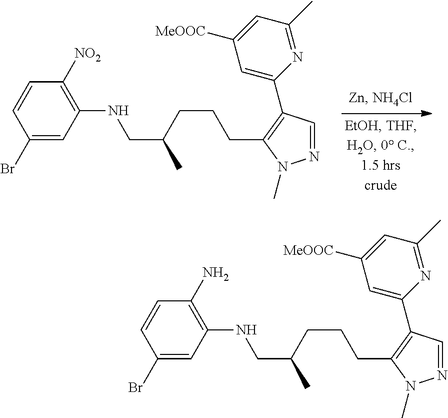 [SMILES: COC(=O)c1cc(C)nc(-c2cnn(C)c2CCC[C@@H](C)CNc2cc(Br)ccc2[N+](=O)[O-])c1 ; COC(=O)c1cc(C)nc(-c2cnn(C)c2CCC[C@@H](C)CNc2cc(Br)ccc2N)c1]

To a solution of methyl 2-[5-[(4R)-5-(5-bromo-2-nitro-anilino)-4-methyl-pentyl]-1-methyl-pyrazol-4-yl]-6-methyl-pyridine-4-carboxylate (1.57 g, 2.96 mmol, 1.0 eq) in EtOH (20.0 mL), THF (20.0 mL) and H₂O (10.0 mL) was added Zn (1.94 g, 29.6 mmol, 10.0 eq) and NH₄Cl (1.58 g, 29.6 mmol, 10.0 eq) at 0° C. The mixture was stirred at 0° C. for 1.5 hours. The reaction mixture was filtered and concentrated. The residue was diluted with H₂O (50 mL) and extracted with EtOAc (70 mL*2). The combined organic layers were washed with brine (50 mL*1), dried over anhydrous Na₂SO₄, filtered and concentrated under reduced pressure to afford methyl 2-[5-[(4R)-5-(2-amino-5-bromo-anilino)-4-methyl-pentyl]-1-methyl-pyrazol-4-yl]-6-methyl-pyridine-4-carboxylate (1.48 g, crude) as a yellow solid.

¹H NMR (400 MHz, CDCl₃) δ ppm 7.87 (s, 1H), 7.79 (s, 1H), 7.48 (s, 1H), 6.74 (dd, J=2.4, 8.0 Hz, 1H), 6.67 (d, J=2.0 Hz, 1H), 6.57 (d, J=8.0 Hz, 1H), 3.96 (s, 3H), 3.88 (s, 3H), 3.11 (t, J=7.6 Hz, 2H), 3.00-2.93 (m, 1H), 2.90-2.82 (m, 1H), 2.59 (s, 3H), 1.85-1.74 (m, 2H), 1.65-1.55 (m, 4H), 1.39-1.30 (m, 1H), 1.00 (d, J=6.4 Hz, 3H).

LCMS (ESI) [M+H]⁺ m/z: calcd 500.2, found 500.1.

### Step 7: Synthesis of methyl 2-[5-[(4R)-5-(2-amino-6-bromo-benzimidazol-1-yl)-4-methyl-pentyl]-1-methyl-pyrazol-4-yl]-6-methyl-pyridine-4-carboxylate

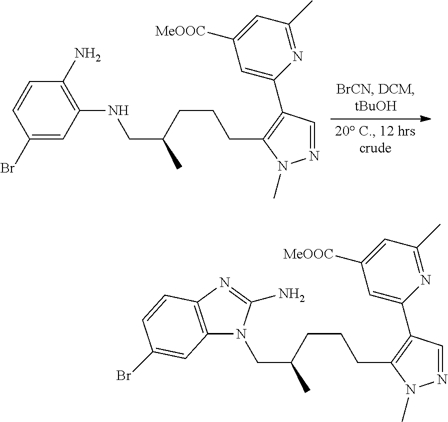 [SMILES: COC(=O)c1cc(C)nc(-c2cnn(C)c2CCC[C@@H](C)CNc2cc(Br)ccc2N)c1 ; COC(=O)c1cc(C)nc(-c2cnn(C)c2CCC[C@@H](C)Cn2c(N)nc3ccc(Br)cc32)c1]

To a solution of methyl 2-[5-[(4R)-5-(2-amino-5-bromo-anilino)-4-methyl-pentyl]-1-methyl-pyrazol-4-yl]-6-methyl-pyridine-4-carboxylate (1.48 g, 2.96 mmol, 1.0 eq) in DCM (20.0 mL) and t-BuOH (4.0 mL) was added CNBr (2 g, 18.9 mmol, 6.38 eq). The mixture was stirred at 20° C. for 12 hours. The reaction mixture was diluted with DCM (100 mL), treated with saturated aqueous NaHCO₃solution (50 mL) and stirred for 10 minutes. The layers were separated, and the organic phase was washed again with saturated aqueous NaHCO₃solution (50 mL). The organic phase was then dried over anhydrous Na₂SO₄, filtered and concentrated under reduced pressure to afford methyl 2-[5-[(4R)-5-(2-amino-6-bromo-benzimidazol-1-yl)-4-methyl-pentyl]-1-methyl-pyrazol-4-yl]-6-methyl-pyridine-4-carboxylate (1.55 g, crude) as a yellow solid.

¹H NMR (400 MHz, CDCl₃) δ ppm 7.88-7.84 (m, 1H), 7.80-7.74 (m, 1H), 7.49-7.44 (m, 1H), 7.27-7.15 (m, 2H), 7.02-6.93 (m, 1H), 3.96 (s, 3H), 3.88-3.85 (m, 3H), 3.70-3.50 (m, 1H), 3.14-3.03 (m, 2H), 2.56-2.50 (m, 3H), 1.89-1.63 (m, 4H), 1.58-1.50 (m, 1H), 1.42-1.31 (m, 1H), 0.99-0.93 (m, 3H).

LCMS (ESI) [M+H]⁺ m/z: calcd 525.2, found 527.1.

### Step 8: Synthesis of 2-[5-[(4R)-5-(2-amino-6-bromo-benzimidazol-1-yl)-4-methyl-pentyl]-1-methyl-pyrazol-4-yl]-6-methyl-pyridine-4-carboxylic acid

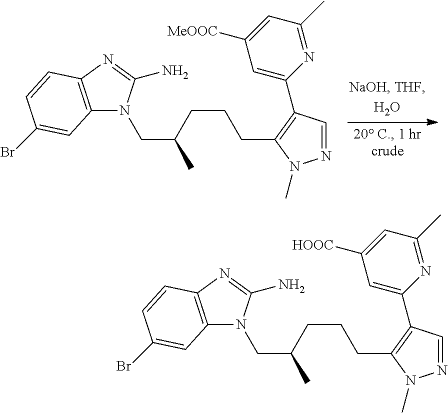 [SMILES: COC(=O)c1cc(C)nc(-c2cnn(C)c2CCC[C@@H](C)Cn2c(N)nc3ccc(Br)cc32)c1 ; Cc1cc(C(=O)O)cc(-c2cnn(C)c2CCC[C@@H](C)Cn2c(N)nc3ccc(Br)cc32)n1]

To a solution of methyl 2-[5-[(4R)-5-(2-amino-6-bromo-benzimidazol-1-yl)-4-methyl-pentyl]-1-methyl-pyrazol-4-yl]-6-methyl-pyridine-4-carboxylate (1.55 g, 2.95 mmol, 1.0 eq) in THF (20.0 mL) and H₂O (10.0 mL) was added NaOH (1.18 g, 29.5 mmol, 10.0 eq). The mixture was stirred at 20° C. for 1 hour. The reaction mixture was concentrated to remove THF, and then added TN HCl to adjust pH-5, the resultant slurry was filtered. The filter cake was washed with Petroleum ether, and then dried under reduced pressure to afford 2-[5-[(4R)-5-(2-amino-6-bromo-benzimidazol-1-yl)-4-methyl-pentyl]-1-methyl-pyrazol-4-yl]-6-methyl-pyridine-4-carboxylic acid (1.5 g, crude) as a red solid.

LCMS (ESI) [M+H]⁺ m/z: calcd 511.1, found 513.0.

### Step 9: Synthesis of (10R,20E)-15-bromo-5,10,25-trimethyl-4,5,12,19,21,26-hexazapentacyclo[21.3.1.02,6.012,20.013,18]heptacosa-1(27),2(6),3,13(18),14,16,20,23,25-nonaen-22-one

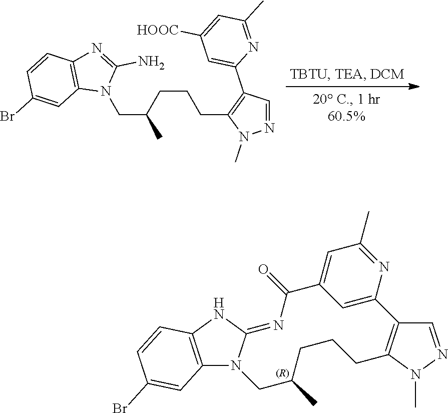

To a solution of 2-[5-[(4R)-5-(2-amino-6-bromo-benzimidazol-1-yl)-4-methyl-pentyl]-1-methyl-pyrazol-4-yl]-6-methyl-pyridine-4-carboxylic acid (1.5 g, 2.93 mmol, 1.0 eq) in DCM (580.0 mL) was added TBTU (1.41 g, 4.40 mmol, 1.5 eq) and Et₃N (2.1 mL, 14.7 mmol, 5.0 eq). The mixture was stirred at 20° C. for 1 hour. The reaction mixture was treated with saturated aqueous NaHCO₃solution (100 mL) and stirred for 10 minutes. The layers were separated, and the organic phase was washed again with saturated aqueous NaHCO₃solution (100 mL). The organic phase was then dried over Na₂SO₄, filtered, and concentrated under reduced pressure. The residue was purified by flash chromatography (ISCO®; 12 g SepaFlash® Silica Flash Column, Petroleum ether/EtOAc with EtOAc from 0˜100%, EtOAc/MeOH with MeOH from 0˜20%, 18 mL/min, 254 nm) to afford (10R,20E)-15-bromo-5,10,25-trimethyl-4,5,12,19,21,26-hexazapentacyclo[21.3.1.02,6.012,20.013,18]heptacosa-1(27),2(6),3,13(18),14,16,20,23,25-nonaen-22-one (1.3 g, 60.5% yield, 67% purity) as a yellow solid.

LCMS (ESI) [M+H]⁺ m/z: calcd 493.1, found 495.0.

### Step 10: Synthesis of (10R,20Z)-15-bromo-5,10,25-trimethyl-19-(2-trimethylsilylethoxymethyl)-4,5,12,19,21,26-hexazapentacyclo[21.3.1.02,6.012,20.013,18]heptacosa-1(27),2(6),3,13(18),14,16,20,23,25-nonaen-22-one

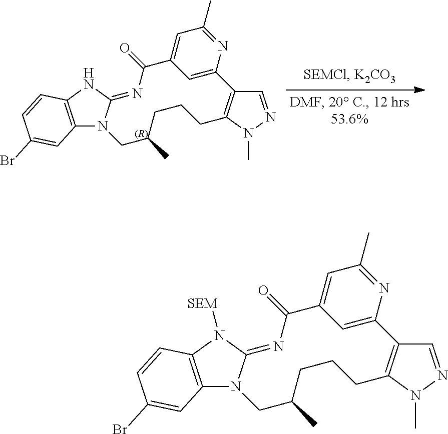

To a solution of (10R,20E)-15-bromo-5,10,25-trimethyl-4,5,12,19,21,26-hexazapentacyclo[21.3.1.02,6.012,20.013,18]heptacosa-1(27),2(6),3,13(18),14,16,20,23,25-nonaen-22-one (1.30 g, 2.63 mmol, 1.0 eq) and 2-(chloromethoxy)ethyl-trimethyl-silane (2.20 g, 13.2 mmol, 5.0 eq) in DMF (30.0 mL) was added K₂CO₃ (2.18 g, 15.8 mmol, 6.0 eq). The mixture was stirred at 20° C. for 12 hours. The reaction mixture was diluted with NH₄Cl (50 mL) and extracted with EtOAc (100 mL*2). The combined organic layers were washed with H₂O (100 mL*1) and brine (100 mL*1), dried over anhydrous Na₂SO₄, filtered and concentrated under reduced pressure. The residue was purified by flash chromatography (ISCO®; 20 g SepaFlash® Silica Flash Column, Petroleum ether/EtOAc with EtOAc from 0˜70%, 40 mL/min, 254 nm) to afford (10R,20Z)-15-bromo-5,10,25-trimethyl-19-(2-trimethylsilylethoxymethyl)-4,5,12,19,21,26-hexazapentacyclo[21.3.1.02,6.012,20.013,18]heptacosa-1(27),2(6),3,13(18),14,16,20,23,25-nonaen-22-one (880 mg, 53.6% yield, 100% purity) as a white solid.

¹H NMR (400 MHz, CDCl₃) δ ppm 8.30 (s, 1H), 7.61 (s, 1H), 7.43-7.34 (m, 3H), 6.11 (d, J=11.6 Hz, 1H), 5.82 (d, J=11.2 Hz, 1H), 4.53 (br dd, J=4.4, 13.6 Hz, 1H), 3.92 (s, 3H), 3.50-3.38 (m, 4H), 3.27-3.17 (m, 1H), 2.73-2.62 (m, 5H), 2.14-1.84 (m, 4H), 1.01 (br d, J=6.8 Hz, 3H), 0.88-0.76 (m, 2H), −0.16 (s, 9H).

LCMS (ESI) [M+H]⁺ m/z: calcd 623.2, found 625.1.

HPLC: 98.33%@220 nm, 99.05%@254 nm.

Chiral SFC: 85.5% ee.

### Step 11: Synthesis of (10R,20Z)-5,10,25-trimethyl-15-(4-methylpiperazin-1-yl)-19-(2-trimethylsilylethoxymethyl)-4,5,12,19,21,26-hexazapentacyclo[21.3.1.02,6.012,20.013,18]-heptacosa-1(27),2(6), 3,13(18),14,16,20,23,25-nonaen-22-one

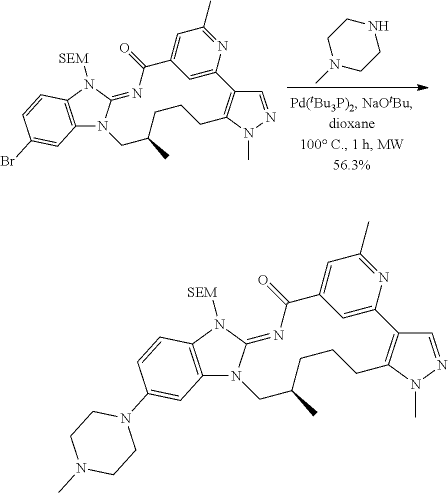

(10R,20Z)-15-bromo-5,10,25-trimethyl-19-(2-trimethylsilylethoxymethyl)-4,5,12,19,21,26-hexazapentacyclo[21.3.1.02,6.012,20.013,18]heptacosa-1(27),2(6),3,13(18),14,16,20,23,25-nonaen-22-one (100 mg, 0.160 mmol, 1.0 eq), 1-methylpiperazine (32 mg, 0.321 mmol, 2.0 eq) and sodium;2-methylpropan-2-olate (39 mg, 0.401 mmol, 2.5 eq) and palladium;tritert-butylphosphane (16 mg, 0.0321 mmol, 0.2 eq) were taken up into a microwave tube in dioxane (3.0 mL). The sealed tube was heated at 100° C. for 1 hour under microwave. The reaction mixture was concentrated under reduced pressure. The residue was purified by flash chromatography (ISCO®; 4 g SepaFlash® Silica Flash Column, Petroleum ether/EtOAc with EtOAc from 0-100%, then DCM/MeOH with MeOH from 0˜20%,18 m/min, 254 nm) to afford (10R,20Z)-5,10,25-trimethyl-15-(4-methylpiperazin-1-yl)-19-(2-trimethylsilylethoxymethyl)-4,5,12,19,21,26-hexazapentacyclo[21.3.1.02,6.012,20.013,18]heptacosa-1(27),2(6),3,13(18),14,16,20,23,25-nonaen-22-one (60 mg, 56.3% yield, 97% purity) as yellow oil.

LCMS (ESI) [M+H]⁺ m/z: calcd 643.4, found 643.4.

### Step 12: Synthesis of (10R,20E)-5,10,25-trimethyl-15-(4-methylpiperazin-1-yl)-4,5,12,19,21,26-hexazapentacyclo[21.3.1.02,6.012,20.013,18]heptacosa-1(27),2(6),3,13(18),14,16,20,23,25-nonaen-22-one (Compound (32))

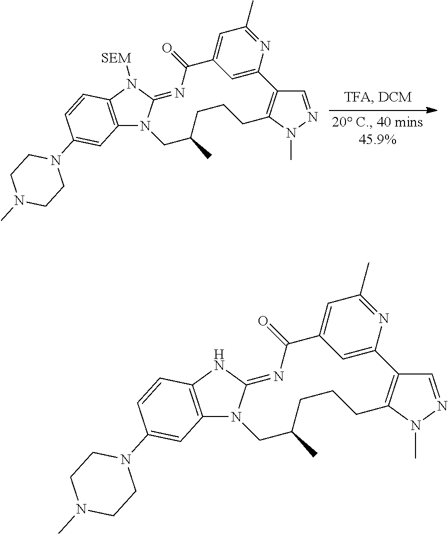 [SMILES: Cc1cc2cc(n1)-c1cnn(C)c1CCC[C@@H](C)CN1/C(=N/C2=O)N(COCC[Si](C)(C)C)c2ccc(N3CCN(C)CC3)cc21 ; Cc1cc2cc(n1)-c1cnn(C)c1CCC[C@@H](C)CN1/C(=N/C2=O)Nc2ccc(N3CCN(C)CC3)cc21]

To a solution of (10R,20Z)-5,10,25-trimethyl-15-(4-methylpiperazin-1-yl)-19-(2-trimethylsilylethoxymethyl)-4,5,12,19,21,26-hexazapentacyclo[21.3.1.02,6.012,20.013,18]heptacosa-1(27),2(6),3,13(18),14,16,20,23,25-nonaen-22-one (50 mg, 0.0778 mmol, 1.0 eq) in DCM (1.0 mL) and TFA (5.0 mL), and then the mixture was stirred at 20° C. for 40 minutes. The reaction mixture was concentrated to remove TFA, and then diluted with MeOH (5 mL), adjust pH-8 with saturated aqueous Na₂CO₃ solution, the reaction mixture was concentrated under reduced pressure. The crude product was purified by prep-HPLC (column: 2_Phenomenex Gemini C18 75*40 mm*3 μm; mobile phase: [water(NH₄HCO₃)-ACN];B %: 38%-68%,7.8 min) to afford (10R,20E)-5,10,25-trimethyl-15-(4-methylpiperazin-1-yl)-4,5,12,19,21,26-hexazapentacyclo[21.3.1.02,6.012,20.013,18]heptacosa-1(27),2(6),3,13(18),14,16,20,23,25-nonaen-22-one (18.6 mg, 45.9% yield) as an off-white solid.

¹H NMR (400 MHz, MeOD) δ ppm 8.51 (s, 1H), 7.93 (s, 1H), 7.55 (br s, 1H), 7.31 (br d, J=8.4 Hz, 1H), 7.04-6.88 (m, 2H), 4.30 (br d, J=12.8 Hz, 1H), 3.89 (s, 3H), 3.53 (br t, J=11.6 Hz, 1H), 3.24 (br s, 4H), 3.17-3.07 (m, 1H), 2.72 (br s, 4H), 2.56 (s, 4H), 2.42 (s, 3H), 2.07-1.85 (m, 2H), 1.79-1.66 (m, 1H), 1.63-1.51 (m, 1H), 1.36-1.29 (m, 1H), 0.90 (br d, J=6.0 Hz, 3H).

LCMS (ESI) [M+H]⁺ m/z: calcd 513.3, found 513.2.

HPLC: 98.77%@220 nm, 99.17%@254 nm.

Chiral SFC: 77.6% ee.

### Example 2: Preparation of Compound (10)

### The synthesis of (10R,20E)-5,10,25-trimethyl-15-(6-methyl-3-pyridyl)-4,5,12,19,21,26-hexazapentacyclo[21.3.1.02,6.012,20.013,18]heptacosa-1(27),2(6),3,13(18),14,16,20,23,25-nonaen-22-one (compound (10))

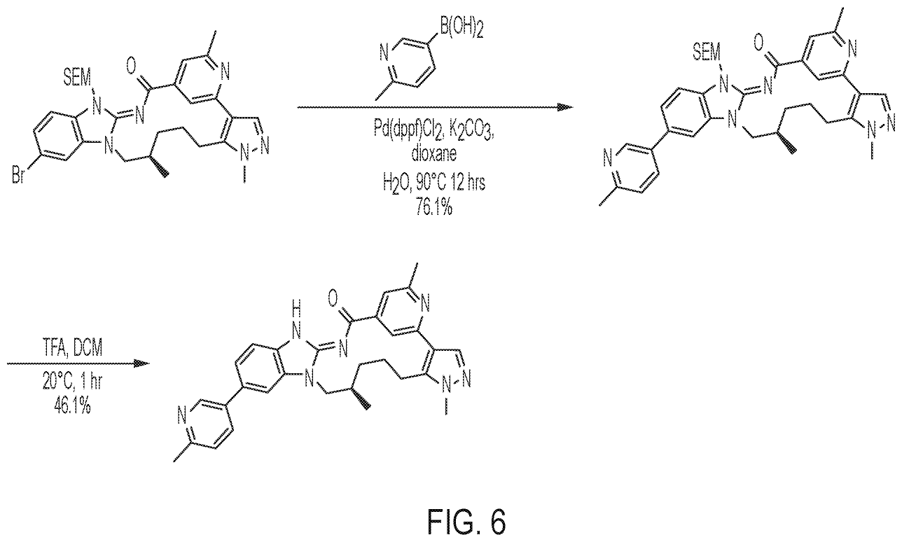

The general synthetic schemes for preparing Compound (10) according to the invention are provided in FIG. 6.

### Step 1: Synthesis of (10R,20Z)-5,10,25-trimethyl-15-(6-methyl-3-pyridyl)-19-(2-trimethylsilylethoxymethyl)-4,5,12,19,21,26-hexazapentacyclo[21.3.1.02,6.012,20.013,18]-heptacosa-1(27),2(6),3,13(18),14,16,20,23,25-nonaen-22-one

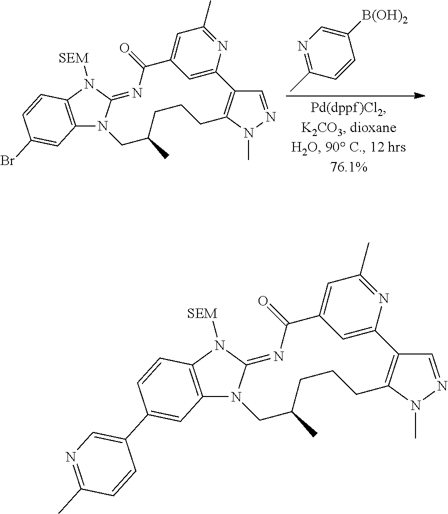

A mixture of (10R,20Z)-15-bromo-5,10,25-trimethyl-19-(2-trimethylsilylethoxymethyl)-4,5,12,19,21,26-hexazapentacyclo[21.3.1.02,6.012,20.013,18]heptacosa-1(27),2(6),3,13(18),14,16,20,23,25-nonaen-22-one (100 mg, 0.160 mmol, 1.0 eq, prepared as described in Example 1), (6-methyl-3-pyridyl)boronic acid (44 mg, 0.321 mmol, 2.0 eq), Pd(dppf)Cl₂ (24 mg, 0.0321 mmol, 0.2 eq) and K₂CO₃ (67 mg, 0.481 mmol, 3.0 eq) in dioxane (4.0 mL) and H₂O (0.8 mL) was degassed and purged with N2 for 3 times, and then the mixture was stirred at 90° C. for 12 hours under N2 atmosphere. The reaction mixture was diluted with H₂O (10 mL) and extracted with EtOAc (20 mL*2). The combined organic layers were washed with brine (20 mL*1), dried over anhydrous Na₂SO₄, filtered and concentrated under reduced pressure. The residue was purified by flash chromatography (ISCO®; 4 g SepaFlash® Silica Flash Column, Petroleum ether/EtOAc with EtOAc from 0-100%, then DCM/MeOH with MeOH from 0˜15% 18 mL/min, 254 nm) to afford (10R,20Z)-5,10,25-trimethyl-15-(6-methyl-3-pyridyl)-19-(2-trimethylsilylethoxymethyl)-4,5,12,19,21,26-hexazapentacyclo[21.3.1.02,6.012,20.013,18]-heptacosa-1(27),2(6),3,13(18),14,16,20,23,25-nonaen-22-one (90 mg, 76.1% yield, 86% purity) as a yellow solid.

¹H NMR (400 MHz, CDCl₃) δ ppm 8.76 (d, J=2.0 Hz, 1H), 8.31 (s, 1H), 8.13 (s, 1H), 7.81 (dd, J=2.4, 8.0 Hz, 1H), 7.61 (s, 1H), 7.58-7.54 (m, 1H), 7.49-7.46 (m, 1H), 7.38 (d, J=1.2 Hz, 1H), 7.30-7.27 (m, 1H), 6.19 (d, J=11.2 Hz, 1H), 5.89 (d, J=11.2 Hz, 1H), 4.59 (dd, J=4.4, 13.6 Hz, 1H), 3.93 (s, 3H), 3.58-3.49 (m, 3H), 3.31-3.20 (m, 1H), 2.78-2.61 (m, 8H), 2.01-1.74 (m, 4H), 1.03 (d, J=6.8 Hz, 3H), 0.91-0.77 (m, 2H), −0.15 (s, 9H).

LCMS (ESI) [M+H]⁺ m/z: calcd 636.3, found 636.4.

### Step 2: Synthesis of (10R,20E)-5,10,25-trimethyl-15-(6-methyl-3-pyridyl)-4,5,12,19,21,26-hexazapentacyclo[21.3.1.02,6.012,20.013,18]heptacosa-1(27),2(6),3,13(18),14,16,20,23,25-nonaen-22-one

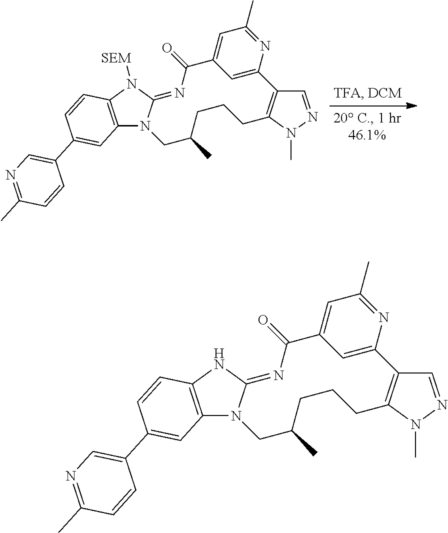 [SMILES: Cc1ccc(-c2ccc3c(c2)N2C[C@H](C)CCCc4c(cnn4C)-c4cc(cc(C)n4)C(=O)/N=C/2N3COCC[Si](C)(C)C)cn1 ; Cc1ccc(-c2ccc3c(c2)N2C[C@H](C)CCCc4c(cnn4C)-c4cc(cc(C)n4)C(=O)/N=C/2N3)cn1]

To a solution of (10R,20Z)-5,10,25-trimethyl-15-(6-methyl-3-pyridyl)-19-(2-trimethylsilylethoxymethyl)-4,5,12,19,21,26-hexazapentacyclo[21.3.1.02,6.012,20.013,18]-heptacosa-1(27),2(6),3,13(18),14,16,20,23,25-nonaen-22-one (90 mg, 0.142 mmol, 1.0 eq) in DCM (0.5 mL) and TFA (2.5 mL). The mixture was stirred at 20° C. for 1 hour. The reaction mixture was concentrated under reduced pressure. The residue was diluted with MeOH (5 mL), adjust pH-8 with saturated aqueous Na₂CO₃ solution, filtered and concentrated under reduced pressure. The crude product was purified by prep-HPLC(column: 2_Phenomenex Gemini C18 75*40 mm*3□m; mobile phase: [water(NH₄HCO₃)-ACN];B %: 42%-72%,9.5 min) to afford (10R,20E)-5,10,25-trimethyl-15-(6-methyl-3-pyridyl)-4,5,12,19,21,26-hexazapentacyclo-[21.3.1.02,6.012,20.013,18]heptacosa-1(27),2(6),3,13(18),14,16,20,23,25-nonaen-22-one (33.2 mg, 46.1% yield, 99% purity) as an off white solid.

¹H NMR (400 MHz, MeOD) δ ppm 8.66 (s, 1H), 8.48 (s, 1H), 8.00-7.94 (m, 1H), 7.92 (s, 1H), 7.62 (s, 1H), 7.59-7.52 (m, 1H), 7.47 (q, J=8.4 Hz, 2H), 7.38 (d, J=8.0 Hz, 1H), 4.34 (br d, J=10.8 Hz, 1H), 3.89 (s, 3H), 3.72-3.58 (m, 1H), 3.17-3.05 (m, 1H), 2.70-2.52 (m, 8H), 2.10-1.87 (m, 2H), 1.84-1.70 (m, 1H), 1.62-1.49 (m, 1H), 0.92 (br d, J=6.4 Hz, 3H).

LCMS (ESI) [M+H]⁺ m/z: calcd 506.3, found 506.2.

HPLC: 97.55%@220 nm, 99.34%@254 nm.

Chiral SFC: 100% ee.

### Example 3: Preparation of Compound (16)

### The synthesis of (10R,20E)-5,10,25-trimethyl-15-[(1-methyl-4-piperidyl)oxy]-4,5,12,19,21,26-hexazapentacyclo[21.3.1.0^{2,6}.0^{12,20}.0^{13,18}]heptacosa-1(27),2(6),3,13(18),14,16,20,23,25-nonaen-22-one (compound (16))

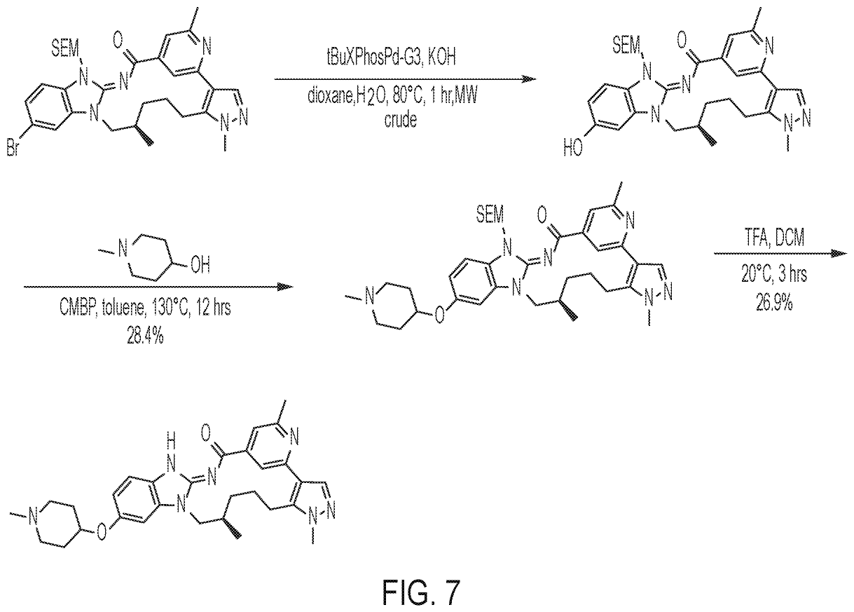

The general synthetic schemes for preparing Compound (16) according to the invention are provided in FIG. 7.

### Step 1: Synthesis of (10R,20Z)-15-hydroxy-5,10,25-trimethyl-19-(2-trimethylsilylethoxymethyl)-4,5,12,19,21,26-hexazapentacyclo[21.3.1.0^{2,6}.0^{12,20}.0^{13,18}]heptacosa-1(27),2(6), 3,13(18),14,16,20,23,25-nonaen-22-one

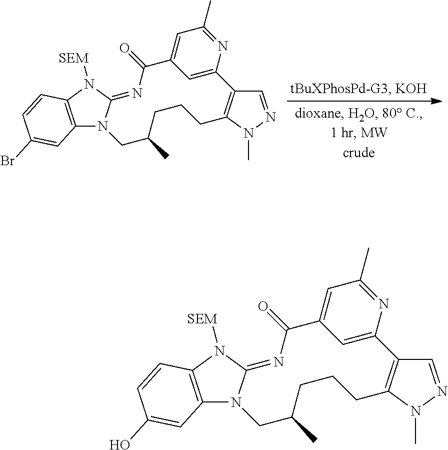 [SMILES: Cc1cc2cc(n1)-c1cnn(C)c1CCC[C@@H](C)CN1/C(=N/C2=O)N(COCC[Si](C)(C)C)c2ccc(Br)cc21 ; Cc1cc2cc(n1)-c1cnn(C)c1CCC[C@@H](C)CN1/C(=N/C2=O)N(COCC[Si](C)(C)C)c2ccc(O)cc21]

(10R,20Z)-15-bromo-5,10,25-trimethyl-19-(2-trimethylsilylethoxymethyl)-4,5,12,19,21,26-hexazapentacyclo[21.3.1.0^{2,6}.0^{12,20}.0^{13,18}]heptacosa-1(27),2(6),3,13(18),14,16,20,23,25-nonaen-22-one (150 mg, 0.240 mmol, 1.0 eq, prepared as described in Example 1), KOH (50 mg, 0.891 mmol, 3.7 eq) and [2-(2-aminophenyl)phenyl]-methylsulfonyloxy-palladium;ditert-butyl-[2-(2,4,6-triisopropylphenyl)phenyl]phosphane (40 mg, 0.0503 mmol, 0.2 eq) were taken up into a microwave tube in dioxane (10.0 mL) and H₂O (2.0 mL). The sealed tube was heated at 80° C. for 60 minute under microwave under N₂. The reaction mixture was filtered and the filter cake was washed with DCM (10 mL*3). The combined filtrate was partitioned between DCM (20 mL) and H₂O (50 mL). The organic phase was separated, dried over Na₂SO₄, filtered and concentrated under reduced pressure. The residue was purified by flash silica gel chromatography (ISCO©; 12 g SepaFlash© Silica Flash Column, Petroleum ether/EtOAc with EtOAc from 0˜100%, DCM/MeOH with MeOH from 0˜20%, flow rate=50 mL/min, 254 nm) to afford (10R,20Z)-15-hydroxy-5,10,25-trimethyl-19-(2-trimethylsilylethoxymethyl)-4,5,12,19,21,26-hexazapentacyclo[21.3.1.0^{2,6}.0^{12,20}.0^{13,18}]heptacosa-1(27),2(6),3,13(18),14,16,20,23,25-nonaen-22-one (60 mg, crude) as a brown oil.

LCMS (ESI) [M+H]⁺ m/z calcd 561.2, found 561.4.

### Step 2: Synthesis of (1R,20Z)-5,10,25-trimethyl-15-[(1-methyl-4-piperidyl)oxy]-19-(2-trimethylsilylethoxymethyl)-4,5,12,19,21,26-hexazapentacyclo[21.3.1.0^{2,6}.0^{12,20}.0^{13,18}]heptacosa-1(27),2(6), 3,13(18),14,16,20,23,25-nonaen-22-one

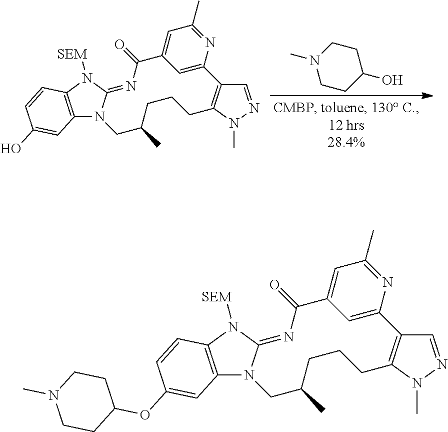

A mixture of (10R,20Z)-15-hydroxy-5,10,25-trimethyl-19-(2-trimethylsilylethoxymethyl)-4,5,12,19,21,26-hexazapentacyclo[21.3.1.0^{2,6}.0^{12,20}O^{13,18}]heptacosa-1(27),2(6),3,13(18),14,16,20,23,25-nonaen-22-one (60 mg, 0.107 mmol, 1.0 eq), 1-methylpiperidin-4-ol (37 mg, 0.321 mmol, 3.0 eq) and 2-(tributyl-λ⁵-phosphanylidene)acetonitrile (130 mg, 0.538 mmol, 5.0 eq) in toluene (10.0 mL) was degassed and purged with N2 for 3 times, and then the mixture was stirred at 130° C. for 12 hours under N2 atmosphere. The reaction mixture was concentrated under reduced pressure. The residue was purified by flash silica gel chromatography (ISCO®; 12 g SepaFlash® Silica Flash Column, Petroleum ether/EtOAc with EtOAc from 0-100%, DCM/MeOH with MeOH from 0-20%, flow rate=50 mL/min, 254 nm) to afford (10R,20Z)-5,10,25-trimethyl-15-[(1-methyl-4-piperidyl)oxy]-19-(2-trimethylsilylethoxymethyl)-4,5,12,19,21,26-hexazapentacyclo[21.3.1.0^{2,6}.0^{12,20}.0^{13,18}]heptacosa-1(27),2(6),3,13(18),14,16,20,23,25-nonaen-22-one (20 mg, 28.4% yield, 100% purity) as a brown oil.

LCMS (ESI) [M+H]⁺ m/z calcd 658.3, found 658.4.

### Step 3: Synthesis of (10R,20E)-5,10,25-trimethyl-5-[(I-methyl-4-piperidyl)oxy]-4,5,12,19,21,26-hexazapentacyclo[21.3.1.0^{2,6}.0^{12,20}.0^{13,18}]heptacosa-1(27),2(6),3,13(18),14,16,20,23,25-nonaen-22-one

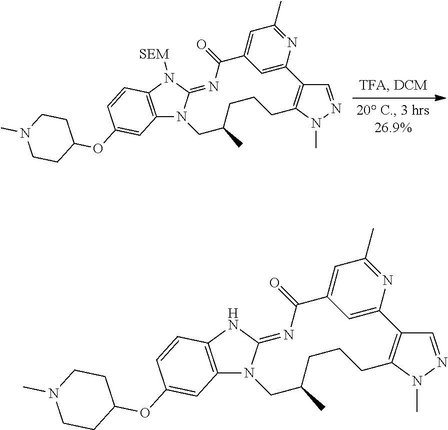 [SMILES: Cc1cc2cc(n1)-c1cnn(C)c1CCC[C@@H](C)CN1/C(=N/C2=O)N(COCC[Si](C)(C)C)c2ccc(OC3CCN(C)CC3)cc21 ; Cc1cc2cc(n1)-c1cnn(C)c1CCC[C@@H](C)CN1/C(=N/C2=O)Nc2ccc(OC3CCN(C)CC3)cc21]

To a solution of (10R,20Z)-5,10,25-trimethyl-15-[(1-methyl-4-piperidyl)oxy]-19-(2-trimethylsilylethoxymethyl)-4,5,12,19,21,26-hexazapentacyclo[21.3.1.0^{2,6}.0^{12,20}.0^{13,18}]-heptacosa-1(27),2(6),3,13(18),14,16,20,23,25-nonaen-22-one (20 mg, 0.0304 mmol, 1.0 eq) in DCM (2.0 mL) was added TFA (2.0 mL, 27.01 mmol, 888.6 eq). The mixture was stirred at 20° C. for 3 hours. The reaction mixture was concentrated under reduced pressure. The crude product was purified by preparative HPLC(column: ACE 5 C₁₈-AR 150*30 mm*5 μm; mobile phase: [water(FA)-ACN];B %: 10%-40%,9.5 min, Column Temp:30° C.) to afford (10R,20E)-5,10,25-trimethyl-15-[(1-methyl-4-piperidyl)oxy]-4,5,12,19,21,26-hexazapentacyclo-[21.3.1.0^{2,6}.0^{12,20}.0^{13,18}]heptacosa-1(27),2(6),3,13(18),14,16,20,23,25-nonaen-22-one (4.7 mg, 26.9% yield, 91.72% purity) as a yellow solid.

¹H NMR (400 MHz, methanol-d₄) δ ppm 8.92-8.89 (m, 1H), 8.10-8.06 (m, 1H), 7.97-7.93 (m, 1H), 7.47-7.43 (m, 1H), 7.29-7.16 (m, 1H), 7.07-6.96 (m, 1H), 4.53-4.43 (m, 1H), 4.00-3.97 (m, 3H), 3.77-3.59 (m, 2H), 3.49-3.37 (m, 3H), 3.25-3.11 (m, 2H), 2.96-2.93 (m, 3H), 2.79 (s, 3H), 2.70-2.59 (m, 1H), 2.47-2.36 (m, 1H), 2.34-2.21 (m, 2H), 2.18-2.02 (m, 4H), 1.90-1.81 (m, 1H), 1.71-1.61 (m, 1H), 0.99-0.95 (m, 3H)

LCMS (ESI) [M+H]⁺ m/z calcd 528.3, found 528.3.

HPLC: 91.72%@220 nm, 92.25%@254 nm.

### Example 4: Preparation of Compound (41)

### The synthesis of (11R)-5,11,26-trimethyl-16-[(4-methylpiperazin-1-yl)methyl]-4,5,13,20,22,27-hexazapentacyclo[22.3.1.0^{2,6}.0^{13,21}.0^{14,19}]octacosa-1(27),2(6),3,14(19),15,17,20,24(28),25-nonaen-23-one (compound (41))

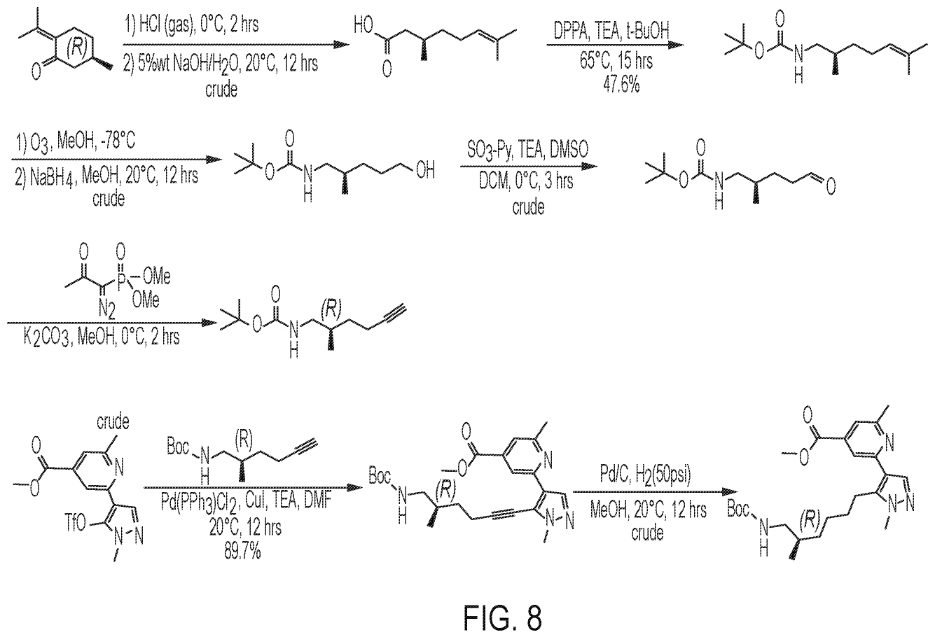

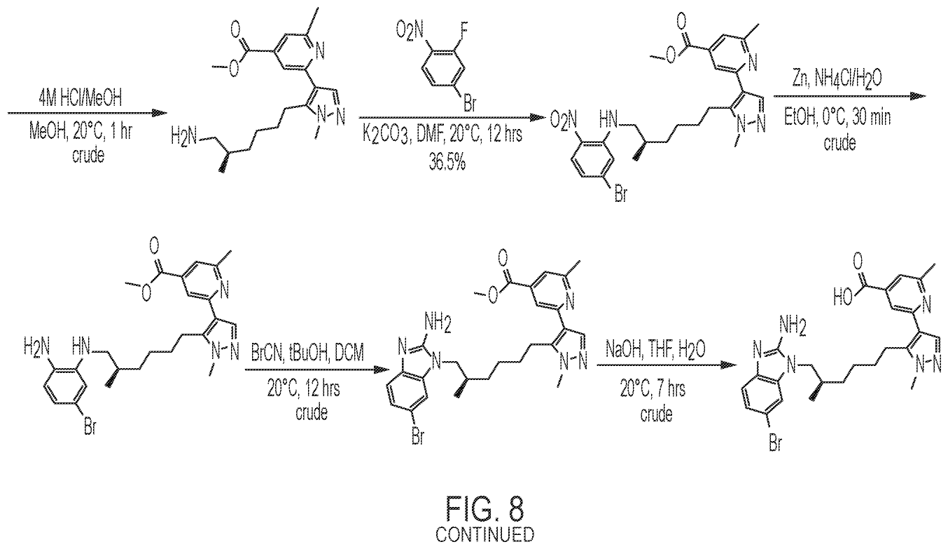

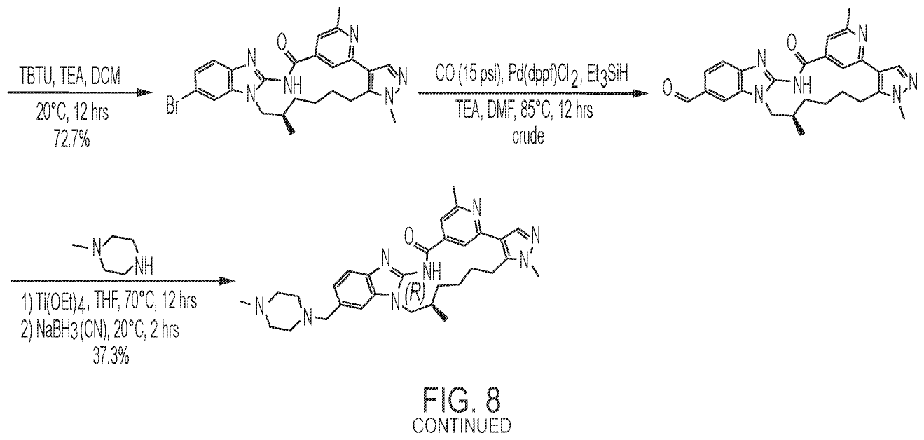

The general synthetic schemes for preparing Compound (41) according to the invention are provided in FIG. 8.

### Step 1: (3R)-3,7-dimethyloct-6-enoic acid

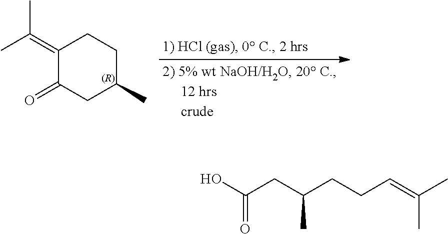

Dry HCl gas passed through (5R)-2-isopropylidene-5-methyl-cyclohexanone (12 g, 78.8 mmol, 1.0 eq) at −30° C. for 3 hours and then without isolating the intermediate pulegone hydrochloride, the reaction mixture was transferred to re-sealable reaction tube and the mixture stirred at 20° C. for 12 hours. The reaction mixture was diluted with H₂O (300 mL) and washed with petroleum ether (300 mL*3). The aqueous layer was again treated with 4N HCl/H₂O (pH=4) and the mixture was extracted with EtOAc (100 mL*3). The organic phase was washed with water (300 mL), brine (300 mL), dried over anhydrous Na₂SO₄, filtered and concentrated under reduced pressure to afford (3R)-3,7-dimethyloct-6-enoic acid (2.9 g, crude) as yellow oil.

¹H NMR (400 MHz, CDCl₃) δ ppm 5.02 (br t, J=7.0 Hz, 1H), 2.34-2.28 (m, 1H), 2.13-2.03 (m, 1H), 2.00-1.81 (m, 3H), 1.61 (s, 3H), 1.53 (s, 3H), 1.37-1.27 (m, 1H), 1.23-1.13 (m, 1H), 0.91 (d, J=6.7 Hz, 3H).

### Step 2: tert-butyl N-[(2R)-2,6-dimethylhept-5-enyl]carbamate

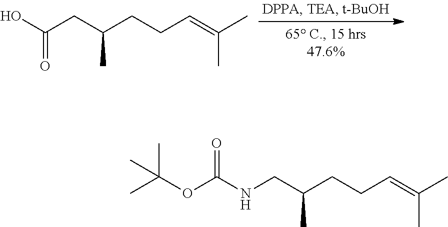 [SMILES: CC(C)=CCC[C@@H](C)CC(=O)O ; CC(C)=CCC[C@@H](C)CNC(=O)OC(C)(C)C]

A mixture of (3R)-3,7-dimethyloct-6-enoic acid (4.0 g, 23.5 mmol, 1.0 eq), DPPA (7.2 g, 26.2 mmol, 1.1 eq), TEA (2.9 g, 28.7 mmol, 1.2 eq) in t-BuOH (20.0 mL) was degassed and purged with N2 for 3 times, and the mixture was stirred at 65° C. for 15 hours under N2 atmosphere. The residue was poured into water (20 mL) and the aqueous phase was extracted with EtOAc (20 mL*3). The combined organic phase was washed with brine (30 mL), dried with anhydrous Na₂SO₄, filtered and concentrated in vacuum. The residue was purified by flash chromatography (ISCO®; 20 g SepaFlash® Silica Flash Column, petroleum ether/EtOAc with EtOAc from 0˜6%, 80 mL/min, 254 nm) to give product tert-butyl N-[(2R)-2,6-dimethylhept-5-enyl]carbamate (3.0 g, 47.6% yield, 90% purity) as colorless oil.

### Step 3: tert-butyl N-[(2R)-5-hydroxy-2-methyl-pentyl]carbamate

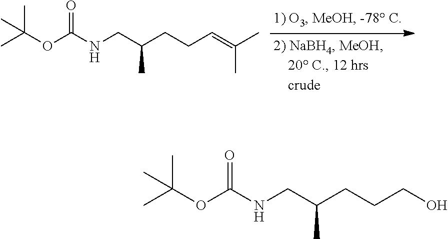 [SMILES: CC(C)=CCC[C@@H](C)CNC(=O)OC(C)(C)C ; C[C@H](CCCO)CNC(=O)OC(C)(C)C]

Tert-butyl N-[(2R)-2,6-dimethylhept-5-enyl]carbamate (3.0 g, 12.4 mmol, 1.0 eq) was dissolved in MeOH (20.0 mL). The solution mixture was cooled to −78° C., at which point 03 (12.4 mmol, 1.0 eq) was bubbled through the solution until the solution turned blue. Solution mixture was then purged with argon until the solution turned colorless. NaBH₄ (1.9 g, 50.3 mmol, 4.0 eq) was added. Upon addition completion, reaction mixture was slowly warmed to 20° C., and was stirred at 20° C. for 12 hours. The reaction was subsequently quenched with H₂O (60 mL), and methanol was removed on the rotary evaporator. The crude mixture was diluted with EtOAc. Organic layer was dried over MgSO₄, filtered and concentrated in vacuum. The residue was purified by flash chromatography (ISCO®; 20 g SepaFlash® Silica Flash Column, EtOAc/MeOH with MeOH from 0˜45%, 40 mL/min, 254 nm) to give product tert-butyl N-[(2R)-5-hydroxy-2-methyl-pentyl]carbamate (1.9 g, crude) as colorless oil

### Step 4: tert-butyl N-[(2R)-2-methyl-5-oxo-pentyl]carbamate

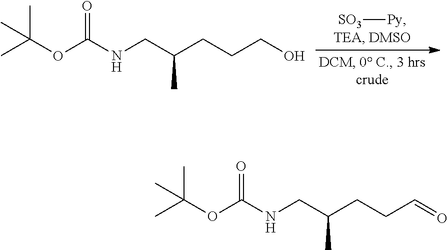

To a solution of tert-butyl N-[(2R)-5-hydroxy-2-methyl-pentyl]carbamate (1.9 g, 8.74 mmol, 1.0 eq) in DCM (20.0 mL) was added DMSO (850 mg, 10.9 mmol, 1.3 eq) and DIEA (4.8 g, 37.1 mmol, 4.2 eq), SO₃-Py (4.3 g, 26.7 mmol, 3.0 eq) at 0° C. The mixture was stirred at 0° C. for 3 hours under N2. Water (30 mL) was added and the mixture was extracted with EtOAc (20 mL*3). The combined organic phase was washed with brine (30 mL), dried with anhydrous Na₂SO₄, filtered and concentrated in vacuum. The crude product tert-butyl N-[(2R)-2-methyl-5-oxo-pentyl]carbamate (1.7 g, crude) was used into the next step without further purification.

### Step 5: tert-butyl N-[(2R)-2-methylhex-5-ynyl]carbamate

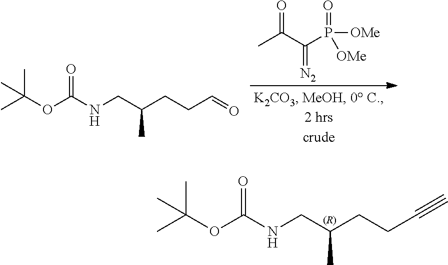

To a solution of tert-butyl N-[(2R)-2-methyl-5-oxo-pentyl]carbamate (1.7 g, 7.90 mmol, 1.0 eq) in MeOH (20.0 mL) were added K₂CO₃ (5.5 g, 39.8 mmol, 5.0 eq) and 1-diazo-1-dimethoxyphosphoryl-propan-2-one (2.2 g, 11.5 mmol, 1.4 eq) at 0° C. The mixture was stirred at 20° C. for 12 hours. Water (50 mL) was added and the mixture was extracted with EtOAc (30 mL*3). The combined organic phase was washed with brine (50 mL), dried with anhydrous Na₂SO₄, filtered and concentrated in vacuum. Compound tert-butyl N-[(2R)-2-methylhex-5-ynyl]carbamate (1.4 g, crude) was obtained as yellow oil.

### Step 6: methyl 2-[5-[(5R)-6-(tert-butoxycarbonylamino)-5-methyl-hex-1-ynyl]-1-methyl-pyrazol-4-yl]-6-methyl-pyridine-4-carboxylate

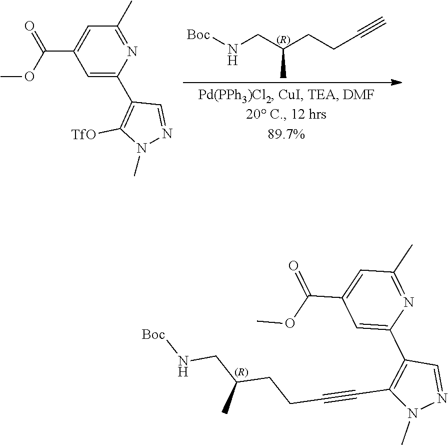

A mixture of methyl 2-methyl-6-[1-methyl-5-(trifluoromethylsulfonyloxy)pyrazol-4-yl]pyridine-4-carboxylate (1.3 g, 3.56 mmol, 1.0 eq), tert-butyl N-[(2R)-2-methylhex-5-ynyl]carbamate (1.4 g, 6.39 mmol, 1.8 eq), CuI (135 mg, 0.709 mmol, 0.2 eq), TEA (1.9 g, 18.7 mmol, 5.3 eq) and Pd(PPh₃)₂Cl₂ (540 mg, 0.770 mmol, 0.2 eq) in DMF (20.0 mL) was degassed and purged with N2 for 3 times, and then the mixture was stirred at 20° C. for 12 hours under N2 atmosphere. The residue was poured into water (150 mL). The aqueous phase was extracted with EtOAc (100 mL*3). The combined organic phase was washed with brine (200 mL), dried with anhydrous Na₂SO₄, filtered and concentrated in vacuum. The residue was purified by flash chromatography (ISCO®; 20 g SepaFlash® Silica Flash Column, Petroleum ether: Ethyl acetate with Ethyl acetate from 0˜27%, 80 mL/min, 254 nm) to give product methyl 2-[5-[(5R)-6-(tert-butoxycarbonylamino)-5-methyl-hex-1-ynyl]-1-methyl-pyrazol-4-yl]-6-methyl-pyridine-4-carboxylate (1.5 g, 89.7% yield, 93.8% purity) as yellow oil.

LCMS [M+H]⁺ m/z: calcd 441.2, found 441.1.

### Step 7: methyl 2-[5-[(5R)-6-(tert-butoxycarbonylamino)-5-methyl-hexyl]-1-methyl-pyrazol-4-yl]-6-methyl-pyridine-4-carboxylate

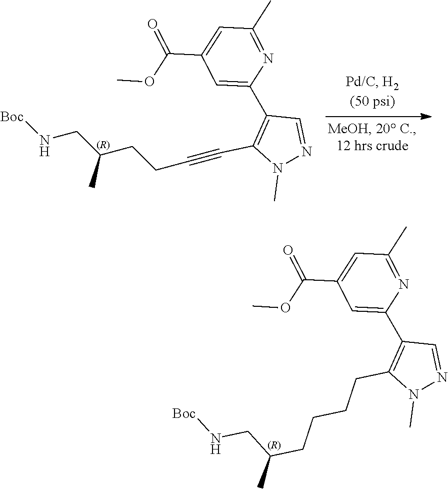

To a solution of methyl 2-[5-[(5R)-6-(tert-butoxycarbonylamino)-5-methyl-hex-1-ynyl]-1-methyl-pyrazol-4-yl]-6-methyl-pyridine-4-carboxylate (1.3 g, 2.95 mmol, 1.0 eq) in MeOH (15.0 mL) was added Pd/C (1.3 g, 10 wt % Pd with 50 wt % water) under N2. The suspension was degassed under vacuum and purged with H₂ several for times. The mixture was stirred under H₂ (50 psi) at 50° C. for 12 hours. The reaction mixture was filtered and the filtrate was concentrated. Compound methyl 2-[5-[(5R)-6-(tert-butoxycarbonylamino)-5-methyl-hexyl]-1-methyl-pyrazol-4-yl]-6-methyl-pyridine-4-carboxylate (800 mg, crude) was obtained as yellow oil.

LCMS [M+H]⁺ m/z: calcd 445.2, found 445.2.

### Step 8: methyl 2-[5-[(5R)-6-amino-5-methyl-hexyl]-1-methyl-pyrazol-4-yl]-6-methyl-pyridine-4-carboxylate:hydrochloride

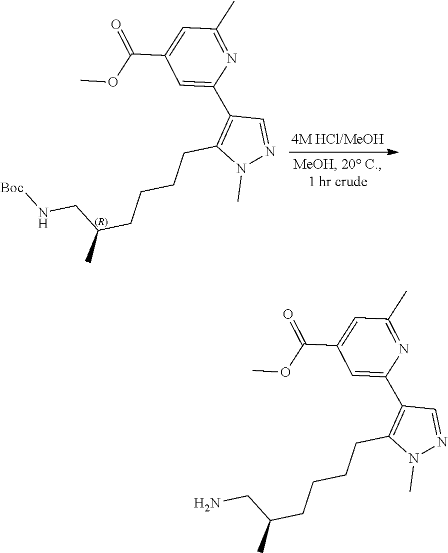

To a solution of methyl 2-[5-[(5R)-6-(tert-butoxycarbonylamino)-5-methyl-hexyl]-1-methyl-pyrazol-4-yl]-6-methyl-pyridine-4-carboxylate (800 mg, 1.80 mmol, 1.0 eq) in MeOH (5.0 mL) was added 4M HCl/MeOH (20.0 mL, 80 mmol, 44.5 eq). The mixture was stirred at 20° C. for 1.5 hours. The reaction mixture was concentrated under reduced pressure. Compound methyl 2-[5-[(5R)-6-amino-5-methyl-hexyl]-1-methyl-pyrazol-4-yl]-6-methyl-pyridine-4-carboxylate;hydrochloride (700 mg, crude) was obtained as a yellow solid.

### Step 9: methyl 2-[5-[(5R)-6-(5-bromo-2-nitro-anilino)-5-methyl-hexyl]-1-methyl-pyrazol-4-yl]-6-methyl-pyridine-4-carboxylate

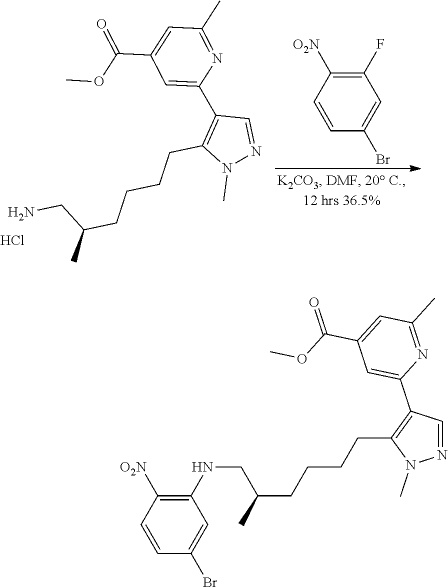 [SMILES: Cl ; COC(=O)c1cc(C)nc(-c2cnn(C)c2CCCC[C@@H](C)CN)c1 ; O=[N+]([O-])c1ccc(Br)cc1F ; COC(=O)c1cc(C)nc(-c2cnn(C)c2CCCC[C@@H](C)CNc2cc(Br)ccc2[N+](=O)[O-])c1]

To a solution of methyl 2-[5-[(5R)-6-amino-5-methyl-hexyl]-1-methyl-pyrazol-4-yl]-6-methyl-pyridine-4-carboxylate;hydrochloride (700 mg, 1.84 mmol, 1.0 eq) and 4-bromo-2-fluoro-1-nitro-benzene (410 mg, 1.86 mmol, 1.0 eq) in DMF (15.0 mL) was added K₂C₀₃ (2.5 g, 18.4 mmol, 10.0 eq). The mixture was stirred at 20° C. for 12 hours. The residue was poured into water (50 mL). The aqueous phase was extracted with ethyl acetate (20 mL*3). The combined organic phase was washed with brine (50 mL), dried with anhydrous Na₂SO₄, filtered and concentrated in vacuum. The residue was purified by flash chromatography (ISCO®; 12 g SepaFlash® Silica Flash Column, Petroleum ether: Ethyl acetate with Ethyl acetate from 0˜53%, 40 mL/min, 254 nm) to give product methyl 2-[5-[(5R)-6-(5-bromo-2-nitro-anilino)-5-methyl-hexyl]-1-methyl-pyrazol-4-yl]-6-methyl-pyridine-4-carboxylate (400 mg, 36.5% yield, 91.4% purity) as yellow oil.

LCMS [M+Na]⁺ m/z: calcd 568.1, found 568.1.

### Step 10: methyl 2-[5-[(5R)-6-(2-amino-5-bromo-anilino)-5-methyl-hexyl]-1-methyl-pyrazol-4-yl]-6-methyl-pyridine-4-carboxylate

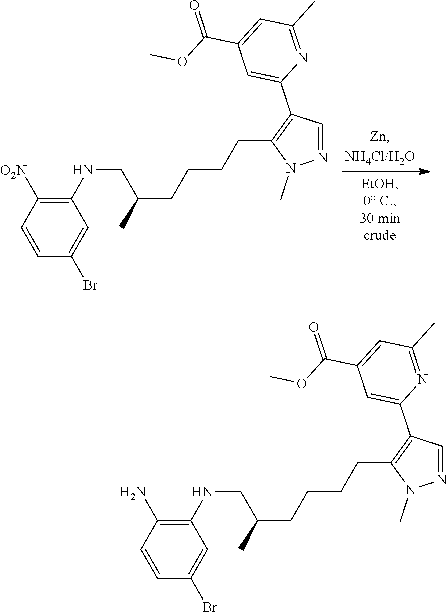 [SMILES: COC(=O)c1cc(C)nc(-c2cnn(C)c2CCCC[C@@H](C)CNc2cc(Br)ccc2[N+](=O)[O-])c1 ; COC(=O)c1cc(C)nc(-c2cnn(C)c2CCCC[C@@H](C)CNc2cc(Br)ccc2N)c1]

To a solution of methyl 2-[5-[(5R)-6-(5-bromo-2-nitro-anilino)-5-methyl-hexyl]-1-methyl-pyrazol-4-yl]-6-methyl-pyridine-4-carboxylate (360 mg, 0.661 mmol, 1.0 eq) in EtOH (20.0 mL) and H₂O (10.0 mL) was added NH₄Cl (360 mg, 6.73 mmol, 10.2 eq) and Zn (360 mg, 5.51 mmol, 8.3 eq). The mixture was stirred at 0° C. for 30 minutes. The reaction mixture was filtered and the filtrate was concentrated. Water (20 mL) was added and the mixture was extracted with DCM (15 mL*3). The combined organic phase was washed with brine (30 mL), dried over anhydrous Na₂SO₄, filtered and concentrated in vacuum. Compound methyl 2-[5-[(5R)-6-(2-amino-5-bromo-anilino)-5-methyl-hexyl]-1-methyl-pyrazol-4-yl]-6-methyl-pyridine-4-carboxylate (310 mg, crude) was obtained as a yellow oil.

### Step 11: methyl 2-[5-[(5R)-6-(2-amino-6-bromo-benzimidazol-1-yl)-5-methyl-hexyl]-1-methyl-pyrazol-4-yl]-6-methyl-pyridine-4-carboxylate

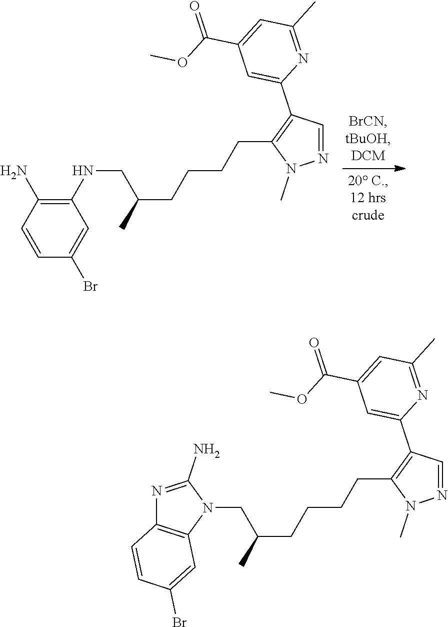 [SMILES: COC(=O)c1cc(C)nc(-c2cnn(C)c2CCCC[C@@H](C)CNc2cc(Br)ccc2N)c1 ; COC(=O)c1cc(C)nc(-c2cnn(C)c2CCCC[C@@H](C)Cn2c(N)nc3ccc(Br)cc32)c1]

To a solution of methyl 2-[5-[(5R)-6-(2-amino-5-bromo-anilino)-5-methyl-hexyl]-1-methyl-pyrazol-4-yl]-6-methyl-pyridine-4-carboxylate (310 mg, 0.602 mmol, 1.0 eq) in t-BuOH (2.0 mL) and DCM (10.0 mL) was added BrCN (496 mg, 4.68 mmol, 7.8 eq). The mixture was stirred at 20° C. for 12 hours. The reaction mixture was treated with saturated aqueous NaHCO₃solution (30 mL) and stirred for 10 min. The layers were separated, and the organic phase was washed again with saturated aqueous NaHCO₃solution. The organic phase was then dried over Na₂SO₄, filtered, and concentrated under reduced pressure. Compound methyl 2-[5-[(5R)-6-(2-amino-6-bromo-benzimidazol-1-yl)-5-methyl-hexyl]-1-methyl-pyrazol-4-yl]-6-methyl-pyridine-4-carboxylate (310 mg, crude) was obtained as a yellow solid.

### Step 12: 2-[5-[(5R)-6-(2-amino-6-bromo-benzimidazol-1-yl)-5-methyl-hexyl]-1-methyl-pyrazol-4-yl]-6-methyl-pyridine-4-carboxylic acid

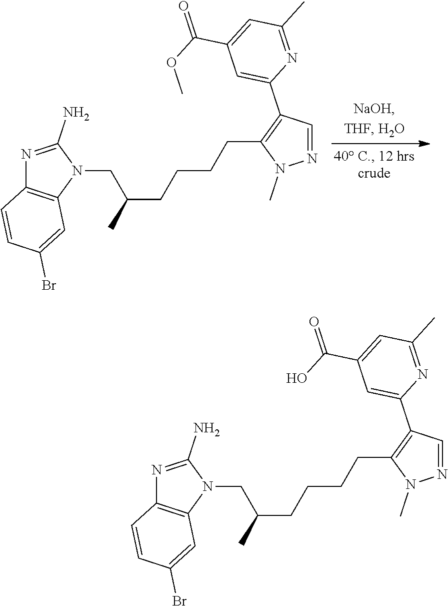 [SMILES: COC(=O)c1cc(C)nc(-c2cnn(C)c2CCCC[C@@H](C)Cn2c(N)nc3ccc(Br)cc32)c1 ; Cc1cc(C(=O)O)cc(-c2cnn(C)c2CCCC[C@@H](C)Cn2c(N)nc3ccc(Br)cc32)n1]

To a solution of methyl 2-[5-[(5R)-6-(2-amino-6-bromo-benzimidazol-1-yl)-5-methyl-hexyl]-1-methyl-pyrazol-4-yl]-6-methyl-pyridine-4-carboxylate (310 mg, 0.575 mmol, 1.0 eq) in THF (10.0 mL) and H₂O (5.0 mL) was added NaOH (300 mg, 7.50 mmol, 13.1 eq) The mixture was stirred at 20° C. for 7 hours. The solution was acidified with 6N aqueous HCl/H₂O to pH=5 and the mixture was extracted with a mixture of DCM and IPA (30 mL*3, 5/1). The combined organic phase was washed with brine (100 mL), dried with anhydrous Na₂SO₄, filtered and concentrated in vacuum. Compound 2-[5-[(5R)-6-(2-amino-6-bromo-benzimidazol-1-yl)-5-methyl-hexyl]-1-methyl-pyrazol-4-yl]-6-methyl-pyridine-4-carboxylic acid (310 mg, crude) was obtained as a yellow solid.

LCMS [M+H]⁺ m/z: calcd 525.1, found 525.1.

### Step 13: (11R)-16-bromo-5,11,26-trimethyl-4,5,13,20,22,27-hexazapentacyclo-[22.3.1.0^{2,6}.0^{12,21}.0^{14,19}]octacosa-1(27),2(6),3,14(19),15,17,20,24(28),25-nonaen-23-one

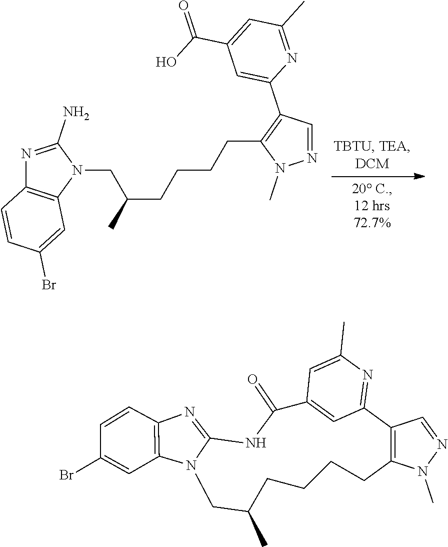 [SMILES: Cc1cc(C(=O)O)cc(-c2cnn(C)c2CCCC[C@@H](C)Cn2c(N)nc3ccc(Br)cc32)n1 ; Cc1cc2cc(n1)-c1cnn(C)c1CCCC[C@@H](C)Cn1c(nc3ccc(Br)cc31)NC2=O]

To a solution of 2-[5-[(5R)-6-(2-amino-6-bromo-benzimidazol-1-yl)-5-methyl-hexyl]-1-methyl-pyrazol-4-yl]-6-methyl-pyridine-4-carboxylic acid (260 mg, 0.495 mmol, 1.0 eq) in DCM (180.0 mL) was added TBTU (260 mg, 0.810 mmol, 1.7 eq) and TEA (363 mg, 3.59 mmol, 7.3 eq) The mixture was stirred at 20° C. for 2 hours. Water (30 mL) was added and the mixture was extracted with DCM (30 mL*3). The combined organic phase was washed with brine (50 mL), dried with anhydrous Na₂SO₄, filtered and concentrated in vacuum. The residue was purified by flash chromatography (ISCO®; 12 g SepaFlash® Silica Flash Column, Petroleum ether: Ethyl acetate with Ethyl acetate from 0˜90%, 40 mL/min, 254 nm) to give product (11R)-16-bromo-5,11,26-trimethyl-4,5,13,20,22,27-hexazapentacyclo[22.3.1.0^{2,6}.0^{13,21}.0^{14,19}]octacosa-1(27),2(6),3,14(19),15,17,20,24(28),25-nonaen-23-one(190 mg, 72.7% yield, 96.1% purity) as an off-white solid.

LCMS [M+H]⁺ m/z: calcd 507.1, found 507.1.

### Step 14: (11R)-5,11,26-trimethyl-23-oxo-4,5,13,20,22,27-hexazapentacyclo-[22.3.1.0^{2,6}.0^{13,21}.0^{14,19}]octacosa-1(27),2(6),3,14(19),15,17,20,24(28),25-nonaene-16-carbaldehyde

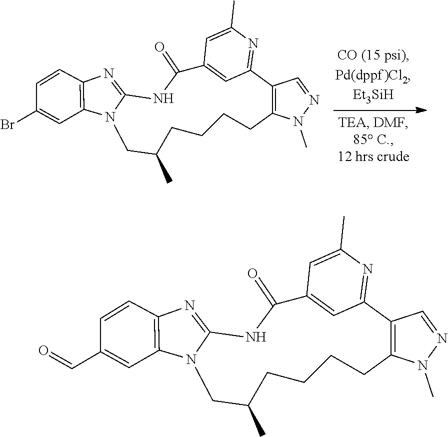 [SMILES: Cc1cc2cc(n1)-c1cnn(C)c1CCCC[C@@H](C)Cn1c(nc3ccc(Br)cc31)NC2=O ; Cc1cc2cc(n1)-c1cnn(C)c1CCCC[C@@H](C)Cn1c(nc3ccc(C=O)cc31)NC2=O]

A mixture of (11R)-16-bromo-5,11,26-trimethyl-4,5,13,20,22,27-hexazapentacyclo[22.3.1.0^{2,6}.0^{13,21}.0^{14,19}]octacosa-1(27),2(6),3,14(19),15,17,20,24(28),25-nonaen-23-one (100 mg, 0.197 mmol, 1.0 eq), Pd(dppf)Cl₂ (30 mg, 0.041 mmol, 0.2 eq), TEA (200 mg, 1.98 mmol, 10.0 eq) and triethylsilane (200 mg, 1.72 mmol, 8.7 eq) in DMF (15.0 mL) was degassed and purged with CO for 3 times, and then the mixture was stirred at 85° C. for 12 hours under CO (15 psi) atmosphere. The reaction mixture was filtered and the filtrated was concentrated. Compound (11R)-5,11,26-trimethyl-23-oxo-4,5,13,20,22,27-hexazapentacyclo[22.3.1.0^{2,6}.0^{13,21}.0^{14,19}]octacosa-1(27),2(6),3,14(19),15,17,20,24(28),25-nonaene-16-carbaldehyde (100 mg, crude) was obtained as a brown oil.

### Step 15: (11R)-5,11,26-trimethyl-16-[(4-methylpiperazin-1-yl)methyl]-4, 5,13, 20, 22,27-hexazapentacyclo[22.3.1.02,6.013,21.014,19]octacosa-1(27),2(6),3,14(19),15,17,20,24(28),25-nonaen-23-one

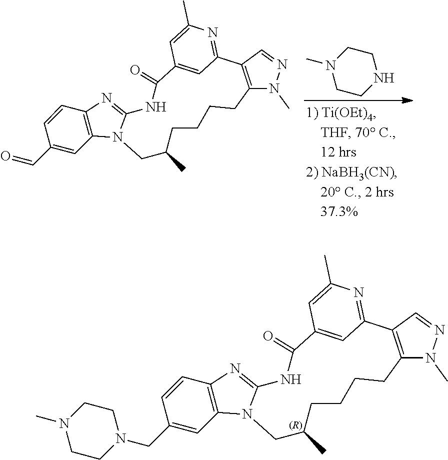

To a solution of (11R)-5,11,26-trimethyl-23-oxo-4,5,13,20,22,27-hexazapentacyclo[22.3.1.0^{2,6}.0^{13,21}.0^{14,19}]octacosa-1(27),2(6),3,14(19),15,17,20,24(28),25-nonaene-16-carbaldehyde (100 mg, 0.219 mmol, 1.0 eq) and 1-methylpiperazine (70 mg, 0.699 mmol, 3.2 eq) in THF (15.0 mL) was added Ti(OEt)₄ (550 mg, 2.41 mmol, 11.0 eq). The mixture was stirred at 70° C. for 12 hours. Then NaBH₃CN (200 mg, 3.18 mmol, 14.5 eq) was added and the mixture was stirred at 20° C. for 1 hour. Water (0.5 mL), Na₂SO₄ (5.0 g) was added and the mixture was stirred at 20° C. for 30 minutes. The reaction mixture was filtered and the filtrate was concentrated in vacuum. The residue was purified by flash chromatography (ISCO®; 12 g SepaFlash® Silica Flash Column, DCM/MeOH with MeOH from 0˜27%, 40 mL/min, 254 nm) to give product (11R)-5,11,26-trimethyl-16-[(4-methylpiperazin-1-yl)methyl]-4,5,13,20,22,27-hexazapentacyclo[22.3.1.0^{2,6}.0^{13,21}.0^{14,19}]octacosa-1(27),2(6),3,14(19),15,17,20,24(28),25-nonaen-23-one (45.6 mg, 37.3% yield) as an off-white solid.

1H NMR (400 MHz, CDCl₃) δ ppm 12.22 (br s, 1H), 8.17 (s, 1H), 8.07 (s, 1H), 7.70 (s, 1H), 7.35-7.29 (m, 2H), 7.26-7.21 (m, 1H), 4.57-4.48 (m, 1H), 3.92 (s, 3H), 3.71 (br s, 2H), 3.57-3.46 (m, 1H), 3.16-3.06 (m, 1H), 2.91-2.69 (m, 5H), 2.54 (br s, 2H), 2.24-2.03 (m, 1H), 1.81-1.66 (m, 4H), 1.56 (br d, J=7.1 Hz, 2H), 0.93 (br d, J=6.6 Hz, 3H).

LCMS [M+H]⁺ m/z: calcd 540.3, found 541.2.

HPLC: 96.9%@220 nm, 96.6%@254 nm.

Chiral SFC: 100% ee.

### Example 5: Preparation of Compound (33)

### The synthesis of (21E)-5,26-dimethyl-16-[(4-methylpiperazin-1-yl)methyl]-10-oxa-4,5,7,13,20,22,27-heptazahexacyclo[22.3.1.1^{7,9}.0^{2,6}.0^{13,21}.0^{18,19}]nonacosa-1(28),2(6),3,14(19),15,17,21,24,26-nonaen-23-one;formic acid (compound (33))

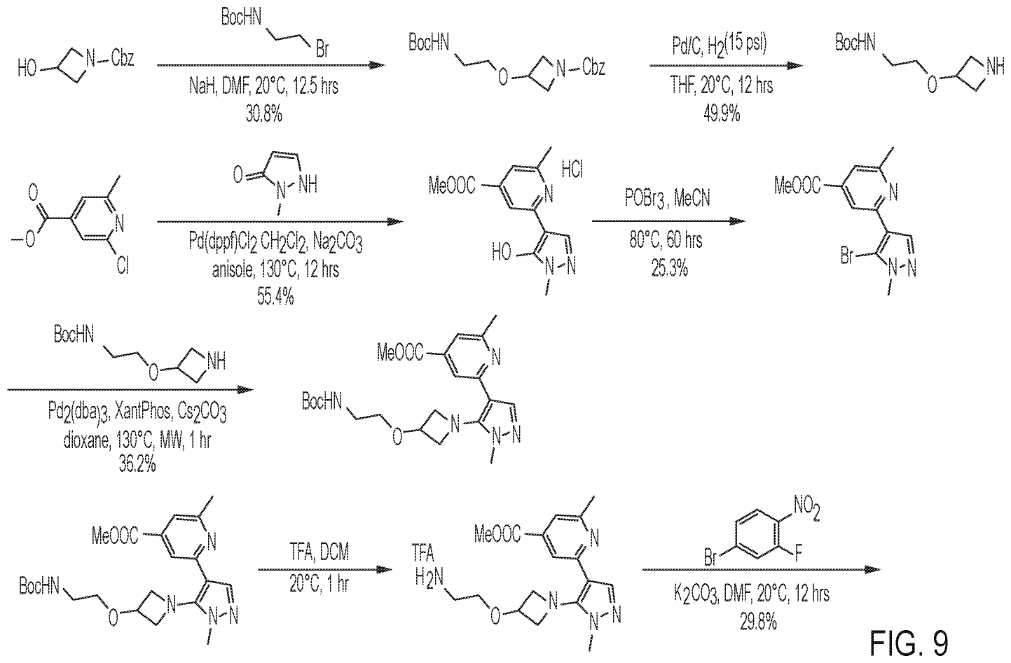

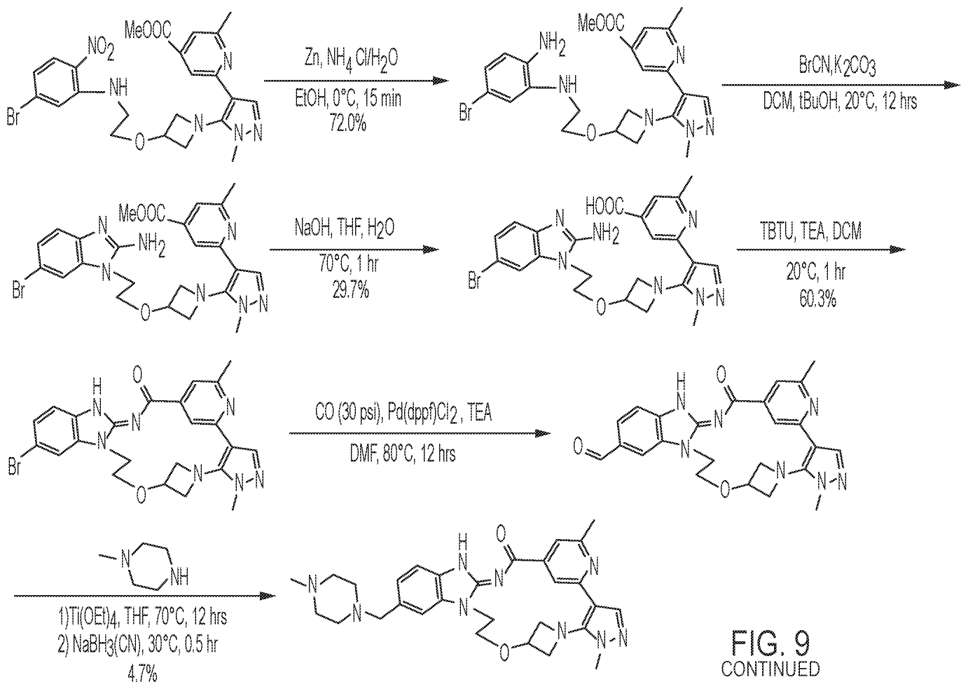

The general synthetic schemes for preparing Compound (33) according to the invention are provided in FIG. 9.

### Step 1: Synthesis of benzyl 3-[2-(tert-butoxycarbonylamino)ethoxy]azetidine-1-carboxylate

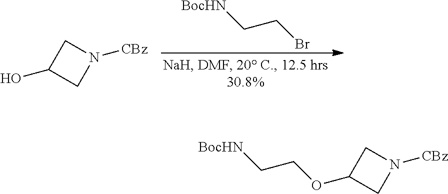

A mixture of benzyl 3-hydroxyazetidine-1-carboxylate (5 g, 24.13 mmol, 1.0 eq) and NAH (3.00 g, 75.01 mmol, 60% purity, 3.1 eq) in DMF (100.0 mL) was stirred at 0° C. for 30 min. Then to the mixture was added tert-butyl N-(2-bromoethyl)carbamate (11 g, 49.09 mmol, 2.0 eq) and the mixture was stirred at 20° C. for 12 hours. The reaction mixture was quenched by addition H₂O 30 mL at 0° C., and then diluted with solvent 20 mL and extracted with EtOAc (100 mL*3). The combined organic layers were washed with brine (200 mL*3), dried over Na₂SO₄, filtered and concentrated under reduced pressure. The residue was purified by flash chromatography (ISCO®; 80 g SepaFlash® Silica Flash Column, petroleum ether/EtOAc with EtOAc from 0˜50%, flow rate: 100 mL/min, 254 nm) to give benzyl 3-[2-(tert-butoxycarbonylamino)ethoxy]azetidine-1-carboxylate (2.6 g, 30.8% yield) as a colorless oil.

LCMS (ESI) [M+H]⁺ m/z: calcd 350.2 found 373.0.

¹H NMR (400 MHz, chloroform-d) δ ppm 7.44-7.30 (m, 5H), 5.10 (s, 2H), 4.25 (br dd, J=6.7, 9.4 Hz, 2H), 4.19-4.03 (m, 2H), 3.96-3.84 (m, 2H), 3.81 (br dd, J=4.5, 9.3 Hz, 1H), 3.43 (br s, 2H), 3.33 (br s, 1H), 1.54-1.28 (m, 9H)

### Step 2: Synthesis of tert-butyl N-[2-(azetidin-3-yloxy)ethyl]carbamate

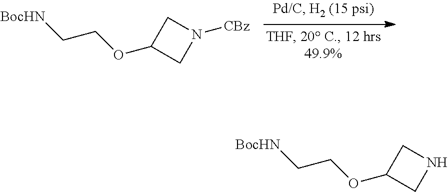

To a solution of benzyl 3-[2-(tert-butoxycarbonylamino)ethoxy]azetidine-1-carboxylate (2.6 g, 7.42 mmol, 1.0 eq) in THF (50.0 mL) was added Pd/C (1.5 g, 10% purity) under N2. The suspension was degassed under vacuum and purged with H₂ several times. The mixture was stirred under H₂ (15 psi) at 20° C. for 12 hours. The reaction mixture was filtered and concentrated under reduced pressure to give tert-butyl N-[2-(azetidin-3-yloxy)ethyl]carbamate (800 mg, 49.9% yield) as a colorless oil.

¹H NMR (400 MHz, chloroform-d) δ ppm 4.88 (br s, 1H), 4.37-4.25 (m, 1H), 4.15-4.08 (m, 2H), 3.84-3.77 (m, 2H), 3.45-3.37 (m, 3H), 3.31 (br s, 2H), 1.44 (s, 9H)

### Step 3: Synthesis of methyl 2-(5-hydroxy-1-methyl-pyrazol-4-yl)-6-methyl-pyridine-4-carboxylate;hydrochloride

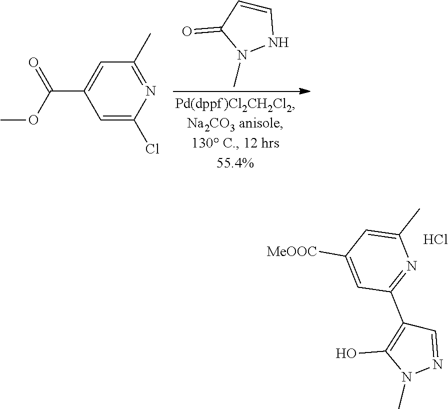

Methyl 2-chloro-6-methyl-pyridine-4-carboxylate (10 g, 53.88 mmol, 1.0 eq), 2-methyl-1H-pyrazol-3-one (10.6 g, 108.05 mmol, 2.0 eq), Na₂CO₃ (12.6 g, 118.88 mmol, 2.2 eq) and Pd(dppf)Cl₂·CH₂Cl₂ (4.4 g, 5.39 mmol, 0.1 eq) in ANISOLE (200.0 mL) was de-gassed and then heated to 130° C. for 12 hours under N2. The reaction mixture was filtered and the filter cake was washed with toluene(400 mL). The dark filtrate was treated with MeOH (30 mL), followed by dropwise addition of 4 M HCl in dioxane(20 mL). The resultant slurry was stirred at room temperature for 1 hour and then was filtered. The filter cake was washed with toluene and heptane, and was then dried under vacuum at 50° C. to give methyl 2-(5-hydroxy-1-methyl-pyrazol-4-yl)-6-methyl-pyridine-4-carboxylate;hydrochloride (9.1 g, 55.4% yield, 93% purity) as a yellow solid.

LCMS (ESI) [M+H]⁺ m/z: calcd 248.1, found 247.9.

¹H NMR (400 MHz, D₂O) δ ppm 8.26 (s, 1H), 8.21 (s, 1H), 7.75 (s, 1H), 3.96 (s, 3H), 3.51 (s, 3H), 2.70 (s, 3H)

### Step 4: Synthesis of methyl 2-(5-bromo-1-methyl-pyrazol-4-yl)-6-methyl-pyridine-4-carboxylate

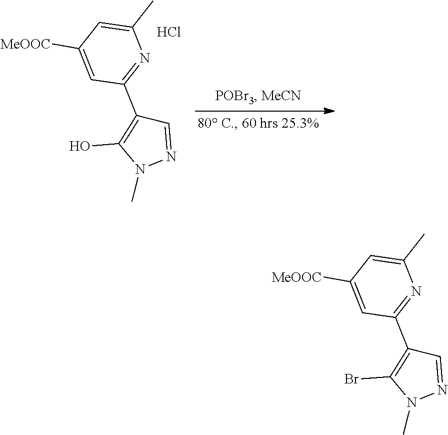 [SMILES: COC(=O)c1cc(C)nc(-c2cnn(C)c2O)c1 ; Cl ; COC(=O)c1cc(C)nc(-c2cnn(C)c2Br)c1]

A mixture of methyl 2-(5-hydroxy-1-methyl-pyrazol-4-yl)-6-methyl-pyridine-4-carboxylate;hydrochloride (7.6 g, 26.79 mmol, 1.0 eq) and POBr₃ (61 g, 212.78 mmol, 7.9 eq) in CH₃CN (20.0 mL) was stirred at 85° C. for 60 hours. The reaction mixture was poured into ice water (200 mL), and then adjusted to pH=7 with saturated aqueous Na₂CO₃ and extracted with EtOAc(100 mL*3). The combined organic layers were washed with brine (200 mL*3), dried over Na₂SO₄, filtered and concentrated under reduced pressure. The residue was purified by flash chromatography (ISCO®; 80 g SepaFlash® Silica Flash Column, petroleum ether/EtOAc with EtOAc from 0˜37%, flow rate:100 mL/min, 254 nm) to afford methyl 2-(5-bromo-1-methyl-pyrazol-4-yl)-6-methyl-pyridine-4-carboxylate (2.1 g, 25.3% yield, 100% purity) as a white solid.

LCMS (ESI) [M+H]⁺ m/z: calcd 310.0, found 311.8.

¹H NMR (400 MHz, chloroform-d) (ppm 8.10 (s, 1H), 8.07 (s, 1H), 7.60 (s, 1H), 3.98 (s, 3H), 3.97 (s, 3H), 2.67 (s, 3H).

### Step 5: Synthesis of methyl 2-[5-[3-[2-(tert-butoxycarbonylamino)ethoxy]azetidin-1-yl]-1-methyl-pyrazol-4-yl]-6-methyl-pyridine-4-carboxylate

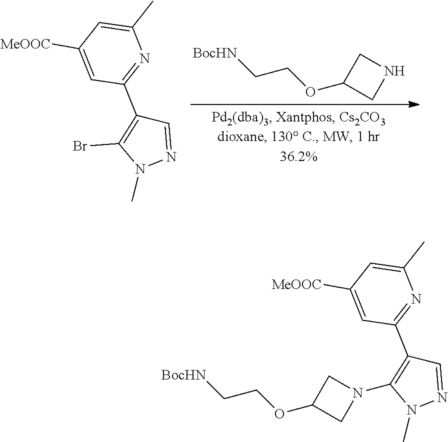 [SMILES: COC(=O)c1cc(C)nc(-c2cnn(C)c2Br)c1 ; CC(C)(C)OC(=O)NCCOC1CNC1 ; COC(=O)c1cc(C)nc(-c2cnn(C)c2N2CC(OCCNC(=O)OC(C)(C)C)C2)c1]

Methyl 2-(5-bromo-1-methyl-pyrazol-4-yl)-6-methyl-pyridine-4-carboxylate (700 mg, 2.26 mmol, 1.0 eq), tert-butyl N-[2-(azetidin-3-yloxy)ethyl]carbamate (800 mg, 3.70 mmol, 1.6 eq), Xantphos (140 mg, 0.242 mmol, 0.1 eq), Cs₂CO₃ (1.8 g, 5.52 mmol, 2.5 eq) and Pd₂(dba)₃ (210 mg, 0.229 mmol, 0.1 eq) were taken up into a microwave tube in dioxane (10.0 mL). The sealed tube was heated at 130° C. for 60 min under microwave under N2. The reaction mixture was filtered and concentrated under reduced pressure. The residue was purified by flash chromatography (ISCO®; 40 g SepaFlash® Silica Flash Column, petroleum ether/EtOAc with EtOAc from 0˜100%, flow rate:100 mL/min, 254 nm) to give methyl 2-[5-[3-[2-(tert-butoxycarbonylamino)ethoxy]azetidin-1-yl]-1-methyl-pyrazol-4-yl]-6-methyl-pyridine-4-carboxylate (520 mg, 36.2% yield, 70% purity) as a yellow oil.

LCMS (ESI) [M+H]⁺ m/z: calcd 446.2, found 468.1.

### Step 6: Synthesis of methyl 2-[5-[3-(2-aminoethoxy)azetidin-1-yl]-1-methyl-pyrazol-4-yl]-6-methyl-pyridine-4-carboxylate; 2,2,2-trifluoroacetic acid

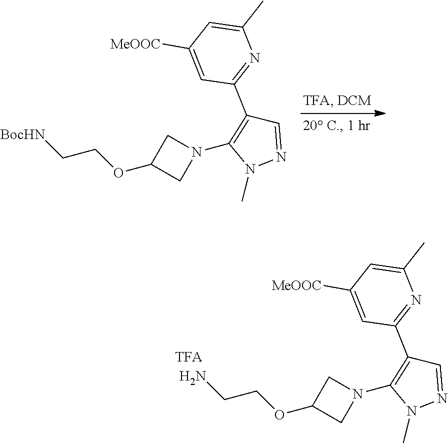 [SMILES: COC(=O)c1cc(C)nc(-c2cnn(C)c2N2CC(OCCNC(=O)OC(C)(C)C)C2)c1 ; COC(=O)c1cc(C)nc(-c2cnn(C)c2N2CC(OCCN)C2)c1]

A mixture of methyl 2-[5-[3-[2-(tert-butoxycarbonylamino)ethoxy]azetidin-1-yl]-1-methyl-pyrazol-4-yl]-6-methyl-pyridine-4-carboxylate (520 mg, 1.17 mmol, 1.0 eq) and TFA (3.0 mL, 40.52 mmol, 34.7 eq) in DCM (9 mL.0) was stirred at 20° C. for 1 hour. The reaction mixture was concentrated under reduced pressure to give methyl 2-[5-[3-(2-aminoethoxy)azetidin-1-yl]-1-methyl-pyrazol-4-yl]-6-methyl-pyridine-4-carboxylate;2,2,2-trifluoroacetic acid (1.2 g, crude) as a yellow oil.

LCMS (ESI) [M+H]⁺ m/z: calcd 346.2, found 346.1.

### Step 7: Synthesis of methyl 2-[5-[3-[2-(5-bromo-2-nitro-anilino)ethoxy]azetidin-1-yl]-1-methyl-pyrazol-4-yl]-6-methyl-pyridine-4-carboxylate

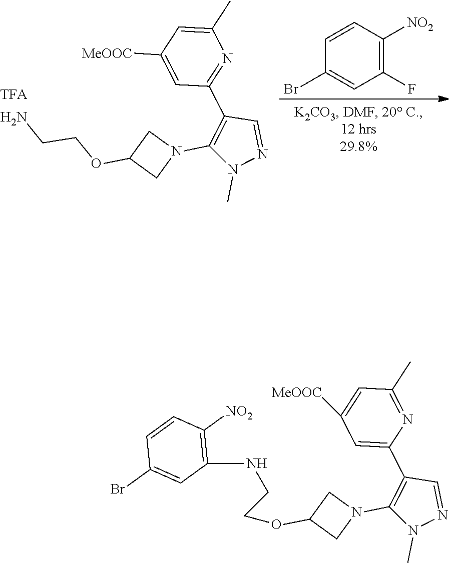 [SMILES: COC(=O)c1cc(C)nc(-c2cnn(C)c2N2CC(OCCN)C2)c1 ; O=[N+]([O-])c1ccc(Br)cc1F ; "TFA" ; COC(=O)c1cc(C)nc(-c2cnn(C)c2N2CC(OCCNc3cc(Br)ccc3[N+](=O)[O-])C2)c1]

A mixture of methyl 2-[5-[3-(2-aminoethoxy)azetidin-1-yl]-1-methyl-pyrazol-4-yl]-6-methyl-pyridine-4-carboxylate;2,2,2-trifluoroacetic acid (1.2 g, 2.61 mmol, 1.0 eq),4-bromo-2-fluoro-1-nitro-benzene (600 mg, 2.73 mmol, 1.0 eq) and K₂CO₃ (2.6 g, 18.81 mmol, 7.2 eq) in DMF (15.0 mL) was stirred at 20° C. for 1 hour. The reaction mixture was partitioned between H₂O (50 mL) and EtOAc (50 mL). The organic phase was separated, washed with brine (100 mL*3), dried over Na₂SO₄, filtered and concentrated under reduced pressure. The residue was purified by flash chromatography (ISCO©; 40 g SepaFlash© Silica Flash Column, petroleum ether/EtOAc with EtOAc from 0˜100%, flow rate:100 mL/min, 254 nm) to give methyl 2-[5-[3-[2-(5-bromo-2-nitro-anilino)ethoxy]azetidin-1-yl]-1-methyl-pyrazol-4-yl]-6-methyl-pyridine-4-carboxylate (500 mg, 29.8% yield, 85% purity) as a yellow solid.

LCMS (ESI) [M+H]⁺ m/z: calcd 545.1, found 569.0.

### Step 8: Synthesis of methyl 2-[5-[3-[2-(2-amino-5-bromo-anilino)ethoxy]azetidin-1-yl]-1-methyl-pyrazol-4-yl]-6-methyl-pyridine-4-carboxylate

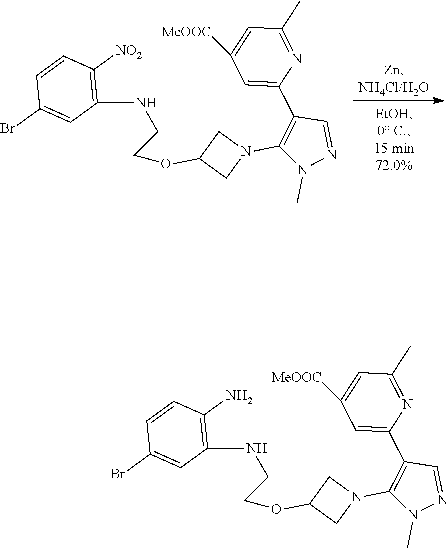 [SMILES: COC(=O)c1cc(C)nc(-c2cnn(C)c2N2CC(OCCNc3cc(Br)ccc3[N+](=O)[O-])C2)c1 ; COC(=O)c1cc(C)nc(-c2cnn(C)c2N2CC(OCCNc3cc(Br)ccc3N)C2)c1]

A mixture of methyl 2-[5-[3-[2-(5-bromo-2-nitro-anilino)ethoxy]azetidin-1-yl]-1-methyl-pyrazol-4-yl]-6-methyl-pyridine-4-carboxylate (500 mg, 0.917 mmol, 1.0 eq), Zn (400 mg, 6.12 mmol, 6.7 eq) and NH₄Cl (350 mg, 6.54 mmol, 7.1 eq) in EtOH (20.0 mL) and H₂O (4.0 mL) was stirred at 0° C. for 15 min. The reaction mixture was filtered and the filtrate was partitioned between saturated aqueous Na₂CO₃ (20 mL) and DCM (40 mL). The organic phase was separated, washed with brine (30 mL*3), dried over Na₂SO₄, filtered and concentrated under reduced pressure to give methyl 2-[5-[3-[2-(2-amino-5-bromo-anilino)ethoxy]azetidin-1-yl]-1-methyl-pyrazol-4-yl]-6-methyl-pyridine-4-carboxylate (400 mg, 72.0% yield, 85% purity) as a yellow solid.

LCMS (ESI) [M+H]⁺ m/z: calcd 515.1, found 516.9.

### Step 9: Synthesis of methyl 2-[5-[3-[2-(2-amino-6-bromo-benzimidazol-1-yl)ethoxy]azetidin-1-yl]-1-methyl-pyrazol-4-yl]-6-methyl-pyridine-4-carboxylate

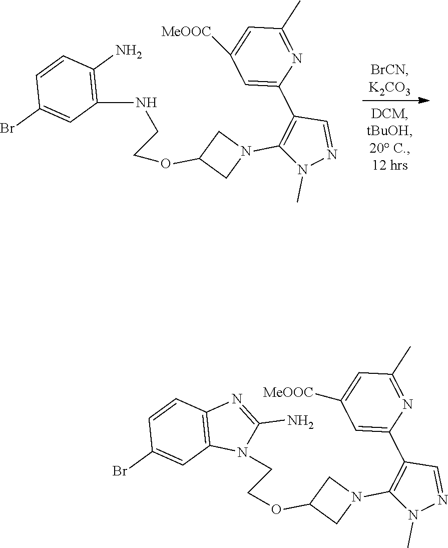 [SMILES: COC(=O)c1cc(C)nc(-c2cnn(C)c2N2CC(OCCNc3cc(Br)ccc3N)C2)c1 ; COC(=O)c1cc(C)nc(-c2cnn(C)c2N2CC(OCCn3c(N)nc4ccc(Br)cc43)C2)c1]

A mixture of methyl 2-[5-[3-[2-(2-amino-5-bromo-anilino)ethoxy]azetidin-1-yl]-1-methyl-pyrazol-4-yl]-6-methyl-pyridine-4-carboxylate (350 mg, 0.679 mmol, 1.0 eq), K₂CO₃ (2 g, 14.47 mmol, 21.3 eq) and carbononitridic bromide (670 mg, 6.33 mmol, 9.3 eq) in DCM (15.0 mL) and t-BuOH (3.0 mL) was stirred at 20° C. for 12 hours. The reaction mixture was partitioned between H₂O 30 mL and DCM 30 mL. The organic phase was separated, washed with brine (30 Ml*3), dried over Na₂SO₄, filtered and concentrated under reduced pressure to give methyl 2-[5-[3-[2-(2-amino-6-bromo-benzimidazol-1-yl)ethoxy]azetidin-1-yl]-1-methyl-pyrazol-4-yl]-6-methyl-pyridine-4-carboxylate (800 mg, crude) as a brown oil

LCMS (ESI) [M+H]⁺ m/z: calcd 540.1, found 540.1.

### Step 10: Synthesis of 2-[5-[3-[2-(2-amino-6-bromo-benzimidazol-1-yl)ethoxy]azetidin-1-yl]-1-methyl-pyrazol-4-yl]-6-methyl-pyridine-4-carboxylic acid

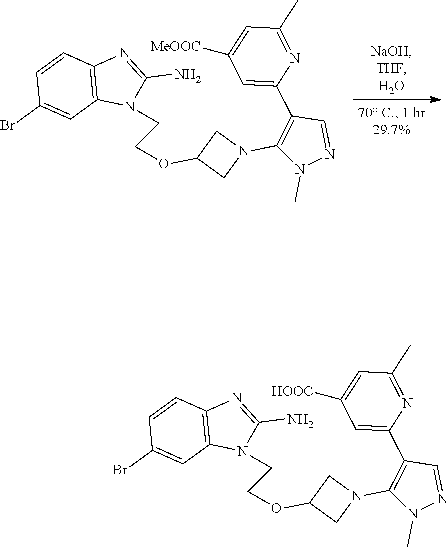 [SMILES: COC(=O)c1cc(C)nc(-c2cnn(C)c2N2CC(OCCn3c(N)nc4ccc(Br)cc43)C2)c1 ; Cc1cc(C(=O)O)cc(-c2cnn(C)c2N2CC(OCCn3c(N)nc4ccc(Br)cc43)C2)n1]

A mixture of methyl 2-[5-[3-[2-(2-amino-6-bromo-benzimidazol-1-yl)ethoxy]azetidin-1-yl]-1-methyl-pyrazol-4-yl]-6-methyl-pyridine-4-carboxylate (880 mg, 1.63 mmol, 1.0 eq) and NaOH (1 g, 25.00 mmol, 15.4 eq) in THF (10.0 mL) and H₂O (5.0 mL) was stirred at 70° C. for 1 hour. The reaction mixture was concentrated under reduced pressure. The residue was diluted with H₂O (5 mL) and adjusted to pH=5 with 3N TFA at 0° C. The mixture was filtered and the filtrate was extracted with DCM: iPrOH (v/v:10/1)(30 mL*5) and the combined filtrate was dried over Na₂SO₄ and concentrated under reduced pressure to give 2-[5-[3-[2-(2-amino-6-bromo-benzimidazol-1-yl)ethoxy]azetidin-1-yl]-1-methyl-pyrazol-4-yl]-6-methyl-pyridine-4-carboxylic acid (260 mg, 29.7% yield, 98% purity) as a yellow solid.

LCMS (ESI) [M+H]⁺ m/z: calcd 526.1, found 528.0.

### Step 11: Synthesis of (21E)-16-bromo-5,26-dimethyl-10-oxa-4,5,7,13,20,22,27-heptazahexacyclo[22.3.1.1^{7,9}.0^{2,6}.0^{13,21}.0^{14,19}]nonacosa-1(28),2(6),3,14(19),15,17,21,24,26-nonaen-23-one

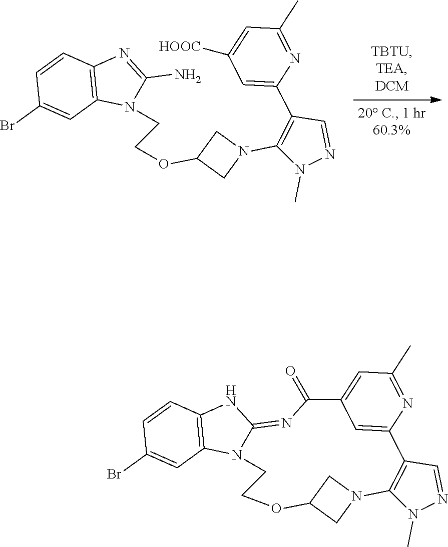 [SMILES: Cc1cc(C(=O)O)cc(-c2cnn(C)c2N2CC(OCCn3c(N)nc4ccc(Br)cc43)C2)n1 ; Cc1cc2cc(n1)-c1cnn(C)c1N1CC(C1)OCCN1/C(=N/C2=O)Nc2ccc(Br)cc21]

A mixture of 2-[5-[3-[2-(2-amino-6-bromo-benzimidazol-1-yl)ethoxy]azetidin-1-yl]-1-methyl-pyrazol-4-yl]-6-methyl-pyridine-4-carboxylic acid (210 mg, 0.399 mmol, 1.0 eq), TBTU (200 mg, 0.623 mmol, 1.6 eq) and TEA (0.4 mL, 2.87 mmol, 7.2 eq) in DCM (100.0 mL) was stirred at 20° C. for 1 hour. The reaction mixture was partitioned DCM (30 mL) and H₂O (50 mL). The organic phase was separated, washed with brine (100 mL*3), dried over Na₂SO₄, filtered and concentrated under reduced pressure. The residue was purified by flash silica gel chromatography (ISCO®; 20 g SepaFlash® Silica Flash Column, MeOH(0.05 v % NH₃·H₂O)/DCM with MeOH(0.05 v % NH₃·H₂O) from 0˜14%, flow rate: 80 mL/min, 254 nm) to afford (21E)-16-bromo-5,26-dimethyl-10-oxa-4,5,7,13,20,22,27-heptazahexacyclo[22.3.1.17,9.02,6.013,21.014,19]nonacosa-1(28),2(6),3,14(19),15,17,21,24,26-nonaen-23-one (130 mg, 60.3% yield, 94% purity) as a yellow solid.

LCMS (ESI) [M+H]⁺ m/z: calcd 508.1, found 509.8.

### Step 12: Synthesis of 21E)-5,26-dimethyl-23-oxo-10-oxa-4,5,7,13,20,22,27-heptazahexacyclo[22.3.1.1^{7,9}.0^{2,6}.0^{13,21}.0^{14,19}]nonacosa-1(28),2(6),3,14(19),15,17,21,24,26-nonaene-16-carbaldehyde

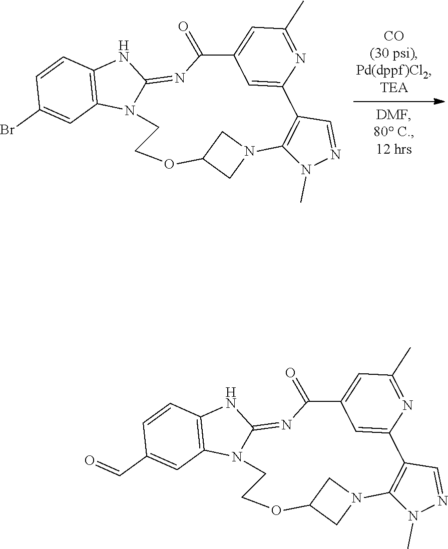 [SMILES: Cc1cc2cc(n1)-c1cnn(C)c1N1CC(C1)OCCN1/C(=N/C2=O)Nc2ccc(Br)cc21 ; Cc1cc2cc(n1)-c1cnn(C)c1N1CC(C1)OCCN1/C(=N/C2=O)Nc2ccc(C=O)cc21]

To a solution of (21E)-16-bromo-5,26-dimethyl-10-oxa-4,5,7,13,20,22,27-heptazahexacyclo[22.3.1.17,9.02,6.013,21.014,19]nonacosa-1(28),2(6),3,14(19),15,17,21,24,26-nonaen-23-one (130 mg, 0.256 mmol, 1.0 eq), triethylsilane (0.2 mL, 1.25 mmol, 4.9 eq) and TEA (0.2 mL, 1.44 mmol, 5.6 eq) in DMF (10.0 mL) was added Pd(dppf)Cl₂ (20 mg, 0.0273 mmol, 0.1 eq) under N2. The suspension was degassed under vacuum and purged with CO several times. The mixture was stirred under CO (30 psi) at 80° C. for 12 hours. The mixture was filtered and the filtrate was diluted with DCM (20 mL) and washed with saturated NH₄Cl (30 mL) and brine (30 mL*3), dried over Na₂SO₄, filtered and concentrated under reduced pressure to give (21E)-5,26-dimethyl-23-oxo-10-oxa-4,5,7,13,20,22,27-heptazahexacyclo[22.3.1.17,9.02,6.013,21.014,19]nonacosa-1(28),2(6),3,14(19),15,17,21,24,26-nonaene-16-carbaldehyde (200 mg, crude) as a yellow solid.

LCMS (ESI) [M+H]⁺ m/z: calcd 458.2, found 458.1.

### Step 13: Synthesis of (21E)-5,26-dimethyl-16-[(4-methylpiperazin-1-yl)methyl]-10-oxa-4,5,7,13,20,22,27-heptazahexacyclo[22.3.1.1^{7,9}.0^{2,6}.0^{13,21}.0^{14,19}]nonacosa-1(28),2(6), 3,14(19),15,17,21,24,26-nonaen-23-one;formic acid

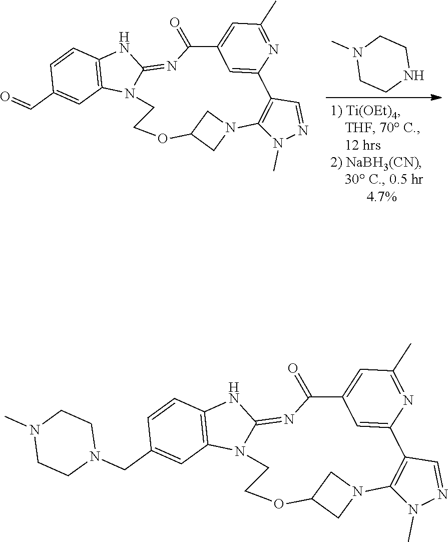

A mixture of (21E)-5,26-dimethyl-23-oxo-10-oxa-4,5,7,13,20,22,27-heptazahexacyclo[22.3.1.17,9.02,6.013,21.014,19]nonacosa-1(28),2(6),3,14(19),15,17,21,24,26-nonaene-16-carbaldehyde (200 mg, 0.437 mmol, 1.0 eq),1-methylpiperazine (150 mg, 1.50 mmol, 3.4 eq) and Ti(OEt)₄ (500 mg, 2.19 mmol, 5.0 eq) in THF (20.0 mL) was stirred at 70° C. for 12 hours. Then to the mixture was added NaBH₃CN (140 mg, 2.23 mmol, 5.1 eq) and the mixture was stirred at 30° C. for 30 min. The reaction mixture was quenched by addition H₂O 0.2 mL then to the mixture was added 0.5 g silica gel and the mixture was stirred at 20° C. for 15 min. The mixture was filtered and the filter cake was washed with DCM: MeOH (v/v:10/1)(20 mL*5) and the combined filtrate was concentrated under reduced pressure to give the crude product. The crude product was purified by preparative HPLC (Column: Welch Xtimate C18 150*25 mm*5 um; Mobile phase: [water(FA)-ACN];B %: 0%-25%,9.5 min, Column Temp. 30° C.) to afford (21E)-5,26-dimethyl-16-[(4-methylpiperazin-1-yl)methyl]-10-oxa-4,5,7,13,20,22,27-heptazahexacyclo[22.3.1.1^{7,9}.0^{2,6}.0^{13,21}.0^{14,19}]nonacosa-1(28),2(6),3,14(19),15,17,21,24,26-nonaen-23-one;formic acid (12.9 mg, 4.7% yield, 92.74% purity) as a yellow solid.

¹H NMR (400 MHz, methanol-d₄) δ ppm 8.59 (br s, 1H), 8.38 (s, 1H), 7.84 (s, 1H), 7.63 (br s, 1H), 7.49-7.41 (m, 2H), 7.28 (br d, J=7.5 Hz, 1H), 4.57 (br s, 1H), 4.49 (br s, 2H), 4.37 (br s, 2H), 4.18 (br s, 2H), 3.92 (br s, 2H), 3.78 (s, 3H), 3.76 (br s, 2H), 3.20 (br s, 4H), 2.98 (s, 1H), 2.84-2.72 (m, 7H), 2.59 (br s, 3H)

LCMS (ESI) [M+H]⁺ m/z: calcd 542.3, found 542.2.

HPLC: 92.74%@220 nm, 92.91%@254 nm.

### Example 6: Preparation of Compound (55)

The Synthesis of (11S,21E)-5,11,26-trimethyl-16-[(4-methylpiperazin-1-yl)methyl]-10-oxa-4,5,13,20,22,27-hexazapentacyclo[22.3.1.02,6.013,21.014,19]octacosa-1(28),2(6),3,14(19),15,17,21,24,26-nonaen-23-one (compound(55))

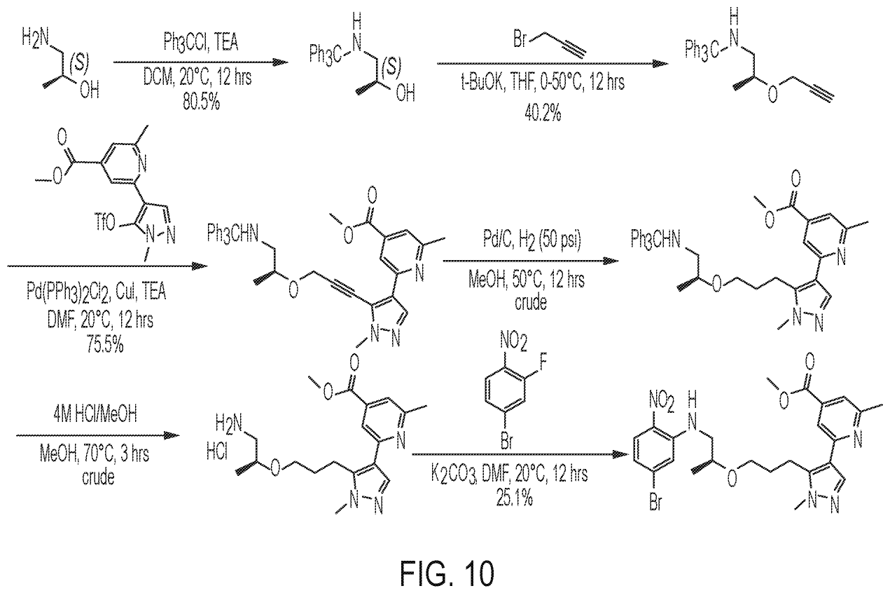

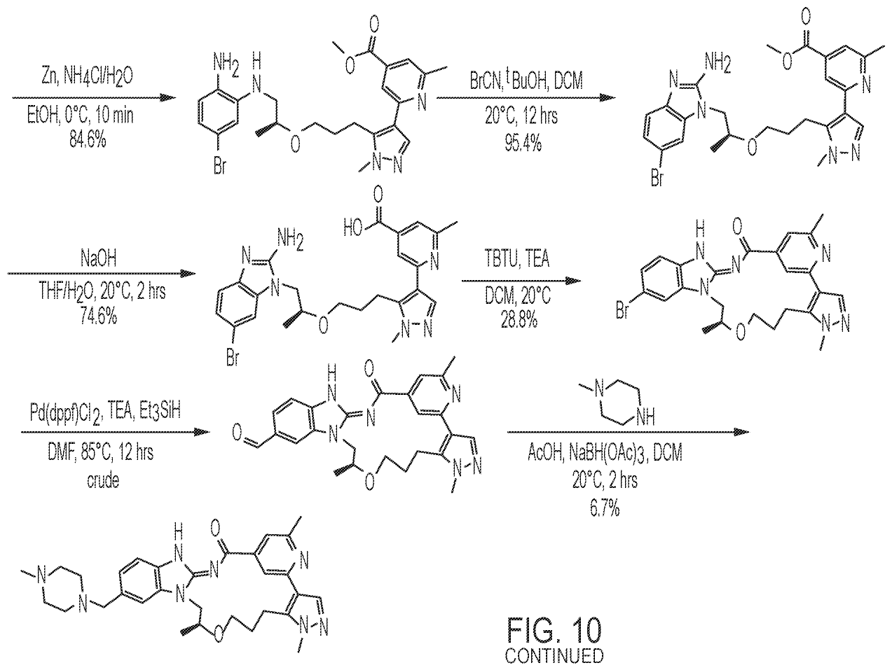

The general synthetic schemes for preparing Compound (55) according to the invention are provided in FIG. 10.

### Step 1: Synthesis of (2S)-1-(tritylamino)propan-2-ol

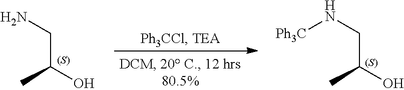

To a solution of (2S)-1-aminopropan-2-ol (10.0 g, 133 mmol, 1.0 eq) in DCM (160 mL) were added TEA (40 mL, 287 mmol, 2.16 eq) and [chloro(diphenyl)methyl]benzene (40 g, 0.143 mol, 1.08 eq) at 0° C. After addition, the mixture was stirred at 20° C. for 12 hours. The reaction mixture was diluted with water (100 mL) and extracted with DCM (150 mL*3). The combined organic layers were dried over Na₂SO₄ and filtered. The filtrate was concentrated under reduced pressure. The residue was purified by flash chromatography (ISCO®; 330 g SepaFlash® Silica Flash Column, petroleum ether/EtOAc with EtOAc from 0-10%, flow rate: 80 mL/min, 254 nm) to give (2S)-1-(tritylamino)propan-2-ol (34 g, 80.5% yield) as colourless oil.

¹H NMR (400 MHz, CDCl₃) δ ppm 7.34-7.43 (m, 6H), 7.17-7.27 (m, 6H), 7.06-7.15 (m, 3H), 3.67-3.78 (m, 1H), 2.06-2.13 (m, 2H), 1.03 (d, J=6.4 Hz, 3H)

### Step 2: Synthesis of (2S)-2-prop-2-ynoxy-N-trityl-propan-1-amine

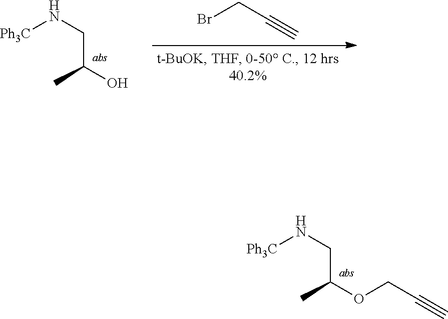

To a solution of (2S)-1-(tritylamino)propan-2-ol (10.0 g, 31.5 mmol, 1.0 eq) in THF (100.0 mL) were added 1M tBuOK/THF (70 mL, 70.0 mmol, 2.22 eq) and 3-bromoprop-1-yne in toluene (10.0 mL, 92.8 mmol, 80% purity, 2.95 eq) at 0° C. The mixture was then stirred at 50° C. for 12 hours. The reaction mixture was quenched with water (50 mL) and extracted with EtOAc (60 mL*3). The combined organic layers were washed with brine (50 mL), dried over Na₂SO₄ and filtered. The filtrate was concentrated under reduced pressure. The residue was purified by flash chromatography (ISCO®; 80 g SepaFlash® Silica Flash Column, petroleum ether/EtOAc with EtOAc from 0˜4%, flow rate: 50 mL/min, 254 nm) to give (2S)-2-prop-2-ynoxy-N-trityl-propan-1-amine (4.5 g, 40.2% yield) as light brown oil.

¹H NMR (400 MHz, CDCl₃) δ ppm 7.37-7.45 (m, 6H), 7.16-7.25 (m, 6H), 7.06-7.14 (m, 3H), 4.03 (t, J=2.4 Hz, 2H), 3.68-3.77 (m, 1H), 2.31 (t, J=2.4 Hz, 1H), 2.07-2.23 (m, 2H), 1.10 (d, J=6.4 Hz, 3H)

### Step 3: Synthesis of methyl 2-methyl-6-[I-methyl-5-[3-[(]S)-1-methyl-2-(tritylamino)ethoxy]prop-1-ynyl]pyrazol-4-yl]pyridine-4-carboxylate

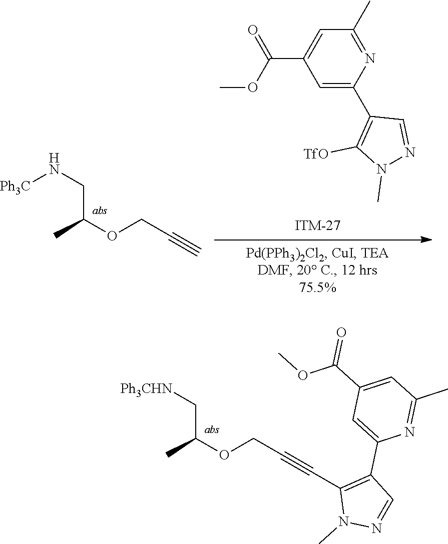

To a solution of methyl 2-methyl-6-[1-methyl-5-(trifluoromethylsulfonyloxy)pyrazol-4-yl]pyridine-4-carboxylate (2.0 g, 5.27 mmol, 1.0 eq) in DMF (40.0 mL) were added (2S)-2-prop-2-ynoxy-N-trityl-propan-1-amine (3.60 g, 10.1 mmol, 1.92 eq), Pd(PPh₃)₂Cl₂ (800 mg, 1.14 mmol, 0.216 eq), CuI (400 mg, 2.10 mmol, 0.398 eq) and TEA (4.0 mL, 28.7 mmol, 5.45 eq). The mixture was stirred at 20° C. for 12 hours. The reaction mixture was diluted with water (60 mL) and extracted with EtOAc(100 mL*3). The combined organic layers were washed with brine (100 mL*3), dried over Na₂SO₄ and filtered. The filtrate was concentrated under reduced pressure. The residue was purified by flash chromatography (ISCO®; 40 g SepaFlash® Silica Flash Column, petroleum ether/EtOAc with EtOAc from 0˜22%, flow rate: 60 mL/min, 254 nm) to give methyl 2-methyl-6-[1-methyl-5-[3-[(1S)-1-methyl-2-(tritylamino)ethoxy]prop-1-ynyl]pyrazol-4-yl]pyridine-4-carboxylate (2.4 g, 75.5% yield, 97% purity) as a brown solid.

¹H NMR (400 MHz, CDCl₃) δ ppm 8.35 (s, 1H), 8.14 (s, 1H), 7.55 (d, J=0.8 Hz, 1H), 7.42-7.46 (m, 6H), 7.13-7.23 (m, 9H), 4.48-4.61 (m, 2H), 3.95-4.04 (m, 4H), 3.89 (s, 3H), 2.63 (s, 3H), 2.28-2.35 (m, 1H), 2.19-2.27 (m, 1H), 1.24 (d, J=6.4 Hz, 3H).

LCMS (ESI) [M+Na]⁺ m/z: calcd 607.3, found 607.2.

### Step 4: Synthesis of methyl 2-methyl-6-[ ]-methyl-5-[3-[(]S)-1-methyl-2-(tritylamino)ethoxy]propyl]pyrazol-4-yl]pyridine-4-carboxylate

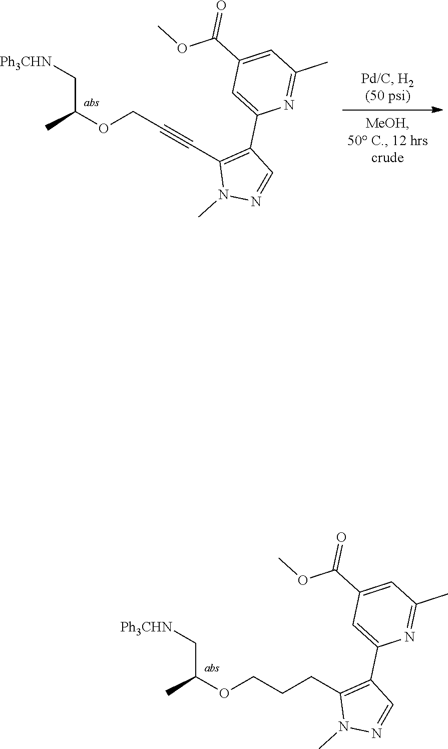

To a solution of methyl 2-methyl-6-[1-methyl-5-[3-[(1S)-1-methyl-2-(tritylamino)ethoxy]prop-1-ynyl]pyrazol-4-yl]pyridine-4-carboxylate (1 g, 1.71 mmol, 1.0 eq) in MeOH (100.0 mL) was added Pd/C (1.0 g, 10% purity). The suspension was degassed under vacuum and purged with H₂ three times. The mixture was stirred under H₂ (50 psi) at 50° C. for 12 hours. The reaction mixture was filtered and the filtrate was concentrated under reduced pressure to give methyl 2-methyl-6-[1-methyl-5-[3-[(1S)-1-methyl-2-(tritylamino)ethoxy]propyl]pyrazol-4-yl]pyridine-4-carboxylate (1 g, crude) as colourless oil.

### Step 5: Synthesis of methyl 2-[5-[3-[(]S)-2-amino-]-methyl-ethoxy]propyl]-1-methyl-pyrazol-4-yl]-6-methyl-pyridine-4-carboxylate;hydrochloride

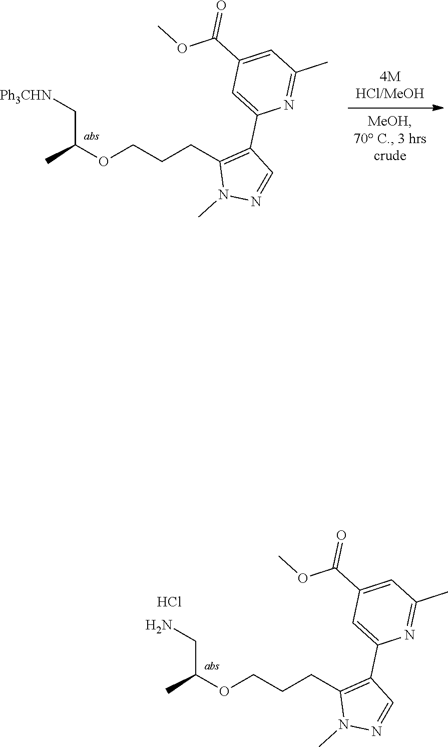

To a solution of methyl 2-methyl-6-[1-methyl-5-[3-[(1S)-1-methyl-2-(tritylamino)ethoxy]propyl]-pyrazol-4-yl]pyridine-4-carboxylate (1 g, 1.70 mmol, 1.0 eq) in MeOH (30.0 mL) was added 4 M HCl/dioxane (15 mL, 60.0 mmol, 35.3 eq) at 0° C. After addition, the mixture was stirred at 20° C. for 12 hours. The mixture was then stirred at 70° C. for 3 hours. The reaction mixture was concentrated under reduced pressure to give methyl 2-[5-[3-[(1S)-2-amino-1-methyl-ethoxy]propyl]-1-methyl-pyrazol-4-yl]-6-methyl-pyridine-4-carboxylate;hydrochloride (1.4 g, crude) as yellow oil. The crude product will be used to next step directly.

### Step 6: Synthesis of methyl 2-[5-[3-[(]S)-2-(5-bromo-2-nitro-anilino)-1-methyl-ethoxy]propyl]-1-methyl-pyrazol-4-yl]-6-methyl-pyridine-4-carboxylate

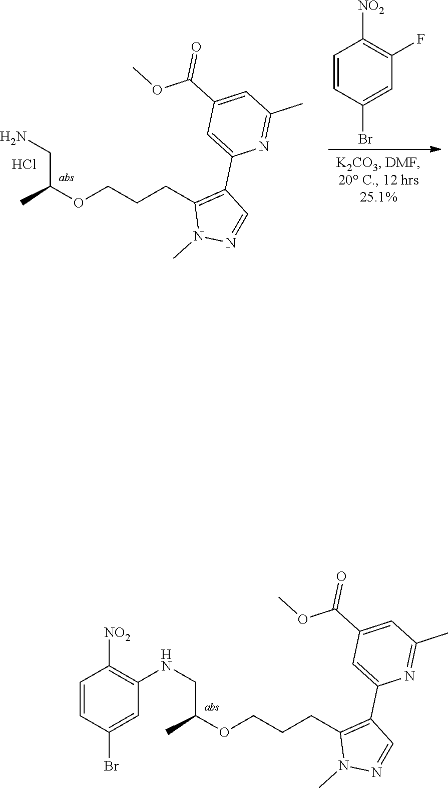

To a solution of methyl 2-[5-[3-[(1S)-2-amino-1-methyl-ethoxy]propyl]-1-methyl-pyrazol-4-yl]-6-methyl-pyridine-4-carboxylate;hydrochloride (1.2 g, 3.13 mmol, 1.0 eq) in DMF (30.0 mL) were added 4-bromo-2-fluoro-1-nitro-benzene (720 mg, 3.27 mmol, 1.04 eq) and K₂CO₃ (2.70 g, 19.5 mmol, 6.23 eq). The mixture was stirred at 20° C. for 12 hours. The reaction mixture was diluted with water (50 mL) and extracted with EtOAc (100 mL*3). The combined organic layers were washed with brine (100 mL*3), dried over Na₂SO₄ and filtered. The filtrate was concentrated under reduced pressure. The residue was purified by flash chromatography (ISCO®; 40 g SepaFlash® Silica Flash Column, petroleum ether/EtOAc with EtOAc from 0-50%, flow rate: 50 mL/min, 254 nm) to give methyl 2-[5-[3-[(1S)-2-(5-bromo-2-nitro-anilino)-1-methyl-ethoxy]propyl]-1-methyl-pyrazol-4-yl]-6-methyl-pyridine-4-carboxylate (430 mg, 25.1% yield, 100% purity) as yellow oil.

¹H NMR (400 MHz, CDCl₃) δ ppm 8.33 (br s, 1H), 8.02 (d, J=9.2 Hz, 1H), 7.88 (s, 1H), 7.80 (s, 1H), 7.47 (s, 1H), 7.03 (d, J=2.0 Hz, 1H), 6.76 (dd, J=9.2, 2.0 Hz, 1H), 3.95 (s, 3H), 3.88 (s, 3H), 3.71-3.78 (m, 1H), 3.67 (dt, J=9.2, 6.4 Hz, 1H), 3.46 (dt, J=9.2, 6.0 Hz, 1H), 3.36 (ddd, J=12.8, 5.6, 4.0 Hz, 1H), 3.17-3.26 (m, 3H), 2.60 (s, 3H), 1.94-2.03 (m, 2H), 1.26 (d, J=6.0 Hz, 3H).

LCMS (ESI) [M+H]⁺ m/z: calcd 548.1, found 548.0.

Chiral SFC: 98.2% ee

2D NMR: The substituent site in the determined structure is confirmed by HMBC correlation between H30 and C16. The chemical shift of C30 (67.69 ppm) also supports the determined structure.

### Step 7: Synthesis of methyl 2-[5-[3-[(]S)-2-(2-amino-5-bromo-anilino)-1-methyl-ethoxy]propyl]-1-methyl-pyrazol-4-yl]-6-methyl-pyridine-4-carboxylate

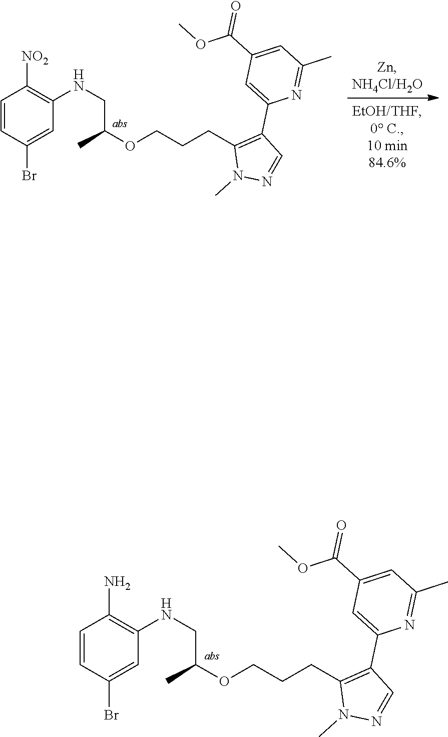

To a solution of methyl 2-[5-[3-[(1S)-2-(5-bromo-2-nitro-anilino)-1-methyl-ethoxy]propyl]-1-methyl-pyrazol-4-yl]-6-methyl-pyridine-4-carboxylate (400 mg, 0.732 mmol, 1.0 eq) in EtOH (8.0 mL), THF (8.0 mL) and H₂O (4.0 mL) were added Zn (480 mg, 7.34 mmol, 10.0 eq) and NH₄Cl (400 mg, 7.48 mmol, 10.2 eq) at 0° C. After addition, the reaction mixture was stirred at 0° C. for 10 minutes. The reaction mixture was filtered and the filtrate was concentrated under reduced pressure. The residue was diluted with water (10 mL) and extracted with EtOAc (30 mL*3). The combined organic layers were washed with brine (30 mL), dried over Na₂SO₄ and filtered. The filtrate was concentrated under reduced pressure. The residue was purified by flash chromatography (ISCO®; 12 g SepaFlash® Silica Flash Column, petroleum ether/EtOAc with EtOAc from 0˜60%, flow rate: 30 mL/min, 254 nm) to give methyl 2-[5-[3-[(1S)-2-(2-amino-5-bromo-anilino)-1-methyl-ethoxy]propyl]-1-methyl-pyrazol-4-yl]-6-methyl-pyridine-4-carboxylate (320 mg, 84.6% yield) as orange oil.

¹H NMR (400 MHz, CDCl₃) δ ppm 7.87 (s, 1H), 7.80 (s, 1H), 7.47 (s, 1H), 6.77 (dd, J=8.4, 2.0 Hz, 1H), 6.72 (d, J=2.4 Hz, 1H), 6.57 (d, J=8.0 Hz, 1H), 4.13 (q, J=7.2 Hz, 2H), 3.95 (s, 3H), 3.87 (s, 3H), 3.70-3.76 (m, 1H), 3.62-3.69 (m, 1H), 3.43 (dt, J=9.2, 6.0 Hz, 1H), 3.13-3.26 (m, 3H), 2.95-3.06 (m, 1H), 2.58 (s, 3H), 1.94 (quin, J=6.8 Hz, 2H), 1.26 (t, J=7.2 Hz, 3H)

### Step 8: Synthesis of methyl 2-[5-[3-[(]S)-2-(2-amino-6-bromo-benzimidazol-1-yl)-1-methyl-ethoxy]propyl]-1-methyl-pyrazol-4-yl]-6-methyl-pyridine-4-carboxylate

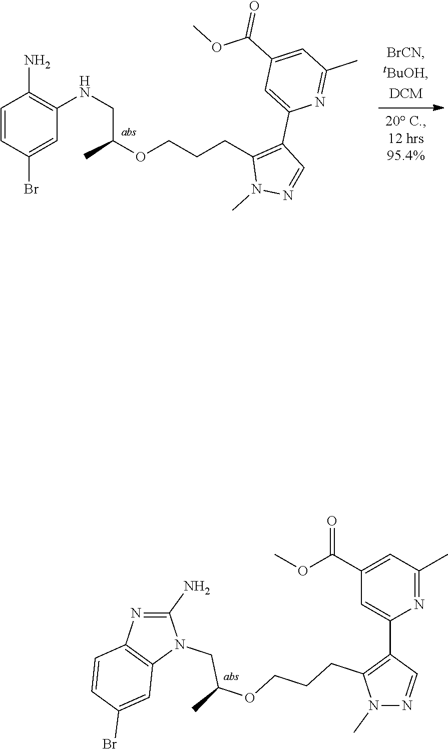

To a solution of methyl 2-[5-[3-[(1S)-2-(2-amino-5-bromo-anilino)-1-methyl-ethoxy]propyl]-1-methyl-pyrazol-4-yl]-6-methyl-pyridine-4-carboxylate (400 mg, 0.775 mmol, 1.0 eq) in DCM (10.0 mL) and t-BuOH (2.0 mL) was added CNBr (470 mg, 4.44 mmol, 5.73 eq). The mixture was stirred at 20° C. for 12 hours. The reaction mixture was treated with saturated aqueous NaHCO₃solution (20 mL) and stirred for 10 minutes. The layers were separated, and the organic phase was washed again with saturated aqueous NaHCO₃solution (20 mL). The organic phase was then dried over Na₂SO₄, filtered, and concentrated under reduced pressure to give methyl 2-[5-[3-[(1S)-2-(2-amino-6-bromo-benzimidazol-1-yl)-1-methyl-ethoxy]propyl]-1-methyl-pyrazol-4-yl]-6-methyl-pyridine-4-carboxylate (400 mg, 95.4% yield) was obtained as a brown solid.

¹H NMR (400 MHz, CDCl₃) δ ppm 7.83-7.88 (m, 1H), 7.73-7.81 (m, 1H), 7.47 (s, 1H), 7.21-7.27 (m, 1H), 7.17 (br d, J=8.0 Hz, 1H), 6.98 (br d, J=8.0 Hz, 1H), 3.93-3.99 (m, 4H), 3.75-3.89 (m, 5H), 3.57-3.72 (m, 2H), 3.27-3.34 (m, 1H), 3.07-3.14 (m, 1H), 3.04 (br s, 1H), 2.48-2.63 (m, 4H), 1.18-1.26 (m, 3H)

### Step 9: Synthesis of 2-[5-[3-[(S)-2-(2-amino-6-bromo-benzimidazol-1-yl)-1-methyl-ethoxy]propyl]-1-methyl-pyrazol-4-yl]-6-methyl-pyridine-4-carboxylic acid

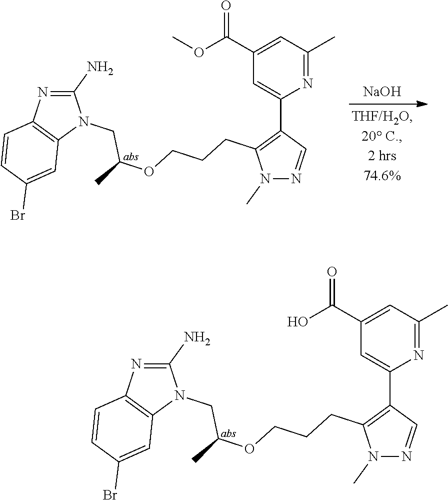 [SMILES: COC(=O)c1cc(C)nc(-c2cnn(C)c2CCCO[C@@H](C)Cn2c(N)nc3ccc(Br)cc32)c1 ; "NaOH" ; Cc1cc(C(=O)O)cc(-c2cnn(C)c2CCCO[C@@H](C)Cn2c(N)nc3ccc(Br)cc32)n1]

To a solution of methyl 2-[5-[3-[(1S)-2-(2-amino-6-bromo-benzimidazol-1-yl)-1-methyl-ethoxy]propyl]-1-methyl-pyrazol-4-yl]-6-methyl-pyridine-4-carboxylate (400 mg, 0.739 mmol, 1.0 eq) in THF (8.0 mL) and H₂O (4.0 mL) was added NaOH (300 mg, 7.50 mmol, 10.2 eq). The mixture was stirred at 20° C. for 2 hours. The reaction mixture was concentrated under reduced pressure to remove organic solvent. The residue was adjusted to pH 5 with 1 N HCl aqueous. The resultant suspension was filtered and the brown cake was dried over reduced pressure to give 2-[5-[3-[(1S)-2-(2-amino-6-bromo-benzimidazol-1-yl)-1-methyl-ethoxy]propyl]-1-methyl-pyrazol-4-yl]-6-methyl-pyridine-4-carboxylic acid (350 mg, 74.6% yield, 83% purity) as a brown solid.

LCMS (ESI) [M+H]⁺ m/z: calcd 527.1; found 527.1.

### Step 10: Synthesis of (11S,21E)-16-bromo-5,11,26-trimethyl-10-oxa-4,5,13,20,22,27-hexazapentacyclo[22.3.1.02,6.013,21.014,19]octacosa-1(28),2(6),3,14(19),15,17,21,24,26-nonaen-23-one

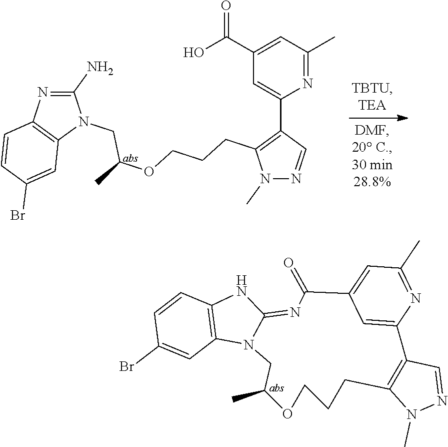 [SMILES: Cc1cc(C(=O)O)cc(-c2cnn(C)c2CCCO[C@@H](C)Cn2c(N)nc3ccc(Br)cc32)n1 ; Cc1cc2cc(n1)-c1cnn(C)c1CCCO[C@@H](C)CN1/C(=N/C2=O)Nc2ccc(Br)cc21]

To a solution of 2-[5-[3-[(1S)-2-(2-amino-6-bromo-benzimidazol-1-yl)-1-methyl-ethoxy]propyl]-1-methyl-pyrazol-4-yl]-6-methyl-pyridine-4-carboxylic acid (300 mg, 0.569 mmol, 1.0 eq) in DMF (3.0 mL) was added TBTU (300 mg, 0.934 mmol, 1.64 eq) and Et₃N (0.5 mL, 3.59 mmol, 6.32 eq). The mixture was stirred at 20° C. for 30 minutes. The reaction mixture was diluted with water (20 mL) and extracted with EtOAc (30 mL*3). The combined organic layers were washed with brine (30 mL*3), dried over Na₂SO₄ and filtered. The filtrate was concentrated under reduced pressure. The residue was purified by preparative TLC(silica, DCM/MeOH=20/1; 254 nm) to give (11S,21E)-16-bromo-5,11,26-trimethyl-10-oxa-4,5,13,20,22,27-hexazapentacyclo[22.3.1.02,6.013,21.014,19]octacosa-1(28),2(6),3,14(19),15,17,21,24,26-nonaen-23-one (85 mg, 28.8% yield, 98.0% purity) as an off-white solid.

¹H NMR (400 MHz, DMSO-d₆) δ ppm 12.83 (br s, 1H), 8.31 (s, 1H), 7.92-8.03 (m, 2H), 7.54 (s, 1H), 7.44-7.49 (m, 1H), 7.38-7.42 (m, 1H), 4.32 (br t, J=12.4 Hz, 2H), 4.14-4.23 (m, 1H), 3.80-3.91 (m, 4H), 3.70 (br d, J=11.6 Hz, 1H), 3.14-3.26 (m, 1H), 3.00 (br t, J=13.2 Hz, 1H), 2.57 (s, 3H), 2.16 (br s, 1H), 1.62 (br s, 1H), 1.10 (br d, J=6.0 Hz, 3H).

LCMS (ESI) [M+H]⁺ m/z: calcd 509.1; found 511.0)

HPLC: 96.67%@220 nm; 97.98% @254 nm)

### Step 11: Synthesis of (11S,21E)-5,11,26-trimethyl-23-oxo-10-oxa-4,5,13,20,22,27-hexazapentacyclo[22.3.1.02,6.013,21.014,19]octacosa-1(28),2(6),3,14(19),15,17,21,24,26-nonaene-16-carbaldehyde

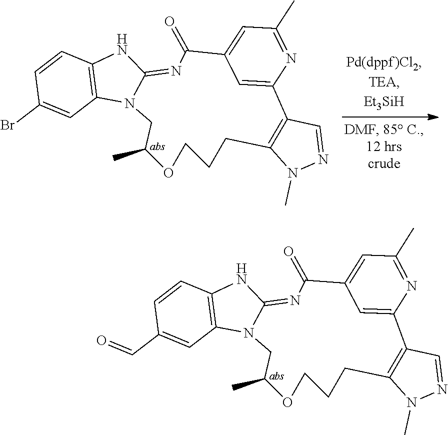

To a solution of (11S,21E)-16-bromo-5,11,26-trimethyl-10-oxa-4,5,13,20,22,27-hexazapentacyclo[22.3.1.02,6.013,21.014,19]octacosa-1(28),2(6),3,14(19),15,17,21,24,26-nonaen-23-one (50 mg, 0.098 mmol, 1.0 eq) in DMF (2.0 mL) were added Pd(dppf)Cl₂ (15 mg, 0.02 mmol, 0.21 eq), TEA (50 mg, 0.49 mmol, 5.03 eq) and triethylsilane (50 mg, 0.43 mmol, 4.38 eq). The mixture was degassed under vacuum and purged with CO three times. The mixture was stirred under CO (15 psi) at 85° C. for 12 hours. The reaction mixture was filtered and the filtrate was diluted with water (20 mL). The mixture was extracted with EtOAc (30 mL*3). The combined organic layers were washed with brine (30 mL*3), dried over Na₂SO₄ and filtered. The filtrate was concentrated under reduced pressure to give (11S,21E)-5,11,26-trimethyl-23-oxo-10-oxa-4,5,13,20,22,27-hexazapentacyclo[22.3.1.02,6.013,21.014,19]octacosa-1(28),2(6),3,14(19),15,17,21,24,26-nonaene-16-carbaldehyde (70 mg, crude) as a brown solid.

### Step 12: Synthesis of (11S,21E)-5,11,26-trimethyl-16-[(4-methylpiperazin-1-yl)methyl]-10-oxa-4,5,13,20,22,27-hexazapentacyclo[22.3.1.02,6.013,21.014,19]octacosa-1(28),2(6),3,14(19),15,17,21,24,26-nonaen-23-one

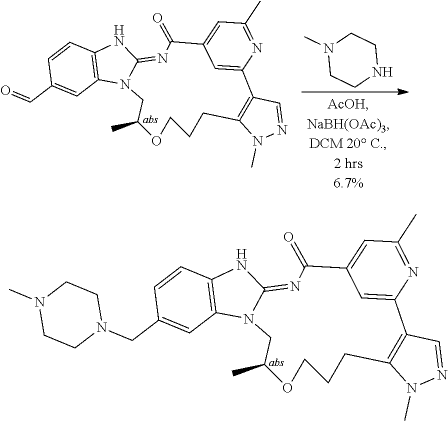

To a solution of (11S,21E)-5,11,26-trimethyl-23-oxo-10-oxa-4,5,13,20,22,27-hexazapentacyclo[22.3.1.02,6.013,21.014,19]octacosa-1(28),2(6),3,14(19),15,17,21,24,26-nonaene-16-carbaldehyde (70 mg, 0.153 mmol, 1.0 eq) in DCM (5.0 mL) were added 1-methylpiperazine (30 mg, 0.30 mmol, 1.96 eq), AcOH (95 mg, 1.58 mmol, 10.4 eq) and NaBH(OAc)₃ (330 mg, 1.56 mmol, 10.2 eq). The mixture was stirred at 20° C. for 2 hours. The reaction mixture was quenched with saturated Na₂CO₃/H₂O (10 mL) and extracted with DCM (30 mL*3). The combined organic layers were dried over Na₂SO₄ and filtered. The filtrate was concentrated under reduced pressure. The residue was purified by flash chromatography (ISCO®; 4 g SepaFlash® Silica Flash Column, DCM/MeOH with MeOH from 0˜20%, flow rate: 18 mL/min, 254 nm) to give a crude product which was further purified by preparative HPLC (Column: Waters Xbridge 150*25 mm*5 μm; mobile phase: [water(FA)-ACN]; B %: 0%-30%, 9.5 min, Temp: 30° C.) to give (11S,21E)-5,11,26-trimethyl-16-[(4-methylpiperazin-1-yl)methyl]-10-oxa-4,5,13,20,22,27-hexazapentacyclo[22.3.1.02,6.013,21.014,19]octacosa-1(28),2(6),3,14(19),15,17,21,24,26-nonaen-23-one (5.6 mg, 6.7% yield) as a yellow solid.

¹H NMR (400 MHz, CD₃OD) (ppm 8.43 (s, 1H), 8.19 (s, 1H), 7.98 (s, 1H), 7.55 (br s, 1H), 7.41 (br s, 2H), 7.25 (br d, J=7.6 Hz, 1H), 4.34 (br d, J=9.6 Hz, 1H), 4.21 (br s, 1H), 4.03 (br s, 1H), 3.86 (s, 4H), 3.67-3.79 (m, 3H), 3.17 (br s, 5H), 2.94 (br s, 1H), 2.78 (s, 7H), 2.53 (s, 3H), 2.16 (br s, 1H), 1.67 (br s, 1H), 1.11 (br d, J=5.2 Hz, 3H).

LCMS (ESI) [M+H]⁺ m/z: calcd 543.3; found 543.2.

HPLC: 97.76%@220 nm; 98.94%@254 nm.

Chiral SFC: 100% ee.

### Example 7: Preparation of Compound (63)

### The synthesis of (22E)-5,12,27-trimethyl-]7-[(4-methylpiperazin-1-yl)methyl]-4,5,14,21,23,28-hexazahexacyclo[23.3.1.1^{7,11}.0^{2,6}.0^{14,22}.0^{15,20}]triaconta-1(29),2(6), 3, 7(30),8,10,15(20),16,18,22,25,27-dodecaen-24-one (compound(63))

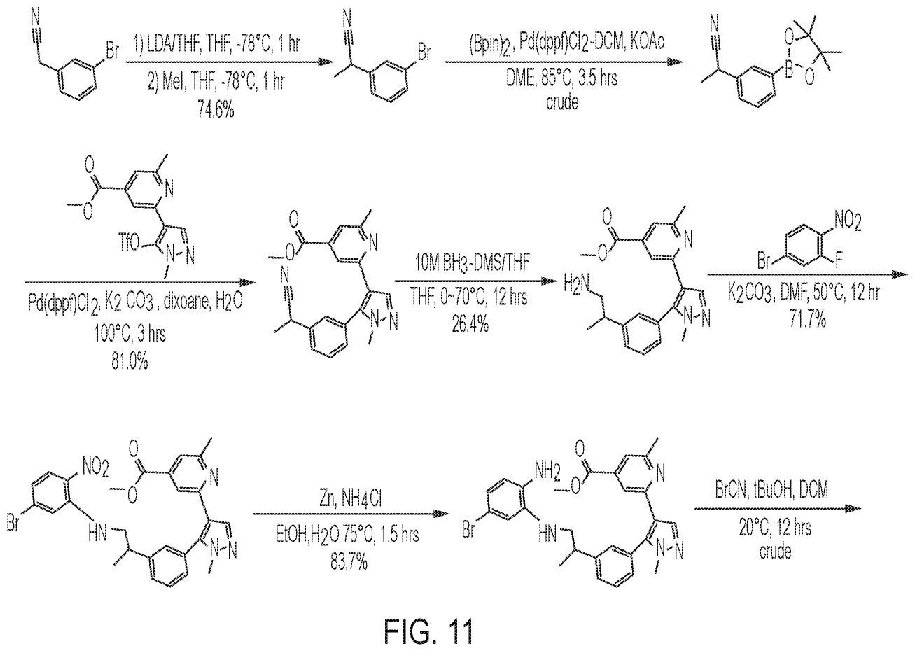

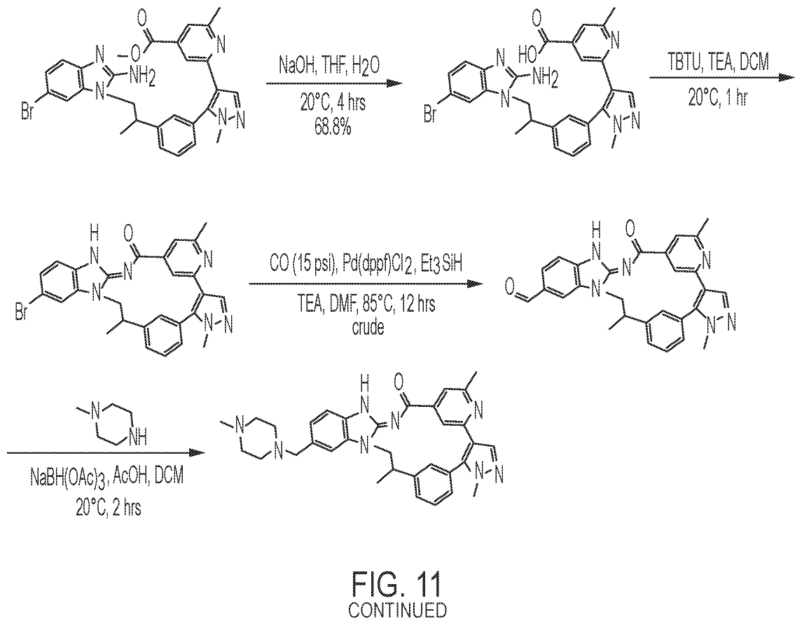

The general synthetic schemes for preparing Compound (63) according to the invention are provided in FIG. 11.

### Step 1: Synthesis of 2-(3-bromophenyl)propanenitrile

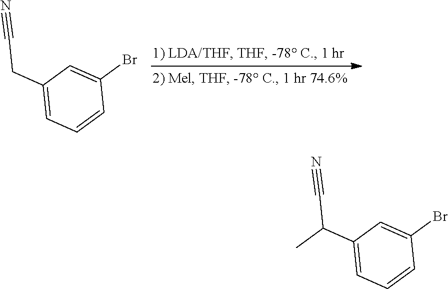

To a mixture of 2-(3-bromophenyl)acetonitrile (1.0 g, 5.10 mmol, 1.0 eq) in THF (15.0 mL) was degassed and purged with N2 for 3 times, and then MeI (0.4 mL, 6.43 mmol, 1.3 eq) was added at −78° C. The mixture was stirred for 1 hour, and then 2 M LDA/THF (3.0 mL, 6 mmol, 1.2 eq) was added dropwise at −78° C. The resulting mixture was stirred at −78° C. for 1 hour. The combined two batches reaction mixture was quenched by addition saturated NH₄Cl/H₂O (30 mL), and then extracted with EtOAc (50 mL*2). The combined organic layers were washed with brine (30 mL), dried over Na₂SO₄, filtered and concentrated under reduced pressure. The residue was purified by flash chromatography (ISCO©; 20 g SepaFlash© Silica Flash Column, petroleum ether/EtOAc with EtOAc from 0˜5%, flow rate=80 mL/min, 254 nm) to afford 2-(3-bromophenyl)propanenitrile (1.6 g, 74.6% yield) as a colorless oil.

¹H NMR (400 MHz, chloroform-d) (ppm 7.51 (d, J=1.4 Hz, 1H), 7.48 (td, J=1.4, 7.6 Hz, 1H), 7.34-7.26 (m, 2H), 3.88 (q, J=7.2 Hz, 1H), 1.65 (d, J=7.2 Hz, 3H).

### Step 2: Synthesis of 2-[3-(4,4,5,5-tetramethyl-1,3,2-dioxaborolan-2-yl)phenyl]propanenitrile

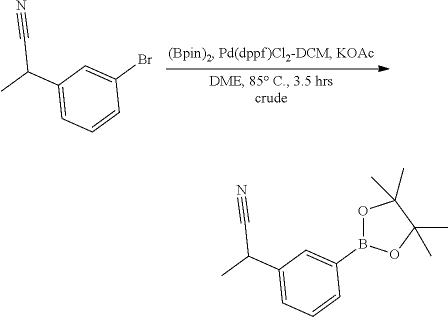 [SMILES: CC(C#N)c1cccc(Br)c1 ; CC(C#N)c1cccc(B2OC(C)(C)C(C)(C)O2)c1]

A mixture of 2-(3-bromophenyl)propanenitrile (1.4 g, 6.66 mmol, 1.0 eq), Pd(dppf)Cl₂—CH₂Cl₂ (560 mg, 0.686 mmol, 0.1 eq), KOAc (1.40 g, 14.3 mmol, 2.1 eq) and (Bpin)₂ (2.10 g, 8.27 mmol, 1.2 eq) in DME (20.0 mL) was degassed and purged with N2 for 3 times, and then the mixture was stirred at 85° C. for 3.5 hours under N2 atmosphere. The reaction mixture was filtered and the filter cake was washed with DCM (50 mL). The combined filtrate was concentrated under reduced pressure to afford 2-[3-(4,4,5,5-tetramethyl-1,3,2-dioxaborolan-2-yl)phenyl]propanenitrile (3.0 g, crude) as a brown oil.

LCMS (ESI) [M+H]⁺ m/z: calcd 258.1, found 258.2.

### Step 3: Synthesis of methyl 2-[5-[3-(I-cyanoethyl)phenyl]-1-methyl-pyrazol-4-yl]-6-methyl-pyridine-4-carboxylate

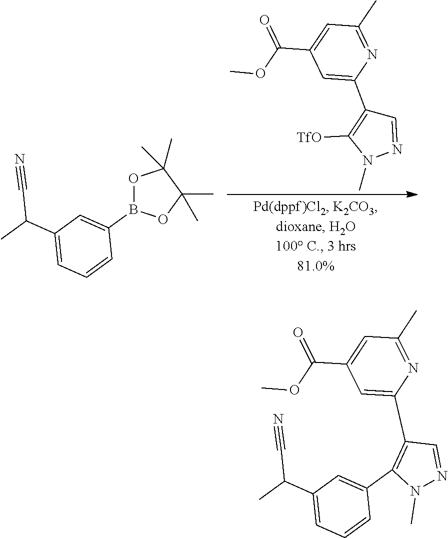 [SMILES: CC(C#N)c1cccc(B2OC(C)(C)C(C)(C)O2)c1 ; COC(=O)c1cc(C)nc(-c2cnn(C)c2OS(=O)(=O)C(F)(F)F)c1 ; COC(=O)c1cc(C)nc(-c2cnn(C)c2-c2cccc(C(C)C#N)c2)c1]

A mixture of methyl 2-methyl-6-[1-methyl-5-(trifluoromethylsulfonyloxy)pyrazol-4-yl]pyridine-4-carboxylate (900 mg, 2.37 mmol, 1.0 eq), 2-[3-(4,4,5,5-tetramethyl-1,3,2-dioxaborolan-2-yl)phenyl]propanenitrile (3.0 g, 11.7 mmol, 4.9 eq), Pd(dppf)Cl₂ (220 mg, 0.301 mmol, 0.1 eq) and K₂CO₃ (1.0 g, 7.24 mmol, 3.1 eq) in dioxane (10.0 mL) and H₂O (2.0 mL) was degassed and purged with N2 for 3 times, and then the mixture was stirred at 100° C. for 3 hours under N2 atmosphere. The reaction mixture was filtered and the filter cake was washed with DCM (100 mL). The combined filtrate was concentrated under reduced pressure. The residue was purified by flash silica gel chromatography (ISCO©; 40 g SepaFlash® Silica Flash Column, Petroleum ether/EtOAc with EtOAc from 0˜59%, flow rate=100 mL/min, 254 nm) methyl 2-[5-[3-(1-cyanoethyl)phenyl]-1-methyl-pyrazol-4-yl]-6-methyl-pyridine-4-carboxylate (900 mg, 81.0% yield, 77% purity) as a yellow oil.

LCMS (ESI) [M+H]⁺ m/z: calcd 361.1, found 361.0.

### Step 4: Synthesis of methyl 2-[5-[3-(2-amino-]-methyl-ethyl)phenyl]-1-methyl-pyrazol-4-yl]-6-methyl-pyridine-4-carboxylate

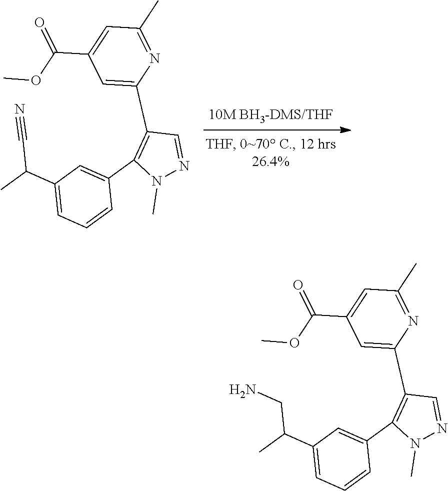 [SMILES: COC(=O)c1cc(C)nc(-c2cnn(C)c2-c2cccc(C(C)C#N)c2)c1 ; COC(=O)c1cc(C)nc(-c2cnn(C)c2-c2cccc(C(C)CN)c2)c1]

A mixture of methyl 2-[5-[3-(1-cyanoethyl)phenyl]-1-methyl-pyrazol-4-yl]-6-methyl-pyridine-4-carboxylate (900 mg, 2.50 mmol, 1.0 eq) in THF (10. mL) was degassed and purged with N2 for 3 times, and then TOM BH₃-Me₂S/THF (600 mg, 0.8 mL, 3.2 eq) was added at 0° C. The mixture was stirred at 70° C. for 12 hours under N2 atmosphere. The reaction mixture was quenched by addition 6N HCl/H₂O (10 mL) and stirred for 70° C. for 30 minutes and then adjusted with saturated Na₂CO₃/H₂O to pH=10 and extracted with DCM (50 mL*2). The combined organic layers were dried over Na₂SO₄, filtered and concentrated under reduced pressure. The residue was purified by flash silica gel chromatography (ISCO©; 12 g SepaFlash® Silica Flash Column, DCM/MeOH (0.05% TEA) with MeOH from 0˜15%, flow rate=40 mL/min, 254 nm) to afford methyl 2-[5-[3-(2-amino-1-methyl-ethyl)phenyl]-1-methyl-pyrazol-4-yl]-6-methyl-pyridine-4-carboxylate (270 mg, 26.4% yield, 89% purity) as a yellow oil.

¹H NMR (400 MHz, chloroform-d) δ ppm 8.12 (s, 1H), 7.50-7.46 (m, 1H), 7.41 (s, 1H), 7.37 (br d, J=7.8 Hz, 1H), 7.24 (d, J=7.6 Hz, 1H), 7.20 (d, J=5.6 Hz, 2H), 3.80 (d, J=7.0 Hz, 6H), 2.92-2.82 (m, 3H), 2.54 (s, 3H), 1.25 (br d, J=6.2 Hz, 5H).

LCMS (ESI) [M+H]⁺ m/z: calcd 365.1, found 365.1.

### Step 5: Synthesis of methyl 2-[5-[3-[2-(5-bromo-2-nitro-anilino)-1-methyl-ethyl]phenyl]-1-methyl-pyrazol-4-yl]-6-methyl-pyridine-4-carboxylate

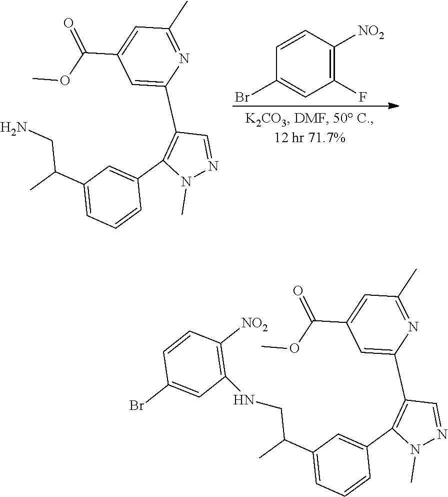 [SMILES: COC(=O)c1cc(C)nc(-c2cnn(C)c2-c2cccc(C(C)CN)c2)c1 ; O=[N+]([O-])c1ccc(Br)cc1F ; COC(=O)c1cc(C)nc(-c2cnn(C)c2-c2cccc(C(C)CNc3cc(Br)ccc3[N+](=O)[O-])c2)c1]

A mixture of methyl 2-[5-[3-(2-amino-1-methyl-ethyl)phenyl]-1-methyl-pyrazol-4-yl]-6-methyl-pyridine-4-carboxylate (270 mg, 0.741 mmol, 1.0 eq), 4-bromo-2-fluoro-1-nitro-benzene (250 mg, 1.14 mmol, 1.5 eq) and K₂CO₃ (300 mg, 2.17 mmol, 2.9 eq) in DMF (5.0 mL) was stirred at 50° C. for 12 hours. The reaction mixture was diluted with water (30 mL) and extracted with EtOAc (50 mL*2). The combined organic layers were washed with brine (30 mL*3), dried over Na₂SO₄, filtered and concentrated under reduced pressure. The residue was purified by flash silica gel chromatography (ISCO®; 12 g SepaFlash® Silica Flash Column, Petroleum ether/EtOAc with EtOAc from 0˜55%, flow rate=40 mL/min, 254 nm) to afford methyl 2-[5-[3-[2-(5-bromo-2-nitro-anilino)-1-methyl-ethyl]phenyl]-1-methyl-pyrazol-4-yl]-6-methyl-pyridine-4-carboxylate (300 mg, 71.7% yield, 100% purity) as a yellow solid.

¹H NMR (400 MHz, chloroform-d) δ ppm 8.12 (s, 1H), 8.01 (br d, J=4.8 Hz, 1H), 7.99 (d, J=9.0 Hz, 1H), 7.54-7.49 (m, 1H), 7.43-7.39 (m, 2H), 7.31-7.28 (m, 2H), 6.98 (d, J=1.8 Hz, 1H), 6.75 (dd, J=1.8, 9.2 Hz, 1H), 3.78 (d, J=3.8 Hz, 6H), 3.50-3.38 (m, 2H), 3.18 (sxt, J=7.0 Hz, 1H), 2.52 (s, 3H), 1.42 (d, J=7.0 Hz, 3H).

LCMS (ESI) [M+H]⁺ m/z: calcd 564.1, found 564.0.

### Step 6: Synthesis of methyl 2-[5-[3-[2-(2-amino-5-bromo-anilino)-1-methyl-ethyl]phenyl]-1-methyl-pyrazol-4-yl]-6-methyl-pyridine-4-carboxylate

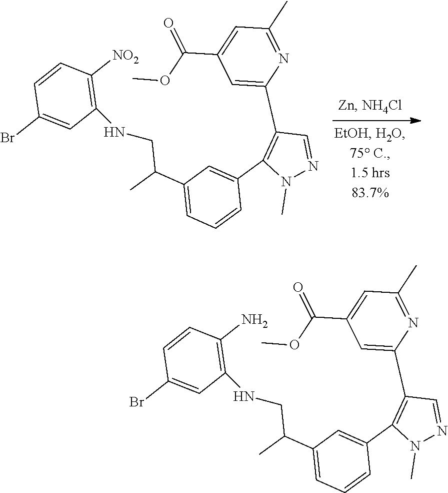 [SMILES: COC(=O)c1cc(C)nc(-c2cnn(C)c2-c2cccc(C(C)CNc3cc(Br)ccc3[N+](=O)[O-])c2)c1 ; COC(=O)c1cc(C)nc(-c2cnn(C)c2-c2cccc(C(C)CNc3cc(Br)ccc3N)c2)c1]

To a mixture of methyl 2-[5-[3-[2-(5-bromo-2-nitro-anilino)-1-methyl-ethyl]phenyl]-1-methyl-pyrazol-4-yl]-6-methyl-pyridine-4-carboxylate (230 mg, 0.407 mmol, 1.0 eq) in EtOH (5.0 mL) and H₂O (2.5 mL) were added Zn (300 mg, 4.59 mmol, 11.3 eq) and NH₄Cl (250 mg, 4.67 mmol, 11.5 eq). The mixture was stirred at 75° C. for 1.5 hours. The reaction mixture was filtered and the filter cake was washed with DCM (100 mL), and then diluted with sat. Na₂CO₃ (50 mL) and extracted with DCM (50 mL). The combined organic layers was dried over Na₂SO₄, filtered and concentrated under reduced pressure. The residue was purified by flash silica gel chromatography (ISCO®; 4 g SepaFlash® Silica Flash Column, Petroleum ether/EtOAc with EtOAc from 0˜70%, flow rate=18 mL/min, 254 nm) to afford methyl 2-[5-[3-[2-(2-amino-5-bromo-anilino)-1-methyl-ethyl]phenyl]-1-methyl-pyrazol-4-yl]-6-methyl-pyridine-4-carboxylate (190 mg, 83.7% yield, 96% purity) as a gray solid.

LCMS (ESI) [M+H]⁺ m/z: calcd 534.1, found 536.1.

### Step 7: Synthesis of methyl 2-[5-[3-[2-(2-amino-6-bromo-benzimidazol-1-yl)-1-methyl-ethyl]phenyl]-1-methyl-pyrazol-4-yl]-6-methyl-pyridine-4-carboxylate

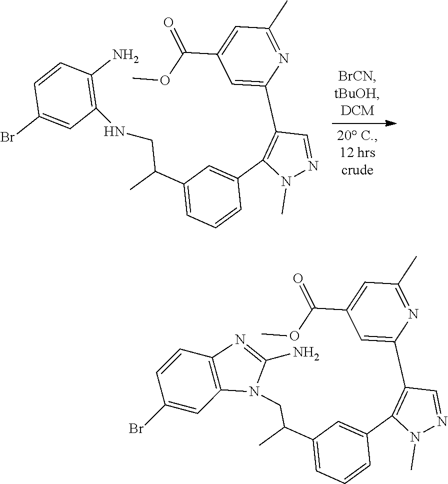 [SMILES: COC(=O)c1cc(C)nc(-c2cnn(C)c2-c2cccc(C(C)CNc3cc(Br)ccc3N)c2)c1 ; COC(=O)c1cc(C)nc(-c2cnn(C)c2-c2cccc(C(C)Cn3c(N)nc4ccc(Br)cc43)c2)c1]

To a mixture of methyl 2-[5-[3-[2-(2-amino-5-bromo-anilino)-1-methyl-ethyl]phenyl]-1-methyl-pyrazol-4-yl]-6-methyl-pyridine-4-carboxylate (140 mg, 0.262 mmol, 1.0 eq) in t-BuOH (5.0 mL) and DCM (1.0 mL) was added BrCN (200 mg, 1.89 mmol, 7.2 eq). The mixture was stirred at 20° C. for 12 hours. The reaction mixture was diluted with water (30 mL) and extracted with DCM (30 mL*2). The combined organic layers were dried over Na₂S04, filtered and concentrated under reduced pressure to afford methyl 2-[5-[3-[2-(2-amino-6-bromo-benzimidazol-1-yl)-1-methyl-ethyl]phenyl]-1-methyl-pyrazol-4-yl]-6-methyl-pyridine-4-carboxylate (180 mg, crude) as a yellow oil.

LCMS (ESI) [M+H]⁺ m/z: calcd 559.1, found 561.1.

### Step 8: Synthesis of 2-[5-[3-[2-(2-amino-6-bromo-benzimidazol-1-yl)-1-methyl-ethyl]phenyl]-1-methyl-pyrazol-4-yl]-6-methyl-pyridine-4-carboxylic acid

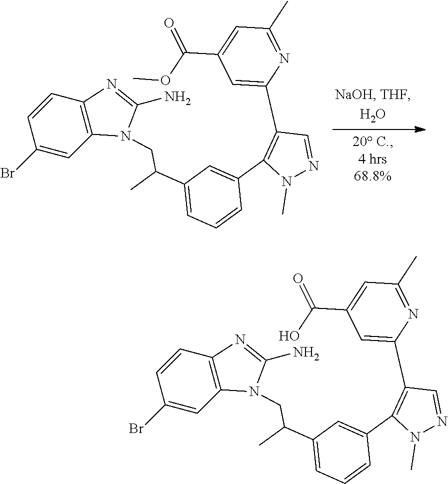 [SMILES: COC(=O)c1cc(C)nc(-c2cnn(C)c2-c2cccc(C(C)Cn3c(N)nc4ccc(Br)cc43)c2)c1 ; Cc1cc(C(=O)O)cc(-c2cnn(C)c2-c2cccc(C(C)Cn3c(N)nc4ccc(Br)cc43)c2)n1]

A mixture of methyl 2-[5-[3-[2-(2-amino-6-bromo-benzimidazol-1-yl)-1-methyl-ethyl]phenyl]-1-methyl-pyrazol-4-yl]-6-methyl-pyridine-4-carboxylate (180 mg, 0.322 mmol, 1.0 eq) and NaOH (130 mg, 3.25 mmol, 10.1 eq) in THF (2.0 mL) and H₂O (1.0 mL) was stirred at 20° C. for 2 hours. The reaction mixture was concentrated under reduced pressure to remove THF, and adjusted with 1N HCl/H₂O to pH=5. The precipitate was collected by filtration, washed with water (2 mL) and dried under reduced pressure to afford 2-[5-[3-[2-(2-amino-6-bromo-benzimidazol-1-yl)-1-methyl-ethyl]phenyl]-1-methyl-pyrazol-4-yl]-6-methyl-pyridine-4-carboxylic acid (130 mg, 68.8% yield, 93% purity) as a yellow solid.

LCMS (ESI) [M+H]⁺ m/z: calcd 545.1, found 545.0.

### Step 9: Synthesis of (22E)-17-bromo-5,12,27-trimethyl-4,5,14,21,23,28-hexazahexacyclo[23.3.1.1^{7,11}.0^{2,6}.0^{14,22}.0^{15,20}]triaconta-1(29),2(6),3, 7(30),8,10,15(20),16,18,22,25,27-dodecaen-24-one

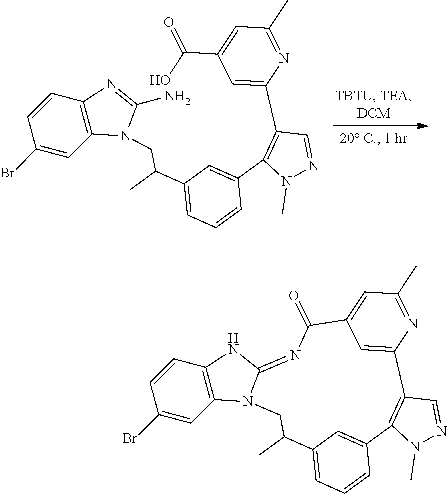 [SMILES: Cc1cc(C(=O)O)cc(-c2cnn(C)c2-c2cccc(C(C)Cn3c(N)nc4ccc(Br)cc43)c2)n1 ; Cc1cc2cc(n1)-c1cnn(C)c1-c1cccc(c1)C(C)CN1/C(=N/C2=O)Nc2ccc(Br)cc21]

To a mixture of 2-[5-[3-[2-(2-amino-6-bromo-benzimidazol-1-yl)-1-methyl-ethyl]phenyl]-1-methyl-pyrazol-4-yl]-6-methyl-pyridine-4-carboxylic acid (150 mg, 0.275 mmol, 1.0 eq) and TBTU (140 mg, 0.436 mmol, 1.6 eq) in DCM (30.0 mL) was added TEA (0.1 mL, 0.718 mmol, 2.6 eq). The mixture was stirred at 20° C. for 1 hour. The reaction mixture was diluted with water (30 mL) and extracted with DCM (20 mL*2). The combined organic layers were washed with brine (20 mL), dried over Na₂SO₄, filtered and concentrated under reduced pressure. The crude product (50 mg) was purified by preparative HPLC (Column: Phenomenex C18 80*40 mm*3 μm; Mobile phase: [water(NH₄HCO₃)-ACN]; B %: 38%-68%, 9.5 min, Column Temp: 30° C.) to afford (22E)-17-bromo-5,12,27-trimethyl-4,5,14,21,23,28-hexazahexacyclo[23.3.1.1^{7,11}.0^{2,6}.0^{14,22}.0^{15,20}]triaconta-1(29),2(6),3,7(30),8,10,15(20),16,18,22,25,27-dodecaen-24-one (8.7 mg) as a white solid and (22E)-17-bromo-5,12,27-trimethyl-4,5,14,21,23,28-hexazahexacyclo[23.3.1.1^{7,11}.0^{2,6}.0^{14,22}.0^{15,20}]triaconta-1(29),2(6),3,7(30),8,10,15(20),16,18,22,25,27-dodecaen-24-one (250 mg, crude) as a yellow solid

¹H NMR (400 MHz, methanol-d₄) δ ppm 8.14 (s, 1H), 8.08 (s, 1H), 7.90 (s, 1H), 7.75 (d, J=1.6 Hz, 1H), 7.46-7.42 (m, 1H), 7.37-7.34 (m, 2H), 7.20-7.15 (m, 1H), 6.90 (dd, J=7.8, 12.8 Hz, 2H), 4.79 (br d, J=3.2 Hz, 1H), 4.13 (br d, J=12.0 Hz, 1H), 3.78 (s, 3H), 3.49-3.40 (m, 1H), 2.57 (s, 3H), 1.63 (d, J=7.0 Hz, 3H).

HPLC: 98.71%@220 nm, 98.68%@254 nm.

LCMS (ESI) [M+H]⁺ m/z: calcd 527.1, found 529.1.

### Step 10: Synthesis of (22E)-5,12,27-trimethyl-24-oxo-4,5,14,21,23,28-hexazahexacyclo[23.3.1.1^{7,11}.0^{2,6}.0^{14,22}.0^{15,20}]triaconta-1(29),2(6),3, 7(30),8,10,15(20),16,18,22,25,27-dodecaene-17-carbaldehyde

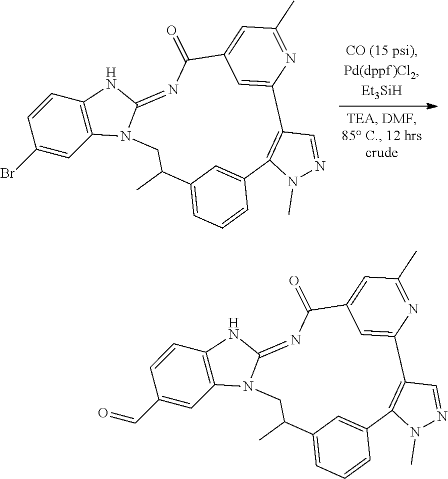 [SMILES: Cc1cc2cc(n1)-c1cnn(C)c1-c1cccc(c1)C(C)CN1/C(=N/C2=O)Nc2ccc(Br)cc21 ; Cc1cc2cc(n1)-c1cnn(C)c1-c1cccc(c1)C(C)CN1/C(=N/C2=O)Nc2ccc(C=O)cc21]

To a solution of (22E)-17-bromo-5,12,27-trimethyl-4,5,14,21,23,28-hexazahexacyclo[23.3.1.1^{7,11}.0^{2,6}.0^{14,22}.0^{15,20}]triaconta-1(29),2(6),3,7(30),8,10,15(20),16,18,22,25,27-dodecaen-24-one (220 mg, 0.417 mmol, 1.0 eq) and Pd(dppf)Cl₂ (35 mg, 0.0478 mmol, 0.1 eq) in DMF (3.0 mL) were added TEA (0.2 mL, 1.44 mmol, 3.4 eq) and triethylsilane (0.2 mL, 1.25 mmol, 3.0 eq) under N2. The suspension was degassed under vacuum and purged with CO several times. The mixture was stirred under CO (15 psi) at 85° C. for 12 hours. The reaction mixture was filtered and the filter cake was washed with DCM (50 mL). The combined filtrate was washed with saturated NaHCO₃/H₂O (20 mL) and brine (20 mL), dried over Na₂SO₄, filtered and concentrated under reduced pressure to afford (22E)-5,12,27-trimethyl-24-oxo-4,5,14,21,23,28-hexazahexacyclo[23.3.1.1^{7,11}.0^{2,6}.0^{14,22}.0^{15,20}]triaconta-1(29),2(6),3,7(30),8,10,15(20),16,18,22,25,27-dodecaene-17-carbaldehyde (200 mg, crude) as a brown solid.

LCMS (ESI) [M+H]⁺ m/z: calcd 477.2, found 477.1.

### Step 11: Synthesis of (22E)-5,12,27-trimethyl-]7-[(4-methylpiperazin-1-yl)methyl]-4,5,14,21,23,28-hexazahexacyclo[23.3.1.1^{7,11}.0^{2,6}.0^{14,22}.0^{15,20}]triaconta-1(29),2(6),3, 7(30),8,10,15(20),16,18,22,25,27-dodecaen-24-one

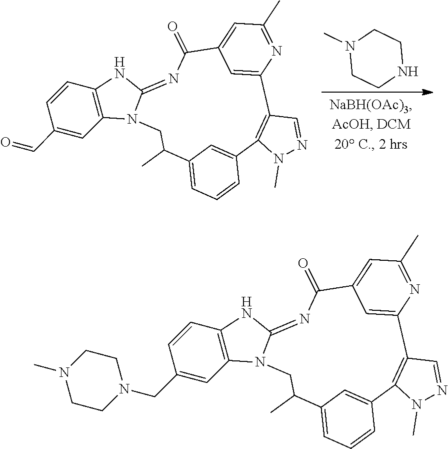

To a solution of (22E)-5,12,27-trimethyl-24-oxo-4,5,14,21,23,28-hexazahexacyclo[23.3.1.1^{7,11}.0^{2,6}.0^{14,22}.0^{15,20}]triaconta-1(29),2(6),3,7(30),8,10,15(20),16,18,22,25,27-dodecaene-17-carbaldehyde (200 mg, 0.420 mmol, 1.0 eq) and 1-methylpiperazine (90 mg, 0.899 mmol, 2.1 eq) in DCM (5.0 mL) were added HOAc (3.50 mmol, 0.2 mL, 8.3 eq) and NaBH(OAc)₃ (500 mg, 2.36 mmol, 5.6 eq). The solution was stirred at 20° C. for 2 hours. The reaction mixture was diluted with DCM (50 mL) and washed with saturated Na₂CO₃ aqueous solution (20 mL). The organic layers was dried over anhydrous Na₂SO₄, filtered and concentrated under reduced pressure. The residue was purified by flash chromatography (ISCO®; 4 g SepaFlash® Silica Flash Column, DCM/MeOH (0.05% TEA) with MeOH from 0˜30%, flow rate=18 mL/min, 254 nm) to give a crude product. The crude product was purified by preparative HPLC (Column: Waters Xbridge 150*25 mm*5 μm; Mobile phase: [water(FA)-ACN]; B %: 5%-35%, 9.5 min, Column Temp: 30° C.) to afford (22E)-5,12,27-trimethyl-17-[(4-methylpiperazin-1-yl)methyl]-4,5,14,21,23,28-hexazahexacyclo[23.3.1.1^{7,11}.0^{2,6}.0^{14,22}.0^{15,20}]triaconta-1(29),2(6),3,7(30),8,10,15(20),16,18,22,25,27-dodecaen-24-one (8.4 mg, 3.4% yield) as a yellow solid.

¹H NMR (400 MHz, methanol-d₄) δ ppm 8.14 (s, 1H), 8.09 (s, 1H), 7.91 (s, 1H), 7.56 (s, 1H), 7.42 (br d, J=8.4 Hz, 1H), 7.36 (s, 1H), 7.32 (br d, J=8.0 Hz, 1H), 7.17 (t, J=7.6 Hz, 1H), 6.91 (br d, J=7.6 Hz, 1H), 6.85 (br d, J=7.6 Hz, 1H), 4.82-4.81 (m, 1H), 4.15 (br d, J=12.6 Hz, 1H), 3.82 (s, 2H), 3.78 (s, 3H), 3.47 (br s, 1H), 3.09 (br s, 4H), 2.94-2.64 (m, 7H), 2.57 (s, 3H), 1.64 (br d, J=7.0 Hz, 3H).

LCMS (ESI) [M+H]⁺ m/z: calcd 561.3, found 561.1.

HPLC: 97.436%@220 nm, 97.010%@254 nm.

### Example 8: In Vitro Assays

The biological activity of compounds described herein were studied according to standard methods known in the art. Methods were used to study inhibition of EGFR, including mutant forms of EGFR comprising L858R, T790M, C797S, and/or Del19 mutations, or any combination thereof (e.g., L858R single, double, or triple mutants). Exemplary, non-limiting methods are described herein.

Kinase Assays

Assays using an in vitro kinase assay kit (HTRF KinEASE-TK kit) were used to study the inhibitory activity of compounds described herein with respect to EGFR mutants such as (1) EGFRL858R, (2) EGFR^{L858R/T790M}, and (3) EGFR^{L858R/T790M/C797S}.

Ba/F3 Viability Assays

Inhibition of cell proliferation was studied using Ba/F3 viability assays, including the Promega CellTiter-Glo cell viability assay. This assay was used to study the effect of compounds described herein in the following assays: (1) Ba/F3 EGFR-Del19/T790M; (2); Ba/F3 EGFR-Del19/C797S; and (3) Ba/F3 EGFR-Del19/T790M/C797S. P-EGFR Signaling Assays

Phosphorylation of EGFR can be studied using multiplex immunoassay kits such as Phospho-EGFR (Tyr1068) Total EGFR MULTI-SPOT® 96 HB 4-Spot Custom EGFR Duplex ANALYTES assay.

Sixty-one compounds were studied using the six kinase (EGFR) and BA/F3 assays described herein, with more than half of the tested compounds providing IC₅₀<50 nM values in all assays. Accordingly, compounds of the invention are a new general class of kinase inhibitors, including potent inhibitors of EGFR mutants.

Exemplary data are provided in Table 2 and are categorized as follows:

TABLE 2

| Application | | | | | | |
|---|---|---|---|---|---|---|
| compound | KINASE LTC | KINASE LT | KINASE L | BAF3 DTC | BAF3 DT | BAF3 DC |
| (2) | A | A | A | A | A | B |
| (3) | A | A | A | A | B | C |
| (4) | A | A | A | A | A | A |
| (7) | A | A | A | A | A | A |
| (9) | A | A | A | A | A | A |
| (10) | A | A | A | A | A | A |
| (11) | A | A | A | A | A | A |
| (15) | A | A | A | A | A | A |
| (18) | A | A | A | A | B | C |
| (24) | A | A | A | A | A | A |
| (25) | A | A | A | A | A | A |
| (28) | A | A | A | A | A | A |
| (35) | A | A | A | A | A | A |
| (40) | A | A | A | A | A | C |
| (46) | A | A | A | A | A | A |
| (55) | A | A | C | B | C | C |
| (57) | A | A | C | C | D | C |
| (61) | A | A | C | C | C | C |
| (62) | C | C | A | C | D | B |
| (64) | A | A | A | A | A | B |
| (69) | A | A | A | A | A | A |

Legend:
A = IC₅₀ <50 nM
B = 50 nM ≤ IC₅₀ < 100 nM
C = 100 nM ≤ IC₅₀ < 1000 nM
D = IC₅₀ ≥1000 nM

From the ongoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

What is claimed is:

1. A compound of Formula I: 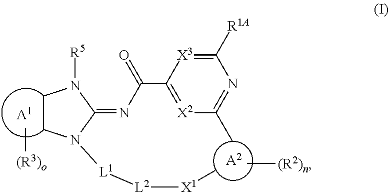
or a pharmaceutically acceptable salt thereof, wherein
A¹ is independently phenylene or 5- or 6-membered heteroarylene;
A² is independently phenyl, naphthyl, or a 5- to 13-membered heteroaryl;
X¹ is independently O or X^{1A};
X^{1A} is a covalent bond, S, NR⁴, C₁₋₆ alkylene, C₂₋₆ alkenylene, or C₂₋₆ alkynylene;
each of X² and X³ is independently N or CR^{1B};
L¹ is independently a covalent bond or C₁₋₆ alkylene;
L² is independently a covalent bond, C₂₋₆ alkenylene, C₂₋₆ alkynylene, C₃₋₆ cycloalkylene, 3- to 10-membered heterocyclylene, phenylene, or 5- or 6-membered heteroarylene;
each R^{1A} and R^{1B} is independently H, OH, CN, halogen, C₁₋₆ aliphatic, C₁₋₆ alkoxy, NR⁶R⁷, C(O)R⁸, CO₂R⁸, C(O)NR⁶R⁷, NR⁹C(O)R⁸, NR⁹CO₂R⁸, NR⁹C(O)NR⁶R⁷, or R¹⁰;
each R² and R³, when present, is independently OH, CN, halogen, C₁₋₆ aliphatic, C₁₋₆ alkoxy, NR⁶R⁷, C(O)R⁸, CO₂R′, C(O)NR⁶R⁷, NR⁹C(O)R⁸, NR⁹CO₂R⁸, NR⁹C(O)NR⁶R⁷, R¹⁰, OR¹⁰, CH₂R¹⁰, CH₂CH₂R¹⁰, OCH₂R¹⁰, or OCH₂CH₂R¹⁰;
each R⁴ is independently H, a N-protecting group, or C₁₋₆ alkyl;
R⁵ is hydrogen;
each R⁶, R⁷, and R⁹ is independently H or C₁₋₆ alkyl; or R⁶ and R⁷, together with the nitrogen atom to which they are attached, form a 3- to 10-membered heterocyclyl, or R⁶ and R⁹, together with the atoms to which they are attached, form a 3- to 10-membered heterocyclyl;
R⁸ is independently C₁₋₆ aliphatic, C₃-C₁₀ cycloaliphatic, 3- to 10-membered heterocyclyl, phenyl, naphthyl, or a 5- to 12-membered heteroaryl, or R⁸ and R⁹, together with the atoms to which they are attached, form a 3- to 10-membered heterocyclyl;
R¹⁰ is independently C₃-C₁₀ cycloaliphatic, 3- to 10-membered heterocyclyl, phenyl, naphthyl, or a 5- to 12-membered heteroaryl;
each of n and o is independently 0, 1, or 2; and
wherein X¹ is O, and both of X² and X³ are not N, then A² is naphthyl or a bicyclic 8- to 12-membered heteroaryl.

2. The compound of claim 1, wherein at least one of X² and X³ is N.

3. The compound of claim 1 or 2, having a structure according to Formula (II), 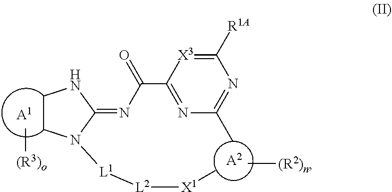
or a pharmaceutically acceptable salt thereof.

4. The compound of claim 1 or 2, having a structure according to Formula (III), 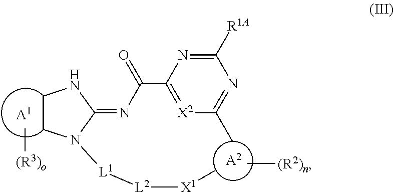
or a pharmaceutically acceptable salt thereof.

5. The compound of claim 1, having a structure according to Formula (IV), 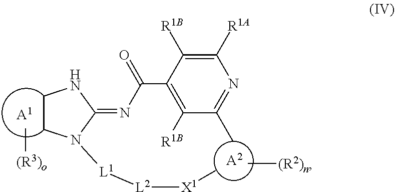
or a pharmaceutically acceptable salt thereof.

6. The compound of any one of claims 1-5, wherein each R^{1B} is H.

7. The compound of any one of claims 1-6, wherein X¹ is XIA.

8. The compound of claim 7, having a structure according to Formula (V), 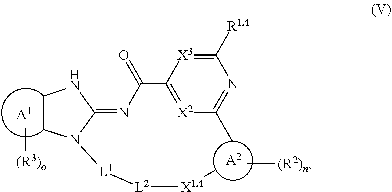
or a pharmaceutically acceptable salt thereof.

9. The compound of claim 1, having a structure according to Formula (VI), 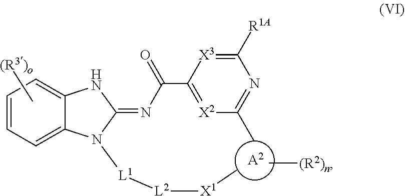
or a pharmaceutically acceptable salt thereof.

10. The compound of claim 9, having a structure according to Formula (VI-1), 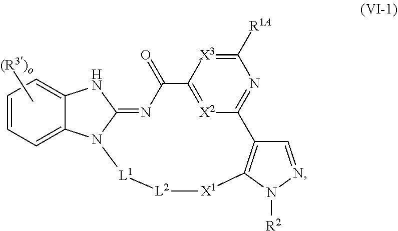
or a pharmaceutically acceptable salt thereof.

11. The compound of claim 9, having a structure according to Formula (VI-2), 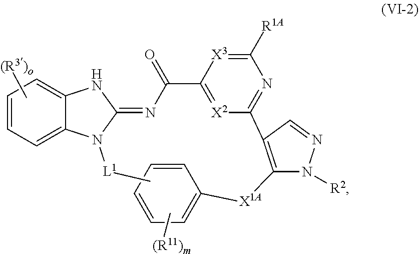
or a pharmaceutically acceptable salt thereof, wherein
each R¹¹ is independently OH, CN, halogen, C₁₋₆ alkyl, or C₁₋₆ alkoxy; and
m is 0, 1, or 2.

12. The compound of claim 9, having a structure according to Formula (VI-3), 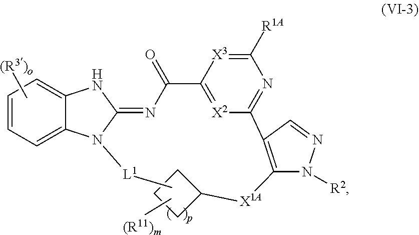
or a pharmaceutically acceptable salt thereof, wherein
each R¹¹ is independently OH, oxo, CN, halogen, C₁₋₆ alkyl, or C₁₋₆ alkoxy;
m is 0, 1, or 2; and
p is 0, 1, 2, or 3.

13. The compound of claim 11 or 12, wherein X^{1A} is a covalent bond or C₁₋₆ alkylene.

14. The compound of claim 9, having a structure according to Formula (VI-4), 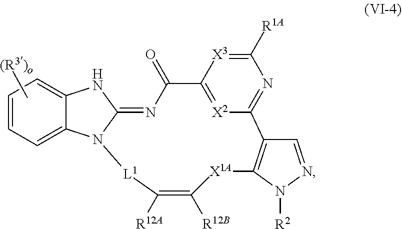
or a pharmaceutically acceptable salt thereof, wherein
X^{1A} is independently a covalent bond or C₁₋₆ alkylene; and
each R^{12A} and R^{12B} is independently H or C₁₋₆ alkyl, or R^{12A} and R^{12B} combine to form a cyclopentene or cyclohexene.

15. The compound of claim 9, having a structure according to Formula (VI-5), 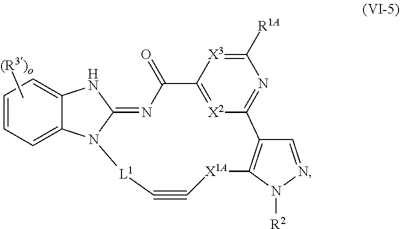
or a pharmaceutically acceptable salt thereof, wherein
X^{1A} is a covalent bond or C₁₋₆ alkylene; and
L¹ is C₁₋₆ alkylene.

16. The compound of any one of claims 9-15, wherein R² is CH₃.

17. The compound of any one of claims 9-16, wherein L¹ is linear or branched C₁₋₆ alkylene, and wherein said alkylene is unsubstituted or comprises a —OH group.

18. The compound of claim 1, having a structure according to Formula (VII), 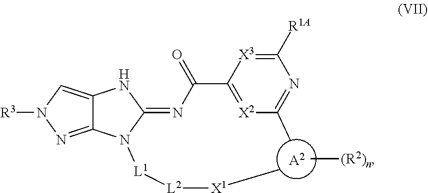
or a pharmaceutically acceptable salt thereof.

19. The compound of claim 1, having a structure according to Formula (VIII), 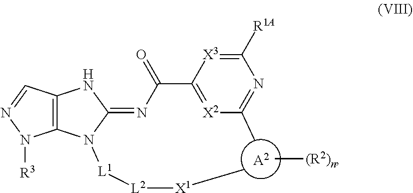
or a pharmaceutically acceptable salt thereof.

20. The compound of any one of claims 1-9 and 18-19, wherein A² is a monocyclic 5-to-6-membered heteroaryl.

21. The compound of claim 20, wherein A² is pyridyl, pyrimidyl, pyrazolyl, thiazolyl, oxazolyl, or imidazolyl, and wherein A² is optionally substituted by a methyl, halogen, or CN.

22. The compound of any one of claims 1-9 and 18-19, wherein A² is phenyl, naphthyl, or a bicyclic 8- to 12-membered heteroaryl.

23. The compound of claim 22, wherein A² is a nitrogen-containing, bicyclic 8- to 12-membered heteroaryl that is indolyl, benzimidazolyl, indazolyl, isoindolyl, pyrrolopyrimidyl, pyrrolopyridinyl, pyrazolopyrimidyl, pyrazolopyridinyl, benzotriazolyl, quinolyl, or isoquinolyl.

24. The compound of any one of claims 1-10 and 18-23, wherein X¹ is X^{1A}.

25. The compound of claim 24, wherein X^{1A} is a covalent bond.

26. The compound of claim 24, wherein X^{1A} is S or NR⁴.

27. The compound of claim 24, wherein X^{1A} is C₁₋₆ alkylene, C₂₋₆ alkenylene, or C₂₋₆ alkynylene.

28. The compound of any one of claims 1-10 and 18-23, wherein X¹ is O.

29. The compound of any one of claims 1 and 8-28, wherein X² and/or X³ is N.

30. The compound of any one of claims 1 and 8-28, wherein each of X² and X³ is CR^{1B}.

31. The compound of claim 30, wherein each of X² and X³ is CH.

32. The compound of any one of claims 1-16 and 18-31, wherein U is a covalent bond, unsubstituted branched C₁₋₆ alkylene, or linear C₁₋₆ alkylene optionally comprising a —OH substituent.

33. The compound of claim 32, wherein L is a covalent bond.

34. The compound of claim 32, wherein L¹ is unsubstituted branched C₁₋₆ alkylene or linear C₁₋₆ alkylene optionally comprising a —OH substituent.

35. The compound of any one of claims 1-10 and 18-32, wherein L² is C₂₋₆ alkenylene or C₂₋₆ alkynylene.

36. The compound of any one of claims 1-10 and 18-32, wherein L² is C₃₋₆ cycloalkylene or 3- to 10-membered heterocyclylene.

37. The compound of any one of claims 1-10 and 18-32, wherein L² is phenylene, or 5- or 6-membered heteroarylene.

38. The compound of any one of claims 1-10 and 18-32, wherein L² is a covalent bond.

39. The compound of any one of claims 1-6, wherein:
X¹ is X^{1A}, wherein X^{1A} is a covalent bond, C₁₋₆ alkylene, C₂₋₆ alkenylene, or C₂₋₆ alkynylene;
L¹ is independently a covalent bond or C₁₋₆ alkylene;
L² is independently a covalent bond, C₂₋₆ alkenylene, C₂₋₆ alkynylene;
C₃₋₆ cycloalkylene, 3- to 10-membered heterocyclylene, phenylene, or 5- or 6-membered heteroarylene; and
wherein at least one of X^{A}, L¹, and L² is a covalent bond.

40. The compound of claim 39, wherein:
one X^{1A} and L¹ is a covalent bond and the other is C₁₋₆ alkylene; and
L² is a covalent bond.

41. The compound of claim 39, wherein each of L¹ and L² is a covalent bond.

42. The compound of claim 39, wherein each of X^{1A} and L² is a covalent bond.

43. The compound of claim 39, wherein each of X^{1A} and L¹ is a covalent bond.

44. The compound of claim 1, wherein said compound has a structure according to Formula (IX), 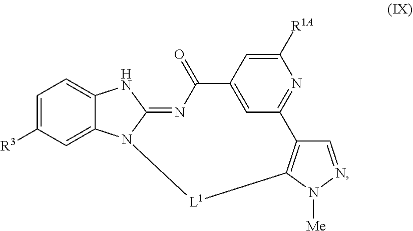
or a pharmaceutically acceptable salt thereof, wherein
L¹ is C₁-C₆ alkylene optionally substituted by 1, 2, or 3 R¹³;
each R¹³ is independently unsubstituted C₁-C₃ alkyl; and
R^{1A} is independently unsubstituted C₁-C₆ alkyl or C₁-C₆ haloalkyl.

45. The compound of claim 1, wherein said compound has a structure according to Formula (X), 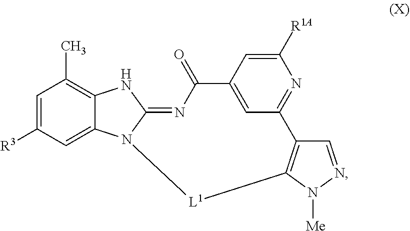
or a pharmaceutically acceptable salt thereof, wherein
L¹ is C₁-C₆ alkylene optionally substituted by 1, 2, or 3 R¹³;
each R¹³ is independently unsubstituted C₁-C₃ alkyl; and
R^{1A} is independently unsubstituted C₁-C₆ alkyl or C₁-C₆ haloalkyl.

46. The compound of claim 1, wherein said compound has a structure according to Formula (XI), 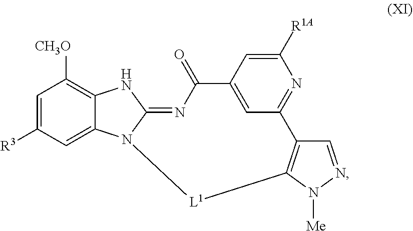
or a pharmaceutically acceptable salt thereof, wherein
L¹ is C₁-C₆ alkylene optionally substituted by 1, 2, or 3 R¹³;
each R¹³ is independently unsubstituted C₁-C₃ alkyl; and
R^{1A} is independently unsubstituted C₁-C₆ alkyl or C₁-C₆ haloalkyl.

47. The compound of any one of claims 44-46, wherein L¹ is selected from the following group of substructures: 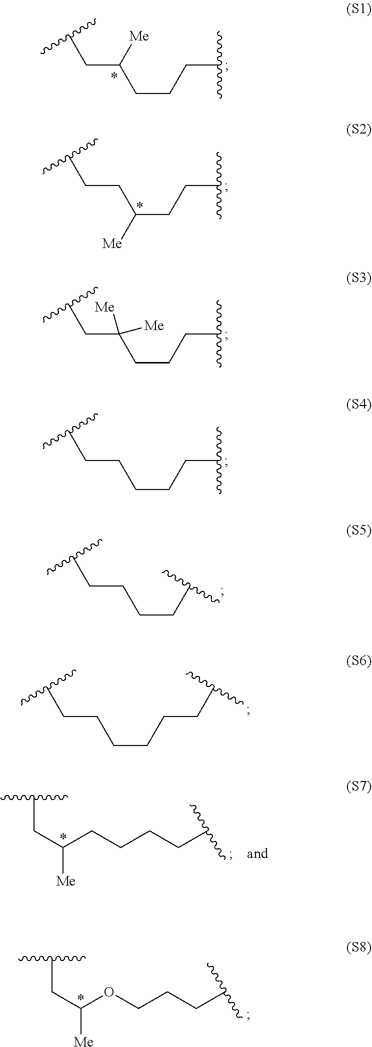
wherein a carbon marked by an asterisk (*) is racemic or has the (R)- or (S)-stereochemistry.

48. The compound of claim 44 or 47, wherein said compound has a structure according to: 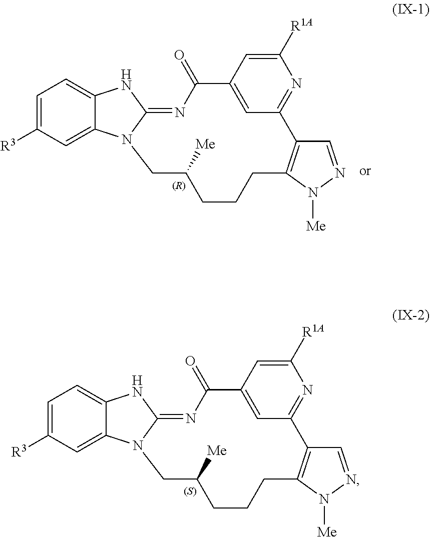
or a pharmaceutically acceptable salt thereof.

49. The compound of claim 44 or 47, wherein said compound has a structure according to: 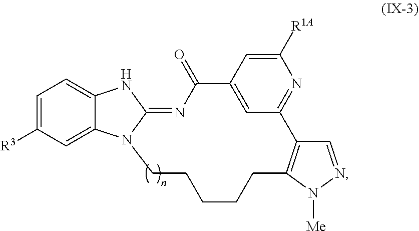
or a pharmaceutically acceptable salt thereof, wherein n is 1, 2, or 3.

50. The compound of claim 44 or 47, wherein said compound has a structure according to: 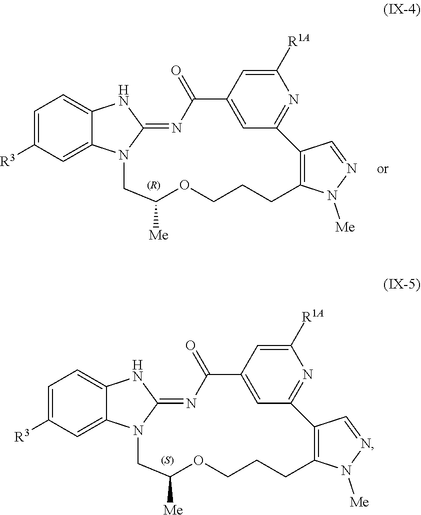
or a pharmaceutically acceptable salt thereof.

51. The compound of claim 1, wherein said compound has a structure according to Formula (XII), 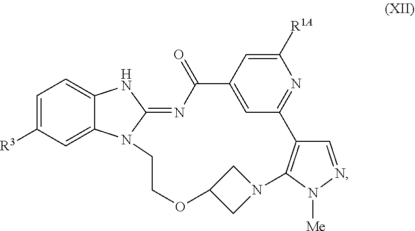
or a pharmaceutically acceptable salt thereof, wherein
R^{1A} is independently unsubstituted C₁-C₆ alkyl or C₁-C₆ haloalkyl.

52. The compound of claim 1, wherein said compound has a structure according to Formula (XIII), 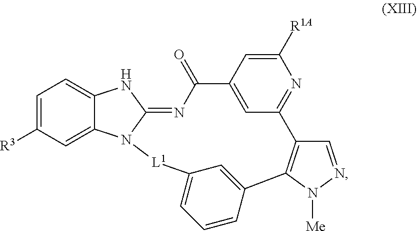
or a pharmaceutically acceptable salt thereof, wherein
L¹ is C₁-C₃ alkylene optionally substituted by 1 or 2 R¹³;
each R¹³ is independently unsubstituted C₁-C₃ alkyl; and
R^{1A} is independently unsubstituted C₁-C₆ alkyl or C₁-C₆ haloalkyl.

53. The compound of claim 52, wherein L is —CH₂— or —CH₂CHCH₃—.

54. The compound of claim 1, wherein said compound has a structure according to Formula (XIV), 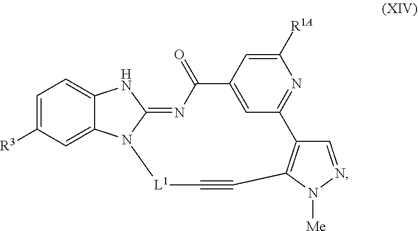
or a pharmaceutically acceptable salt thereof, wherein
L¹ is C₂-C₄ alkylene optionally substituted by 1 or 2 R¹³;
each R¹³ is independently unsubstituted C₁-C₃ alkyl; and
R^{1A} is independently unsubstituted C₁-C₆ alkyl or C₁-C₆ haloalkyl.

55. The compound of claim 54, wherein L¹ is —(CH₂)₃— or —(CH₂)₄—.

56. The compound of any one of claims 44-55, wherein R^{1A} is CH₃, CH₂F, CHF₂, or CF₃.

57. The compound of claim 56, wherein R^{1A} is CH₃.

58. The compound of any one of claims 1 and 44-57, wherein R³ is halogen;
NR⁶R⁷, wherein R⁶ and R⁷, together with the nitrogen atom to which they are attached, form a 5- to 7-membered heterocyclyl;
NR⁶R⁷, wherein each R⁶ and R⁷ is independently C₁-C₆ alkyl;
phenyl;
pyridyl;
C(O)R⁸, wherein R¹ is a 5- to 6-membered nitrogen-containing heterocyclyl;
R¹⁰, wherein R¹⁰ is a 5- to 6-membered nitrogen-containing heterocyclyl;
OR¹⁰, wherein R¹⁰ is a 5- to 6-membered nitrogen-containing heterocyclyl;
CH₂R¹⁰, wherein R¹⁰ is a 5- to 6-membered nitrogen-containing heterocyclyl;
CH₂CH₂R¹⁰, wherein R¹⁰ is a 5- to 6-membered nitrogen-containing heterocyclyl; or
OCH₂CH₂R¹⁰, wherein R¹⁰ is a 5- to 6-membered nitrogen-containing heterocyclyl.

59. The compound of claim 58, wherein R³ is halogen.

60. The compound of claim 58, wherein R³ is NR⁶R⁷, wherein R⁶ and R⁷, together with the nitrogen atom to which they are attached, form a 5- to 6-membered heterocyclyl.

61. The compound of claim 60, wherein R³ is unsubstituted or substituted pyrrolidine, morpholine, piperidine, or piperazine.

62. The compound of claim 58, wherein R³ is C(O)R⁸, wherein R⁸ is unsubstituted or substituted pyrrolidine, morpholine, piperidine, or piperazine.

63. The compound of claim 58, wherein R³ is unsubstituted or substituted phenyl or pyridyl.

64. The compound of claim 58, wherein R³ is R¹⁰, OR¹⁰, CH₂R¹⁰, CH₂CH₂R¹⁰, or OCH₂CH₂R¹⁰, wherein R¹⁰ is unsubstituted or substituted pyrrolidine, morpholine, piperidine, or piperazine.

65. The compound of claim 58, wherein R³ is selected from the group consisting of.

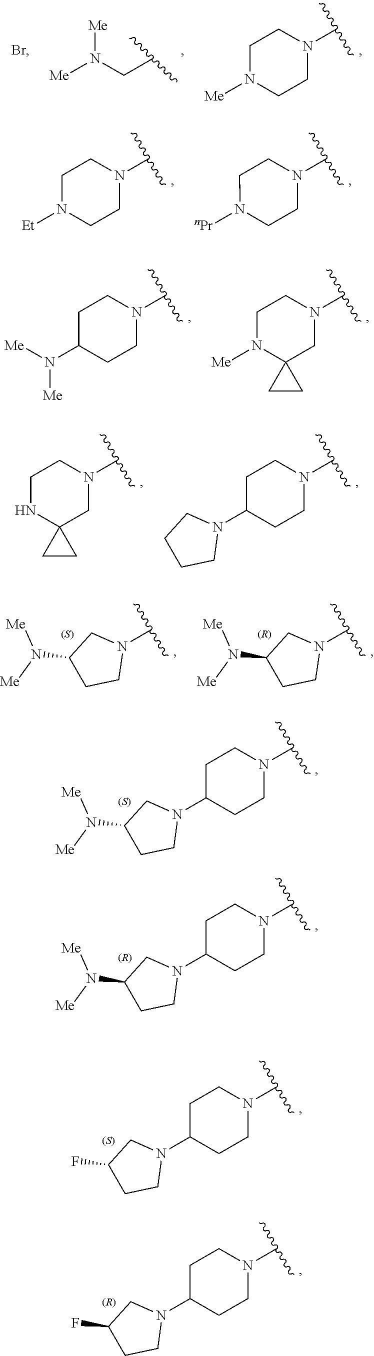

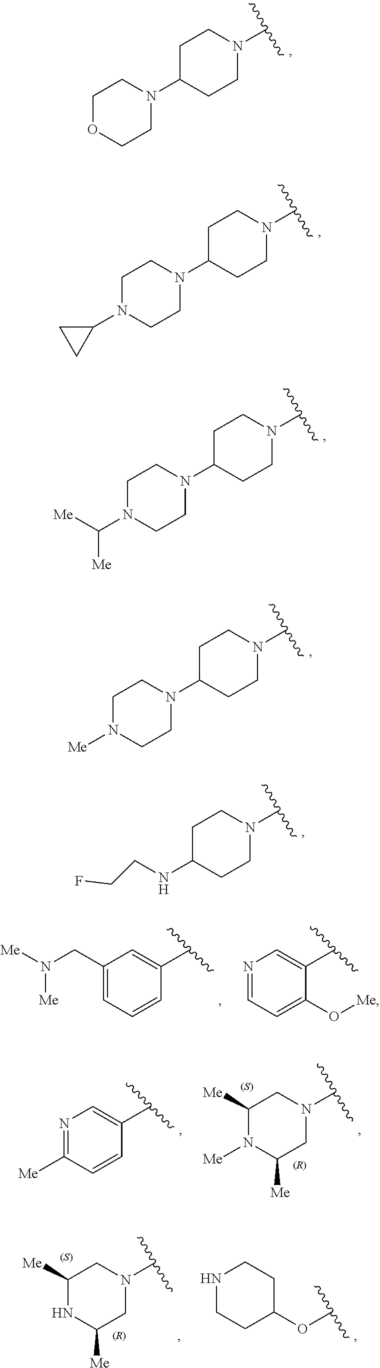

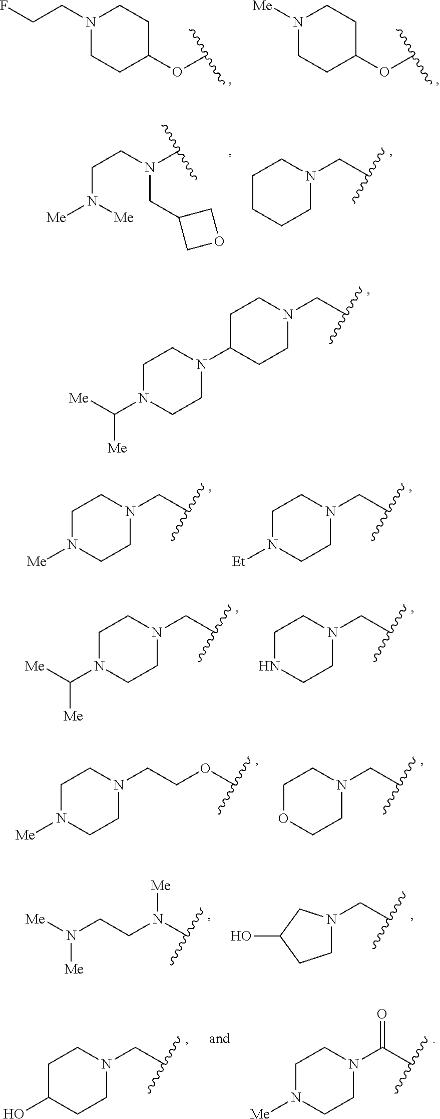

66. The compound of claim 1, selected from the group consisting of Compounds (1)-(71), or a pharmaceutically acceptable salt thereof.

67. A pharmaceutical composition comprising a compound according to any one of claims 1-66, or a pharmaceutically acceptable salt thereof.

68. A method of treating cancer comprising administering to a human in need thereof an effective amount of a compound according to any one of claims 1-66 or a pharmaceutically acceptable salt thereof in a pharmaceutical composition.

69. The method of claim 68, wherein said cancer is a lung cancer.

70. The method of claim 68 or 69, wherein said cancer is non-small cell lung cancer.

71. The method ofany one of claims 68-70, wherein said cancer is an EGFR-driven cancer.

72. The method of any one of claims 68-71, wherein said cancer is characterized by an EGFR mutation.
